SEQUENCE LISTING (139 sequences)

SEQ ID NO: 1 - protein, 377 aa, Artificial Sequence
  Description of Artificial Sequence: Synthetic polypeptide
MEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGVSCKDVYDEAFCWTGGGGSLSGRSDNHGSSGTKQEVTQIPAALSVPEGENLVLNCSFTDSAIYNLQWFRQDPGKGLTSLLYVRPYQREQTSGRLNASLDKSSGRSTLYIAASQPGDSATYLCAVRPGGAGPFFVVFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSGGHHHHHHHH

SEQ ID NO: 2 - protein, 116 aa, Artificial Sequence
  Description of Artificial Sequence: Synthetic polypeptide
MEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSS

SEQ ID NO: 3 - protein, 14 aa, Artificial Sequence
  Description of Artificial Sequence: Synthetic peptide
VSCKDVYDEAFCWT

SEQ ID NO: 4 - protein, 8 aa, Artificial Sequence
  Description of Artificial Sequence: Synthetic peptide
LSGRSDNH

SEQ ID NO: 5 - protein, 208 aa, Artificial Sequence
  Description of Artificial Sequence: Synthetic polypeptide
KQEVTQIPAALSVPEGENLVLNCSFTDSAIYNLQWFRQDPGKGLTSLLYVRPYQREQTSGRLNASLDKSSGRSTLYIAASQPGDSATYLCAVRPGGAGPFFVVFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 6 - protein, 8 aa, Artificial Sequence
  Description of Artificial Sequence: Synthetic 8xHis tag
HHHHHHHH

SEQ ID NO: 7 - protein, 518 aa, Artificial Sequence
  Description of Artificial Sequence: Synthetic polypeptide
MAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFNMATGQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQNPRNHFRCQVQFYGLSENDEWTQDRAKPVTQIVSAEAWGRADGGGLNDIFEAQKIEWHE

SEQ ID NO: 8 - protein, 254 aa, Artificial Sequence
  Description of Artificial Sequence: Synthetic polypeptide
MAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 9 - protein, 242 aa, Artificial Sequence
  Description of Artificial Sequence: Synthetic polypeptide
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSFNMATGQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQNPRNHFRCQVQFYGLSENDEWTQDRAKPVTQIVSAEAWGRAD

SEQ ID NO: 10 - protein, 15 aa, Artificial Sequence
  Description of Artificial Sequence: Synthetic peptide
GLNDIFEAQKIEWHE

SEQ ID NO: 11 - protein, 7 aa, Artificial Sequence
  Description of Artificial Sequence: Synthetic peptide
DVYDEAF

SEQ ID NO: 12 - protein, 16 aa, Artificial Sequence
  Description of Artificial Sequence: Synthetic peptide
GGVSCKDVYDEAFCWT

SEQ ID NO: 13 - protein, 16 aa, Artificial Sequence
  Description of Artificial Sequence: Synthetic peptide
GGESCQSVYDSSFCYD

SEQ ID NO: 14 - protein, 16 aa, Artificial Sequence
  Description of Artificial Sequence: Synthetic peptide
GGNACEMTYDHTFCDP

SEQ ID NO: 15 - protein, 16 aa, Artificial Sequence
  Description of Artificial Sequence: Synthetic peptide
GGRICEEVYDWIFCES

SEQ ID NO: 16 - protein, 16 aa, Artificial Sequence
  Description of Artificial Sequence: Synthetic peptide
GGRRCVDVYDNAFCLI

SEQ ID NO: 17 - protein, 16 aa, Artificial Sequence
  Description of Artificial Sequence: Synthetic peptide
GGTSCAQIYDFEFCYS

SEQ ID NO: 18 - protein, 16 aa, Artificial Sequence
  Description of Artificial Sequence: Synthetic peptide
GGSLCSLVYDQDFCES

SEQ ID NO: 19 - protein, 16 aa, Artificial Sequence
  Description of Artificial Sequence: Synthetic peptide
GGNSCSLVYDKAFCLF

SEQ ID NO: 20 - protein, 16 aa, Artificial Sequence
  Description of Artificial Sequence: Synthetic peptide
GGNQCWEVYDQEFCSL

SEQ ID NO: 21 - protein, 16 aa, Artificial Sequence
  Description of Artificial Sequence: Synthetic peptide
GGSACSRIYDFAFCHT

SEQ ID NO: 22 - protein, 16 aa, Artificial Sequence
  Description of Artificial Sequence: Synthetic peptide
GGTFCYFDHGLVNCQW

SEQ ID NO: 23 - protein, 16 aa, Artificial Sequence
  Description of Artificial Sequence: Synthetic peptide
GGHCFVSPASGEWWCV

SEQ ID NO: 24 - protein, 16 aa, Artificial Sequence
  Description of Artificial Sequence: Synthetic peptide
GGCSWIFDGLRYFSKC

SEQ ID NO: 25 - protein, 12 aa, Artificial Sequence
  Description of Artificial Sequence: Synthetic peptide
VRTWFEKFPELV

SEQ ID NO: 26 - protein, 12 aa, Artificial Sequence
  Description of Artificial Sequence: Synthetic peptide
LVWGCIWDDMCS

SEQ ID NO: 27 - protein, 12 aa, Artificial Sequence
  Description of Artificial Sequence: Synthetic peptide
WHWEPSMVWGML

SEQ ID NO: 28 - protein, 16 aa, Artificial Sequence
  Description of Artificial Sequence: Synthetic peptide
GGGCFVSPATGFTWCV

SEQ ID NO: 29 - protein, 16 aa, Artificial Sequence
  Description of Artificial Sequence: Synthetic peptide
GGDCQPDSVWSYWYCR

SEQ ID NO: 30 - protein, 16 aa, Artificial Sequence
  Description of Artificial Sequence: Synthetic peptide
GGCTFVDWWVLGSPYC

SEQ ID NO: 31 - protein, 16 aa, Artificial Sequence
  Description of Artificial Sequence: Synthetic peptide
GGCLMNDYYYLWGGHC

SEQ ID NO: 32 - protein, 16 aa, Artificial Sequence
  Description of Artificial Sequence: Synthetic peptide
GGASCKDVYDEAFCWT

SEQ ID NO: 33 - protein, 16 aa, Artificial Sequence
  Description of Artificial Sequence: Synthetic peptide
GGVACKDVYDEAFCWT

SEQ ID NO: 34 - protein, 16 aa, Artificial Sequence
  Description of Artificial Sequence: Synthetic peptide
GGVSAKDVYDEAFCWT

SEQ ID NO: 35 - protein, 16 aa, Artificial Sequence
  Description of Artificial Sequence: Synthetic peptide
GGVSCADVYDEAFCWT

SEQ ID NO: 36 - protein, 16 aa, Artificial Sequence
  Description of Artificial Sequence: Synthetic peptide
GGVSCKAVYDEAFCWT

SEQ ID NO: 37 - protein, 16 aa, Artificial Sequence
  Description of Artificial Sequence: Synthetic peptide
GGVSCKDAYDEAFCWT

SEQ ID NO: 38 - protein, 16 aa, Artificial Sequence
  Description of Artificial Sequence: Synthetic peptide
GGVSCKDVADEAFCWT

SEQ ID NO: 39 - protein, 16 aa, Artificial Sequence
  Description of Artificial Sequence: Synthetic peptide
GGVSCKDVYAEAFCWT

SEQ ID NO: 40 - protein, 16 aa, Artificial Sequence
  Description of Artificial Sequence: Synthetic peptide
GGVSCKDVYDAAFCWT

SEQ ID NO: 41 - protein, 16 aa, Artificial Sequence
  Description of Artificial Sequence: Synthetic peptide
GGVSCKDVYDEAACWT

SEQ ID NO: 42 - protein, 16 aa, Artificial Sequence
  Description of Artificial Sequence: Synthetic peptide
GGVSCKDVYDEAFAWT

SEQ ID NO: 43 - protein, 16 aa, Artificial Sequence
  Description of Artificial Sequence: Synthetic peptide
GGVSCKDVYDEAFCAT

SEQ ID NO: 44 - protein, 16 aa, Artificial Sequence
  Description of Artificial Sequence: Synthetic peptide
GGVSCKDVYDEAFCWA

SEQ ID NO: 45 - protein, 9 aa, Artificial Sequence
  Description of Artificial Sequence: Synthetic peptide
EVDPIGHLY

SEQ ID NO: 46 - protein, 9 aa, Artificial Sequence
  Description of Artificial Sequence: Synthetic peptide
ESDPIVAQY

SEQ ID NO: 47 - protein, 16 aa, Artificial Sequence
  Description of Artificial Sequence: Synthetic peptide
GGASCAASASAAACAS

SEQ ID NO: 48 - protein, 9 aa, Artificial Sequence
  Description of Artificial Sequence: Synthetic peptide
  MISC_FEATURE (1)..9): This sequence may encompass 1-3 "Gly Gly Ser" repeating units
GGSGGSGGS

SEQ ID NO: 49 - protein, 5 aa, Artificial Sequence
  Description of Artificial Sequence: Synthetic peptide
  See specification as filed for detailed description of substitutions and preferred embodiments
GSGGS

SEQ ID NO: 50 - protein, 4 aa, Artificial Sequence
  Description of Artificial Sequence: Synthetic peptide
  See specification as filed for detailed description of substitutions and preferred embodiments
GGGS

SEQ ID NO: 51 - protein, 5 aa, Artificial Sequence
  Description of Artificial Sequence: Synthetic peptide
  See specification as filed for detailed description of substitutions and preferred embodiments
GGGGS

SEQ ID NO: 52 - protein, 6 aa, Artificial Sequence
  Description of Artificial Sequence: Synthetic peptide
  See specification as filed for detailed description of substitutions and preferred embodiments
GSSGGS

SEQ ID NO: 53 - protein, 18 aa, Artificial Sequence
  Description of Artificial Sequence: Synthetic peptide
GGGGSLSGRSDNHGSSGT

SEQ ID NO: 54 - protein, 26 aa, Artificial Sequence
  Description of Artificial Sequence: Synthetic peptide
GGGGSSGGSGGSGLSGRSDNHGSSGT

SEQ ID NO: 55 - protein, 8 aa, Artificial Sequence
  Description of Artificial Sequence: Synthetic peptide
ASGRSDNH

SEQ ID NO: 56 - protein, 8 aa, Artificial Sequence
  Description of Artificial Sequence: Synthetic peptide
LAGRSDNH

SEQ ID NO: 57 - protein, 13 aa, Artificial Sequence
  Description of Artificial Sequence: Synthetic peptide
ISSGLASGRSDNH

SEQ ID NO: 58 - protein, 13 aa, Artificial Sequence
  Description of Artificial Sequence: Synthetic peptide
ISSGLLAGRSDNH

SEQ ID NO: 59 - protein, 13 aa, Artificial Sequence
  Description of Artificial Sequence: Synthetic peptide
ISSGLLSGRSDNP

SEQ ID NO: 60 - protein, 13 aa, Artificial Sequence
  Description of Artificial Sequence: Synthetic peptide
ISSGLLSGRSDNH

SEQ ID NO: 61 - protein, 16 aa, Artificial Sequence
  Description of Artificial Sequence: Synthetic peptide
LSGRSDNHSPLGLAGS

SEQ ID NO: 62 - protein, 16 aa, Artificial Sequence
  Description of Artificial Sequence: Synthetic peptide
SPLGLAGSLSGRSDNH

SEQ ID NO: 63 - protein, 12 aa, Artificial Sequence
  Description of Artificial Sequence: Synthetic peptide
SPLGLSGRSDNH

SEQ ID NO: 64 - protein, 16 aa, Artificial Sequence
  Description of Artificial Sequence: Synthetic peptide
LAGRSDNHSPLGLAGS

SEQ ID NO: 65 - protein, 16 aa, Artificial Sequence
  Description of Artificial Sequence: Synthetic peptide
LSGRSDNHVPLSLKMG

SEQ ID NO: 66 - protein, 16 aa, Artificial Sequence
  Description of Artificial Sequence: Synthetic peptide
LSGRSDNHVPLSLSMG

SEQ ID NO: 67 - protein, 11 aa, Artificial Sequence
  Description of Artificial Sequence: Synthetic peptide
SSGGGGSGGGS

SEQ ID NO: 68 - protein, 26 aa, Artificial Sequence
  Description of Artificial Sequence: Synthetic peptide
GSSGGSGGSGGSGLSGRSDNHGSSGT

SEQ ID NO: 69 - protein, 26 aa, Artificial Sequence
  Description of Artificial Sequence: Synthetic peptide
GSSGGSGGSGGSGGGSGGGSGGSSGT

SEQ ID NO: 70 - protein, 18 aa, Artificial Sequence
  Description of Artificial Sequence: Synthetic peptide
GGGGSGGSGGGSGGSSGT

SEQ ID NO: 71 - protein, 9 aa, Artificial Sequence
  Description of Artificial Sequence: Synthetic peptide
GGGGSGGGS

SEQ ID NO: 72 - protein, 115 aa, Artificial Sequence
  Description of Artificial Sequence: Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSS

SEQ ID NO: 73 - protein, 219 aa, Artificial Sequence
  Description of Artificial Sequence: Synthetic polypeptide
MKQEVTQIPAALSVPEGENLVLNCSFTDSAIYNLQWFRQDPGKGLTSLLYVRPYQREQTSGRLNASLDKSSGRSTLYIAASQPGDSATYLCAVRPGGAGPFFVVFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSGGHHHHHHHH

SEQ ID NO: 74 - protein, 260 aa, Artificial Sequence
  Description of Artificial Sequence: Synthetic polypeptide
MKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFNMATGQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQNPRNHFRCQVQFYGLSENDEWTQDRAKPVTQIVSAEAWGRADGGGLNDIFEAQKIEWHE

SEQ ID NO: 75 - protein, 219 aa, Artificial Sequence
  Description of Artificial Sequence: Synthetic polypeptide
MKQEVTQIPAALSVPEGENLVLNCSFTDSAIYNLQWFRQDPGKGLTSLLLVRPYQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSGGHHHHHHHH

SEQ ID NO: 76 - protein, 208 aa, Artificial Sequence
  Description of Artificial Sequence: Synthetic polypeptide
KQEVTQIPAALSVPEGENLVLNCSFTDSAIYNLQWFRQDPGKGLTSLLLVRPYQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 77 - protein, 260 aa, Artificial Sequence
  Description of Artificial Sequence: Synthetic polypeptide
MKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPNMADEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADGGGLNDIFEAQKIEWHE

SEQ ID NO: 78 - protein, 242 aa, Artificial Sequence
  Description of Artificial Sequence: Synthetic polypeptide
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSPNMADEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 79 - protein, 219 aa, Artificial Sequence
  Description of Artificial Sequence: Synthetic polypeptide
MKQEVTQIPAALSVPEGENLVLNCSFTDSAIYNLQWFRQDPGKGLTSLLLIQSSQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSGGHHHHHHHH

SEQ ID NO: 80 - protein, 208 aa, Artificial Sequence
  Description of Artificial Sequence: Synthetic polypeptide
KQEVTQIPAALSVPEGENLVLNCSFTDSAIYNLQWFRQDPGKGLTSLLLIQSSQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 81 - protein, 260 aa, Artificial Sequence
  Description of Artificial Sequence: Synthetic polypeptide
MKAGVTQTPRYLIKTRGQQVTLSCSPISGHRSVSWYQQTPGQGLQFLFEYTDMTLRNKGNFPGRFSGRQFSNSRSEMNVSTLELGDSALYLCASSPNMADEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADGGGLNDIFEAQKIEWHE

SEQ ID NO: 82 - protein, 242 aa, Artificial Sequence
  Description of Artificial Sequence: Synthetic polypeptide
KAGVTQTPRYLIKTRGQQVTLSCSPISGHRSVSWYQQTPGQGLQFLFEYTDMTLRNKGNFPGRFSGRQFSNSRSEMNVSTLELGDSALYLCASSPNMADEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 83 - protein, 260 aa, Artificial Sequence
  Description of Artificial Sequence: Synthetic polypeptide
MKAGVTQTPRYLIKTRGQQVTLSCSPISGHRSVSWYQQTPGQGLQFLFEYFDMLLRNKGNFPGRFSGRQFSNSRSEMNVSTLELGDSALYLCASSPNMADEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADGGGLNDIFEAQKIEWHE

SEQ ID NO: 84 - protein, 242 aa, Artificial Sequence
  Description of Artificial Sequence: Synthetic polypeptide
KAGVTQTPRYLIKTRGQQVTLSCSPISGHRSVSWYQQTPGQGLQFLFEYFDMLLRNKGNFPGRFSGRQFSNSRSEMNVSTLELGDSALYLCASSPNMADEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 85 - protein, 218 aa, Artificial Sequence
  Description of Artificial Sequence: Synthetic polypeptide
KQEVTQIPAALSVPEGENLVLNCSFTDSAIYNLQWFRQDPGKGLTSLLYVRPYQREQTSGRLNASLDKSSGRSTLYIAASQPGDSATYLCAVRPGGAGPFFVVFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSGGHHHHHHHH

SEQ ID NO: 86 - protein, 253 aa, Artificial Sequence
  Description of Artificial Sequence: Synthetic polypeptide
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 87 - protein, 295 aa, Artificial Sequence
  Description of Artificial Sequence: Synthetic polypeptide
MEVDPIGHLYGSSGGSGGSGGSGLSGRSDNHGSSGTKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFNMATGQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQNPRNHFRCQVQFYGLSENDEWTQDRAKPVTQIVSAEAWGRADGGGLNDIFEAQKIEWHE

SEQ ID NO: 88 - protein, 295 aa, Artificial Sequence
  Description of Artificial Sequence: Synthetic polypeptide
MEVDPIGHLYGSSGGSGGSGGSGGGSGGGSGGSSGTKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFNMATGQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQNPRNHFRCQVQFYGLSENDEWTQDRAKPVTQIVSAEAWGRADGGGLNDIFEAQKIEWHE

SEQ ID NO: 89 - protein, 294 aa, Artificial Sequence
  Description of Artificial Sequence: Synthetic polypeptide
MGGVSCKDVYDEAFCWTGGGGSLSGRSDNHGSSGTKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFNMATGQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQNPRNHFRCQVQFYGLSENDEWTQDRAKPVTQIVSAEAWGRADGGGLNDIFEAQKIEWHE

SEQ ID NO: 90 - protein, 302 aa, Artificial Sequence
  Description of Artificial Sequence: Synthetic polypeptide
MGGVSCKDVYDEAFCWTGGGGSSGGSGGSGLSGRSDNHGSSGTKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFNMATGQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQNPRNHFRCQVQFYGLSENDEWTQDRAKPVTQIVSAEAWGRADGGGLNDIFEAQKIEWHE

SEQ ID NO: 91 - protein, 253 aa, Artificial Sequence
  Description of Artificial Sequence: Synthetic polypeptide
MGGVSCKDVYDEAFCWTGGGGSLSGRSDNHGSSGTKQEVTQIPAALSVPEGENLVLNCSFTDSAIYNLQWFRQDPGKGLTSLLYVRPYQREQTSGRLNASLDKSSGRSTLYIAASQPGDSATYLCAVRPGGAGPFFVVFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSGGHHHHHHHH

SEQ ID NO: 92 - protein, 261 aa, Artificial Sequence
  Description of Artificial Sequence: Synthetic polypeptide
MGGVSCKDVYDEAFCWTGGGGSSGGSGGSGLSGRSDNHGSSGTKQEVTQIPAALSVPEGENLVLNCSFTDSAIYNLQWFRQDPGKGLTSLLYVRPYQREQTSGRLNASLDKSSGRSTLYIAASQPGDSATYLCAVRPGGAGPFFVVFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSGGHHHHHHHH

SEQ ID NO: 93 - protein, 518 aa, Artificial Sequence
  Description of Artificial Sequence: Synthetic polypeptide
MKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFNMATGQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQNPRNHFRCQVQFYGLSENDEWTQDRAKPVTQIVSAEAWGRADGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGLNDIFEAQKIEWHE

SEQ ID NO: 94 - protein, 560 aa, Artificial Sequence
  Description of Artificial Sequence: Synthetic polypeptide
MGGVSCKDVYDEAFCWTGGGGSSGGSGGSGLSGRSDNHGSSGTKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFNMATGQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQNPRNHFRCQVQFYGLSENDEWTQDRAKPVTQIVSAEAWGRADGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGLNDIFEAQKIEWHE

SEQ ID NO: 95 - protein, 477 aa, Artificial Sequence
  Description of Artificial Sequence: Synthetic polypeptide
MAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSKQEVTQIPAALSVPEGENLVLNCSFTDSAIYNLQWFRQDPGKGLTSLLYVRPYQREQTSGRLNASLDKSSGRSTLYIAASQPGDSATYLCAVRPGGAGPFFVVFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSGGHHHHHHHH

SEQ ID NO: 96 - protein, 252 aa, Artificial Sequence
  Description of Artificial Sequence: Synthetic polypeptide
MGGVSCKDVYDEAFCWTGGGGSGGSGGGSGGSSGTKQEVTQIPAALSVPEGENLVLNCSFTDSAIYNLQWFRQDPGKGLTSLLYVRPYQREQTSGRLNASLDKSSGRSTLYIAASQPGDSATYLCAVRPGGAGPFFVVFGKGTKLSVIPNIQNPDPAVYQLRDSKSSDKSVCLFTDFDSQTNVSQSKDSDVYITDKCVLDMRSMDFKSNSAVAWSNKSDFACANAFNNSHPEDTFFPSPESSGGHHHHHHHH

SEQ ID NO: 97 - protein, 377 aa, Artificial Sequence
  Description of Artificial Sequence: Synthetic polypeptide
MEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGVSCKDVYDEAFCWTGGGGSGGSGGGSGGSSGTKQEVTQIPAALSVPEGENLVLNCSFTDSAIYNLQWFRQDPGKGLTSLLYVRPYQREQTSGRLNASLDKSSGRSTLYIAASQPGDSATYLCAVRPGGAGPFFVVFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSGGHHHHHHHH

SEQ ID NO: 98 - protein, 377 aa, Artificial Sequence
  Description of Artificial Sequence: Synthetic polypeptide
MEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGASCAASASAAACASGGGGSLSGRSDNHGSSGTKQEVTQIPAALSVPEGENLVLNCSFTDSAIYNLQWFRQDPGKGLTSLLYVRPYQREQTSGRLNASLDKSSGRSTLYIAASQPGDSATYLCAVRPGGAGPFFVVFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSGGHHHHHHHH

SEQ ID NO: 99 - protein, 6 aa, Artificial Sequence
  Description of Artificial Sequence: Synthetic 6xHis tag
HHHHHH

SEQ ID NO: 100 - protein, 21 aa, Artificial Sequence
  Description of Artificial Sequence: Synthetic peptide
GGESCQSVYDSSFCYDGGGGS

SEQ ID NO: 101 - protein, 21 aa, Artificial Sequence
  Description of Artificial Sequence: Synthetic peptide
GGNACEMTYDHTFCDPGGGGS

SEQ ID NO: 102 - protein, 21 aa, Artificial Sequence
  Description of Artificial Sequence: Synthetic peptide
GGRICEEVYDWIFCESGGGGS

SEQ ID NO: 103 - protein, 21 aa, Artificial Sequence
  Description of Artificial Sequence: Synthetic peptide
GGRRCVDVYDNAFCLIGGGGS

SEQ ID NO: 104 - protein, 21 aa, Artificial Sequence
  Description of Artificial Sequence: Synthetic peptide
GGVSCKDVYDEAFCWTGGGGS

SEQ ID NO: 105 - protein, 21 aa, Artificial Sequence
  Description of Artificial Sequence: Synthetic peptide
GGTSCAQIYDFEFCYSGGGGS

SEQ ID NO: 106 - protein, 21 aa, Artificial Sequence
  Description of Artificial Sequence: Synthetic peptide
GGSLCSLVYDQDFCESGGGGS

SEQ ID NO: 107 - protein, 21 aa, Artificial Sequence
  Description of Artificial Sequence: Synthetic peptide
GGNSCSLVYDKAFCLFGGGGS

SEQ ID NO: 108 - protein, 21 aa, Artificial Sequence
  Description of Artificial Sequence: Synthetic peptide
GGNQCWEVYDQEFCSLGGGGS

SEQ ID NO: 109 - protein, 21 aa, Artificial Sequence
  Description of Artificial Sequence: Synthetic peptide
GGSACSRIYDFAFCHTGGGGS

SEQ ID NO: 110 - protein, 21 aa, Artificial Sequence
  Description of Artificial Sequence: Synthetic peptide
GGTFCYFDHGLVNCQWGGGGS

SEQ ID NO: 111 - protein, 21 aa, Artificial Sequence
  Description of Artificial Sequence: Synthetic peptide
GGHCFVSPASGEWWCVGGGGS

SEQ ID NO: 112 - protein, 21 aa, Artificial Sequence
  Description of Artificial Sequence: Synthetic peptide
GGCSWIFDGLRYFSKCGGGGS

SEQ ID NO: 113 - protein, 17 aa, Artificial Sequence
  Description of Artificial Sequence: Synthetic peptide
VRTWFEKFPELVGGGGS

SEQ ID NO: 114 - protein, 17 aa, Artificial Sequence
  Description of Artificial Sequence: Synthetic peptide
LVWGCIWDDMCSGGGGS

SEQ ID NO: 115 - protein, 17 aa, Artificial Sequence
  Description of Artificial Sequence: Synthetic peptide
WHWEPSMVWGMLGGGGS

SEQ ID NO: 116 - protein, 21 aa, Artificial Sequence
  Description of Artificial Sequence: Synthetic peptide
GGGCFVSPATGFTWCVGGGGS

SEQ ID NO: 117 - protein, 21 aa, Artificial Sequence
  Description of Artificial Sequence: Synthetic peptide
GGDCQPDSVWSYWYCRGGGGS

SEQ ID NO: 118 - protein, 21 aa, Artificial Sequence
  Description of Artificial Sequence: Synthetic peptide
GGCTFVDWWVLGSPYCGGGGS

SEQ ID NO: 119 - protein, 21 aa, Artificial Sequence
  Description of Artificial Sequence: Synthetic peptide
GGCLMNDYYYLWGGHCGGGGS

SEQ ID NO: 120 - protein, 10 aa, Artificial Sequence
  Description of Artificial Sequence: Synthetic peptide
  MOD_RES (2)..(4): Any amino acid
  MOD_RES (7)..(8): Any amino acid
CXXXYDXXFC

SEQ ID NO: 121 - protein, 21 aa, Artificial Sequence
  Description of Artificial Sequence: Synthetic peptide
GGASCKDVYDEAFCWTGGGGS

SEQ ID NO: 122 - protein, 21 aa, Artificial Sequence
  Description of Artificial Sequence: Synthetic peptide
GGVACKDVYDEAFCWTGGGGS

SEQ ID NO: 123 - protein, 21 aa, Artificial Sequence
  Description of Artificial Sequence: Synthetic peptide
GGVSAKDVYDEAFCWTGGGGS

SEQ ID NO: 124 - protein, 21 aa, Artificial Sequence
  Description of Artificial Sequence: Synthetic peptide
GGVSCADVYDEAFCWTGGGGS

SEQ ID NO: 125 - protein, 21 aa, Artificial Sequence
  Description of Artificial Sequence: Synthetic peptide
GGVSCKAVYDEAFCWTGGGGS

SEQ ID NO: 126 - protein, 21 aa, Artificial Sequence
  Description of Artificial Sequence: Synthetic peptide
GGVSCKDAYDEAFCWTGGGGS

SEQ ID NO: 127 - protein, 21 aa, Artificial Sequence
  Description of Artificial Sequence: Synthetic peptide
GGVSCKDVADEAFCWTGGGGS

SEQ ID NO: 128 - protein, 21 aa, Artificial Sequence
  Description of Artificial Sequence: Synthetic peptide
GGVSCKDVYAEAFCWTGGGGS

SEQ ID NO: 129 - protein, 21 aa, Artificial Sequence
  Description of Artificial Sequence: Synthetic peptide
GGVSCKDVYDAAFCWTGGGGS

SEQ ID NO: 130 - protein, 21 aa, Artificial Sequence
  Description of Artificial Sequence: Synthetic peptide
GGVSCKDVYDEAACWTGGGGS

SEQ ID NO: 131 - protein, 21 aa, Artificial Sequence
  Description of Artificial Sequence: Synthetic peptide
GGVSCKDVYDEAFAWTGGGGS

SEQ ID NO: 132 - protein, 21 aa, Artificial Sequence
  Description of Artificial Sequence: Synthetic peptide
GGVSCKDVYDEAFCATGGGGS

SEQ ID NO: 133 - protein, 21 aa, Artificial Sequence
  Description of Artificial Sequence: Synthetic peptide
GGVSCKDVYDEAFCWAGGGGS

SEQ ID NO: 134 - protein, 14 aa, Artificial Sequence
  Description of Artificial Sequence: Synthetic peptide
EVDPIGHLYGGGGS

SEQ ID NO: 135 - protein, 14 aa, Artificial Sequence
  Description of Artificial Sequence: Synthetic peptide
ESDPIVAQYGGGGS

SEQ ID NO: 136 - protein, 14 aa, Artificial Sequence
  Description of Artificial Sequence: Synthetic peptide
ASCAASASAAACAS

SEQ ID NO: 137 - DNA, 12 bp, Artificial Sequence
  Description of Artificial Sequence: Synthetic oligonucleotide
cgcttggcatta

SEQ ID NO: 138 - protein, 4 aa, Artificial Sequence
  Description of Artificial Sequence: Synthetic peptide
RLAL

SEQ ID NO: 139 - protein, 5 aa, Artificial Sequence
  Description of Artificial Sequence: Synthetic peptide
GGGGS

(19) United States
(12) Patent Application Publication
(10) Pub. No.: US 2023/0220105 A1
(43) Pub. Date: Jul. 13, 2023

(54) TUMOR ACTIVATED T CELL ENGAGERS AND METHODS OF USE THEREOF

(71) Applicant: Janux Therapeutics, Inc., La Jolla, CA (US)

(72) Inventors: David CAMPBELL, La Jolla, CA (US); Ramesh BHATT, La Jolla, CA (US); Thomas R. DIRAIMONDO, La Jolla, CA (US)

(21) Appl. No.: 17/616,278

(22) PCT Filed: Jun. 5, 2020
(86) PCT No.: PCT/US2020/036489

§ 371 Date: Dec. 3, 2021

Related U.S. Application Data
(60) Provisional application No. 62/978,662, filed on Feb. 19, 2020.
(60) Provisional application No. 62/858,254, filed on Jun. 6, 2019.

(51) Int. Cl. C07K 16/30; C07K 16/32; A61K 47/68

(52) U.S. Cl. CPC C07K 16/30; A61K 47/68; C07K 16/32; C07K 2317/24; C07K 2317/565; C07K 2317/622

(57) ABSTRACT

Provided herein are modified T cell engagers, pharmaceutical compositions thereof, as well as nucleic acids, and methods for making and discovering the same. The modified T cell engagers described herein are modified with a peptide and a half-life extending molecule.

### CROSS-REFERENCE

This application claims the benefit of U.S. Provisional Application No. 62/858,254, filed Jun. 6, 2019, and U.S. Provisional Application No. 62/978,662, filed Feb. 19, 2020, which applications are incorporated herein by reference.

### SEQUENCE LISTING

The instant application contains a Sequence Listing which has been submitted electronically in ASCII format and is hereby incorporated by reference in its entirety. Said ASCII copy, created on Jun. 4, 2020, is named 52426-711_601_SL.txt and is 116,335 bytes in size.

### BACKGROUND

Protein-based therapies, such as modified T-cell engagers, have proven effective as treatments for a variety of diseases. As with any therapeutic class, there is a need to improve toxicity and side effects of such treatments, along with improving the half-life of the therapeutic molecules.

### SUMMARY

Modified T-cell engagers can be used for selective destruction of an individual cell or cell type such as cancer cells of a tumor. Such modified T-cell engagers induce an immune response against the tumor to clear the tumor. However, current therapies using modified T-cell engagers can be toxic and inefficacious. Further, such modified T-cell engagers can have poor pharmacokinetic properties (PK). Provided herein are modified T-cell engagers that reduce toxicity in healthy tissue and thus improving safety while having improved PK properties and efficacy in eliminating the tumor. In some embodiments, the modified T-cell engagers described herein are linked to a peptide that blocks interactions of the T-cell engager with its target in healthy tissue thereby reducing target mediated drug disposition (TMDD). The modified T-cell engagers as described herein are also linked to half-life extending molecule, such as single-domain antibody, which improves the PK profile of the modified T-cell engager as compared to an unmodified T-cell engager.

Disclosed herein, in certain embodiments, are polypeptide complexes comprising a structural arrangement according to the configuration shown in FIG. 70Y, wherein the polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen at an N-terminus of the soluble TCR with a linking moiety that is a substrate for a tumor specific protease, and the peptide is further linked to a half-life extending molecule; and a single chain variable fragment (scFv) that is linked to the soluble TCR wherein the scFv comprises a light chain variable domain and heavy chain variable domain and the scFv binds to an effector cell antigen. In some instances, the peptide is linked to an N-terminus of the alpha TCR polypeptide. In some instances, the peptide is linked to an N-terminus of the alpha TCR polypeptide and the beta TCR polypeptide is linked to a C-terminus of the heavy chain variable domain. In some instances, the peptide is linked to an N-terminus of the alpha TCR polypeptide and the beta TCR polypeptide is linked to a C-terminus of the light chain variable domain. In some instances, the peptide is linked to an N-terminus of the beta TCR polypeptide. In some instances, the peptide is linked to an N-terminus of the beta TCR polypeptide and the alpha TCR polypeptide is linked to a C-terminus of the heavy chain variable domain. In some instances, the peptide is linked to an N-terminus of the beta TCR polypeptide and the alpha TCR polypeptide is linked to a C-terminus of the light chain variable domain. In some instances, the tumor cell antigen comprises MAGEA3. In some instances, the alpha TCR polypeptide comprises a TCR alpha extracellular domain and the beta TCR polypeptide comprises a TCR beta extracellular domain. In some instances, the alpha TCR polypeptide comprises an amino acid sequence that has at least 85% sequence identity to SEQ ID NOs: 5, 73, 75, 76, 79, 80, 85, or 91. In some instances, the beta TCR polypeptide comprises an amino acid sequence that has at least 85% sequence identity to SEQ ID NOs: 9, 74, 77, 78, 81, 82, 83, or 84. In some instances, the peptide has less than 70% sequence identity to an amino acid sequence of the tumor cell antigen. In some instances, the peptide has less than 70% sequence identity to an amino acid sequence of the MAGEA3. In some instances, the peptide is bound to the soluble TCR through ionic interactions, electrostatic interactions, hydrophobic interactions, Pi-stacking interactions, and H-bonding interactions. In some instances, the peptide is a cyclic peptide. In some instances, the peptide is at least 10 amino acids in length. In some instances, the peptide is no more than 40 amino acids in length. In some instances, the peptide comprises an amino acid sequence of at least 10 amino acids in length and no more than 20 amino acids in length. In some instances, the peptide comprises an amino acid sequence according to SEQ ID NOs: 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44. In some instances, the linking moiety comprises a urokinase cleavable amino acid sequence, a matriptase cleavable amino acid sequence, matrix metalloprotease cleavable amino acid sequence, or a legumain cleavable amino acid sequence. In some instances, the linking moiety has a formula comprising (G₂S)ₙ, (GS)ₙ, (GSGGS)ₙ (SEQ ID NO: 49), (GGGS)ₙ (SEQ ID NO: 50), (GGGGS)ₙ (SEQ ID NO: 51), or (GSSGGS)ₙ (SEQ ID NO: 52), wherein n is an integer of at least 1. In some instances, the linking moiety comprises an amino acid sequence according to SEQ ID NOs: 4, 53, 54, 55, 56, 57, 58, 59, 60, 61, 62, 63, 64, 65, 66, 68, 69, or 70. In some instances, the half-life extending molecule comprises a linking moiety (L₃) that connects the half-life extending molecule to the peptide. In some instances, L₃ has a formula selected from the group consisting of (G₂S)ₙ, (GS)ₙ, (GSGGS)ₙ (SEQ ID NO: 49), (GGGS)ₙ (SEQ ID NO: 50), (GGGGS)ₙ (SEQ ID NO: 51), and (GSSGGS)ₙ (SEQ ID NO: 52), wherein n is an integer of at least 1. In some instances, L₃ comprises an amino acid sequence according to SEQ ID NO: 71. In some instances, the half-life extending molecule comprises an antibody. In some instances, the antibody comprises a single domain antibody, a single chain variable fragment, or a Fab. In some instances, the single domain antibody binds to albumin. In some instances, the single domain antibody comprises 10G or 10GE. In some instances, the single domain antibody comprises 10G, and the single domain antibody comprises an amino acid sequence according SEQ ID NOs: 2 or 72. In some instances, the effector cell antigen comprises cluster of differentiation 3 (CD3). In some instances, the scFv comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19. In some instances, the scFv comprises CDRs of UCHT1. In some instances, the scFv comprises an amino acid sequence that has at least 85% sequence identity to SEQ ID NO: 86 or SEQ ID NO: 8.

Disclosed herein, in certain embodiments, are pharmaceutical compositions comprising: (i) the polypeptide complex described herein; and (ii) a pharmaceutically acceptable excipient.

Disclosed herein, in certain embodiments, are isolated recombinant nucleic acid molecules encoding the polypeptide or polypeptide complex described herein.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is an exemplary schematic of a T cell receptor (TCR) that does not comprise a peptide modification or a half-life extending molecule. Such TCRs bind to unique antigens that exist in abundance in tumor tissue. But, the unique antigens are also found in some healthy tissues, which can trigger systemic immune activation in a subject, and cause toxicity.

FIG. 2 is an exemplary ribbon diagram of an alpha polypeptide chain and a beta polypeptide chain of a TCR. The N-termini are highlighted as exemplary points of attachment for inserting the peptides described herein.

FIG. 3 depicts the modified TCR heterodimer in the soluble format conjugated to an anti-CD3 single-chain variable fragment (scFv) effector.

FIGS. 4A- 4F illustrate exemplary configurations for the TCR-Bispecific constructs where the peptide binds the TCR.

FIGS. 5A- 5B illustrate exemplary configurations for the TCR-Bispecific constructs where the peptide binds the anti-CD3 moiety.

FIG. 6 shows an exemplary modified TCR. In this example, the modified TCR is linked to a peptide which is conjugated to a half-life extending molecule (e.g. anti-albumin moiety). The peptide binds at or near the antigen binding site of the modified TCR. A tumor protease cleaves the cleavable linker disrupting the interaction of the peptide with the modified TCR. The single cleavage simultaneously removes the peptide and the half-life extending molecule. The antigen binding site of the modified TCR is exposed, and the modified TCR selectively binds to its target antigen.

FIG. 7A- FIG. 7B illustrate modified TCR exhibits tumor protease dependent binding to target antigen. FIG. 7A illustrates the complex detected by the assay. FIG. 7B illustrates binding comparison of the modified TCR-20 with the peptide bound to the antigen binding site, and modified TCR-20 with the peptide cleaved (TCR-20 + cleaved), thereby exposing the antigen binding site.

FIGS. 8A-8C illustrate characterization of TCR-20. FIG. 8A illustrates intact MS measurement with upper panel showing a single dominant mass of the TCR-20 bispecific TCR detected and the lower table showing correlation from measured to expected theoretical mass. FIG. 8B illustrates Analytical Size Exclusion HPLC with upper panel showing protein elution profile of the TCR-20 bispecific TCR and lower table the resulting quantitative disposition as a single identifiable peak. FIG. 8C illustrates SDS-PAGE analysis of non-reduced and reduced of masked bispecific. Lanes 1-3 are increasing amounts of purified non-reduced TCR-20 bispecific TCR, Lane 4 is a molecular weight marker corresponding the molecular weights indicated to left of the SDS-PAGE gel. Lanes 4-6 contain increasing amounts of protein as indicated above each lane.

FIG. 9-FIG. 9B illustrate BLI based kinetic binding of masked bispecific TCR constructs to cognate pMHC tumor antigen.

FIG. 10 exemplifies binding of masked bispecific TCR to cognate pMHC tumor antigen by ELISA.

FIG. 11 exemplifies binding of masked bispecific TCR to albumin by ELISA.

FIG. 12A-FIG. 12B illustrate binding of masked bispecific TCR to CD3 on the surface of human T cells by flow cytometry.

FIG. 13 illustrates masked bispecific TCR mediated T cell activation.

FIG. 14 illustrates masked bispecific TCR mediated tumor cytotoxicity.

FIGS. 15A-15C illustrate the analytical characterization of a soluble MAGE- A3 TCR (TCR-1). FIG. 15A depicts an SDS-PAGE demonstrating the MAGE-A3 TCR exists as a heterodimer comprising a β-chain and an α-chain. FIG. 15B depicts size exclusion-high-performance liquid chromatography (SEC-HPLC) chromatogram of the MAGE-A3 TCR showing the correct size of the protein with minimal degradation and aggregation products. FIG. 15C depicts liquid chromatography mass spectrometry (LC/MS) chromatogram of the MAGE-A3 TCR confirming correct molecular weight and TCR heterodimerization.

FIG. 16 illustrates a bio-layer interferometry (BLI) sensorgram of binding between the MAGE-A3 TCR (TCR-1) and the MAGE-A3 peptide-major histocompatibility complex (pMHC) at four different concentrations of the MAGE-A3 TCR (TCR-1): 50 nM, 25 nM, 12.5 nM, and 6.25 nM. For each concentration, a pair of lines is shown representing the raw data as well as a curve fit for rate constant calculations. FIG. 16 also shows the equilibrium dissociation constant (Kd), association rate constant (kon), and dissociation rate constant (koff) of binding between MAGE-A3 TCR (TCR-1) and MAGE-A3 pMHC.

FIGS. 17A-17C illustrate peptide panning using phage display enables discovery of TCR inhibitory peptides. Peptides were displayed via p3 phage protein fusion and biopanned against MAGE-A3 TCR (TCR-1).FIG. 17A illustrates the panning process involving standard bind, wash, elute, and amplify cycles. The eluted phage after 3 rounds of panning were used to infect bacteria, plated on agar, individual colonies picked and amplified, followed by binding assessments and sequencing. Figure discloses SEQ ID NOS 137-138, respectively, in order of appearance. FIG. 17B illustrates that binding of clonal phagemid to plate captured MAGE-A3 TCR were characterized by ELISA. Biotinylated TCR was captured on neutravidin coated plates followed by incubation with phage. Bound phage was detected using an anti-M13 HRP antibody conjugate. Phage binding to neutravidin captured biotinylated MAGE-A3 TCR was compared to phage binding to neutravidin alone. FIG. 17C illustrates clonal phage binders of MAGE-A3 TCR that did not bind neutravidin were evaluated for their ability to bind in the presence and absence of the cognate MAGE-A3 pMHC. Inhibition of phage binding using MAGE-A3 pMHC was used as an indicator that clonal phage bound within or near the TCR binding sites responsible for pMHC recognition.

FIGS. 18A-18P illustrate bio-layer interferometry (BLI) binding of various MAGE-A3 TCR (TCR-1) to various peptides. FIG. 18A illustrates that MAGE-A3 TCR (TCR-1) binds to peptide Peptide-5. FIG. 18B illustrates that MAGE-A3 TCR (TCR-1) binds to peptide Peptide-1. FIG. 18C illustrates that MAGE-A3 TCR (TCR-1) binds to peptide Peptide-2. FIG. 18D illustrates that MAGE-A3 TCR (TCR-1) binds to peptide Peptide-3. FIG. 18E illustrates that MAGE-A3 TCR (TCR-1) binds to peptide Peptide-4. FIG. 18F illustrates that MAGE-A3 TCR (TCR-1) binds to peptide Peptide-5. FIG. 18G illustrates that MAGE-A3 TCR (TCR-1) binds to peptide Peptide-6. FIG. 18H illustrates that MAGE-A3 TCR (TCR-1) binds to peptide Peptide-7. FIG. 18I illustrates that MAGE-A3 TCR (TCR-1) binds to peptide Peptide-8. FIG. 18J illustrates that MAGE-A3 TCR (TCR-1) binds to peptide Peptide-9. FIG. 18K illustrates that MAGE-A3 TCR (TCR-1) binds to peptide Peptide-10. FIG. 18L illustrates that MAGE-A3 TCR (TCR-1) binds to peptide Peptide-11. FIG. 18M illustrates that MAGE-A3 TCR (TCR-1) binds to peptide Peptide-12. FIG. 18N illustrates that MAGE-A3 TCR (TCR-1) binds to peptide Peptide-13. FIG. 18O illustrates that MAGE-A3 TCR (TCR-1) binds to peptide Peptide-14. FIG. 18P illustrates that MAGE-A3 TCR (TCR-1) binds to a peptide blank.

FIGS. 19A-19B illustrate that MAGE-A3 TCR (TCR-1) binds to both MAGE-A3 pMHC and various peptide in an ELISA format. FIG. 19A illustrates that MAGE-A3 TCR (TCR-1) binds to example peptide Peptide-5 in an ELISA format. FIG. 19B illustrates that MAGE-A3 TCR (TCR-1) binds to example peptides Peptide-1-Peptide-8 and Peptide-9-Peptide-16 in an ELISA format.

FIGS. 20A-20H illustrate that Peptide-5 inhibits kinetic binding of MAGE-A3 TCR to MAGE-A3 pMHC in a dose dependent fashion. FIG. 20A illustrates inhibition of kinetic binding of MAGE-A3 TCR to MAGE-A3 pMHC following a dose of 0 uM Peptide-5. FIG. 20B illustrates inhibition of kinetic binding of MAGE-A3 TCR to MAGE-A3 pMHC following a dose of 1.63 uM Peptide-5. FIG. 20C illustrates inhibition of kinetic binding of MAGE-A3 TCR to MAGE-A3 pMHC following a dose of 3.13 uM Peptide-5. FIG. 20D illustrates inhibition of kinetic binding of MAGE-A3 TCR to MAGE-A3 pMHC following a dose of 6.25 uM Peptide-5. FIG. 20E illustrates inhibition of kinetic binding of MAGE-A3 TCR to MAGE-A3 pMHC following a dose of 12.5 uM Peptide-5. FIG. 20F illustrates inhibition of kinetic binding of MAGE-A3 TCR to MAGE-A3 pMHC following a dose of 25 uM Peptide-5. FIG. 20G illustrates inhibition of kinetic binding of MAGE-A3 TCR to MAGE-A3 pMHC following a dose of 50 uM Peptide-5. FIG. 20H illustrates inhibition of kinetic binding of MAGE-A3 TCR to MAGE-A3 pMHC following a dose of 100 uM Peptide-5.

FIGS. 21A-21M illustrate that various peptides inhibits kinetic binding of MAGE-A3 TCR to MAGE-A3 pMHC in a dose dependent fashion. FIG. 21A illustrates inhibition of kinetic binding of MAGE-A3 TCR to MAGE-A3 pMHC following various doses of Peptide-1. FIG. 21B illustrates inhibition of kinetic binding of MAGE-A3 TCR to MAGE-A3 pMHC following various doses of Peptide-2. FIG. 21C illustrates inhibition of kinetic binding of MAGE-A3 TCR to MAGE-A3 pMHC following various doses of Peptide-3. FIG. 21D illustrates inhibition of kinetic binding of MAGE-A3 TCR to MAGE-A3 pMHC following various doses of Peptide-4. FIG. 21E illustrates inhibition of kinetic binding of MAGE-A3 TCR to MAGE-A3 pMHC following various doses of Peptide-6. FIG. 21F illustrates inhibition of kinetic binding of MAGE-A3 TCR to MAGE-A3 pMHC following various doses of Peptide-7. FIG. 21G illustrates inhibition of kinetic binding of MAGE-A3 TCR to MAGE-A3 pMHC following various doses of Peptide-9. FIG. 21H illustrates inhibition of kinetic binding of MAGE-A3 TCR to MAGE-A3 pMHC following various doses of Peptide-15. FIG. 21I illustrates inhibition of kinetic binding of MAGE-A3 TCR to MAGE-A3 pMHC following various doses of Peptide-16. FIG. 21J illustrates inhibition of kinetic binding of MAGE-A3 TCR to MAGE-A3 pMHC following various doses of Peptide-18. FIG. 21K illustrates inhibition of kinetic binding of MAGE-A3 TCR to MAGE-A3 pMHC in the abs. FIG. 21L illustrates kinetic binding of MAGE-A3 TCR (TCR-1) to MAGE-A3 pMHC. FIG. 21M illustrates buffer only.

FIG. 22 illustrates that multiple peptides inhibit MAGE-A3 TCR (TCR-1) from binding its cognate MAGE-A3 pMHC by ELISA.

FIGS. 23A-23E illustrate TCR binding specificity of peptide Peptide-5. FIG. 23A illustrates specificity of peptide Peptide-5 for TCR TCR-1. FIG. 23B illustrates specificity of peptide Peptide-5 for TCR-2. FIG. 23C illustrates specificity of peptide Peptide-5 for TCR TCR-3. FIG. 23D illustrates specificity of peptide Peptide-5 for TCR TCR-4. FIG. 23E illustrates a blank.

FIG. 24 illustrates the equilibrium binding of TCR-1 to Ala mutated peptides.

FIGS. 25A-25F illustrate exemplary kinetic binding of TCR-1 (50 nM, 25 nM, 12.5 nM, 6.25 nM) to Peptide-5Ala mutants. FIG. 25A illustrates kinetic binding of TCR-1 to Peptide-24. FIG. 25B illustrates kinetic binding of TCR-1 to Peptide-21. FIG. 25C illustrates kinetic binding of TCR-1 to Peptide-22. FIG. 25D illustrates kinetic binding of TCR-1 to Peptide-33. FIG. 25E illustrates kinetic binding of TCR-1 to Peptide-25. FIG. 25F illustrates kinetic binding of TCR-1 to Peptide-29.

FIG. 26 depicts a masked TCR design.

FIGS. 27A-27C illustrate characterization of masked TCR, TCR-8. FIG. 27A illustrates an SDS-PAGE of masked TCR, TCR-8. FIG. 27B illustrates an SEC-FPLC of masked TCR, TCR-8. FIG. 27C illustrates a mass spec analysis of masked TCR, TCR-8.

FIGS. 28A-28C illustrate characterization of masked TCR, TCR-9. FIG. 28A illustrates an SDS-PAGE of masked TCR, TCR-9. FIG. 28B illustrates an SEC-FPLC of masked TCR, TCR-9. FIG. 28C illustrates a mass spec analysis of masked TCR, TCR-9.

FIGS. 29A-29C illustrate characterization of masked TCR, TCR-10. FIG. 29A illustrates an SDS-PAGE of masked TCR, TCR-10. FIG. 29B illustrates an SEC-FPLC of masked TCR, TCR-10. FIG. 29C illustrates a mass spec analysis of masked TCR, TCR-10.

FIGS. 30A-30C illustrate characterization of masked TCR, TCR-11. FIG. 30A illustrates an SDS-PAGE of masked TCR, TCR-11. FIG. 30B illustrates an SEC-FPLC of masked TCR, TCR-11. FIG. 30C illustrates a mass spec analysis of masked TCR, TCR-11.

FIGS. 31A-31L illustrate kinetic binding of 50 nM TCRs to MAGE-A3 pMHC by BLI. FIG. 31A illustrates kinetic binding of 50 nM of parental non-masked TCR, TCR-1, to MAGE-A3 pMHC. FIG. 31B illustrates kinetic binding of 50 nM of parental non-masked TCR, TCR-1, pre-treated with MTSP1 to MAGE-A3 pMHC. FIG. 31C illustrates kinetic binding of 50 nM of masked TCR, TCR-10, to MAGE-A3 pMHC. FIG. 31D illustrates kinetic binding of 50 nM of masked TCR, TCR-10, pre-treated with MTSP1 to MAGE-A3 pMHC. FIG. 31E illustrates kinetic binding of 50 nM of masked TCR, TCR-11, to MAGE-A3 pMHC. FIG. 31F illustrates kinetic binding of 50 nM of masked TCR, TCR-11, pre-treated with MTSP1 to MAGE-A3 pMHC. FIG. 31G illustrates kinetic binding of 50 nM of parental non-masked TCR, TCR-1, to MAGE-A3 pMHC. FIG. 31H illustrates kinetic binding of 50 nM of parental non-masked TCR, TCR-1, pre-treated with uPa to MAGE-A3 pMHC. FIG. 31I illustrates kinetic binding of 50 nM of masked TCR, TCR-8, to MAGE-A3 pMHC. FIG. 31J illustrates kinetic binding of 50 nM of masked TCR, TCR-8, pre-treated with uPa to MAGE-A3 pMHC. FIG. 31K illustrates kinetic binding of 50 nM of masked TCR, TCR-9, to MAGE-A3 pMHC. FIG. 31L illustrates kinetic binding of 50 nM of masked TCR, TCR-9, pre-treated with uPa to MAGE-A3 pMHC.

FIG. 32 illustrates equilibrium binding of TCRs to MAGE-A3 pMHC by ELISA.

FIG. 33 illustrates equilibrium binding of TCRs to Titin pMHC by ELISA.

FIGS. 34A-34C illustrate kinetic binding of TCR TCR-1 with and without protease cleavage sites to MAGE-A3 pMHC. FIG. 34A illustrates kinetic binding of TCR TCR-1 without a protease cleavage site to MAGE-A3 pMHC. FIG. 34B illustrates kinetic binding of TCR TCR-1 with protease cleavage site TCR-6 to MAGE-A3 pMHC. FIG. 34C illustrates kinetic binding of TCR TCR-1 with protease cleavage site TCR-7 to MAGE-A3 pMHC.

FIGS. 35A-35C depict the high resolution crystal structure of masked TCR TCR-10. The structure was solved via crystallization followed by X-ray diffraction. The X-ray diffraction pattern enabled the structure solution to 2.3 Å resolution. FIG. 35A depicts the monomeric crystal structure of the TCR masked by Peptide-5 clearly located within the CDR binding site. FIG. 35B highlights the Peptide-5 interaction within the alpha chain CDR domains. FIG. 35C highlights the Peptide-5 interaction within the beta chain CDR domains.

FIG. 36 depicts different TCR bispecific configurations.

FIGS. 37A-37C illustrate characterization of non-masked TCR bispecific, TCR-14. FIG. 37A illustrates an SDS-PAGE of non-masked TCR bispecific, TCR-14. FIG. 37B illustrates an SEC-FPLC of non-masked TCR bispecific, TCR-14. FIG. 37C illustrates a mass spec analysis of non-masked TCR bispecific, TCR-14.

FIGS. 38A-38C illustrate characterization of masked TCR bispecific, TCR-15. FIG. 38A illustrates an SDS-PAGE of masked TCR bispecific, TCR-15. FIG. 38B illustrates an SEC-FPLC of masked TCR bispecific, TCR-15. FIG. 38C illustrates a mass spec analysis of masked TCR bispecific, TCR-15.

FIGS. 39A-39C illustrate characterization of masked TCR bispecific, TCR-16. FIG. 39A illustrates an SDS-PAGE of masked TCR bispecific, TCR-16. FIG. 39B illustrates an SEC-FPLC of masked TCR bispecific, TCR-16. FIG. 39C illustrates a mass spec analysis of masked TCR bispecific, TCR-16.

FIGS. 40A-40C illustrate characterization of non-masked TCR bispecific, TCR-17. FIG. 40A illustrates an SDS-PAGE of non-masked TCR bispecific, TCR-17. FIG. 40B illustrates an SEC-FPLC of non-masked TCR bispecific, TCR-17. FIG. 40C illustrates a mass spec analysis of non-masked TCR bispecific, TCR-17.

FIGS. 41A-41C illustrate characterization of masked TCR bispecific, TCR-18. FIG. 41A illustrates an SDS-PAGE of masked TCR bispecific, TCR-18. FIG. 41B illustrates an SEC-FPLC of masked TCR bispecific, TCR-18. FIG. 41C illustrates a mass spec analysis of masked TCR bispecific, TCR-18.

FIGS. 42A-42C illustrate characterization of masked TCR bispecific, TCR-19. FIG. 42A illustrates an SDS-PAGE of masked TCR bispecific, TCR-19. FIG. 42B illustrates an SEC-FPLC of masked TCR bispecific, TCR-19. FIG. 42C illustrates a mass spec analysis of masked TCR bispecific, TCR-19.

FIGS. 43A-43C illustrate characterization of non-masked TCR bispecific, TCR-12. FIG. 43A illustrates an SDS-PAGE of non-masked TCR bispecific, TCR-12. FIG. 43B illustrates an SEC-FPLC of non-masked TCR bispecific, TCR-12. FIG. 43C illustrates a mass spec analysis of non-masked TCR bispecific, TCR-12.

FIGS. 44A-44C illustrate characterization of masked TCR bispecific, TCR-13. FIG. 44A illustrates an SDS-PAGE of masked TCR bispecific, TCR-13. FIG. 44B illustrates an SEC-FPLC of masked TCR bispecific, TCR-13. FIG. 44C illustrates a mass spec analysis of masked TCR bispecific, TCR-13.

FIGS. 45A-45L illustrate kinetic binding of TCR bispecifics to MAGE-A3 pMHC by BLI. FIG. 45A illustrates kinetic binding of TCR-14. FIG. 45B illustrates kinetic binding of TCR-14 treated with uPa to MAGE-A3 pMHC. FIG. 45C illustrates kinetic binding of TCR-15. FIG. 45D illustrates kinetic binding of TCR-15 treated with uPa to MAGE-A3 pMHC. FIG. 45E illustrates kinetic binding of TCR-19. FIG. 45F illustrates kinetic binding of TCR-19 treated with uPa to MAGE-A3 pMHC. FIG. 45G illustrates kinetic binding of TCR-12. FIG. 45H illustrates kinetic binding of TCR-12 treated with uPa to MAGE-A3 pMHC. FIG. 45I illustrates kinetic binding of TCR-13. FIG. 45J illustrates kinetic binding of TCR-13 treated with uPa to MAGE-A3 pMHC. FIG. 45K illustrates kinetic binding of TCR-18. FIG. 45L illustrates kinetic binding of TCR-18 treated with uPa to MAGE-A3 pMHC.

FIGS. 46A-46G illustrate equilibrium binding of TCR bispecifics to MAGE-A3 pMHC by ELISA. FIG. 46A illustrates equilibrium binding of TCR-14 to MAGE-A3 pMHC. FIG. 46B illustrates equilibrium binding of TCR-15 to MAGE-A3 pMHC. FIG. 46C illustrates equilibrium binding of TCR-16 to MAGE-A3 pMHC. FIG. 46D illustrates equilibrium binding of TCR-17 to MAGE-A3 pMHC. FIG. 46E illustrates equilibrium binding of TCR-18 to MAGE-A3 pMHC. FIG. 46F illustrates equilibrium binding of TCR-12 to MAGE-A3 pMHC. FIG. 46G illustrates equilibrium binding of TCR-13 to MAGE-A3 pMHC.

FIGS. 47A-47G illustrate equilibrium binding of TCR bispecifics to Titin pMHC by ELISA. FIG. 47A illustrates equilibrium binding of TCR-14 to Titin pMHC. FIG. 47B illustrates equilibrium binding of TCR-15 to Titin pMHC. FIG. 47C illustrates equilibrium binding of TCR-16 Titin pMHC. FIG. 47D illustrates equilibrium binding of TCR-17 to Titin pMHC. FIG. 47E illustrates equilibrium binding of TCR-18 to Titin pMHC. FIG. 47F illustrates equilibrium binding of TCR-12 to Titin pMHC. FIG. 47G illustrates equilibrium binding of TCR-13 to Titin pMHC.

FIGS. 48A-48B illustrate cellular CD3, TCR bispecific, and MAGE-A3 pMHC tetramer ternary complex formation on the surface of human T cells by flow cytometry. FIG. 48A illustrates cellular CD3, TCR bispecific (TCR-14, TCR-15, TCR-19), and MAGE-A3 tetramer ternary complex formation on the surface of human T cells by flow cytometry. FIG. 48B illustrates cellular CD3, TCR bispecific (TCR-17, TCR-18), and MAGE-A3 tetramer ternary complex formation on the surface of human T cells by flow cytometry. FIG. 48C illustrates cellular CD3, TCR bispecific (TCR-12, TCR-13), and MAGE-A3 tetramer ternary complex formation on the surface of human T cells by flow cytometry.

FIGS. 49A-49D illustrate TCR bispecific mediated cytotoxicity and T cell activation against tumor target cells, A375. FIG. 49A illustrates TCR-15 and TCR-19 mediated % cell lysis of tumor target cells, A375. FIG. 49B illustrates TCR-15 and TCR-19 mediated T cell activation, as measured by IFNγ release. FIG. 49C illustrates TCR-18 mediated % cell lysis of tumor target cells, A375. FIG. 49D illustrates T cell activation, as measured by IFNγ release.

FIGS. 50A-50F illustrate TCR bispecific mediated cytotoxicity and T cell activation against tumor target cells, HCT116. FIG. 50A illustrates TCR-15 and TCR-19 mediated % cell lysis of tumor target cells, HCT116. FIG. 50B illustrates TCR-15 and TCR-19 mediated T cell activation, as measured by IFNγ release. FIG. 50C illustrates TCR-18 mediated % cell lysis of tumor target cells, HCT116. FIG. 50D illustrates TCR-18 mediated T cell activation, as measured by IFNγ release. FIG. 50Eillustrates TCR-13 mediated % cell lysis of tumor target cells, HCT116. FIG. 50F illustrates TCR-13 mediated T cell activation, as measured by IFNγ release.

FIGS. 51A-51B illustrate TCR bispecific mediated cytotoxicity and T cell activation against tumor target cells, HT29. FIG. 51A illustrates TCR-15 mediated % cell lysis of tumor target cells, HT29. FIG. 51B illustrates TCR-15 mediated T cell activation, as measured by IFNγ release.

FIGS. 52A-52B illustrate TCR bispecific mediated cytotoxicity and T cell activation against human skeletal muscle myoblasts, HSMM. FIG. 52A illustrates TCR-14, TCR-15, and TCR-18 mediated % cell lysis of HSMM. FIG. 52B illustrates TCR-14, TCR-15, and TCR-18 mediated T cell activation, as measured by IFNγ release.

FIG. 53 depicts the tumor specific activity and cross over PK concepts within TCR bispecific molecules.

FIGS. 54A-54B depict general TCR bispecific designs. FIG. 54A depicts general dual mask TCR bispecific design. FIG. 54B depicts general single mask TCR bispecific design.

FIG. 55 depicts examples of TCR bispecific constructs.

FIGS. 56A-56D illustrate characterization of non-masked TCR bispecific, TCR-20. FIG. 56A illustrates an SDS-PAGE of non-masked TCR bispecific, TCR-20. FIG. 56B illustrates an SEC-FPLC of non-masked TCR bispecific, TCR-20. FIG. 56C illustrates a mass spec analysis of non-masked TCR bispecific, TCR-20. FIG. 56D illustrates a mass spec analysis of non-masked TCR bispecific, TCR-20.

FIGS. 57A-57C illustrate characterization of masked TCR bispecific, TCR-21. FIG. 56A illustrates an SDS-PAGE of masked TCR bispecific, TCR-21. FIG. 56B illustrates an SEC-FPLC of masked TCR bispecific, TCR-21. FIG. 56C illustrates a mass spec analysis of masked TCR bispecific, TCR-21.

FIGS. 58A-58C illustrate characterization of non-masked TCR bispecific, TCR-22. FIG. 56A illustrates an SDS-PAGE of non-masked TCR bispecific, TCR-22. FIG. 56B illustrates an SEC-FPLC of non-masked TCR bispecific, TCR-22. FIG. 56C illustrates a mass spec analysis of non-masked TCR bispecific, TCR-22.

FIG. 59 illustrates TCR-20 binding albumin from species indicated by ELISA.

FIGS. 60A-60I illustrate TCR bispecific kinetic binding to MAGE-A3 pMHC in the presence of bovine serum albumin (BSA) or human serum albumin (HSA) containing buffer by BLI. FIG. 60A illustrates TCR-20 kinetic binding to MAGE-A3 pMHC in the presence of BSA containing albumin. FIG. 60B illustrates MTSP1 treated TCR-20 kinetic binding to MAGE-A3 pMHC in the presence of BSA containing albumin.. FIG. 60C illustrates TCR-20 kinetic binding to MAGE-A3 pMHC in the presence of HSA containing albumin. FIG. 60D illustrates MTSP1 treated TCR-20 kinetic binding to MAGE-A3 pMHC in the presence of HSA containing albumin. FIG. 60E illustrates TCR-20 kinetic binding to MAGE-A3 pMHC in the presence of BSA containing albumin. FIG. 60F illustrates uPa treated TCR-20 kinetic binding to MAGE-A3 pMHC in the presence of BSA containing albumin. FIG. 60G illustrates TCR-21 kinetic binding to MAGE-A3 pMHC in the presence of BSA containing albumin. 60H illustrates uPa treated TCR-21 kinetic binding to MAGE-A3 pMHC in the presence of BSA containing albumin. 60I illustrates TCR-22 kinetic binding to MAGE-A3 pMHC in the presence of BSA containing albumin.

FIGS. 61A-61L illustrate TCR bispecific kinetic binding MAGE-A3 pMHC in buffer containing fetal bovine serum (BS), mouse serum (MS), cynomolgus monkey serum (CS) or human serum (HS) by BLI. FIG. 61A illustrates TCR-21 kinetic binding MAGE-A3 pMHC in buffer containing MS. FIG. 61B illustrates TCR-21 kinetic binding MAGE-A3 pMHC in buffer containing BS. FIG. 61C illustrates TCR-21 kinetic binding MAGE-A3 pMHC in buffer containing CS. FIG. 61D illustrates TCR-21 kinetic binding MAGE-A3 pMHC in buffer containing HS. FIG. 61E illustrates TCR-20 kinetic binding MAGE-A3 pMHC in buffer containing MS. FIG. 61F illustrates MTSP1 treated TCR-20 kinetic binding MAGE-A3 pMHC in buffer containing MS. FIG. 61G illustrates TCR-20 kinetic binding MAGE-A3 pMHC in buffer containing BS. FIG. 61H illustrates MTSP1 TCR-20 kinetic binding MAGE-A3 pMHC in buffer containing BS. FIG. 61E illustrates TCR-20 kinetic binding MAGE-A3 pMHC in buffer containing CS. FIG. 61F illustrates MTSP1 treated TCR-20 kinetic binding MAGE-A3 pMHC in buffer containing CS. FIG. 61G illustrates TCR-20 kinetic binding MAGE-A3 pMHC in buffer containing HS. FIG. 61H illustrates MTSP1 TCR-20 kinetic binding MAGE-A3 pMHC in buffer containing HS.

FIGS. 62A-62B illustrates TCR-20 equilibrium binding MAGE-A3 pMHC by ELISA. FIG. 62A illustrates TCR-20 equilibrium binding MAGE-A3 pMHC in buffer containing bovine albumin by ELISA. FIG. 62B illustrates TCR-20 equilibrium binding MAGE-A3 pMHC in buffer containing human albumin by ELISA.

FIG. 63 illustrates cellular CD3, TCR bispecific, and MAGE-A3 pMHC tetramer ternary complex formation on the surface of human T cells by flow cytometry.

FIGS. 64A-64B illustrates TCR bispecific mediated cytotoxicity and T cell activation against tumor target cells, A375, using fetal bovine serum supplemented medium. FIG. 64A illustrates TCR-20 mediated % cell lysis of tumor target cells, A375, using BSA supplemented medium. FIG. 64B illustrates TCR-20 mediated T cell activation, as measured by IFNγ release, using BSA supplemented medium.

FIGS. 65A-65B illustrates TCR bispecific mediated cytotoxicity and T cell activation against tumor target cells, A375, using human serum supplemented medium. FIG. 65A illustrates TCR-20 mediated % cell lysis of tumor target cells, A375, using HSA supplemented medium. FIG. 65B illustrates TCR-20 mediated T cell activation, as measured by IFNγ release, using HSA supplemented medium.

FIGS. 66A-66B illustrates TCR bispecific mediated cytotoxicity and T cell activation against tumor target cells, HCT116, using fetal bovine serum supplemented medium. FIG. 66A illustrates TCR-20 mediated % cell lysis of tumor target cells, HCT116, using BSA supplemented medium. FIG. 66B illustrates TCR-20 mediated T cell activation, as measured by IFNγ release, using BSA supplemented medium.

FIGS. 67A-67B illustrates TCR bispecific mediated cytotoxicity and T cell activation against tumor target cells, HCT116, using human serum supplemented medium. FIG. 67A illustrates TCR-20 mediated % cell lysis of tumor target cells, HCT116, using HSA supplemented medium. FIG. 67B illustrates TCR-20 mediated T cell activation, as measured by IFNγ release, using HSA supplemented medium.

FIG. 68 illustrates Balb/c mouse pharmacokinetics of TCR bispecific, TCR-20, compared to non-masked parental TCR bispecific T cell engager, TCR-14.

FIG. 69 illustrates cynomolgus monkey pharmacokinetics of TCR bispecific, TCR-20, compared to non-masked parental TCR bispecific T cell engager, TCR-14.

FIGS. 70A-70Y illustrate exemplary TCR bispecific constructs described herein.

### DETAILED DESCRIPTION

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

“Transmembrane domain”, as used herein, refers to the region of a receptor which crosses the plasma membrane. Examples include the transmembrane region of a transmembrane protein (for example a Type 1 transmembrane protein), an artificial hydrophobic sequence, and a combination thereof.

“Fragment” as used herein refers to a peptide or a polypeptide that comprises less than the full length amino acid sequence.

“Antigen-binding site” as used herein refers to the region of a polypeptide that interacts with an antigen. The antigen binding site includes amino acid residues that interact directly with an antigen and those amino acid residues that are within proximity to the antigen but that may not interact directly with the antigen.

### T Cell Receptor (TCR)

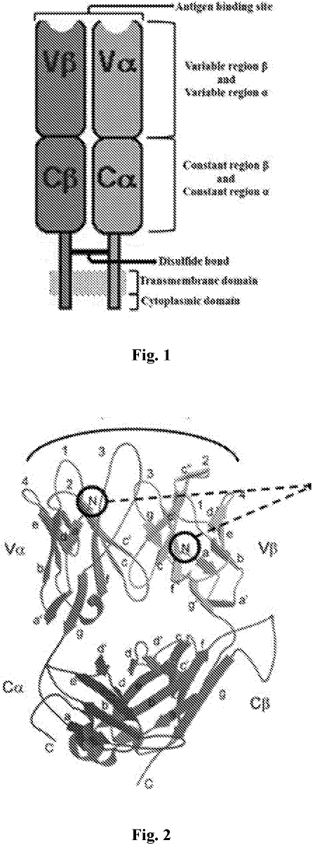

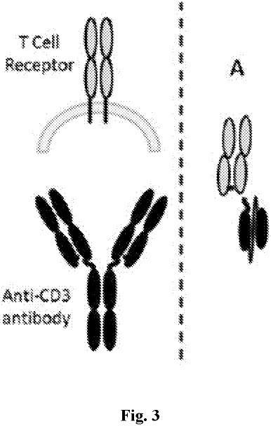

Native TCRs are transmembrane receptors expressed on the surface of T cells that recognize antigens bound to major histocompatibility complex molecules (MHC). Native TCRs are heterodimeric and comprise an alpha polypeptide chain and a beta polypeptide chain linked through a disulfide bond (FIG. 1). The alpha polypeptide chain and the beta polypeptide chain are expressed as part of a complex with accessory proteins which include, for example, two CD3 epsilon polypeptides, one CD3 gamma polypeptide, one CD3 delta polypeptide, and two CD3 zeta polypeptides. When a TCR engages with a target antigen and MHC, the T cell is activated resulting in a series of signaling events mediated by associated enzymes, co-receptors, adapter molecules, and activated or released transcription factors.

In native TCRs, the alpha polypeptide chain and the beta polypeptide chain comprise an extracellular domain, a transmembrane domain, and a cytoplasmic domain. Each extracellular domain comprises a variable region (V), a joining region (J), and a constant region (C). The constant region is N-terminal to the transmembrane domain, and the transmembrane domain is N-terminal to the cytoplasmic domain. The variable regions of both the alpha polypeptide chain and the beta polypeptide chain comprise three hypervariable or complementarity determining regions (CDRs). The beta polypeptide chain usually contains a short diversity region between the variable and joining regions. The three CDRs are embedded into a framework sequence, with one CDR being the hypervariable region named CDR3. The alpha chain variable region (Vα) and the beta chain variable region (Vβ) are of several types that are distinguished by their framework sequences, CDR1 and CDR2 sequences, and a partly defined CDR3 sequence.

TCRs are described using the International Immunogenetics (IMGT) TCR nomenclature. The Vα in IMGT nomenclature is referred to by a unique “TRAV” number. In the same way, Vβ is referred to by a unique “TRBV” number. The corresponding joining and constant regions are referred to as TRAJ and TRAC, respectively for the α joining and constant regions, and TRBJ and TRBC, respectively for the β joining and constant regions. The sequences defined by the IMGT nomenclature are known in the art and are contained within the online IMGT public database.

### Polypeptides or Polypeptide Complexes

Disclosed herein, in some embodiments, are modified T cell engager polypeptides or polypeptide complexes comprising a half-life extending molecule. In some embodiments, the polypeptides or polypeptide complexes comprise a T cell receptor (TCR). In some embodiments, the polypeptides or polypeptide complexes comprise an antibody or an antibody fragment. In some embodiments, the polypeptides or polypeptide complexes comprise a T cell receptor (TCR) and an antibody or an antibody fragment.

Disclosed herein, in some embodiments, are polypeptides or polypeptide complexes according to Formula I:

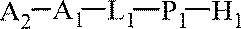

wherein A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to a second target antigen. Disclosed herein, in some embodiments, are polypeptides or polypeptide complexes comprising Formula I:

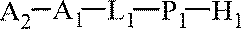

wherein A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to a second target antigen. Disclosed herein, in some embodiments, are polypeptides or polypeptide complexes comprising Formula I:

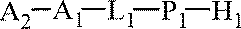

wherein A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to a second target antigen. Disclosed herein, in some embodiments, are polypeptides or polypeptide complexes according to Formula I:

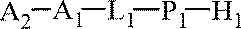

wherein A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to a second target antigen. In some embodiments, the first target antigen comprises a tumor cell antigen and the second target antigen comprises an effector cell antigen. In some embodiments, the first target antigen comprises an effector cell antigen and the second target antigen comprises a tumor cell antigen. In some embodiments, the polypeptide or polypeptide complex of formula I binds to a target cell when L₁ is cleaved by the tumor specific protease. In some embodiments, the polypeptide of formula I binds to an effector cell when L₁ is cleaved by the tumor specific protease.

Disclosed herein, in some embodiments, are polypeptides or polypeptide complexes according to Formula Ia:

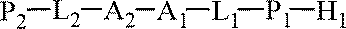

wherein: A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; A₂ comprises a second antigen recognizing molecule that binds to a second target antigen; P₂ comprises a peptide that binds to A₂; and L₂ comprises a linking moiety that connects A₂ to P₂ and is a substrate for a tumor specific protease.

Disclosed herein, in some embodiments, are polypeptides or polypeptide complexes according to Formula Ia:

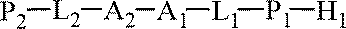

wherein: A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; A₂ is a second antigen recognizing molecule that binds to a second target antigen; P₂ is a peptide that binds to A₂; and L₂ is a linking moiety that connects A₂ to P₂ and is a substrate for a tumor specific protease.

Disclosed herein, in some embodiments, are polypeptides or polypeptide complexes comprising Formula Ia:

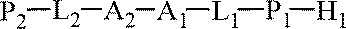

wherein: A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; A₂ comprises a second antigen recognizing molecule that binds to a second target antigen; P₂ comprises a peptide that binds to A₂; and L₂ comprises a linking moiety that connects A₂ to P₂ and is a substrate for a tumor specific protease.

Disclosed herein, in some embodiments, are polypeptides or polypeptide complexes comprising Formula Ia:

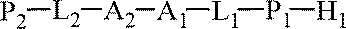

wherein: A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; A₂ is a second antigen recognizing molecule that binds to a second target antigen P₂ is a peptide that binds to A₂; and L₂ is a linking moiety that connects A₂ to P₂ and is a substrate for a tumor specific protease.

In some embodiments, the polypeptide or polypeptide complex has an increased EC₅₀ in a T-cell cytolysis assay as compared to the EC₅₀ in a T-cell cytolysis assay of a polypeptide or polypeptide complex that does not have P₁ or L₁. In some embodiments, the polypeptide or polypeptide complex has an increased EC₅₀ in a T-cell cytolysis assay that is at least 10X higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 20X higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 30X higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 40X higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 50X higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 60X higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 70X higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 80X higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 90X higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 100X higher than the EC₅₀ in a T-cell cytolysis assay of a polypeptide or polypeptide complex that does not have P₁ or L₁.

In some embodiments, the polypeptide or polypeptide complex has an increased EC₅₀ in a T-cell cytolysis assay as compared to the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 10X higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 20X higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 30X higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 40X higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 50X higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 60X higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 70X higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 80X higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 90X higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 100X higher than the EC₅₀ in a T-cell cytolysis assay of the polypeptide or polypeptide complex in which L₁ has been cleaved by the tumor specific protease.

In some embodiments, the polypeptide or polypeptide complex has an increased EC₅₀ in an IFNγ release T-cell activation assay as compared to the EC₅₀ in an IFNγ release T-cell activation assay of a polypeptide or polypeptide complex that does not have P₁ or L₁. In some embodiments, the polypeptide or polypeptide complex has an increased EC₅₀ in an IFNγ release T-cell activation assay that is at least 10X higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 20X higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 30X higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 40X higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 50X higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 60X higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 70X higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 80X higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 90X higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 100X higher than the EC₅₀ in an IFNγ release T-cell activation assay of a form of the polypeptide or polypeptide complex that does not have P₁ or L₁.

In some embodiments, the polypeptide or polypeptide complex has an increased EC₅₀ in an IFNγ release T-cell activation assay as compared to the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 10X higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 20X higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 30X higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 40X higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 50X higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 60X higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 70X higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 80X higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 90X higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 100X higher than the EC₅₀ in an IFNγ release T-cell activation assay of the polypeptide or polypeptide complex in which L₁ has been cleaved by the tumor specific protease.

In some embodiments, the polypeptide or polypeptide complex comprises a modified amino acid, a non-natural amino acid, a modified non-natural amino acid, or a combination thereof. In some embodiments, the modified amino acid or modified non-natural amino acid comprises a post-translational modification.

Further disclosed herein, in some embodiments, are polypeptides or polypeptide complexes according to Formula II:

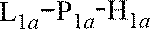

wherein: L₁ₐ comprises a tumor specific protease-cleaved linking moiety that when uncleaved connects P₁ₐ to an antigen recognizing molecule that binds to a target antigen and; P₁ₐ comprises a peptide that binds to the antigen recognizing molecule when L₁ₐ is uncleaved; and H₁ₐ comprises a half-life extending molecule. Further disclosed herein, in some embodiments, are polypeptides or polypeptide complexes comprising Formula II:

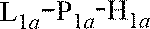

wherein: L₁ₐ comprises a tumor specific protease-cleaved linking moiety that when uncleaved connects P₁ₐ to an antigen recognizing molecule that binds to a target antigen and; P₁ₐ comprises a peptide that binds to the antigen recognizing molecule when L₁ₐ is uncleaved; and H₁ₐ comprises a half-life extending molecule. Further disclosed herein, in some embodiments, are polypeptides or polypeptide complexes comprising Formula II:

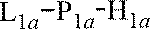

wherein: L₁ₐ is a tumor specific protease-cleaved linking moiety that when uncleaved connects P₁ₐ to an antigen recognizing molecule that binds to a target antigen and; P₁ₐ is a peptide that binds to the antigen recognizing molecule when L₁ₐ is uncleaved; and H₁ₐ is a half-life extending molecule. Further disclosed herein, in some embodiments, are polypeptides or polypeptide complexes according to Formula II:

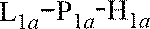

wherein: L₁ₐ is a tumor specific protease-cleaved linking moiety that when uncleaved connects P₁ₐ to an antigen recognizing molecule that binds to a target antigen and; P₁ₐ is a peptide that binds to the antigen recognizing molecule when L₁ₐ is uncleaved; and H₁ₐ is a half-life extending molecule. In some embodiments, the antigen recognizing molecule comprises a soluble TCR that comprises an alpha TCR polypeptide comprising a TCR alpha extracellular domain and a beta TCR polypeptide comprising a TCR beta extracellular domain. In some embodiments, the antigen recognizing molecule comprises an antibody or antibody fragment. In some embodiments, the target antigen is an anti-CD3 effector cell antigen.

### Antigen Recognizing Molecule (A₁)

In some embodiments, A₁ is a soluble T cell receptor (TCR). In some embodiments, the soluble TCR is a single chain TCR comprising a variable region of a TCR alpha extracellular domain, or fragment thereof, and a variable region of a TCR beta extracellular domain, or fragment thereof. In some embodiments, the soluble TCR comprises an alpha TCR polypeptide comprising a TCR alpha extracellular domain and a beta TCR polypeptide comprising a TCR beta extracellular domain. In some embodiments, L₁ is bound to N-terminus of the alpha TCR polypeptide. In some embodiments, L₁ is bound to N-terminus of the beta TCR polypeptide. In some embodiments, A₂ is bound to C-terminus of the alpha TCR polypeptide. In some embodiments, A₂ is bound to N-terminus of the alpha TCR polypeptide. In some embodiments, A₂ is bound to C-terminus of the beta TCR polypeptide. In some embodiments, A₂ is bound to N-terminus of the beta TCR polypeptide. In some embodiments, L₁ is bound to N-terminus of the alpha TCR polypeptide and A₂ is bound to N-terminus of the beta TCR polypeptide. In some embodiments, L₁ is bound to N-terminus of the alpha TCR polypeptide and A₂ is bound to C-terminus of the beta TCR polypeptide. In some embodiments, L₁ is bound to N-terminus of the alpha TCR polypeptide and A₂ is bound to C-terminus of the alpha TCR polypeptide. In some embodiments, L₁ is bound to N-terminus of the beta TCR polypeptide and A₂ is bound to N-terminus of the alpha TCR polypeptide. In some embodiments, L₁ is bound to N-terminus of the beta TCR polypeptide and A₂ is bound to C-terminus of the beta TCR polypeptide. In some embodiments, L₁ is bound to N-terminus of the beta TCR polypeptide and A₂ is bound to C-terminus of the alpha TCR polypeptide.

In some embodiments, A₁ comprises a MAGEA3 binding TCR alpha domain. In some embodiments, A₁ comprises a MAGEA3 binding TCR beta domain. In some embodiments, A₁ comprises a MART1 binding TCR alpha domain. In some embodiments, A₁ comprises a MART1 binding TCR beta domain. In some embodiments, the tumor cell antigen comprises MAGEA3. In some embodiments, the tumor cell antigen comprises MART1.

In some embodiments, the polypeptide or polypeptide complex comprises an amino acid sequence according to SEQ ID NO: 1. In some embodiments, the polypeptide or polypeptide complex comprises an amino acid sequence according to SEQ ID NO: 7. In some embodiments, the TCR alpha extracellular domain comprises three hypervariable complementarity determining regions (CDRs). In some embodiments, at least one CDR comprises a mutation to increase binding affinity or binding specificity to the first target antigen. In some embodiments, at least one CDR comprises a mutation to increase binding affinity and binding specificity to the first target antigen. In some embodiments, the TCR beta extracellular domain comprises three hypervariable complementarity determining regions (CDRs). In some embodiments, at least one CDR comprises a mutation to increase binding affinity or binding specificity to the first target antigen. In some embodiments, at least one CDR comprises a mutation to increase binding affinity and binding specificity to the first target antigen. In some embodiments, there are 2-20, 3-15, 4-12, or 4-10 mutation in one or two CDRs. In some embodiments, the TCR alpha extracellular domain, or fragment thereof, and the TCR beta extracellular domain, or fragment thereof, are connected by a disulfide bond.

In some embodiments, the polypeptide or polypeptide complex has weaker binding affinity for its pMHC as compared to the binding affinity for the pMHC of a polypeptide or polypeptide complex that does not have P₁ or L₁. In some embodiments, the polypeptide or polypeptide complex has weaker binding affinity for its pMHC that is at least 5X higher than the binding affinity for the pMHC of a form of the polypeptide or polypeptide complex that does not have P₁ or L₁. In some embodiments, the polypeptide or polypeptide complex has weaker binding affinity for its pMHC that is at least 8X higher than the binding affinity for the pMHC of a form of the polypeptide or polypeptide complex that does not have P₁ or L₁. In some embodiments, the polypeptide or polypeptide complex has weaker binding affinity for its pMHC that is at least 10X higher than the binding affinity for the pMHC of a form of the polypeptide or polypeptide complex that does not have P₁ or L₁. In some embodiments, the polypeptide or polypeptide complex has weaker binding affinity for its pMHC that is at least 20X higher than the binding affinity for the pMHC of a form of the polypeptide or polypeptide complex that does not have P₁ or L₁. In some embodiments, the polypeptide or polypeptide complex has weaker binding affinity for its pMHC that is at least 25X higher than the binding affinity for the pMHC of a form of the polypeptide or polypeptide complex that does not have P₁ or L₁. In some embodiments, the polypeptide or polypeptide complex has weaker binding affinity for its pMHC that is at least 30X higher than the binding affinity for the pMHC of a form of the polypeptide or polypeptide complex that does not have P₁ or L₁. In some embodiments, the polypeptide or polypeptide complex has weaker binding affinity for its pMHC that is at least 35X higher than the binding affinity for the pMHC of a form of the polypeptide or polypeptide complex that does not have P₁ or L₁. In some embodiments, the polypeptide or polypeptide complex has weaker binding affinity for its pMHC that is at least 40X higher than the binding affinity for the pMHC of a form of the polypeptide or polypeptide complex that does not have P₁ or L₁. In some embodiments, the polypeptide or polypeptide complex has weaker binding affinity for its pMHC that is at least 45 higher than the binding affinity for the pMHC of a form of the polypeptide or polypeptide complex that does not have P₁ or L₁. In some embodiments, the polypeptide or polypeptide complex has weaker binding affinity for its pMHC that is at least 50X higher than the binding affinity for the pMHC of a form of the polypeptide or polypeptide complex that does not have P₁ or L₁. In some embodiments, the polypeptide or polypeptide complex has weaker binding affinity for its pMHC that is at least 55X higher than the binding affinity for the pMHC of a form of the polypeptide or polypeptide complex that does not have P₁ or L₁. In some embodiments, the polypeptide or polypeptide complex has weaker binding affinity for its pMHC that is at least 60X higher than the binding affinity for the pMHC of a form of the polypeptide or polypeptide complex that does not have P₁ or L₁. In some embodiments, the polypeptide or polypeptide complex has weaker binding affinity for its pMHC that is at least 65X higher than the binding affinity for the pMHC of a form of the polypeptide or polypeptide complex that does not have P₁ or L₁. In some embodiments, the polypeptide or polypeptide complex has weaker binding affinity for its pMHC that is at least 70X higher than the binding affinity for the pMHC of a form of the polypeptide or polypeptide complex that does not have P₁ or L₁. In some embodiments, the polypeptide or polypeptide complex has weaker binding affinity for its pMHC that is at least 75X higher than the binding affinity for the pMHC of a form of the polypeptide or polypeptide complex that does not have P₁ or L₁. In some embodiments, the polypeptide or polypeptide complex has weaker binding affinity for its pMHC that is at least 80X higher than the binding affinity for the pMHC of a form of the polypeptide or polypeptide complex that does not have P₁ or L₁. In some embodiments, the polypeptide or polypeptide complex has weaker binding affinity for its pMHC that is at least 85X higher than the binding affinity for the pMHC of a form of the polypeptide or polypeptide complex that does not have P₁ or L₁. In some embodiments, the polypeptide or polypeptide complex has weaker binding affinity for its pMHC that is at least 90X higher than the binding affinity for the pMHC of a form of the polypeptide or polypeptide complex that does not have P₁ or L₁. In some embodiments, the polypeptide or polypeptide complex has weaker binding affinity for its pMHC that is at least 95X higher than the binding affinity for the pMHC of a form of the polypeptide or polypeptide complex that does not have P₁ or L₁. In some embodiments, the polypeptide or polypeptide complex has weaker binding affinity for its pMHC that is at least 100X higher than the binding affinity for the pMHC of a form of the polypeptide or polypeptide complex that does not have P₁ or L₁. In some embodiments, the polypeptide or polypeptide complex has weaker binding affinity for its pMHC that is at least 120X higher than the binding affinity for the pMHC of a form of the polypeptide or polypeptide complex that does not have P₁ or L₁. In some embodiments, the polypeptide or polypeptide complex has weaker binding affinity for its pMHC that is at least 150X higher than the binding affinity for the pMHC of a form of the polypeptide or polypeptide complex that does not have P₁ or L₁.

In some embodiments, the polypeptide or polypeptide complex has weaker binding affinity for its pMHC as compared to the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 5X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 8X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 10X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 15X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 20X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 25X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 30X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 35X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 40X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 45X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 50X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 55X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 60X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 65X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 70X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 75X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 80X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 85X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 90X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 95X higher than the binding affinity for the pMHC of the polypeptide or polypeptide complex in which L₁ has been cleaved by the tumor specific protease. In some embodiments, the polypeptide or polypeptide complex has weaker binding affinity for its pMHC that is at least 100X higher than the binding affinity for the pMHC of the polypeptide or polypeptide complex in which L₁ has been cleaved by the tumor specific protease.

In some embodiments, A₁ is an antibody or an antibody fragment. In some embodiments, the antibody or the antibody fragment thereof comprises a single chain variable fragment, a single domain antibody, or a Fab. In some embodiments, wherein the antibody or antibody fragment thereof comprises a single chain variable fragment (scFv), a heavy chain variable domain (VH domain), a light chain variable domain (VL domain), or a variable domain (VHH) of a camelid derived single domain antibody. In some embodiments, the antibody or antibody fragment thereof comprises a single-chain variable fragment. In some embodiments, the antibody or antibody fragment thereof is humanized or human. In some embodiments, L₁ is bound to N-terminus of antibody or antibody fragment. In some embodiments, A₂ is bound to C-terminus of antibody or antibody fragment. In some embodiments, L₁ is bound to N-terminus of antibody or antibody fragment and A₂ is bound to C-terminus of antibody or antibody fragment.

In some embodiments, A₁ is the Fab. In some embodiments, the Fab comprises (a) a Fab light chain polypeptide comprising a light chain variable domain and a constant domain; and (b) a Fab heavy chain polypeptide comprising a heavy chain variable domain and a constant domain. In some embodiments, L₁ is bound to N-terminus of the Fab light chain polypeptide. In some embodiments, L₁ is bound to N-terminus of the Fab heavy chain polypeptide. In some embodiments, A₂ is bound to C-terminus of the Fab light chain polypeptide. In some embodiments, A₂ is bound to N-terminus of the Fab light chain polypeptide. In some embodiments, A₂ is bound to C-terminus of the Fab heavy chain polypeptide. In some embodiments, A₂ is bound to N-terminus of the Fab heavy chain polypeptide. In some embodiments, L₁ is bound to N-terminus of the Fab light chain polypeptide and A₂ is bound to N-terminus of the Fab heavy chain polypeptide. In some embodiments, L₁ is bound to N-terminus of the Fab light chain polypeptide and A₂ is bound to C-terminus of the Fab heavy chain polypeptide. In some embodiments, L₁ is bound to N-terminus of the Fab light chain polypeptide and A₂ is bound to C-terminus of the Fab light chain polypeptide. In some embodiments, L₁ is bound to N-terminus of the Fab heavy chain polypeptide and A₂ is bound to N-terminus of the Fab light chain polypeptide. In some embodiments, L₁ is bound to N-terminus of the Fab heavy chain polypeptide and A₂ is bound to C-terminus of the Fab heavy chain polypeptide. In some embodiments, L₁ is bound to N-terminus of the Fab heavy chain polypeptide and A₂ is bound to C-terminus of the Fab light chain polypeptide. In some embodiments, A₂ is bound to the N-terminus of the Fab heavy chain polypeptide and A₂ further comprises P₂ and L₂, wherein P₂ comprises a peptide that binds to A₂; and L₂ comprises a linking moiety that connects A₂ to P₂ and is a substrate for a tumor specific protease.

In some embodiments, the polypeptide or polypeptide complex has weaker binding affinity for the tumor cell antigen as compared to the binding affinity for the tumor cell antigen of a polypeptide or polypeptide complex that does not have P₁ or L₁. In some embodiments, the polypeptide or polypeptide complex has weaker binding affinity for the tumor cell antigen that is at least 5X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 8X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 10X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 15X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 20X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 25X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 30X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 35X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 40X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 45X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 50X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 55X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 60X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 65X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 70X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 75X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 80X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 85X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 90X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 95X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 100X higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 120X higher than the binding affinity for the tumor cell antigen of a form of the polypeptide or polypeptide complex that does not have P₁ or L₁.

In some embodiments, the polypeptide or polypeptide complex has weaker binding affinity for the tumor cell antigen as compared to the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 5X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 8X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 10X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 15X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 20X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 25X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 30X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 35X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 40X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 45X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 50X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 55X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 60X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 65X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 70X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 75X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 80X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 85X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 90X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 95X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 100X higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 120X higher than the binding affinity for the tumor cell antigen of the polypeptide or polypeptide complex in which L₁ has been cleaved by the tumor specific protease.

In some embodiments, the polypeptide or polypeptide complex of formula I binds to a target cell when L₁ is cleaved by the tumor specific protease and A₂ binds to an effector cell. In some embodiments, the effector cell is a T cell. In some embodiments, A₂ binds to a polypeptide that is part of a TCR-CD3 complex on the effector cell. In some embodiments, the polypeptide that is part of the TCR-CD3 complex is human CD3ε. In some embodiments, A₁ comprises an anti-CD3e single-chain variable fragment. In some embodiments, A₁ comprises an anti-CD3e single-chain variable fragment that has a K_{D} binding of 1 µM or less to CD3 on CD3 expressing cells. In some embodiments, A₁ comprises a variable light chain and variable heavy chain each of which is capable of specifically binding to human CD3. In some embodiments, A₁ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19. In some embodiments, the polypeptide or polypeptide complex of formula I binds to an effector cell when L₁ is cleaved by the tumor specific protease and A₁ binds to the effector cell. In some embodiments, the effector cell is a T cell. In some embodiments, A₁ binds to a polypeptide that is part of a TCR-CD3 complex on the effector cell. In some embodiments, the polypeptide that is part of the TCR-CD3 complex is human CD3ε. In some embodiments, the effector cell antigen comprises CD3, and the scFv comprises an amino acid sequence according to SEQ ID NO: 86 or 8.

### Antigen Recognizing Molecule (A₂)

In some embodiments, A₂ comprises an antibody or antibody fragment. In some embodiments, A₂ comprises an antibody or antibody fragment that is human or humanized. In some embodiments, A₂ comprises a single chain variable fragment, a single domain antibody, or a Fab. In some embodiments, A₂ comprises a single chain variable fragment (scFv), a heavy chain variable domain (VH domain), a light chain variable domain (VL domain), or a variable domain (VHH) of a camelid derived single domain antibody. In some embodiments, A₂ comprises an anti-CD3e single-chain variable fragment. In some embodiments, A₂ comprises an anti-CD3e single-chain variable fragment that has a K_{D} binding of 1 µM or less to CD3 on CD3 expressing cells. In some embodiments, A₂ comprises a variable light chain and variable heavy chain each of which is capable of specifically binding to human CD3. In some embodiments, A₂ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19. In some embodiments, A₁ is bound to the N-terminus of A₂.

In some embodiments, A₂ is a soluble T cell receptor (TCR). In some embodiments, the soluble TCR is a single chain TCR comprising a variable region of a TCR alpha extracellular domain, or fragment thereof, and a variable region of a TCR beta extracellular domain, or fragment thereof. In some embodiments, the soluble TCR comprises an alpha TCR polypeptide comprising a TCR alpha extracellular domain and a beta TCR polypeptide comprising a TCR beta extracellular domain. In some embodiments, A₁ is bound to C-terminus of the alpha TCR polypeptide. In some embodiments, A₁ is bound to C-terminus of the beta TCR polypeptide. In some embodiments, A₁ is bound to N-terminus of the beta TCR polypeptide. In some embodiments, the alpha TCR polypeptide further comprises P₂ and L₂, wherein P₂ comprises a peptide that binds to A₂; and L₂ comprises a linking moiety that connects A₂ to P₂ and is a substrate for a tumor specific protease. In some embodiments, A₁ is bound to N-terminus of the alpha TCR polypeptide. In some embodiments, the beta TCR polypeptide further comprises P₂ and L₂, wherein P₂ comprises a peptide that binds to A₂; and L₂ comprises a linking moiety that connects A₂ to P₂ and is a substrate for a tumor specific protease. In some embodiments, A₂ comprises a MAGEA3 binding TCR alpha domain. In some embodiments, A₂ comprises a MAGEA3 binding TCR beta domain. In some embodiments, A₂ comprises a MART1 binding TCR alpha domain. In some embodiments, A₂ comprises a MART1 binding TCR beta domain. In some embodiments, the tumor cell antigen comprises MAGEA3 or MART1. In some embodiments, A₂ comprises an amino acid sequence according to SEQ ID NO: 5. In some embodiments, A₂ comprises an amino acid sequence according to SEQ ID NO: 9. In some embodiments, the TCR alpha extracellular domain comprises three hypervariable complementarity determining regions (CDRs). In some embodiments, at least one CDR comprises a mutation to increase binding affinity or binding specificity to the first target antigen. In some embodiments, at least one CDR comprises a mutation to increase binding affinity and binding specificity to the first target antigen. In some embodiments, the TCR beta extracellular domain comprises three hypervariable complementarity determining regions (CDRs). In some embodiments, at least one CDR comprises a mutation to increase binding affinity or binding specificity to the first target antigen. In some embodiments, at least one CDR comprises a mutation to increase binding affinity and binding specificity to the first target antigen. In some embodiments, the TCR alpha extracellular domain, or fragment thereof, and the TCR beta extracellular domain, or fragment thereof, are connected by a disulfide bond. In some embodiments, the tumor cell antigen comprises MAGEA3, and the alpha TCR polypeptide comprises an alpha chain of TCR-1, TCR-2, TCR-3, TCR-4, TCR-5, TCR-6, TCR-7, TCR-8, TCR-9, TCR-10, TCR-11, TCR-12, TCR-13, TCR-14, TCR-15, TCR-16, TCR-17, TCR-18, TCR-19, TCR-20, TCR-21, or TCR-22. In some embodiments, the tumor cell antigen comprises MAGEA3, and the beta TCR polypeptide comprises a beta chain of TCR-1, TCR-2, TCR-3, TCR-4, TCR-5, TCR-6, TCR-7, TCR-8, TCR-9, TCR-10, TCR-11, TCR-12, TCR-13, TCR-14, TCR-15, TCR-16, TCR-17, TCR-18, TCR-19, TCR-20, TCR-21, or TCR-22. In some embodiments, the tumor cell antigen comprises MAGEA3, and the alpha TCR polypeptide comprises an amino acid sequence according to SEQ ID NOs: 1, 5, 73, 75, 76, 79, 80, 85, 91, 92, 95, 96, 97, or 98. In some embodiments, the tumor cell antigen comprises MAGEA3, and the beta TCR polypeptide comprises an amino acid sequence according to SEQ ID NOs: 7, 9, 74, 77, 78, 81, 82, 83, 84, 87, 88, 89, 90, 93, or 94.

### Peptide (P₁ and P₂ and P₁ₐ)

In some embodiments, P₁ impairs binding of A₁ to the first target antigen. In some embodiments, P₁ is bound to A₁ through ionic interactions, electrostatic interactions, hydrophobic interactions, Pi-stacking interactions, and H-bonding interactions, or a combination thereof. In some embodiments, P₁ is bound to A₁ at or near an antigen binding site. In some embodiments, P₁ becomes unbound from A₁ when L₁ is cleaved by the tumor specific protease thereby exposing A₁ to the first target antigen. In some embodiments, P₁ has less than 70% sequence homology to the first target antigen. In some embodiments, P₁ has less than 75% sequence homology to the first target antigen. In some embodiments, P₁ has less than 80% sequence homology to the first target antigen. In some embodiments, P₁ has less than 85% sequence homology to the first target antigen. In some embodiments, P₁ has less than 90% sequence homology to the first target antigen. In some embodiments, P₁ has less than 95% sequence homology to the first target antigen. In some embodiments, P₁ has less than 98% sequence homology to the first target antigen. In some embodiments, P₁ has less than 99% sequence homology to the first target antigen.

In some embodiments, P₂ impairs binding of A₂ to the second target antigen. In some embodiments, P₂ is bound to A₂ through ionic interactions, electrostatic interactions, hydrophobic interactions, Pi-stacking interactions, and H-bonding interactions, or a combination thereof. In some embodiments, P₂ is bound to A₂ at or near an antigen binding site. In some embodiments, P₂ becomes unbound from A₂ when L₂ is cleaved by the tumor specific protease thereby exposing A₂ to the second target antigen. In some embodiments, P₂ has less than 70% sequence homology to the second target antigen. In some embodiments, P₂ has less than 75% sequence homology to the second target antigen. In some embodiments, P₂ has less than 80% sequence homology to the second target antigen. In some embodiments, P₂ has less than 85% sequence homology to the second target antigen. In some embodiments, P₂ has less than 90% sequence homology to the second target antigen. In some embodiments, P₂ has less than 95% sequence homology to the second target antigen. In some embodiments, P₂ has less than 98% sequence homology to the second target antigen. In some embodiments, P₂ has less than 99% sequence homology to the second target antigen.

In some embodiments, P₁ₐ when L₁ₐ is uncleaved impairs binding of the antigen recognizing molecule to the target antigen. In some embodiments, P₁ₐ has less than 70% sequence homology to the target antigen. In some embodiments, P₁ₐ has less than 75% sequence homology to the target antigen. In some embodiments, P₁ₐ has less than 80% sequence homology to the target antigen. In some embodiments, P₁ₐ has less than 85% sequence homology to the target antigen. In some embodiments, P₁ₐ has less than 90% sequence homology to the target antigen. In some embodiments, P₁ₐ has less than 95% sequence homology to the target antigen. In some embodiments, P₁ₐ has less than 98% sequence homology to the target antigen. In some embodiments, P₁ₐ has less than 99% sequence homology to the target antigen.

In some embodiments, P₁, P₂, or P₁ₐ comprises a peptide sequence of at least 5 amino acids in length. In some embodiments, P₁, P₂, or P₁ₐ comprises a peptide sequence of at least 6 amino acids in length. In some embodiments, P₁, P₂, or P₁ₐ comprises a peptide sequence of at least 10 amino acids in length. In some embodiments, P₁, P₂, or P₁ₐ comprises a peptide sequence of at least 10 amino acids in length and no more than 20 amino acids in length. In some embodiments, P₁, P₂, or P₁ₐ comprises a peptide sequence of at least 16 amino acids in length. In some embodiments, P₁, P₂, or P₁ₐ comprises a peptide sequence of no more than 40 amino acids in length. In some embodiments, P₁, P₂, or P₁ₐ comprises at least two cysteine amino acid residues. In some embodiments, P₁, P₂, or P₁ₐ comprises an amino acid sequence YDXXF, wherein X is any amino acid. In some embodiments, P₁, P₂, or P₁ₐ comprises an amino acid sequence YDXXF, wherein X is any amino acid except for cysteine. In some embodiments, P₁, P₂, or P₁ₐ comprises an amino acid sequence DVYDEAF (SEQ ID NO: 11). In some embodiments, P₁, P₂, or P₁ₐ comprises an amino sequence according to GGVSCKDVYDEAFCWT (SEQ ID NO: 12) (Peptide-5). In some embodiments, P₁, P₂, or P₁ₐ comprises a cyclic peptide or a linear peptide. In some embodiments, P₁, P₂, or P₁ₐ comprises a cyclic peptide. In some embodiments, P₁, P₂, or P₁ₐ comprises a linear peptide. In some embodiments, the tumor cell antigen comprises MAGEA3, and the and the P₁ or P₂ comprises Peptide-1, Peptide-2, Peptide-3, Peptide-4, Peptide-5, Peptide-6, Peptide-7, Peptide-8, Peptide-9, Peptide-10, Peptide-11, Peptide-12, Peptide-13, Peptide-14, Peptide-15, Peptide-16, Peptide-17, Peptide-18, Peptide-19, Peptide-20, Peptide-21, Peptide-22, Peptide-23, Peptide-24, Peptide-25, Peptide-26, Peptide-27, Peptide-28, Peptide-29, Peptide-30, Peptide-31, Peptide-32, Peptide-33, Peptide-34, Peptide-35, or Peptide-36. In some embodiments, the tumor cell antigen comprises MAGEA3, and the and the P₁ or P₂ comprises an amino acid sequence selected from the group consisting of GGESCQSVYDSSFCYD (SEQ ID NO: 13), GGNACEMTYDHTFCDP (SEQ ID NO: 14), GGRICEEVYDWIFCES (SEQ ID NO: 15), GGRRCVDVYDNAFCLI (SEQ ID NO: 16), GGVSCKDVYDEAFCWT (SEQ ID NO: 12), GGTSCAQIYDFEFCYS (SEQ ID NO: 17), GGSLCSLVYDQDFCES (SEQ ID NO: 18), GGNSCSLVYDKAFCLF (SEQ ID NO: 19), GGNQCWEVYDQEFCSL (SEQ ID NO: 20), GGSACSRIYDFAFCHT (SEQ ID NO: 21), GGTFCYFDHGLVNCQW (SEQ ID NO: 22), GGHCFVSPASGEWWCV (SEQ ID NO: 23), GGCSWIFDGLRYFSKC (SEQ ID NO: 24), VRTWFEKFPELV (SEQ ID NO: 25), LVWGCIWDDMCS (SEQ ID NO: 26), WHWEPSMVWGML (SEQ ID NO: 27), GGGCFVSPATGFTWCV (SEQ ID NO: 28), GGDCQPDSVWSYWYCR (SEQ ID NO: 29), GGCTFVDWWVLGSPYC (SEQ ID NO: 30), GGCLMNDYYYLWGGHC (SEQ ID NO: 31), GGASCKDVYDEAFCWT (SEQ ID NO: 32), GGVACKDVYDEAFCWT (SEQ ID NO: 33), GGVSAKDVYDEAFCWT (SEQ ID NO: 34), GGVSCADVYDEAFCWT (SEQ ID NO: 35), GGVSCKAVYDEAFCWT (SEQ ID NO: 36), GGVSCKDAYDEAFCWT (SEQ ID NO: 37), GGVSCKDVADEAFCWT (SEQ ID NO: 38), GGVSCKDVYAEAFCWT (SEQ ID NO: 39), GGVSCKDVYDAAFCWT (SEQ ID NO: 40), GGVSCKDVYDEAACWT (SEQ ID NO: 41), GGVSCKDVYDEAFAWT (SEQ ID NO: 42), GGVSCKDVYDEAFCAT (SEQ ID NO: 43), GGVSCKDVYDEAFCWA (SEQ ID NO: 44), EVDPIGHLY (SEQ ID NO: 45), ESDPIVAQY (SEQ ID NO: 46), and GGASCAASASAAACAS (SEQ ID NO: 47).

In some embodiments, P₁, P₂, or P₁ₐ or P₁, P₂, and P₁ₐ comprise a modified amino acid or non-natural amino acid, or a modified non-natural amino acid, or a combination thereof. In some embodiments, the modified amino acid or a modified non-natural amino acid comprises a post-translational modification. In some embodiments P₁, P₂, or P₁ₐ or P₁, P₂, and P₁ₐ comprise a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to P₁, P₂, or P₁ₐ or P₁, P₂, and P₁ₐ including the peptide backbone, the amino acid side chains, and the terminus.

In some embodiments, P₁, P₂, or P₁ₐ does not comprise albumin or an albumin fragment. In some embodiments, P₁, P₂, or P₁ₐ does not comprise an albumin binding domain.

### Linking Moiety (L₁, L₂, and L₃)

In some embodiments, L₁, L₂, or L₃ is a peptide sequence having at least 5 to no more than 50 amino acids. In some embodiments, L₁, L₂, or L₃ is a peptide sequence having at least 10 to no more than 30 amino acids. In some embodiments, L₁, L₂, or L₃ is a peptide sequence having at least 10 amino acids. In some embodiments, L₁, L₂, or L₃ is a peptide sequence having at least 18 amino acids. In some embodiments, L₁, L₂, or L₃ is a peptide sequence having at least 26 amino acids. In some embodiments, L₁, L₂, or L₃ has a formula comprising (G₂S)ₙ, wherein n is an integer from 1 to 3 (SEQ ID NO: 48). In some embodiments, L₁, L₂, or L₃ has a formula comprising (G₂S)ₙ, wherein n is an integer of at least 1. In some embodiments, L₁, L₂, or L₃ has a formula selected from the group consisting of (G₂S)ₙ, (GS)ₙ, (GSGGS)ₙ (SEQ ID NO: 49), (GGGS)ₙ (SEQ ID NO: 50), (GGGGS)ₙ (SEQ ID NO: 51), and (GSSGGS)ₙ (SEQ ID NO: 52), wherein n is an integer of at least 1. In some embodiments, the tumor specific protease is selected from the group consisting of metalloprotease, serine protease, cysteine protease, threonine protease, and aspartic protease. In some embodiments, L₁, L₂, or L₃ comprises a urokinase cleavable amino acid sequence, a matriptase cleavable amino acid sequence, or a legumain cleavable amino acid sequence. In some embodiments, L₁, L₂, or L₃ comprises an amino acid sequence selected from the group consisting of GGGGSLSGRSDNHGSSGT (SEQ ID NO: 53), GGGGSSGGSGGSGLSGRSDNHGSSGT (SEQ ID NO: 54), ASGRSDNH (SEQ ID NO: 55), LAGRSDNH (SEQ ID NO: 56), ISSGLASGRSDNH (SEQ ID NO: 57), ISSGLLAGRSDNH (SEQ ID NO: 58), LSGRSDNH (SEQ ID NO: 4), ISSGLLSGRSDNP (SEQ ID NO: 59), ISSGLLSGRSDNH (SEQ ID NO: 60), LSGRSDNHSPLGLAGS (SEQ ID NO: 61), SPLGLAGSLSGRSDNH (SEQ ID NO: 62), SPLGLSGRSDNH (SEQ ID NO: 63), LAGRSDNHSPLGLAGS (SEQ ID NO: 64), LSGRSDNHVPLSLKMG (SEQ ID NO: 65), and LSGRSDNHVPLSLSMG (SEQ ID NO: 66). In some embodiments, L₁, L₂, or L₃ comprises an amino acid sequence ASGRSDNH (SEQ ID NO: 55), LAGRSDNH (SEQ ID NO: 56), ISSGLASGRSDNH (SEQ ID NO: 57), and ISSGLLAGRSDNH (SEQ ID NO: 58). In some embodiments, L₁, L₂, or L₃ comprises an amino acid sequence SSGGGGSGGGS (SEQ ID NO: 67). In some embodiments, L₁, L₂, or L₃ is Linker-1, Linker-2, Linker-3, Linker-4, Linker-5, Linker-6, Linker-7, Linker-8, Linker-9, Linker-10, Linker-11, Linker-12, Linker-13, Linker-14, Linker-15, Linker-16, Linker-17, Linker-18, or Linker-19. In some embodiments, L₁, L₂, or L₃ comprises an amino acid sequence GGGGSLSGRSDNHGSSGT (SEQ ID NO: 53), GGGGSSGGSGGSGLSGRSDNHGSSGT (SEQ ID NO: 54), ASGRSDNH (SEQ ID NO: 55), LAGRSDNH (SEQ ID NO: 56), ISSGLASGRSDNH (SEQ ID NO: 57), ISSGLLAGRSDNH (SEQ ID NO: 58), LSGRSDNH (SEQ ID NO: 4), ISSGLLSGRSDNP (SEQ ID NO: 59), ISSGLLSGRSDNH (SEQ ID NO: 60), LSGRSDNHSPLGLAGS (SEQ ID NO: 61), SPLGLAGSLSGRSDNH (SEQ ID NO: 62), SPLGLSGRSDNH (SEQ ID NO: 63), LAGRSDNHSPLGLAGS (SEQ ID NO: 64), LSGRSDNHVPLSLKMG (SEQ ID NO: 65), LSGRSDNHVPLSLSMG (SEQ ID NO: 66), GSSGGSGGSGGSGLSGRSDNHGSSGT (SEQ ID NO: 68), GSSGGSGGSGGSGGGSGGGSGGSSGT (SEQ ID NO: 69), GGGGSGGSGGGSGGSSGT (SEQ ID NO: 70), and GGGGSGGGS (SEQ ID NO: 71).

In some embodiments, L₁, L₂, or L₃ or L₁, L₂, and L₃ comprise a modified amino acid or non-natural amino acid, or a modified non-natural amino acid, or a combination thereof. In some embodiments, the modified amino acid or a modified non-natural amino acid comprises a post-translational modification. In some embodiments, L₁, L₂, or L₃ or L₁, L₂, and L₃ comprise a modification including, but not limited, to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to L₁, L₂, or L₃ or L₁, L₂, and L₃ including the peptide backbone, or the amino acid side chains.

### Half-life Extending Molecule (H₁ and H₁ₐ₎

In some embodiments, H₁ does not block A₁ binding to the first target antigen. In some embodiments, H₁ₐ does not block antigen recognizing molecule binding to the target antigen. In some embodiments, half-life extending molecule (H₁ or H₁ₐ) does not have binding affinity to antigen recognizing molecule. In some embodiments, half-life extending molecule (H₁ or H₁ₐ) does not have binding affinity to the target antigen. In some embodiments, half-life extending molecule (H₁ or H₁ₐ) does not shield antigen recognizing molecule from the target antigen. In some embodiments, half-life extending molecule (H₁ or H₁ₐ) is not directly linked to antigen recognizing molecule.

In some embodiments, H₁ or H₁ₐ comprise an amino acid sequence that has repetitive sequence motifs. In some embodiments, H₁ or H₁ₐ comprises an amino acid sequence that has highly ordered secondary structure. “Highly ordered secondary structure,” as used in this context, means that at least about 50%, or about 70%, or about 80%, or about 90%, of amino acid residues of H₁ or H₁ₐ contribute to secondary structure, as measured or determined by means, including, but not limited to, spectrophotometry (e.g. by circular dichroism spectroscopy in the “far-UV” spectral region (190-250 nm), and computer programs or algorithms, such as the Chou-Fasman algorithm and the Garnier-Osguthorpe-Robson (“GOR”) algorithm.

In some embodiments, H₁ or H₁ₐ comprises a polymer. In some embodiments, the polymer is polyethylene glycol (PEG). In some embodiments, H₁ or H₁ₐ comprises albumin. In some embodiments, H₁ or H₁ₐ comprises an Fc domain. In some embodiments, the albumin is serum albumin. In some embodiments, the albumin is human serum albumin. In some embodiments, H₁ or H₁ₐ comprises a polypeptide, a ligand, or a small molecule. In some embodiments, the polypeptide, the ligand or the small molecule binds serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgG1, IgG2, IgG3, IgG4, slgA, IgM or IgD. In some embodiments, the serum protein is albumin. In some embodiments, the polypeptide is an antibody. In some embodiments, the antibody comprises a single domain antibody, a single chain variable fragment or a Fab. In some embodiments, the antibody is a human or humanized antibody. In some embodiments, the antibody is selected from the group consisting of 645gH1gL1, 645dsgH5gL4, 23-13-A01 -sc02, A10m3 or a fragment thereof, DOM7r-31, DOM7h-11-15, Alb-1, Alb-8, Alb-23, 10G, 10GE, and SA21. In some embodiments, the single domain antibody is 10G, and the single domain antibody comprises an amino acid sequence

MEVQLVESGGGLVQPGNSLRLSCAASGFTFSKFGMSWVRQAPGKGLEWVS
SISGSGRDTLYADSVKGRFTISRDNAKTTLYLQMNSLRPEDTAVYYCTIG
GSLSVSSQGTLVTVSS (SEQ ID NO: 2).

In some embodiments, the single domain antibody is 10G, and the single domain antibody comprises an amino acid sequence

EVQLVESGGGLVQPGNSLRLSCAASGFTFSKFGMSWVRQAPGKGLEWVSS
ISGSGRDTLYADSVKGRFTISRDNAKTTLYLQMNSLRPEDTAVYYCTIGG
SLSVSSQGTLVTVSS (SEQ ID NO: 72).

In some embodiments, H₁ or H₁ₐ or H₁ and H₁ₐ comprise a modified amino acid or non-natural amino acid, or a modified non-natural amino acid, or a combination thereof. In some embodiments, the modified amino acid or a modified non-natural amino acid comprises a post-translational modification. In some embodiments H₁ or H₁ₐ or H₁ and H₁ₐ comprise a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to H₁ or H₁ₐ or H₁ and H₁ₐ including the peptide backbone, the amino acid side chains, and the terminus.

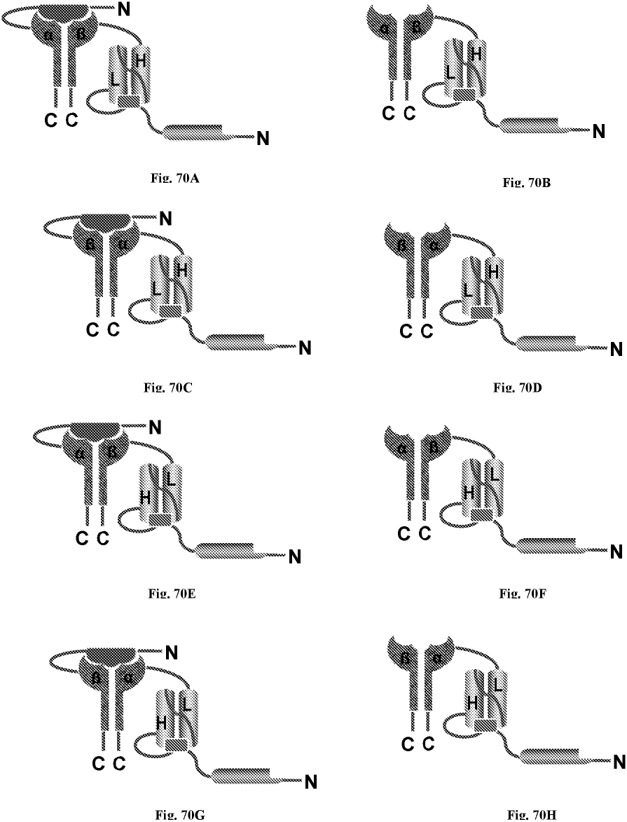

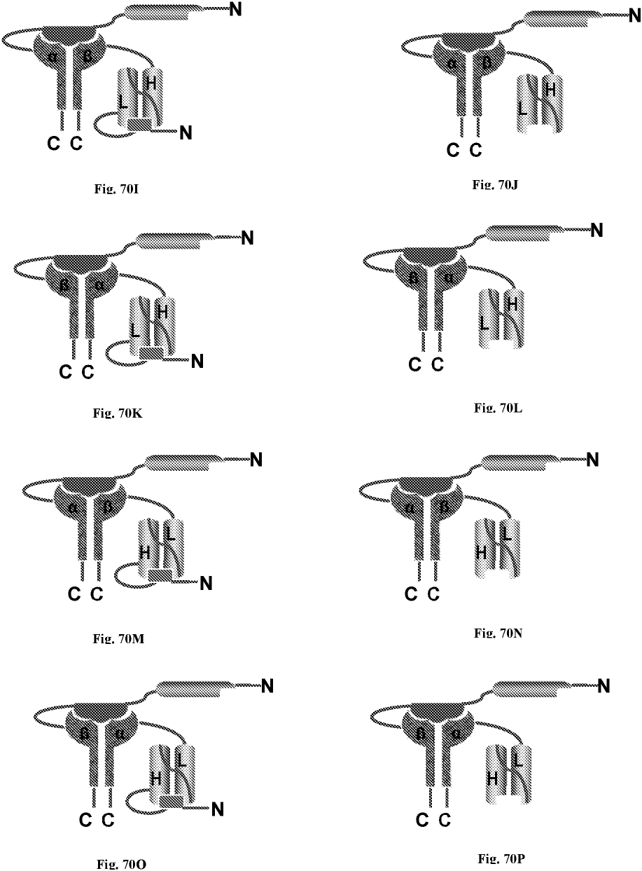

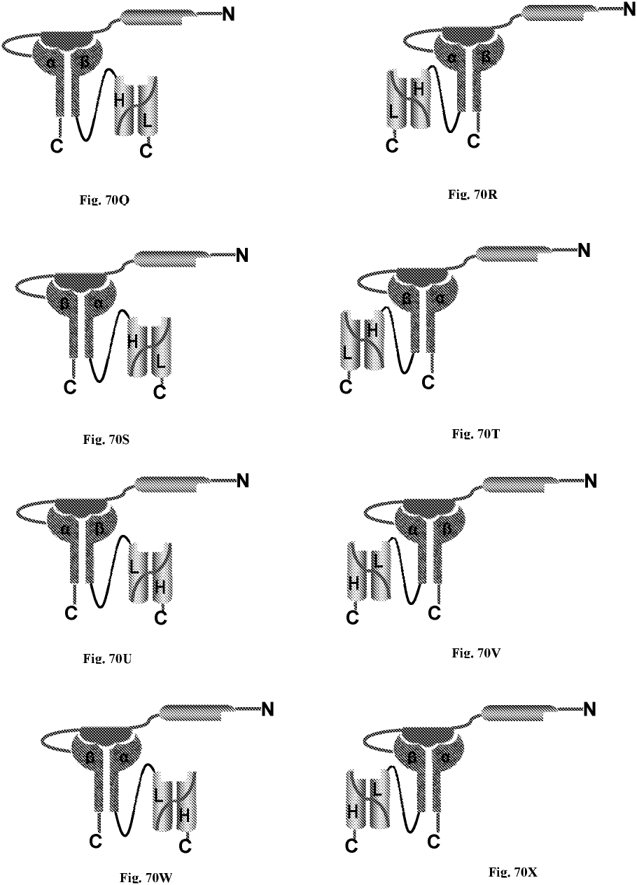

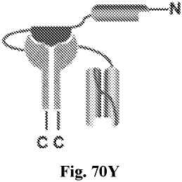

Disclosed herein, in some embodiments, are polypeptides or polypeptide complexes comprising a structural arrangement according to the configuration shown in FIG. 70A, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the beta TCR polypeptide is linked to a C terminus of the heavy chain variable domain of the scFv, and wherein the soluble TCR is linked to P₂ and L₂, wherein P₂ comprises a peptide that impairs binding of the soluble TCR to the tumor cell antigen; and L₂ comprises a linking moiety that connects the alpha TCR polypeptide to P₂ and is a substrate for a tumor specific protease.

Disclosed herein, in some embodiments, are polypeptides or polypeptide complexes comprising a structural arrangement according to the configuration shown in FIG. 70B, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the beta TCR polypeptide is linked to a C terminus of the heavy chain variable domain of the scFv.

Disclosed herein, in some embodiments, are polypeptides or polypeptide complexes comprising a structural arrangement according to the configuration shown in FIG. 70C, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the alpha TCR polypeptide is linked to a C terminus of the heavy chain variable domain of the scFv, and wherein the soluble TCR is linked to P₂ and L₂, wherein P₂ comprises a peptide that impairs binding of the soluble TCR to the tumor cell antigen; and L₂ comprises a linking moiety that connects the beta TCR polypeptide to P₂ and is a substrate for a tumor specific protease.

Disclosed herein, in some embodiments, are polypeptides or polypeptide complexes comprising a structural arrangement according to the configuration shown in FIG. 70D, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the alpha TCR polypeptide is linked to a C terminus of the heavy chain variable domain of the scFv.

Disclosed herein, in some embodiments, are polypeptides or polypeptide complexes comprising a structural arrangement according to the configuration shown in FIG. 70E, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the beta TCR polypeptide is linked to a C terminus of the light chain variable domain of the scFv, and wherein the soluble TCR is linked to P₂ and L₂, wherein P₂ comprises a peptide that impairs binding of the soluble TCR to the tumor cell antigen; and L₂ comprises a linking moiety that connects the alpha TCR polypeptide to P₂ and is a substrate for a tumor specific protease.

Disclosed herein, in some embodiments, are polypeptides or polypeptide complexes comprising a structural arrangement according to the configuration shown in FIG. 70F, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the beta TCR polypeptide is linked to a C terminus of the light chain variable domain of the scFv.

Disclosed herein, in some embodiments, are polypeptides or polypeptide complexes comprising a structural arrangement according to the configuration shown in FIG. 70G, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the alpha TCR polypeptide is linked to a C terminus of the light chain variable domain of the scFv, and wherein the soluble TCR is linked to P₂ and L₂, wherein P₂ comprises a peptide that impairs binding of the soluble TCR to the tumor cell antigen; and L₂ comprises a linking moiety that connects the beta TCR polypeptide to P₂ and is a substrate for a tumor specific protease.

Disclosed herein, in some embodiments, are polypeptides or polypeptide complexes comprising a structural arrangement according to the configuration shown in FIG. 70H, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the alpha TCR polypeptide is linked to a C terminus of the light chain variable domain of the scFv.

Disclosed herein, in some embodiments, are polypeptides or polypeptide complexes comprising a structural arrangement according to the configuration shown in FIG. 70I, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to an N-terminus of the beta TCR polypeptide, and wherein the scFv is linked to P₂ and L₂, wherein P₂ comprises a peptide that impairs binding of the scFv to an effector cell antigen; and L₂ comprises a linking moiety that connects the light chain variable domain of the scFv to P₂ and is a substrate for a tumor specific protease.

Disclosed herein, in some embodiments, are polypeptides or polypeptide complexes comprising a structural arrangement according to the configuration shown in FIG. 70J, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to an N-terminus of the beta TCR polypeptide and the scFv binds to an effector cell antigen.

Disclosed herein, in some embodiments, are polypeptides or polypeptide complexes comprising a structural arrangement according to the configuration shown in FIG. 70K, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to an N-terminus of the alpha TCR polypeptide, and wherein the scFv is linked to P₂ and L₂, wherein P₂ comprises a peptide that impairs binding of the scFv to an effector cell antigen; and L₂ comprises a linking moiety that connects the light chain variable domain of the scFv to P₂ and is a substrate for a tumor specific protease.

Disclosed herein, in some embodiments, are polypeptides or polypeptide complexes comprising a structural arrangement according to the configuration shown in FIG. 70L, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to an N-terminus of the alpha TCR polypeptide and the scFv binds to an effector cell antigen.

Disclosed herein, in some embodiments, are polypeptides or polypeptide complexes comprising a structural arrangement according to the configuration shown in FIG. 70M, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to an N-terminus of the beta TCR polypeptide, and wherein the scFv is linked to P₂ and L₂, wherein P₂ comprises a peptide that impairs binding of the scFv to an effector cell antigen; and L₂ comprises a linking moiety that connects the heavy chain variable domain of the scFv to P₂ and is a substrate for a tumor specific protease.

Disclosed herein, in some embodiments, are polypeptides or polypeptide complexes comprising a structural arrangement according to the configuration shown in FIG. 70N, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to an N-terminus of the beta TCR polypeptide and the scFv binds to an effector cell antigen.

Disclosed herein, in some embodiments, are polypeptides or polypeptide complexes comprising a structural arrangement according to the configuration shown in FIG. 70O, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to an N-terminus of the alpha TCR polypeptide, and wherein the scFv is linked to P₂ and L₂, wherein P₂ comprises a peptide that impairs binding of the scFv to an effector cell antigen; and L₂ comprises a linking moiety that connects the heavy chain variable domain of the scFv to P₂ and is a substrate for a tumor specific protease.

Disclosed herein, in some embodiments, are polypeptides or polypeptide complexes comprising a structural arrangement according to the configuration shown in FIG. 70P, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to an N-terminus of the alpha TCR polypeptide and the scFv binds to an effector cell antigen.

Disclosed herein, in some embodiments, are polypeptides or polypeptide complexes comprising a structural arrangement according to the configuration shown in FIG. 70Q, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to a C-terminus of the beta TCR polypeptide and the scFv binds to an effector cell antigen.

Disclosed herein, in some embodiments, are polypeptides or polypeptide complexes comprising a structural arrangement according to the configuration shown in FIG. 70R, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to a C-terminus of the alpha TCR polypeptide and the scFv binds to an effector cell antigen.

Disclosed herein, in some embodiments, are polypeptides or polypeptide complexes comprising a structural arrangement according to the configuration shown in FIG. 70S, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to a C-terminus of the alpha TCR polypeptide and the scFv binds to an effector cell antigen.

Disclosed herein, in some embodiments, are polypeptides or polypeptide complexes comprising a structural arrangement according to the configuration shown in FIG. 70T, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to a C-terminus of the beta TCR polypeptide and the scFv binds to an effector cell antigen.

Disclosed herein, in some embodiments, are polypeptides or polypeptide complexes comprising a structural arrangement according to the configuration shown in FIG. 70U, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to a C-terminus of the beta TCR polypeptide and the scFv binds to an effector cell antigen.

Disclosed herein, in some embodiments, are polypeptides or polypeptide complexes comprising a structural arrangement according to the configuration shown in FIG. 70V, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to a C-terminus of the alpha TCR polypeptide and the scFv binds to an effector cell antigen.

Disclosed herein, in some embodiments, are polypeptides or polypeptide complexes comprising a structural arrangement according to the configuration shown in FIG. 70W, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to a C-terminus of the alpha TCR polypeptide and the scFv binds to an effector cell antigen.

Disclosed herein, in some embodiments, are polypeptides or polypeptide complexes comprising a structural arrangement according to the configuration shown in FIG. 70X, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to a C-terminus of the beta TCR polypeptide and the scFv binds to an effector cell antigen.

### Polynucleotides Encoding Polypeptides or Polypeptide Complexes

Disclosed herein, in some embodiments, are isolated recombinant nucleic acid molecules encoding polypeptides or polypeptide complexes as disclosed herein. In some embodiments, the polypeptides or polypeptide complexes comprise a T cell receptor (TCR). In some embodiments, the polypeptides or polypeptide complexes comprise an antibody or an antibody fragment. In some embodiments, the polypeptides or polypeptide complexes comprise a T cell receptor (TCR) and an antibody or an antibody fragment.

Disclosed herein, in some embodiments, are isolated recombinant nucleic acid molecules encoding polypeptides or polypeptide complexes according to Formula I:

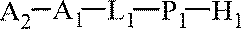

wherein A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to a second target antigen. Disclosed herein, in some embodiments, are isolated recombinant nucleic acid molecules encoding polypeptides or polypeptide complexes comprising Formula I:

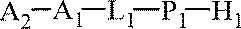

wherein A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to a second target antigen. Disclosed herein, in some embodiments, are isolated recombinant nucleic acid molecules encoding polypeptides or polypeptide complexes comprising Formula I:

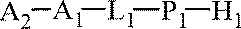

wherein A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to a second target antigen. Disclosed herein, in some embodiments, are isolated recombinant nucleic acid molecules encoding polypeptides or polypeptide complexes according to Formula I:

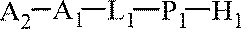

wherein A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to a second target antigen.

Disclosed herein, in some embodiments, are isolated recombinant nucleic acid molecules encoding polypeptides or polypeptide complexes according to Formula Ia:

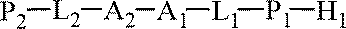

wherein: A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; A₂ is a second antigen recognizing molecule that binds to a second target antigen; P₂ is a peptide that binds to A₂; and L₂ is a linking moiety that connects A₂ to P₂ and is a substrate for a tumor specific protease.

Disclosed herein, in some embodiments, are isolated recombinant nucleic acid molecules encoding polypeptides or polypeptide complexes comprising Formula Ia:

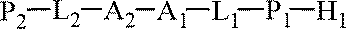

wherein: A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; A₂ comprises a second antigen recognizing molecule that binds to a second target antigen; P₂ comprises a peptide that binds to A₂; and L₂ comprises a linking moiety that connects A₂ to P₂ and is a substrate for a tumor specific protease.

Disclosed herein, in some embodiments, are isolated recombinant nucleic acid molecules encoding polypeptides or polypeptide complexes comprising Formula Ia:

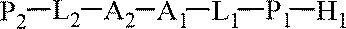

wherein: A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; A₂ is a second antigen recognizing molecule that binds to a second target antigen P₂ is a peptide that binds to A₂; and L₂ is a linking moiety that connects A₂ to P₂ and is a substrate for a tumor specific protease.

In some embodiments, L₁ is bound to N-terminus of the alpha TCR polypeptide. In some embodiments, L₁ is bound to N-terminus of the beta TCR polypeptide. In some embodiments, A₂ is bound to C-terminus of the alpha TCR polypeptide. In some embodiments, A₂ is bound to N-terminus of the alpha TCR polypeptide. In some embodiments, A₂ is bound to C-terminus of the beta TCR polypeptide. In some embodiments, A₂ is bound to N-terminus of the beta TCR polypeptide. In some embodiments, L₁ is bound to N-terminus of the alpha TCR polypeptide and A₂ is bound to N-terminus of the beta TCR polypeptide. In some embodiments, L₁ is bound to N-terminus of the alpha TCR polypeptide and A₂ is bound to C-terminus of the beta TCR polypeptide. In some embodiments, L₁ is bound to N-terminus of the alpha TCR polypeptide and A₂ is bound to C-terminus of the alpha TCR polypeptide. In some embodiments, L₁ is bound to N-terminus of the beta TCR polypeptide and A₂ is bound to N-terminus of the alpha TCR polypeptide. In some embodiments, L₁ is bound to N-terminus of the beta TCR polypeptide and A₂ is bound to C-terminus of the beta TCR polypeptide. In some embodiments, L₁ is bound to N-terminus of the beta TCR polypeptide and A₂ is bound to C-terminus of the alpha TCR polypeptide. In some embodiments, the alpha TCR polypeptide of A₁ is bound to a C-terminus of the single chain variable fragment (scFv) of A₂. In some embodiments, the beta TCR polypeptide of A₁ is bound to a C-terminus of the single chain variable fragment (scFv) A₂. In some embodiments, the alpha TCR polypeptide of A₁ is bound to a N-terminus of the single chain variable fragment (scFv) of A₂. In some embodiments, the beta TCR polypeptide of A₁ is bound to a N-terminus of the single chain variable fragment (scFv) A₂. In some embodiments, the beta TCR polypeptide of A₁ is bound to the scFv heavy chain polypeptide of A₂ and L₁ is bound to the alpha TCR polypeptide of A₁. In some embodiments, the beta TCR polypeptide of A₁ is bound to the scFv heavy chain polypeptide of A₂ at the C-terminus of the scFv and L₁ is bound to the alpha TCR polypeptide of A₁ at the N-terminus of the alpha TCR polypeptide. In some embodiments, the beta TCR polypeptide of A₁ is bound to the scFv heavy chain polypeptide of A₂ at the C-terminus of the scFv and L₁ is bound to the alpha TCR polypeptide of A₁ at the N-terminus of the alpha TCR polypeptide and the polypeptide complex comprises amino acid sequences of (TCR-20-alpha and TCR-20 -beta. In some embodiments, the beta TCR polypeptide of A₁ is bound to the scFv heavy chain polypeptide of A₂ at the N-terminus of the scFv and L₁ is bound to the alpha TCR polypeptide of A₁ at the N-terminus of the alpha TCR polypeptide. In some embodiments, the alpha TCR polypeptide of A₁ is bound to the scFv heavy chain polypeptide of A₂ and L₁ is bound to the beta TCR polypeptide of A₁ In some embodiments, the alpha TCR polypeptide of A₁ is bound to the scFv heavy chain polypeptide of A₂ at the C-terminus of the scFv and L₁ is bound to the beta TCR polypeptide of A₁ at the N-terminus of the beta TCR polypeptide. In some embodiments, the alpha TCR polypeptide of A₁ is bound to the scFv heavy chain polypeptide of A₂ at the N-terminus of the scFv and L₁ is bound to the beta TCR polypeptide of A₁ at the N-terminus of the beta TCR polypeptide. In some embodiments, the beta TCR polypeptide of A₁ is bound to the scFv light chain polypeptide of A₂ and L₁ is bound to the alpha TCR polypeptide of A₁ In some embodiments, the beta TCR polypeptide of A₁ is bound to the scFv light chain polypeptide of A₂ at the C-terminus of the scFv and L₁ is bound to the alpha TCR polypeptide of A₁ at the N-terminus of the alpha TCR polypeptide. In some embodiments, the beta TCR polypeptide of A₁ is bound to the scFv light chain polypeptide of A₂ at the N-terminus of the scFv and L₁ is bound to the alpha TCR polypeptide of A₁ at the N-terminus of the alpha TCR polypeptide In some embodiments, the alpha TCR polypeptide of A₁ is bound to the scFv light chain polypeptide of A₂ and L₁ is bound to the beta TCR polypeptide of A₁. In some embodiments, the alpha TCR polypeptide of A₁ is bound to the scFv light chain polypeptide of A₂ at the C-terminus of the scFv and L₁ is bound to the beta TCR polypeptide of A₁ at the N-terminus of the beta TCR polypeptide. In some embodiments, the alpha TCR polypeptide of A₁ is bound to the scFv light chain polypeptide of A₂ at the N-terminus of the scFv and L₁ is bound to the beta TCR polypeptide of A₁ at the N-terminus of the beta TCR polypeptide. In some embodiments, the alpha TCR polypeptide of A₁ is bound to the scFv heavy chain polypeptide of A₂ at the N-terminus of the scFv and L₁ is bound to the alpha TCR polypeptide of A₁ at the N-terminus of the alpha TCR polypeptide. In some embodiments, the beta TCR polypeptide of A₁ is bound to the scFv heavy chain polypeptide of A₂ at the N-terminus of the scFv and L₁ is bound to the beta TCR polypeptide of A₁ at the N-terminus of the beta TCR polypeptide. In some embodiments, the alpha TCR polypeptide of A₁ is bound to the scFv light chain polypeptide of A₂ at the N-terminus of the scFv and L₁ is bound to the alpha TCR polypeptide of A₁ at the N-terminus of the alpha TCR polypeptide. In some embodiments, the beta TCR polypeptide of A₁ is bound to the scFv light chain polypeptide of A₂ at the N-terminus of the scFv and L₁ is bound to the beta TCR polypeptide of A₁ at the N-terminus of the beta TCR polypeptide. In some embodiments, A₂ further comprises P₂ and L₂, wherein P₂ comprises a peptide that binds to A₂; and L₂ comprises a linking moiety that connects A₂ to P₂ and is a substrate for a tumor specific protease.

Disclosed herein, in some embodiments, are isolated recombinant nucleic acid molecules encoding polypeptides or polypeptide complexes according to Formula Ia:

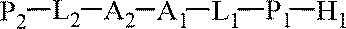

Disclosed herein, in some embodiments, are isolated recombinant nucleic acid molecules encoding polypeptides or polypeptide complexes comprising Formula Ia:

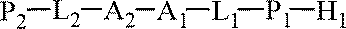

In some embodiments, the beta TCR polypeptide of A₁ is bound to the scFv heavy chain polypeptide of A₂ and L₁ is bound to the alpha TCR polypeptide of A₁ and L₂ is bound to the scFv light chain polypeptide of A₂. In some embodiments, the beta TCR polypeptide of A₁ is bound to the scFv heavy chain polypeptide of A₂ at the C-terminus of the scFv and L₁ is bound to the alpha TCR polypeptide of A₁ at the N-terminus of the alpha TCR polypeptide and L₂ is bound to the scFv light chain polypeptide of A₂ at the N-terminus of the scFv. In some embodiments, the alpha TCR polypeptide of A₁ is bound to the scFv heavy chain polypeptide of A₂ and L₁ is bound to the beta TCR polypeptide of A₁ and L₂ is bound to the scFv light chain polypeptide of A₂. In some embodiments, the alpha TCR polypeptide of A₁ is bound to the scFv heavy chain polypeptide of A₂ at the C-terminus of the scFv and L₁ is bound to the beta TCR polypeptide of A₁ at the N-terminus of the beta TCR polypeptide and L₂ is bound to the scFv light chain polypeptide of A₂ at the N-terminus of the scFv. In some embodiments, the beta TCR polypeptide of A₁ is bound to the scFv light chain polypeptide of A₂ and L₁ is bound to the alpha TCR polypeptide of A₁ and L₂ is bound to the scFv heavy chain polypeptide of A₂. In some embodiments, the beta TCR polypeptide of A₁ is bound to the scFv light chain polypeptide of A₂ at the C-terminus of the scFv and L₁ is bound to the alpha TCR polypeptide of A₁ at the N-terminus of the alpha TCR polypeptide and L₂ is bound to the scFv heavy chain polypeptide of A₂ at the N-terminus of the scFv. In some embodiments, the alpha TCR polypeptide of A₁ is bound to the scFv light chain polypeptide of A₂ and L₁ is bound to the beta TCR polypeptide of A₁ and L₂ is bound to the scFv heavy chain polypeptide of A₂. In some embodiments, the alpha TCR polypeptide of A₁ is bound to the scFv light chain polypeptide of A₂ at the C-terminus of the scFv and L₁ is bound to the beta TCR polypeptide of A₁ at the N-terminus of the beta TCR polypeptide and L₂ is bound to the scFv heavy chain polypeptide of A₂ at the N-terminus of the scFv.

Disclosed herein, in some embodiments, are isolated recombinant nucleic acid molecules encoding polypeptides or polypeptide complexes comprising a structural arrangement according to the configuration shown in FIG. 70A, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the beta TCR polypeptide is linked to a C terminus of the heavy chain variable domain of the scFv, and wherein the soluble TCR is linked to P₂ and L₂, wherein P₂ comprises a peptide that impairs binding of the soluble TCR to the tumor cell antigen; and L₂ comprises a linking moiety that connects the alpha TCR polypeptide to P₂ and is a substrate for a tumor specific protease.

Disclosed herein, in some embodiments, are isolated recombinant nucleic acid molecules encoding polypeptides or polypeptide complexes comprising a structural arrangement according to the configuration shown in FIG. 70B, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the beta TCR polypeptide is linked to a C terminus of the heavy chain variable domain of the scFv.

Disclosed herein, in some embodiments, are isolated recombinant nucleic acid molecules encoding polypeptides or polypeptide complexes comprising a structural arrangement according to the configuration shown in FIG. 70C, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the alpha TCR polypeptide is linked to a C terminus of the heavy chain variable domain of the scFv, and wherein the soluble TCR is linked to P₂ and L₂, wherein P₂ comprises a peptide that impairs binding of the soluble TCR to the tumor cell antigen; and L₂ comprises a linking moiety that connects the beta TCR polypeptide to P₂ and is a substrate for a tumor specific protease.

Disclosed herein, in some embodiments, are isolated recombinant nucleic acid molecules encoding polypeptides or polypeptide complexes comprising a structural arrangement according to the configuration shown in FIG. 70D, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the alpha TCR polypeptide is linked to a C terminus of the heavy chain variable domain of the scFv.

Disclosed herein, in some embodiments, are isolated recombinant nucleic acid molecules encoding polypeptides or polypeptide complexes comprising a structural arrangement according to the configuration shown in FIG. 70E, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the beta TCR polypeptide is linked to a C terminus of the light chain variable domain of the scFv, and wherein the soluble TCR is linked to P₂ and L₂, wherein P₂ comprises a peptide that impairs binding of the soluble TCR to the tumor cell antigen; and L₂ comprises a linking moiety that connects the alpha TCR polypeptide to P₂ and is a substrate for a tumor specific protease.

Disclosed herein, in some embodiments, are isolated recombinant nucleic acid molecules encoding polypeptides or polypeptide complexes comprising a structural arrangement according to the configuration shown in FIG. 70F, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the beta TCR polypeptide is linked to a C terminus of the light chain variable domain of the scFv.

Disclosed herein, in some embodiments, are isolated recombinant nucleic acid molecules encoding polypeptides or polypeptide complexes comprising a structural arrangement according to the configuration shown in FIG. 70G, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the alpha TCR polypeptide is linked to a C terminus of the light chain variable domain of the scFv, and wherein the soluble TCR is linked to P₂ and L₂, wherein P₂ comprises a peptide that impairs binding of the soluble TCR to the tumor cell antigen; and L₂ comprises a linking moiety that connects the beta TCR polypeptide to P₂ and is a substrate for a tumor specific protease.

Disclosed herein, in some embodiments, are isolated recombinant nucleic acid molecules encoding polypeptides or polypeptide complexes comprising a structural arrangement according to the configuration shown in FIG. 70H, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the alpha TCR polypeptide is linked to a C terminus of the light chain variable domain of the scFv.

Disclosed herein, in some embodiments, are isolated recombinant nucleic acid molecules encoding polypeptides or polypeptide complexes comprising a structural arrangement according to the configuration shown in FIG. 70I, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to an N-terminus of the beta TCR polypeptide, and wherein the scFv is linked to P₂ and L₂, wherein P₂ comprises a peptide that impairs binding of the scFv to an effector cell antigen; and L₂ comprises a linking moiety that connects the light chain variable domain of the scFv to P₂ and is a substrate for a tumor specific protease.

Disclosed herein, in some embodiments, are isolated recombinant nucleic acid molecules encoding polypeptides or polypeptide complexes comprising a structural arrangement according to the configuration shown in FIG. 70J, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to an N-terminus of the beta TCR polypeptide and the scFv binds to an effector cell antigen.

Disclosed herein, in some embodiments, are isolated recombinant nucleic acid molecules encoding polypeptides or polypeptide complexes comprising a structural arrangement according to the configuration shown in FIG. 70K, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to an N-terminus of the alpha TCR polypeptide, and wherein the scFv is linked to P₂ and L₂, wherein P₂ comprises a peptide that impairs binding of the scFv to an effector cell antigen; and L₂ comprises a linking moiety that connects the light chain variable domain of the scFv to P₂ and is a substrate for a tumor specific protease.

Disclosed herein, in some embodiments, are isolated recombinant nucleic acid molecules encoding polypeptides or polypeptide complexes comprising a structural arrangement according to the configuration shown in FIG. 70L, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to an N-terminus of the alpha TCR polypeptide and the scFv binds to an effector cell antigen.

Disclosed herein, in some embodiments, are isolated recombinant nucleic acid molecules encoding polypeptides or polypeptide complexes comprising a structural arrangement according to the configuration shown in FIG. 70M, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to an N-terminus of the beta TCR polypeptide, and wherein the scFv is linked to P₂ and L₂, wherein P₂ comprises a peptide that impairs binding of the scFv to an effector cell antigen; and L₂ comprises a linking moiety that connects the heavy chain variable domain of the scFv to P₂ and is a substrate for a tumor specific protease.

Disclosed herein, in some embodiments, are isolated recombinant nucleic acid molecules encoding polypeptides or polypeptide complexes comprising a structural arrangement according to the configuration shown in FIG. 70N, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to an N-terminus of the beta TCR polypeptide and the scFv binds to an effector cell antigen.

Disclosed herein, in some embodiments, are isolated recombinant nucleic acid molecules encoding polypeptides or polypeptide complexes comprising a structural arrangement according to the configuration shown in FIG. 70O, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to an N-terminus of the alpha TCR polypeptide, and wherein the scFv is linked to P₂ and L₂, wherein P₂ comprises a peptide that impairs binding of the scFv to an effector cell antigen; and L₂ comprises a linking moiety that connects the heavy chain variable domain of the scFv to P₂ and is a substrate for a tumor specific protease.

Disclosed herein, in some embodiments, are isolated recombinant nucleic acid molecules encoding polypeptides or polypeptide complexes comprising a structural arrangement according to the configuration shown in FIG. 70P, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to an N-terminus of the alpha TCR polypeptide and the scFv binds to an effector cell antigen.

Disclosed herein, in some embodiments, are isolated recombinant nucleic acid molecules encoding polypeptides or polypeptide complexes comprising a structural arrangement according to the configuration shown in FIG. 70Q, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to a C-terminus of the beta TCR polypeptide and the scFv binds to an effector cell antigen.

Disclosed herein, in some embodiments, are isolated recombinant nucleic acid molecules encoding polypeptides or polypeptide complexes comprising a structural arrangement according to the configuration shown in FIG. 70R, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to a C-terminus of the alpha TCR polypeptide and the scFv binds to an effector cell antigen.

Disclosed herein, in some embodiments, are isolated recombinant nucleic acid molecules encoding polypeptides or polypeptide complexes comprising a structural arrangement according to the configuration shown in FIG. 70S, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to a C-terminus of the alpha TCR polypeptide and the scFv binds to an effector cell antigen.

Disclosed herein, in some embodiments, are isolated recombinant nucleic acid molecules encoding polypeptides or polypeptide complexes comprising a structural arrangement according to the configuration shown in FIG. 70T, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to a C-terminus of the beta TCR polypeptide and the scFv binds to an effector cell antigen.

Disclosed herein, in some embodiments, are isolated recombinant nucleic acid molecules encoding polypeptides or polypeptide complexes comprising a structural arrangement according to the configuration shown in FIG. 70U, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to a C-terminus of the beta TCR polypeptide and the scFv binds to an effector cell antigen.

Disclosed herein, in some embodiments, are isolated recombinant nucleic acid molecules encoding polypeptides or polypeptide complexes comprising a structural arrangement according to the configuration shown in FIG. 70V, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to a C-terminus of the alpha TCR polypeptide and the scFv binds to an effector cell antigen.

Disclosed herein, in some embodiments, are isolated recombinant nucleic acid molecules encoding polypeptides or polypeptide complexes comprising a structural arrangement according to the configuration shown in FIG. 70W, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to a C-terminus of the alpha TCR polypeptide and the scFv binds to an effector cell antigen.

Disclosed herein, in some embodiments, are isolated recombinant nucleic acid molecules encoding polypeptides or polypeptide complexes comprising a structural arrangement according to the configuration shown in FIG. 70X, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to a C-terminus of the beta TCR polypeptide and the scFv binds to an effector cell antigen.

### Pharmaceutical Compositions

Disclosed herein, in some embodiments, are pharmaceutical compositions comprising: (a) the polypeptides or polypeptide complexes as disclosed herein; and (b) a pharmaceutically acceptable excipient.

In some embodiments, the pharmaceutical composition comprises (a) polypeptides or polypeptide complexes according to Formula I:

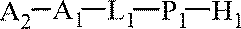

wherein A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to a second target antigen; and (b) a pharmaceutically acceptable excipient. In some embodiments, the pharmaceutical composition comprises (a) polypeptides or polypeptide complexes comprising Formula I:

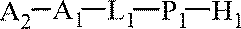

wherein A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to a second target antigen; and (b) a pharmaceutically acceptable excipient. In some embodiments, the pharmaceutical composition comprises (a) polypeptides or polypeptide complexes comprising Formula I:

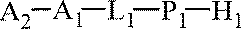

wherein A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to a second target antigen; and (b) a pharmaceutically acceptable excipient. In some embodiments, the pharmaceutical composition comprises (a) polypeptides or polypeptide complexes according to Formula I:

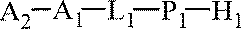

wherein A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to a second target antigen; and (b) a pharmaceutically acceptable excipient.

In some embodiments, the pharmaceutical composition comprises (a) polypeptides or polypeptide complexes according to Formula Ia:

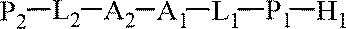

wherein: A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; A₂ comprises a second antigen recognizing molecule that binds to a second target antigen; P₂ comprises a peptide that binds to A₂; and L₂ comprises a linking moiety that connects A₂ to P₂ and is a substrate for a tumor specific protease; and (b) a pharmaceutically acceptable excipient.

In some embodiments, the pharmaceutical composition comprises (a) polypeptides or polypeptide complexes according to Formula Ia:

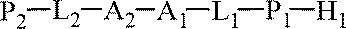

wherein: A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; A₂ is a second antigen recognizing molecule that binds to a second target antigen; P₂ is a peptide that binds to A₂; and L₂ is a linking moiety that connects A₂ to P₂ and is a substrate for a tumor specific protease; and (b) a pharmaceutically acceptable excipient.

In some embodiments, the pharmaceutical composition comprises (a) polypeptides or polypeptide complexes comprising Formula Ia:

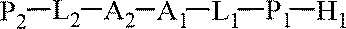

wherein: A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; A₂ comprises a second antigen recognizing molecule that binds to a second target antigen; P₂ comprises a peptide that binds to A₂; and L₂ comprises a linking moiety that connects A₂ to P₂ and is a substrate for a tumor specific protease; and (b) a pharmaceutically acceptable excipient.

In some embodiments, the pharmaceutical composition comprises (a) polypeptides or polypeptide complexes comprising Formula Ia:

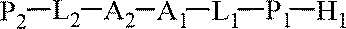

wherein: A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; A₂ is a second antigen recognizing molecule that binds to a second target antigen P₂ is a peptide that binds to A₂; and L₂ is a linking moiety that connects A₂ to P₂ and is a substrate for a tumor specific protease; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70A, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the beta TCR polypeptide is linked to a C terminus of the heavy chain variable domain of the scFv, and wherein the soluble TCR is linked to P₂ and L₂, wherein P₂ comprises a peptide that impairs binding of the soluble TCR to the tumor cell antigen; and L₂ comprises a linking moiety that connects the alpha TCR polypeptide to P₂ and is a substrate for a tumor specific protease; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70B, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the beta TCR polypeptide is linked to a C terminus of the heavy chain variable domain of the scFv; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70C, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the alpha TCR polypeptide is linked to a C terminus of the heavy chain variable domain of the scFv, and wherein the soluble TCR is linked to P₂ and L₂, wherein P₂ comprises a peptide that impairs binding of the soluble TCR to the tumor cell antigen; and L₂ comprises a linking moiety that connects the beta TCR polypeptide to P₂ and is a substrate for a tumor specific protease; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70D, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the alpha TCR polypeptide is linked to a C terminus of the heavy chain variable domain of the scFv; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70E, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the beta TCR polypeptide is linked to a C terminus of the light chain variable domain of the scFv, and wherein the soluble TCR is linked to P₂ and L₂, wherein P₂ comprises a peptide that impairs binding of the soluble TCR to the tumor cell antigen; and L₂ comprises a linking moiety that connects the alpha TCR polypeptide to P₂ and is a substrate for a tumor specific protease; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70F, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the beta TCR polypeptide is linked to a C terminus of the light chain variable domain of the scFv; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70G, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the alpha TCR polypeptide is linked to a C terminus of the light chain variable domain of the scFv, and wherein the soluble TCR is linked to P₂ and L₂, wherein P₂ comprises a peptide that impairs binding of the soluble TCR to the tumor cell antigen; and L₂ comprises a linking moiety that connects the beta TCR polypeptide to P₂ and is a substrate for a tumor specific protease; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70H, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the alpha TCR polypeptide is linked to a C terminus of the light chain variable domain of the scFv; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70I, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to an N-terminus of the beta TCR polypeptide, and wherein the scFv is linked to P₂ and L₂, wherein P₂ comprises a peptide that impairs binding of the scFv to an effector cell antigen; and L₂ comprises a linking moiety that connects the light chain variable domain of the scFv to P₂ and is a substrate for a tumor specific protease; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70J, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to an N-terminus of the beta TCR polypeptide and the scFv binds to an effector cell antigen; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70K, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to an N-terminus of the alpha TCR polypeptide, and wherein the scFv is linked to P₂ and L₂, wherein P₂ comprises a peptide that impairs binding of the scFv to an effector cell antigen; and L₂ comprises a linking moiety that connects the light chain variable domain of the scFv to P₂ and is a substrate for a tumor specific protease; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70L, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to an N-terminus of the alpha TCR polypeptide and the scFv binds to an effector cell antigen; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70M, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to an N-terminus of the beta TCR polypeptide, and wherein the scFv is linked to P₂ and L₂, wherein P₂ comprises a peptide that impairs binding of the scFv to an effector cell antigen; and L₂ comprises a linking moiety that connects the heavy chain variable domain of the scFv to P₂ and is a substrate for a tumor specific protease; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70N, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to an N-terminus of the beta TCR polypeptide and the scFv binds to an effector cell antigen; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70O, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to an N-terminus of the alpha TCR polypeptide, and wherein the scFv is linked to P₂ and L₂, wherein P₂ comprises a peptide that impairs binding of the scFv to an effector cell antigen; and L₂ comprises a linking moiety that connects the heavy chain variable domain of the scFv to P₂ and is a substrate for a tumor specific protease; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70P, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to an N-terminus of the alpha TCR polypeptide and the scFv binds to an effector cell antigen; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70Q, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to a C-terminus of the beta TCR polypeptide and the scFv binds to an effector cell antigen; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70R, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to a C-terminus of the alpha TCR polypeptide and the scFv binds to an effector cell antigen; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70S, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to a C-terminus of the alpha TCR polypeptide and the scFv binds to an effector cell antigen; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70T, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to a C-terminus of the beta TCR polypeptide and the scFv binds to an effector cell antigen; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70U, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to a C-terminus of the beta TCR polypeptide and the scFv binds to an effector cell antigen; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70V, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to a C-terminus of the alpha TCR polypeptide and the scFv binds to an effector cell antigen; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70W, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to a C-terminus of the alpha TCR polypeptide and the scFv binds to an effector cell antigen; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 70X, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to a C-terminus of the beta TCR polypeptide and the scFv binds to an effector cell antigen; and (b) a pharmaceutically acceptable excipient.

In some embodiments, the polypeptide or polypeptide complex further comprises a detectable label, a therapeutic agent, or a pharmacokinetic modifying moiety. In some embodiments, the detectable label comprises a fluorescent label, a radiolabel, an enzyme, a nucleic acid probe, or a contrast agent.

For administration to a subject, the polypeptide or polypeptide complex as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

Table 1 provides the amino acid sequences of constructs described herein.

TABLE 1

Summary of Amino Acid Sequences

| Construct ID | Construct Description | Amino Acid Sequence (N to C) | SEQ ID NO: |
|---|---|---|---|
| PEPTIDE MASK SEQUENCES | | | |
| Peptide-1 | anti-IC3 TCR peptide mask | GGESCQSVYDSSFCYD | 13 |
| Peptide-2 | anti-IC3 TCR peptide mask | GGNACEMTYDHTFCDP | 14 |
| Peptide-3 | anti-IC3 TCR peptide mask | GGRICEEVYDWIFCES | 15 |
| Peptide-4 | anti-IC3 TCR peptide mask | GGRRCVDVYDNAFCLI | 16 |
| Peptide-5 | anti-IC3 TCR peptide mask | GGVSCKDVYDEAFCWT | 12 |
| Peptide-6 | anti-IC3 TCR peptide mask | GGTSCAQIYDFEFCYS | 17 |
| Peptide-7 | anti-IC3 TCR peptide mask | GGSLCSLVYDQDFCES | 18 |
| Peptide-8 | anti-IC3 TCR peptide mask | GGNSCSLVYDKAFCLF | 19 |
| Peptide-9 | anti-IC3 TCR peptide mask | GGNQCWEVYDQEFCSL | 20 |
| Peptide-10 | anti-IC3 TCR peptide mask | GGSACSRIYDFAFCHT | 21 |
| Peptide-11 | anti-IC3 TCR peptide mask | GGTFCYFDHGLVNCQW | 22 |
| Peptide-12 | anti-IC3 TCR peptide mask | GGHCFVSPASGEWWCV | 23 |
| Peptide-13 | anti-IC3 TCR peptide mask | GGCSWIFDGLRYFSKC | 24 |
| Peptide-14 | anti-IC3 TCR peptide mask | VRTWFEKFPELV | 25 |
| Peptide-15 | anti-IC3 TCR peptide mask | LVWGCIWDDMCS | 26 |
| Peptide-16 | anti-IC3 TCR peptide mask | WHWEPSMVWGML | 27 |
| Peptide-17 | anti-IC3 TCR peptide mask | GGGCFVSPATGFTWCV | 28 |
| Peptide-18 | anti-IC3 TCR peptide mask | GGDCQPDSVWSYWYCR | 29 |
| Peptide-19 | anti-IC3 TCR peptide mask | GGCTFVDWWVLGSPYC | 30 |
| Peptide-20 | anti-IC3 TCR peptide mask | GGCLMNDYYYLWGGHC | 31 |
| Peptide-21 | anti-IC3 TCR peptide mask | GGASCKDVYDEAFCWT | 32 |
| Peptide-22 | anti-IC3 TCR peptide | GGVACKDVYDEAFCWT | 33 |
| | mask | | |
| Peptide-23 | anti-IC3 TCR peptide mask | GGVSAKDVYDEAFCWT | 34 |
| Peptide-24 | anti-IC3 TCR peptide mask | GGVSCADVYDEAFCWT | 35 |
| Peptide-25 | anti-IC3 TCR peptide mask | GGVSCKAVYDEAFCWT | 36 |
| Peptide-26 | anti-IC3 TCR peptide mask | GGVSCKDAYDEAFCWT | 37 |
| Peptide-27 | anti-IC3 TCR peptide mask | GGVSCKDVADEAFCWT | 38 |
| Peptide-28 | anti-IC3 TCR peptide mask | GGVSCKDVYAEAFCWT | 39 |
| Peptide-29 | anti-IC3 TCR peptide mask | GGVSCKDVYDAAFCWT | 40 |
| Peptide-30 | anti-IC3 TCR peptide mask | GGVSCKDVYDEAACWT | 41 |
| Peptide-31 | anti-IC3 TCR peptide mask | GGVSCKDVYDEAFAWT | 42 |
| Peptide-32 | anti-IC3 TCR peptide mask | GGVSCKDVYDEAFCAT | 43 |
| Peptide-33 | anti-IC3 TCR peptide mask | GGVSCKDVYDEAFCWA | 44 |
| Peptide-34 | anti-IC3 TCR peptide mask (MAGE-A3) | EVDPIGHLY | 45 |
| Peptide-35 | anti-IC3 TCR peptide mask (titin) | ESDPIVAQY | 46 |
| Peptide-36 | Mock Mask | GGASCAASASAAACAS | 47 |
| Linker-1 | linker | GGGGSLSGRSDNHGSSGT | 53 |
| Linker-2 | linker | GGGGSSGGSGGSGLSGRSDNHGSSGT | 54 |
| Linker-3 | linker | ASGRSDNH | 55 |
| Linker-4 | linker | LAGRSDNH | 56 |
| Linker-5 | linker | ISSGLASGRSDNH | 57 |
| Linker-6 | linker | ISSGLLAGRSDNH | 58 |
| Linker-7 | linker | LSGRSDNH | 4 |
| Linker-8 | linker | ISSGLLSGRSDNP | 59 |
| Linker-9 | linker | ISSGLLSGRSDNH | 60 |
| Linker-10 | linker | LSGRSDNHSPLGLAGS | 61 |
| Linker-11 | linker | SPLGLAGSLSGRSDNH | 62 |
| Linker-12 | linker | SPLGLSGRSDNH | 63 |
| Linker-13 | linker | LAGRSDNHSPLGLAGS | 64 |
| Linker-14 | linker | LSGRSDNHVPLSLKMG | 65 |
| Linker-15 | linker | LSGRSDNHVPLSLSMG | 66 |
| Linker-16 | linker | GSSGGSGGSGGSGLSGRSDNHGSSGT | 68 |
| Linker-17 | linker | GSSGGSGGSGGSGGGSGGGSGGSSGT | 69 |
| Linker-18 | linker | GGGGSGGSGGGSGGSSGT | 70 |
| Linker-19 | linker | GGGGSGGGS | 71 |
| TCR-1 | IC3 TCR alpha chain | MKQEVTQIPAALSVPEGENLVLNCSFTDSAIYNLQ WFRQDPGKGLTSLLYVRPYQREQTSGRLNASLDK SSGRSTLYIAASQPGDSATYLCAVRPGGAGPFFVV FGKGTKLSVIPNIQNPDPAVYQLRDSKSSDKSVCLF TDFDSQTNVSQSKDSDVYITDKCVLDMRSMDFKS | 73 |
| | | NSAVAWSNKSDFACANAFNNSIIPEDTFFPSPESSG GHHHHHHHH | |
| TCR-1 | | KQEVTQIPAALSVPEGENLVLNCSFTDSAIYNLQW FRQDPGKGLTSLLYVRPYQREQTSGRLNASLDKSS GRSTLYIAASQPGDSATYLCAVRPGGAGPFFVVFG KGTKLSVIPNIQNPDPAVYQLRDSKSSDKSVCLFTD FDSQTNVSQSKDSDVYITDKCVLDMRSMDFKSNS AVAWSNKSDFACANAFNNSIIPEDTFFPSPESS | 5 |
| TCR-1 | IC3 TCR beta chain | MKAGVTQTPRYLIKTRGQQVTLSCSPISGHRSVSW YQQTPGQGLQFLFEYFSETQRNKGNFPGRFSGRQF SNSRSEMNVSTLELGDSALYLCASSFNMATGQYFG PGTRLTVTEDLKNVFPPEVAVFEPSEAEISHTQKAT LVCLATGFYPDHVELSWWVNGKEVHSGVCTDPQP LKEQPALNDSRYALSSRLRVSATFWQNPRNHFRC QVQFYGLSENDEWTQDRAKPVTQIVSAEAWGRA DGGGLNDIFEAQKIEWHE | 74 |
| TCR-1 | | KAGVTQTPRYLIKTRGQQVTLSCSPISGHRSVSWY QQTPGQGLQFLFEYFSETQRNKGNFPGRFSGRQFS NSRSEMNVSTLELGDSALYLCASSFNMATGQYFGP GTRLTVTEDLKNVFPPEVAVFEPSEAEISHTQKATL VCLATGFYPDHVELSWWVNGKEVHSGVCTDPQPL KEQPALNDSRYALSSRLRVSATFWQNPRNHFRCQ VQFYGLSENDEWTQDRAKPVTQIVSAEAWGRAD | 9 |
| TCR-2 | A3A-3 TCR alpha chain | MKQEVTQIPAALSVPEGENLVLNCSFTDSAIYNLQ WFRQDPGKGLTSLLLVRPYQREQTSGRLNASLDKS SGRSTLYIAASQPGDSATYLCAVRPGGAGSYQLTF GKGTKLSVIPNIQNPDPAVYQLRDSKSSDKSVCLFT DFDSQTNVSQSKDSDVYITDKCVLDMRSMDFKSN SAVAWSNKSDFACANAFNNSIIPEDTFFPSPESSGG HHHHHHHH | 75 |
| TCR-2 | A3A-3 TCR alpha chain | KQEVTQIPAALSVPEGENLVLNCSFTDSAIYNLQW FRQDPGKGLTSLLLVRPYQREQTSGRLNASLDKSS GRSTLYIAASQPGDSATYLCAVRPGGAGSYQLTFG KGTKLSVIPNIQNPDPAVYQLRDSKSSDKSVCLFTD FDSQTNVSQSKDSDVYITDKCVLDMRSMDFKSNS AVAWSNKSDFACANAFNNSIIPEDTFFPSPESS | 76 |
| TCR-2 | A3A-3 TCR beta chain | MKAGVTQTPRYLIKTRGQQVTLSCSPISGHRSVSW YQQTPGQGLQFLFEYFSETQRNKGNFPGRFSGRQF SNSRSEMNVSTLELGDSALYLCASSPNMADEQYFG PGTRLTVTEDLKNVFPPEVAVFEPSEAEISHTQKAT LVCLATGFYPDHVELSWWVNGKEVHSGVCTDPQP LKEQPALNDSRYALSSRLRVSATFWQDPRNHFRC QVQFYGLSENDEWTQDRAKPVTQIVSAEAWGRA DGGGLNDIFEAQKIEWHE | 77 |
| TCR-2 | A3A-3 TCR beta chain | KAGVTQTPRYLIKTRGQQVTLSCSPISGHRSVSWY QQTPGQGLQFLFEYFSETQRNKGNFPGRFSGRQFS NSRSEMNVSTLELGDSALYLCASSPNMADEQYFGP GTRLTVTEDLKNVFPPEVAVFEPSEAEISHTQKATL VCLATGFYPDHVELSWWVNGKEVHSGVCTDPQPL KEQPALNDSRYALSSRLRVSATFWQDPRNHFRCQ VQFYGLSENDEWTQDRAKPVTQIVSAEAWGRAD | 78 |
| TCR-3 | A3A-4 TCR alpha chain | MKQEVTQIPAALSVPEGENLVLNCSFTDSAIYNLQ WFRQDPGKGLTSLLLIQSSQREQTSGRLNASLDKS SGRSTLYIAASQPGDSATYLCAVRPGGAGSYQLTF GKGTKLSVIPNIQNPDPAVYQLRDSKSSDKSVCLFT | 79 |
| | | DFDSQTNVSQSKDSDVYITDKCVLDMRSMDFKSN SAVAWSNKSDFACANAFNNSIIPEDTFFPSPESSGG HHHHHHHH | |
| TCR-3 | A3A-4 TCR alpha chain | KQEVTQIPAALSVPEGENLVLNCSFTDSAIYNLQW FRQDPGKGLTSLLLIQSSQREQTSGRLNASLDKSSG RSTLYIAASQPGDSATYLCAVRPGGAGSYQLTFGK GTKLSVIPNIQNPDPAVYQLRDSKSSDKSVCLFTDF DSQTNVSQSKDSDVYITDKCVLDMRSMDFKSNSA VAWSNKSDFACANAFNNSIIPEDTFFPSPESS | 80 |
| TCR-3 | A3A-4 TCR beta chain | MKAGVTQTPRYLIKTRGQQVTLSCSPISGHRSVSW YQQTPGQGLQFLFEYTDMTLRNKGNFPGRFSGRQ FSNSRSEMNVSTLELGDSALYLCASSPNMADEQYF GPGTRLTVTEDLKNVFPPEVAVFEPSEAEISHTQKA TLVCLATGFYPDHVELSWWVNGKEVHSGVCTDP QPLKEQPALNDSRYALSSRLRVSATFWQDPRNHFR CQVQFYGLSENDEWTQDRAKPVTQIVSAEAWGRA DGGGLNDIFEAQKIEWHE | 81 |
| TCR-3 | A3A-4 TCR beta chain | KAGVTQTPRYLIKTRGQQVTLSCSPISGHRSVSWY QQTPGQGLQFLFEYTDMTLRNKGNFPGRFSGRQFS NSRSEMNVSTLELGDSALYLCASSPNMADEQYFGP GTRLTVTEDLKNVFPPEVAVFEPSEAEISHTQKATL VCLATGFYPDHVELSWWVNGKEVHSGVCTDPQPL KEQPALNDSRYALSSRLRVSATFWQDPRNHFRCQ VQFYGLSENDEWTQDRAKPVTQIVSAEAWGRAD | 82 |
| TCR-4 | A3A-5 TCR alpha chain | MKQEVTQIPAALSVPEGENLVLNCSFTDSAIYNLQ WFRQDPGKGLTSLLLIQSSQREQTSGRLNASLDKS SGRSTLYIAASQPGDSATYLCAVRPGGAGSYQLTF GKGTKLSVIPNIQNPDPAVYQLRDSKSSDKSVCLFT DFDSQTNVSQSKDSDVYITDKCVLDMRSMDFKSN SAVAWSNKSDFACANAFNNSIIPEDTFFPSPESSGG HHHHHHHH | 79 |
| TCR-4 | A3A-5 TCR alpha chain | KQEVTQIPAALSVPEGENLVLNCSFTDSAIYNLQW FRQDPGKGLTSLLLIQSSQREQTSGRLNASLDKSSG RSTLYIAASQPGDSATYLCAVRPGGAGSYQLTFGK GTKLSVIPNIQNPDPAVYQLRDSKSSDKSVCLFTDF DSQTNVSQSKDSDVYITDKCVLDMRSMDFKSNSA VAWSNKSDFACANAFNNSIIPEDTFFPSPESS | 80 |
| TCR-4 | A3A-5 TCR beta chain | MKAGVTQTPRYLIKTRGQQVTLSCSPISGHRSVSW YQQTPGQGLQFLFEYFDMLLRNKGNFPGRFSGRQF SNSRSEMNVSTLELGDSALYLCASSPNMADEQYFG PGTRLTVTEDLKNVFPPEVAVFEPSEAEISHTQKAT LVCLATGFYPDHVELSWWVNGKEVHSGVCTDPQP LKEQPALNDSRYALSSRLRVSATFWQDPRNHFRC QVQFYGLSENDEWTQDRAKPVTQIVSAEAWGRA DGGGLNDIFEAQKIEWHE | 83 |
| TCR-4 | A3A-5 TCR beta chain | KAGVTQTPRYLIKTRGQQVTLSCSPISGHRSVSWY QQTPGQGLQFLFEYFDMLLRNKGNFPGRFSGRQFS NSRSEMNVSTLELGDSALYLCASSPNMADEQYFGP GTRLTVTEDLKNVFPPEVAVFEPSEAEISHTQKATL VCLATGFYPDHVELSWWVNGKEVHSGVCTDPQPL KEQPALNDSRYALSSRLRVSATFWQDPRNHFRCQ VQFYGLSENDEWTQDRAKPVTQIVSAEAWGRAD | 84 |
| TCR-5 | | KQEVTQIPAALSVPEGENLVLNCSFTDSAIYNLQW FRQDPGKGLTSLLYVRPYQREQTSGRLNASLDKSS GRSTLYIAASQPGDSATYLCAVRPGGAGPFFVVFG | 85 |
| | | KGTKLSVIPNIQNPDPAVYQLRDSKSSDKSVCLFTD FDSQTNVSQSKDSDVYITDKCVLDMRSMDFKSNS AVAWSNKSDFACANAFNNSIIPEDTFFPSPESSGGH HHHHHHH | |
| TCR-5 | | KQEVTQIPAALSVPEGENLVLNCSFTDSAIYNLQW FRQDPGKGLTSLLYVRPYQREQTSGRLNASLDKSS GRSTLYIAASQPGDSATYLCAVRPGGAGPFFVVFG KGTKLSVIPNIQNPDPAVYQLRDSKSSDKSVCLFTD FDSQTNVSQSKDSDVYITDKCVLDMRSMDFKSNS AVAWSNKSDFACANAFNNSIIPEDTFFPSPESS | 5 |
| Ab-1 | UCHT1 scFv [N-(light-heavy)-C] | AIQMTQSPSSLSASVGDRVTITCRASQDIRNYLNW YQQKPGKAPKLLIYYTSRLESGVPSRFSGSGSGTD YTLTISSLQPEDFATYYCQQGNTLPWTFGQGTKVE IKGGGGSGGGGSGGGGSGGGGSGGGSEVQLVESG GGLVQPGGSLRLSCAASGYSFTGYTMNWVRQAPG KGLEWVALINPYKGVSTYNQKFKDRFTISVDKSKN TAYLQMNSLRAEDTAVYYCARSGYYGD SDWYFD VWGQGTLVTVSS | 86 |
| Ab-2 | N-[UCHT1 scFv [N-(light-heavy-C] | MAIQMTQSPSSLSASVGDRVTITCRASQDIRNYLN WYQQKPGKAPKLLIYYTSRLESGVPSRFSGSGSGT DYTLTISSLQPEDFATYYCQQGNTLPWTFGQGTKV EIKGGGGSGGGGSGGGGSGGGGSGGGSEVQLVES GGGLVQPGGSLRLSCAASGYSFTGYTMNWVRQAP GKGLEWVALINPYKGVSTYNQKFKDRFTISVDKSK NTAYLQMNSLRAEDTAVYYCARSGYYGDSDWYF DVWGQGTLVTVSS | 8 |
| HE-1 | 10G VHH | MEVQLVESGGGLVQPGNSLRLSCAASGFTFSKFG MSWVRQAPGKGLEWVSSISGSGRDTLYADSVKGR FTISRDNAKTTLYLQMNSLRPEDTAVYYCTIGGSLS VSSQGTLVTVSS | 2 |
| HE-2 | 10G VHH | EVQLVESGGGLVQPGNSLRLSCAASGFTFSKFGMS WVRQAPGKGLEWVSSISGSGRDTLYADSVKGRFTI SRDNAKTTLYLQMNSLRPEDTAVYYCTIGGSLSVS SQGTLVTVSS | 72 |
| TCR-6 | IC3 TCR alpha chain | MKQEVTQIPAALSVPEGENLVLNCSFTDSAIYNLQ WFRQDPGKGLTSLLYVRPYQREQTSGRLNASLDK SSGRSTLYIAASQPGDSATYLCAVRPGGAGPFFVV FGKGTKLSVIPNIQNPDPAVYQLRDSKSSDKSVCLF TDFDSQTNVSQSKDSDVYITDKCVLDMRSMDFKS NSAVAWSNKSDFACANAFNNSIIPEDTFFPSPESSG GHHHHHHHH | 73 |
| TCR -6 | N-[Peptide-34]-[linker]-[IC3 TCR beta chain]-C | MEVDPIGHLYGSSGGSGGSGGSGLSGRSDNHGSSG TKAGVTQTPRYLIKTRGQQVTLSCSPISGHRSVSW YQQTPGQGLQFLFEYFSETQRNKGNFPGRFSGRQF SNSRSEMNVSTLELGDSALYLCASSFNMATGQYFG PGTRLTVTEDLKNVFPPEVAVFEPSEAEISHTQKAT LVCLATGFYPDHVELSWWVNGKEVHSGVCTDPQP LKEQPALNDSRYALSSRLRVSATFWQNPRNHFRC QVQFYGLSENDEWTQDRAKPVTQIVSAEAWGRA DGGGLNDIFEAQKIEWHE | 87 |
| TCR -7 | IC3 TCR alpha chain | MKQEVTQIPAALSVPEGENLVLNCSFTDSAIYNLQ WFRQDPGKGLTSLLYVRPYQREQTSGRLNASLDK | 73 |
| | | SSGRSTLYIAASQPGDSATYLCAVRPGGAGPFFVV FGKGTKLSVIPNIQNPDPAVYQLRDSKSSDKSVCLF TDFDSQTNVSQSKDSDVYITDKCVLDMRSMDFKS NSAVAWSNKSDFACANAFNNSIIPEDTFFPSPESSG GHHHHHHHH | |
| TCR-7 | N-[Peptide-34]-[linker]-[IC3 TCR beta chain]-C | MEVDPIGHLYGSSGGSGGSGGSGGGSGGGSGGSS GTKAGVTQTPRYLIKTRGQQVTLSCSPISGHRSVS WYQQTPGQGLQFLFEYFSETQRNKGNFPGRFSGR QFSNSRSEMNVSTLELGDSALYLCASSFNMATGQY FGPGTRLTVTEDLKNVFPPEVAVFEPSEAEISHTQK ATLVCLATGFYPDHVELSWWVNGKEVHSGVCTD PQPLKEQPALNDSRYALSSRLRVSATFWQNPRNHF RCQVQFYGLSENDEWTQDRAKPVTQIVSAEAWGR ADGGGLNDIFEAQKIEWHE | 88 |
| TCR-8 | IC3 TCR alpha chain | MKQEVTQIPAALSVPEGENLVLNCSFTDSAIYNLQ WFRQDPGKGLTSLLYVRPYQREQTSGRLNASLDK SSGRSTLYIAASQPGDSATYLCAVRPGGAGPFFVV FGKGTKLSVIPNIQNPDPAVYQLRDSKSSDKSVCLF TDFDSQTNVSQSKDSDVYITDKCVLDMRSMDFKS NSAVAWSNKSDFACANAFNNSIIPEDTFFPSPESSG GHHHHHHHH | 73 |
| TCR-8 | N-[Peptide-5]-[linker]-[IC3 TCR beta chain]-C | MGGVSCKDVYDEAFCWTGGGGSLSGRSDNHGSS GTKAGVTQTPRYLIKTRGQQVTLSCSPISGHRSVS WYQQTPGQGLQFLFEYFSETQRNKGNFPGRFSGR QFSNSRSEMNVSTLELGDSALYLCASSFNMATGQY FGPGTRLTVTEDLKNVFPPEVAVFEPSEAEISHTQK ATLVCLATGFYPDHVELSWWVNGKEVHSGVCTD PQPLKEQPALNDSRYALSSRLRVSATFWQNPRNHF RCQVQFYGLSENDEWTQDRAKPVTQIVSAEAWGR ADGGGLNDIFEAQKIEWHE | 89 |
| TCR-9 | IC3 TCR alpha chain | MKQEVTQIPAALSVPEGENLVLNCSFTDSAIYNLQ WFRQDPGKGLTSLLYVRPYQREQTSGRLNASLDK SSGRSTLYIAASQPGDSATYLCAVRPGGAGPFFVV FGKGTKLSVIPNIQNPDPAVYQLRDSKSSDKSVCLF TDFDSQTNVSQSKDSDVYITDKCVLDMRSMDFKS NSAVAWSNKSDFACANAFNNSIIPEDTFFPSPESSG GHHHHHHHH | 73 |
| TCR-9 | N-[Peptide-5]-[linker]-[IC3 TCR beta chain]-C | MGGVSCKDVYDEAFCWTGGGGSSGGSGGSGLSG RSDNHGSSGTKAGVTQTPRYLIKTRGQQVTLSCSPI SGHRSVSWYQQTPGQGLQFLFEYFSETQRNKGNFP GRFSGRQFSNSRSEMNVSTLELGDSALYLCASSFN MATGQYFGPGTRLTVTEDLKNVFPPEVAVFEPSEA EISHTQKATLVCLATGFYPDHVELSWWVNGKEVH SGVCTDPQPLKEQPALNDSRYALSSRLRVSATFWQ NPRNHFRCQVQFYGLSENDEWTQDRAKPVTQIVS AEAWGRADGGGLNDIFEAQKIEWHE | 90 |
| TCR-10 | N-[Peptide-5]-[linker]-[IC3 TCR alpha chain]-C | MGGVSCKDVYDEAFCWTGGGGSLSGRSDNHGSS GTKQEVTQIPAALSVPEGENLVLNCSFTDSAIYNLQ WFRQDPGKGLTSLLYVRPYQREQTSGRLNASLDK SSGRSTLYIAASQPGDSATYLCAVRPGGAGPFFVV FGKGTKLSVIPNIQNPDPAVYQLRDSKSSDKSVCLF TDFDSQTNVSQSKDSDVYITDKCVLDMRSMDFKS NSAVAWSNKSDFACANAFNNSIIPEDTFFPSPESSG G HHHHHHHH | 91 |
| TCR-10 | IC3 TCR beta chain | MKAGVTQTPRYLIKTRGQQVTLSCSPISGHRSVSW YQQTPGQGLQFLFEYFSETQRNKGNFPGRFSGRQF | 74 |
| | | SNSRSEMNVSTLELGDSALYLCASSFNMATGQYFG PGTRLTVTEDLKNVFPPEVAVFEPSEAEISHTQKAT LVCLATGFYPDHVELSWWVNGKEVHSGVCTDPQP LKEQPALNDSRYALSSRLRVSATFWQNPRNHFRC QVQFYGLSENDEWTQDRAKP VTQI V SAEAW GRA DGGGLNDIFEAQKIEWHE | |
| TCR-11 | N-[Peptide-5]-[linker]-[IC3 TCR alpha chain]-C | MGGVSCKDVYDEAFCWTGGGGSSGGSGGSGLSG RSDNHGSSGTKQEVTQIPAALSVPEGENLVLNCSF TDSAIYNLQWFRQDPGKGLTSLLYVRPYQREQTSG RLNASLDKSSGRSTLYIAASQPGDSATYLCAVRPG GAGPFFVVFGKGTKLSVIPNIQNPDPAVYQLRDSK SSDKSVCLFTDFDSQTNVSQSKDSDVYITDKCVLD MRSMDFKSNSAVAWSNKSDFACANAFNNSIIPEDT FFPSPESSGGHHHHHHHH | 92 |
| TCR-11 | IC3 TCR beta chain | MKAGVTQTPRYLIKTRGQQVTLSCSPISGHRSVSW YQQTPGQGLQFLFEYFSETQRNKGNFPGRFSGRQF SNSRSEMNVSTLELGDSALYLCASSFNMATGQYFG PGTRLTVTEDLKNVFPPEVAVFEPSEAEISHTQKAT LVCLATGFYPDHVELSWWVNGKEVHSGVCTDPQP LKEQPALNDSRYALSSRLRVSATFWQNPRNHFRC QVQFYGLSENDEWTQDRAKPVTQIVSAEAWGRA DGGGLNDIFEAQKIEWHE | 74 |
| TCR-12 | IC3 TCR alpha chain | MKQEVTQIPAALSVPEGENLVLNCSFTDSAIYNLQ WFRQDPGKGLTSLLYVRPYQREQTSGRLNASLDK SSGRSTLYIAASQPGDSATYLCAVRPGGAGPFFVV FGKGTKLSVIPNIQNPDPAVYQLRDSKSSDKSVCLF TDFDSQTNVSQSKDSDVYITDKCVLDMRSMDFKS NSAVAWSNKSDFACANAFNNSIIPEDTFFPSPESSG GHHHHHHHH | 73 |
| TCR-12 | N-[IC3 TCR beta chain]-[UCHT1 scFv (light-heavy)]-C | MKAGVTQTPRYLIKTRGQQVTLSCSPISGHRSVSW YQQTPGQGLQFLFEYFSETQRNKGNFPGRFSGRQF SNSRSEMNVSTLELGDSALYLCASSFNMATGQYFG PGTRLTVTEDLKNVFPPEVAVFEPSEAEISHTQKAT LVCLATGFYPDHVELSWWVNGKEVHSGVCTDPQP LKEQPALNDSRYALSSRLRVSATFWQNPRNHFRC QVQFYGLSENDEWTQDRAKPVTQIVSAEAWGRA DGGGGSAIQMTQSPSSLSASVGDRVTITCRASQDIR NYLNWYQQKPGKAPKLLIYYTSRLESGVPSRFSGS GSGTDYTLTISSLQPEDFATYYCQQGNTLPWTFGQ GTKVEIKGGGGSGGGGSGGGGSGGGGSGGGSEVQ LVESGGGLVQPGGSLRLSCAASGYSFTGYTMNWV RQAPGKGLEWVALINPYKGVSTYNQKFKDRFTISV DKSKNTAYLQMNSLRAEDTAVYYCARSGYYGDS DWYFDVWGQGTLVTVSSGGGLNDIFEAQKIEWHE | 93 |
| TCR-13 | IC3 TCR alpha chain | MKQEVTQIPAALSVPEGENLVLNCSFTDSAIYNLQ WFRQDPGKGLTSLLYVRPYQREQTSGRLNASLDK SSGRSTLYIAASQPGDSATYLCAVRPGGAGPFFVV FGKGTKLSVIPNIQNPDPAVYQLRDSKSSDKSVCLF TDFDSQTNVSQSKDSDVYITDKCVLDMRSMDFKS NSAVAWSNKSDFACANAFNNSIIPEDTFFPSPESSG GHHHHHHHH | 73 |
| TCR-13 | N-[Peptide-5]-[linker]-[IC3 TCR beta chain]-[UCHT1 scFv (light-heavy)]-C | MGGVSCKDVYDEAFCWTGGGGSSGGSGGSGLSG RSDNHGSSGTKAGVTQTPRYLIKTRGQQVTLSCSPI SGHRSVSWYQQTPGQGLQFLFEYFSETQRNKGNFP GRFSGRQFSNSRSEMNVSTLELGDSALYLCASSFN MATGQYFGPGTRLTVTEDLKNVFPPEVAVFEPSEA | 94 |
| | | EISHTQKATLVCLATGFYPDHVELSWWVNGKEVH SGVCTDPQPLKEQPALNDSRYALSSRLRVSATFWQ NPRNHFRCQVQFYGLSENDEWTQDRAKPVTQIVS AEAWGRADGGGGSAIQMTQSPSSLSASVGDRVTIT CRASQDIRNYLNWYQQKPGKAPKLLIYYTSRLESG VPSRFSGSGSGTDYTLTISSLQPEDFATYYCQQGNT LPWTFGQGTKVEIKGGGGSGGGGSGGGGSGGGGS GGGSEVQLVESGGGLVQPGGSLRLSCAASGYSFTG YTMNWVRQAPGKGLEWVALINPYKGVSTYNQKF KDRFTISVDKSKNTAYLQMNSLRAEDTAVYYCAR SGYYGDSDWYF DVWGQGTLVTVSSGGGLNDIFEAQKIEWHE | |
| TCR-14 | IC3 TCR alpha chain | MKQEVTQIPAALSVPEGENLVLNCSFTDSAIYNLQ WFRQDPGKGLTSLLYVRPYQREQTSGRLNASLDK SSGRSTLYIAASQPGDSATYLCAVRPGGAGPFFVV FGKGTKLSVIPNIQNPDPAVYQLRDSKSSDKSVCLF TDFDSQTNVSQSKDSDVYITDKCVLDMRSMDFKS NSAVAWSNKSDFACANAFNNSIIPEDTFFPSPESSG GHHHHHHHH | 73 |
| TCR-14 | N-[UCHT1 scFv (light-heavy)]-[IC3 TCR beta chain]-C | MAIQMTQSPSSLSASVGDRVTITCRASQDIRNYLN WYQQKPGKAPKLLIYYTSRLESGVPSRFSGSGSGT DYTLTISSLQPEDFATYYCQQGNTLPWTFGQGTKV EIKGGGGSGGGGSGGGGSGGGGSGGGSEVQLVES GGGLVQPGGSLRLSCAASGYSFTGYTMNWVRQAP GKGLEWVALINPYKGVSTYNQKFKDRFTISVDKSK NTAYLQMNSLRAEDTAVYYCARSGYYGDSDWYF DVWGQGTLVTVSSGGGGSKAGVTQTPRYLIKTRG QQVTLSCSPISGHRSVSWYQQTPGQGLQFLFEYFSE TQRNKGNFPGRFSGRQFSNSRSEMNVSTLELGDSA LYLCASSFNMATGQYFGPGTRLTVTEDLKNVFPPE VAVFEPSEAEISHTQKATLVCLATGFYPDHVELSW WVNGKEVHSGVCTDPQPLKEQPALNDSRYALSSR LRVSATFWQNPRNHFRCQVQFYGLSENDEWTQDR AKPVTQIVSAEAWGRADGGGLNDIFEAQKIEWHE | 7 |
| TCR-15 | N-[Peptide-5]-[linker]-[IC3 TCR alpha chain] | MGGVSCKDVYDEAFCWTGGGGSLSGRSDNHGSS GTKQEVTQIPAALSVPEGENLVLNCSFTDSAIYNLQ WFRQDPGKGLTSLLYVRPYQREQTSGRLNASLDK SSGRSTLYIAASQPGDSATYLCAVRPGGAGPFFVV FGKGTKLSVIPNIQNPDPAVYQLRDSKSSDKSVCLF TDFDSQTNVSQSKDSDVYITDKCVLDMRSMDFKS NSAVAWSNKSDFACANAFNNSIIPEDTFFPSPESSG GH HHHHHHH | 91 |
| TCR-15 | N-[UCHT1 scFv (light-heavy)]-[IC3 TCR beta chain]-C | MAIQMTQSPSSLSASVGDRVTITCRASQDIRNYLN WYQQKPGKAPKLLIYYTSRLESGVPSRFSGSGSGT DYTLTISSLQPEDFATYYCQQGNTLPWTFGQGTKV EIKGGGGSGGGGSGGGGSGGGGSGGGSEVQLVES GGGLVQPGGSLRLSCAASGYSFTGYTMNWVRQAP GKGLEWVALINPYKGVSTYNQKFKDRFTISVDKSK NTAYLQMNSLRAEDTAVYYCARSGYYGDSDWYF DVWGQGTLVTVSSGGGGSKAGVTQTPRYLIKTRG QQVTLSCSPISGHRSVSWYQQTPGQGLQFLFEYFSE TQRNKGNFPGRFSGRQFSNSRSEMNVSTLELGDSA LYLCASSFNMATGQYFGPGTRLTVTEDLKNVFPPE VAVFEPSEAEISHTQKATLVCLATGFYPDHVELSW WVNGKEVHSGVCTDPQPLKEQPALNDSRYALSSR LRVSATFWQNPRNHFRCQVQFYGLSENDEWTQDR | 7 |
| | | AKPVTQIVSAEAWGRADGGGLNDIFEAQKIEWHE | |
| TCR-16 | N-[Peptide-5]-[linker]-[IC3 TCR alpha chain] | MGGVSCKDVYDEAFCWTGGGGSSGGSGGSGLSG RSDNHGSSGTKQEVTQIPAALSVPEGENLVLNCSF TDSAIYNLQWFRQDPGKGLTSLLYVRPYQREQTSG RLNASLDKSSGRSTLYIAASQPGDSATYLCAVRPG GAGPFFVVFGKGTKLSVIPNIQNPDPAVYQLRDSK SSDKSVCLFTDFDSQTNVSQSKDSDVYITDKCVLD MRSMDFKSNSAVAWSNKSDFACANAFNNSIIPEDT FFPSPESSGGHHHHHHHH | 92 |
| TCR-16 | N-[UCHT1 scFv (light-heavy)]-[IC3 TCR beta chain]-C | MAIQMTQSPSSLSASVGDRVTITCRASQDIRNYLN WYQQKPGKAPKLLIYYTSRLESGVPSRFSGSGSGT DYTLTISSLQPEDFATYYCQQGNTLPWTFGQGTKV EIKGGGGSGGGGSGGGGSGGGGSGGGSEVQLVES GGGLVQPGGSLRLSCAASGYSFTGYTMNWVRQAP GKGLEWVALINPYKGVSTYNQKFKDRFTISVDKSK NTAYLQMNSLRAEDTAVYYCARSGYYGDSDWYF DVWGQGTLVTVSSGGGGSKAGVTQTPRYLIKTRG QQVTLSCSPISGHRSVSWYQQTPGQGLQFLFEYFSE TQRNKGNFPGRFSGRQFSNSRSEMNVSTLELGDSA LYLCASSFNMATGQYFGPGTRLTVTEDLKNVFPPE VAVFEPSEAEISHTQKATLVCLATGFYPDHVELSW WVNGKEVHSGVCTDPQPLKEQPALNDSRYALSSR LRVSATFWQNPRNHFRCQVQFYGLSENDEWTQDR AKPVTQIVSAEAWGRADGGGLNDIFEAQKIEWHE | 7 |
| TCR-17 | N-[UCHT1 scFv (light-heavy)]-[IC3 TCR alpha chain]-C | MAIQMTQSPSSLSASVGDRVTITCRASQDIRNYLN WYQQKPGKAPKLLIYYTSRLESGVPSRFSGSGSGT DYTLTISSLQPEDFATYYCQQGNTLPWTFGQGTKV EIKGGGGSGGGGSGGGGSGGGGSGGGSEVQLVES GGGLVQPGGSLRLSCAASGYSFTGYTMNWVRQAP GKGLEWVALINPYKGVSTYNQKFKDRFTISVDKSK NTAYLQMNSLRAEDTAVYYCARSGYYGDSDWYF DVWGQGTLVTVSSGGGGSKQEVTQIPAALSVPEG ENLVLNCSFTDSAIYNLQWFRQDPGKGLTSLLYVR PYQREQTSGRLNASLDKSSGRSTLYIAASQPGDSA TYLCAVRPGGAGPFFVVFGKGTKLSVIPNIQNPDP AVYQLRDSKSSDKSVCLFTDFDSQTNVSQSKDSDV YITDKCVLDMRSMDFKSNSAVAWSNKSDFACAN AFNNSIIPEDTFFPSPESSGGHHHHHHHH | 95 |
| TCR-17 | IC3 TCR beta chain | MKAGVTQTPRYLIKTRGQQVTLSCSPISGHRSVSW YQQTPGQGLQFLFEYFSETQRNKGNFPGRFSGRQF SNSRSEMNVSTLELGDSALYLCASSFNMATGQYFG PGTRLTVTEDLKNVFPPEVAVFEPSEAEISHTQKAT LVCLATGFYPDHVELSWWVNGKEVHSGVCTDPQP LKEQPALNDSRYALSSRLRVSATFWQNPRNHFRC QVQFYGLSENDEWTQDRAKPVTQIVSAEAWGRA DGGGLNDIFEAQKIEWHE | 74 |
| TCR-18 | N-[UCHT1 scFv (light-heavy)]-[IC3 TCR alpha chain]-C | MAIQMTQSPSSLSASVGDRVTITCRASQDIRNYLN WYQQKPGKAPKLLIYYTSRLESGVPSRFSGSGSGT DYTLTISSLQPEDFATYYCQQGNTLPWTFGQGTKV EIKGGGGSGGGGSGGGGSGGGGSGGGSEVQLVES GGGLVQPGGSLRLSCAASGYSFTGYTMNWVRQAP GKGLEWVALINPYKGVSTYNQKFKDRFTISVDKSK NTAYLQMNSLRAEDTAVYYCARSGYYGDSDWYF DVWGQGTLVTVSSGGGGSKQEVTQIPAALSVPEG ENLVLNCSFTDSAIYNLQWFRQDPGKGLTSLLYVR PYQREQTSGRLNASLDKSSGRSTLYIAASQPGDSA | 95 |
| | | TYLCAVRPGGAGPFFVVFGKGTKLSVIPNIQNPDP AVYQLRDSKSSDKSVCLFTDFDSQTNVSQSKDSDV YITDKCVLDMRSMDFKSNSAVAWSNKSDFACAN AFNNSIIPEDTFFPSPESSGGHHHHHHHH | |
| TCR-18 | N-[Peptide-5]-[linker]-[IC3 TCR beta chain]-C | MGGVSCKDVYDEAFCWTGGGGSSGGSGGSGLSG RSDNHGSSGTKAGVTQTPRYLIKTRGQQVTLSCSPI SGHRSVSWYQQTPGQGLQFLFEYFSETQRNKGNFP GRFSGRQFSNSRSEMNVSTLELGDSALYLCASSFN MATGQYFGPGTRLTVTEDLKNVFPPEVAVFEPSEA EISHTQKATLVCLATGFYPDHVELSWWVNGKEVH SGVCTDPQPLKEQPALNDSRYALSSRLRVSATFWQ NPRNHFRCQVQFYGLSENDEWTQDRAKPVTQIVS AEAWGRADGGGLNDIFEAQKIEWHE | 90 |
| TCR-19 | N-[Peptide-5]-[noncleavable linker]-[IC3 TCR alpha chain]-C | MGGVSCKDVYDEAFCWTGGGGSGGSGGGSGGSS GTKQEVTQIPAALSVPEGENLVLNCSFTDSAIYNLQ WFRQDPGKGLTSLLYVRPYQREQTSGRLNASLDK SSGRSTLYIAASQPGDSATYLCAVRPGGAGPFFVV FGKGTKLSVIPNIQNPDPAVYQLRDSKSSDKSVCLF TDFDSQTNVSQSKDSDVYITDKCVLDMRSMDFKS NSAVAWSNKSDFACANAFNNSHPEDTFFPSPESSG G HHHHHHHH | 96 |
| TCR-19 | N-[UCHT1 scFv (light-heavy)]-[IC3 TCR beta chain]-C | MAIQMTQSPSSLSASVGDRVTITCRASQDIRNYLN WYQQKPGKAPKLLIYYTSRLESGVPSRFSGSGSGT DYTLTISSLQPEDFATYYCQQGNTLPWTFGQGTKV EIKGGGGSGGGGSGGGGSGGGGSGGGSEVQLVES GGGLVQPGGSLRLSCAASGYSFTGYTMNWVRQAP GKGLEWVALINPYKGVSTYNQKFKDRFTISVDKSK NTAYLQMNSLRAEDTAVYYCARSGYYGDSDWYF DVWGQGTLVTVSSGGGGSKAGVTQTPRYLIKTRG QQVTLSCSPISGHRSVSWYQQTPGQGLQFLFEYFSE TQRNKGNFPGRFSGRQFSNSRSEMNVSTLELGDSA LYLCASSFNMATGQYFGPGTRLTVTEDLKNVFPPE VAVFEPSEAEISHTQKATLVCLATGFYPDHVELSW WVNGKEVHSGVCTDPQPLKEQ PALNDSRYALSSRLRVSATFWQNPRNHFRCQVQF YGLSENDEWTQDRAKPVTQIVSAEAWGRADGGG LNDIFEAQKIEWHE | 7 |
| TCR-20 | N-[10G VHH]-[Peptide-5]-[cleavable linker]-[IC3 TCR alpha chain]-C | MEVQLVESGGGLVQPGNSLRLSCAASGFTFSKFG MSWVRQAPGKGLEWVSSISGSGRDTLYADSVKGR FTISRDNAKTTLYLQMNSLRPEDTAVYYCTIGGSLS VSSQGTLVTVSSGGGGSGGGSGGVSCKDVYDEAF CWTGGGGSLSGRSDNHGSSGTKQEVTQIPAALSVP EGENLVLNCSFTDSAIYNLQWFRQDPGKGLTSLLY VRPYQREQTSGRLNASLDKSSGRSTLYIAASQPGD SATYLCAVRPGGAGPFFVVFGKGTKLSVIPNIQNP DPAVYQLRDSKSSDKSVCLFTDFDSQTNVSQSKDS DVYITDKCVLDMRSMDFKSNSAVAWSNKSDFAC ANAFNNSIIPEDTFFPSPESSGGHHHHHHHH | 1 |
| TCR-20 | N-[UCHT1 scFv (light-heavy)]-[IC3 TCR beta chain]-C | MAIQMTQSPSSLSASVGDRVTITCRASQDIRNYLN WYQQKPGKAPKLLIYYTSRLESGVPSRFSGSGSGT DYTLTISSLQPEDFATYYCQQGNTLPWTFGQGTKV EIKGGGGSGGGGSGGGGSGGGGSGGGSEVQLVES GGGLVQPGGSLRLSCAASGYSFTGYTMNWVRQAP GKGLEWVALINPYKGVSTYNQKFKDRFTISVDKSK NTAYLQMNSLRAEDTAVYYCARSGYYGDSDWYF DVWGQGTLVTVSSGGGGSKAGVTQTPRYLIKTRG | 7 |
| | | QQVTLSCSPISGHRSVSWYQQTPGQGLQFLFEYFSE TQRNKGNFPGRFSGRQFSNSRSEMNVSTLELGDSA LYLCASSFNMATGQYFGPGTRLTVTEDLKNVFPPE VAVFEPSEAEISHTQKATLVCLATGFYPDHVELSW WVNGKEVHSGVCTDPQPLKEQPALNDSRYALSSR LRVSATFWQNPRNHFRCQVQFYGLSENDEWTQDR AKPVTQIVSAEAWGRADGGGLNDIFEAQKIEWHE | |
| TCR-21 | N-[10G VHH]-[Peptide-5]-[noncleavable linker]-[IC3 TCR alpha chain]-C | MEVQLVESGGGLVQPGNSLRLSCAASGFTFSKFG MSWVRQAPGKGLEWVSSISGSGRDTLYADSVKGR FTISRDNAKTTLYLQMNSLRPEDTAVYYCTIGGSLS VSSQGTLVTVSSGGGGSGGGSGGVSCKDVYDEAF CWTGGGGSGGSGGGSGGSSGTKQEVTQIPAALSV PEGENLVLNCSFTDSAIYNLQWFRQDPGKGLTSLL YVRPYQREQTSGRLNASLDKSSGRSTLYIAASQPG DSATYLCAVRPGGAGPFFVVFGKGTKLSVIPNIQN PDPAVYQLRDSKSSDKSVCLFTDFDSQTNVSQSKD SDVYITDKCVLDMRSMDFKSNSAVAWSNKSDFAC ANAFNNSIIPEDTFFPSPESSGGHHHHHHHH | 97 |
| TCR-21 | N-[UCHT1 scFv (light-heavy)]-[IC3 TCR beta chain]-C | MAIQMTQSPSSLSASVGDRVTITCRASQDIRNYLN WYQQKPGKAPKLLIYYTSRLESGVPSRFSGSGSGT DYTLTISSLQPEDFATYYCQQGNTLPWTFGQGTKV EIKGGGGSGGGGSGGGGSGGGGSGGGSEVQLVES GGGLVQPGGSLRLSCAASGYSFTGYTMNWVRQAP GKGLEWVALINPYKGVSTYNQKFKDRFTISVDKSK NTAYLQMNSLRAEDTAVYYCARSGYYGDSDWYF DVWGQGTLVTVSSGGGGSKAGVTQTPRYLIKTRG QQVTLSCSPISGHRSVSWYQQTPGQGLQFLFEYFSE TQRNKGNFPGRFSGRQFSNSRSEMNVSTLELGDSA LYLCASSFNMATGQYFGPGTRLTVTEDLKNVFPPE VAVFEPSEAEISHTQKATLVCLATGFYPDHVELSW WVNGKEVHSGVCTDPQPLKEQPALNDSRYALSSR LRVSATFWQNPRNHFRCQVQFYGLSENDEWTQDR AKPVTQIVSAEAWGRADGGGLNDIFEAQKIEWHE | 7 |
| TCR-22 | N-[10G VHH]-[Peptide-36]-[cleavable linker]-[IC3 TCR alpha chain]-C | MEVQLVESGGGLVQPGNSLRLSCAASGFTFSKFG MSWVRQAPGKGLEWVSSISGSGRDTLYADSVKGR FTISRDNAKTTLYLQMNSLRPEDTAVYYCTIGGSLS VSSQGTLVTVSSGGGGSGGGSGGASCAASASAAA CASGGGGSLSGRSDNHGSSGTKQEVTQIPAALSVP EGENLVLNCSFTDSAIYNLQWFRQDPGKGLTSLLY VRPYQREQTSGRLNASLDKSSGRSTLYIAASQPGD SATYLCAVRPGGAGPFFVVFGKGTKLSVIPNIQNP DPAVYQLRDSKSSDKSVCLFTDFDSQTNVSQSKDS DVYITDKCVLDMRSMDFKSNSAVAWSNKSDFAC ANAFNNSIIPEDTFFPSPESSGGHHHHHHHH | 98 |
| TCR-22 | N-[UCHT1 scFv (light-heavy)]-[IC3 TCR beta chain]-C | MAIQMTQSPSSLSASVGDRVTITCRASQDIRNYLN WYQQKPGKAPKLLIYYTSRLESGVPSRFSGSGSGT DYTLTISSLQPEDFATYYCQQGNTLPWTFGQGTKV EIKGGGGSGGGGSGGGGSGGGGSGGGSEVQLVES GGGLVQPGGSLRLSCAASGYSFTGYTMNWVRQAP GKGLEWVALINPYKGVSTYNQKFKDRFTISVDKSK NTAYLQMNSLRAEDTAVYYCARSGYYGDSDWYF DVWGQGTLVTVSSGGGGSKAGVTQTPRYLIKTRG QQVTLSCSPISGHRSVSWYQQTPGQGLQFLFEYFSE TQRNKGNFPGRFSGRQFSNSRSEMNVSTLELGDSA LYLCASSFNMATGQYFGPGTRLTVTEDLKNVFPPE VAVFEPSEAEISHTQKATLVCLATGFYPDHVELSW | 7 |
| | | WVNGKEVHSGVCTDPQPLKEQPALNDSRYALSSR LRVSATFWQNPRNHFRCQVQFYGLSENDEWTQDR AKPVTQIVSAEAWGRADGGGLNDIFEAQKIEWHE | |

Polypeptides or polypeptide complexes, in some embodiments, comprise a sequence set forth in Table 1. In some embodiments, the sequence comprises at least or about 70%, 80%, 85%, 90%, 91%, 92%, 93%, 94%, 95%, 96%, 97%, 98%, 99%, or 100% sequence identity to SEQ ID NOs: 1, 2, 4, 5, 7, 8, 9, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 53, 54, 55, 56, 57, 58, 59, 60, 61, 62, 63, 64, 65, 66, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, or 98. In some instances, the sequence comprises at least or about 95% homology to SEQ ID NOs: 1, 2, 4, 5, 7, 8, 9, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 53, 54, 55, 56, 57, 58, 59, 60, 61, 62, 63, 64, 65, 66, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, or 98. In some instances, the sequence comprises at least or about 97% homology to SEQ ID NOs: 1, 2, 4, 5, 7, 8, 9, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 53, 54, 55, 56, 57, 58, 59, 60, 61, 62, 63, 64, 65, 66, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, or 98. In some instances, the sequence comprises at least or about 99% homology to SEQ ID NOs: 1, 2, 4, 5, 7, 8, 9, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 53, 54, 55, 56, 57, 58, 59, 60, 61, 62, 63, 64, 65, 66, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, or 98. In some instances, the sequence comprises at least or about 100% homology to SEQ ID NOs: 1, 2, 4, 5, 7, 8, 9, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 53, 54, 55, 56, 57, 58, 59, 60, 61, 62, 63, 64, 65, 66, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, or 98. In some instances, the sequence comprises at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, or more than 400 amino acids of SEQ ID NOs: 1, 2, 4, 5, 7, 8, 9, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 53, 54, 55, 56, 57, 58, 59, 60, 61, 62, 63, 64, 65, 66, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, or 98.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. Typically, techniques for determining sequence identity include comparing two nucleotide or amino acid sequences and the determining their percent identity. Sequence comparisons, such as for the purpose of assessing identities, may be performed by any suitable alignment algorithm, including but not limited to the Needleman-Wunsch algorithm (see, e.g., the EMBOSS Needle aligner available at www.ebi.ac.uk/Tools/psa/emboss_needle/, optionally with default settings), the BLAST algorithm (see, e.g., the BLAST alignment tool available at blast.ncbi.nlm.nih.gov/Blast.cgi, optionally with default settings), and the Smith-Waterman algorithm (see, e.g., the EMBOSS Water aligner available at www.ebi.ac.uk/Tools/psa/emboss_water/, optionally with default settings). Optimal alignment may be assessed using any suitable parameters of a chosen algorithm, including default parameters. The “percent identity”, also referred to as “percent homology”, between two sequences may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin and Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the sequences being compared. Default parameters are provided to optimize searches with short query sequences, for example, with the blastp program. The program also allows use of an SEG filter to mask-off segments of the query sequences as determined by the SEG program of Wootton and Federhen, Computers and Chemistry 17: 149-163 (1993). High sequence identity generally includes ranges of sequence identity of approximately 80% to 100% and integer values there between.

### EMBODIMENTS

Embodiment 1 comprises a polypeptide or polypeptide complex according to Formula I:

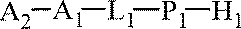

wherein: A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to a second target antigen.

Embodiment 2 comprises a polypeptide or polypeptide complex of embodiment 1, wherein the first target antigen comprises an effector cell antigen and the second target antigen comprises a tumor cell antigen.

Embodiment 3 comprises a polypeptide or polypeptide complex of any one of embodiments 1-2, wherein the effector cell antigen comprises CD3.

Embodiment 4 comprises a polypeptide or polypeptide complex of any one of embodiments 1-3, wherein the tumor cell antigen comprises MAGEA3 or MART1.

Embodiment 5 comprises a polypeptide or polypeptide complex of any one of embodiments 1-4, wherein A₁ comprises an antibody or antibody fragment.

Embodiment 6 comprises a polypeptide or polypeptide complex of any one of embodiments 1-5, wherein A₁ comprises an antibody or antibody fragment that is human or humanized.

Embodiment 7 comprises a polypeptide or polypeptide complex of any one of embodiments 1-6, wherein L₁ is bound to N-terminus of the antibody or antibody fragment.

Embodiment 8 comprises a polypeptide or polypeptide complex of any one of embodiments 1-7, wherein A₂ is bound to C-terminus of the antibody or antibody fragment.

Embodiment 9 comprises a polypeptide or polypeptide complex of any one of embodiments 1-8, wherein L₁ is bound to C-terminus of the antibody or antibody fragment.

Embodiment 10 comprises a polypeptide or polypeptide complex of any one of embodiments 1-9, wherein A₂ is bound to N-terminus of the antibody or antibody fragment.

Embodiment 11 comprises a polypeptide or polypeptide complex of any one of embodiments 1-10, wherein the antibody or antibody fragment comprises a single chain variable fragment, a single domain antibody, or a Fab fragment.

Embodiment 12 comprises a polypeptide or polypeptide complex of any one of embodiments 1-11, wherein A₁ is the single chain variable fragment (scFv).

Embodiment 13 comprises a polypeptide or polypeptide complex of any one of embodiments 1-12, wherein the scFv comprises a scFv heavy chain polypeptide and a scFv light chain polypeptide.

Embodiment 14 comprises a polypeptide or polypeptide complex of any one of embodiments 1-13, wherein A₁ is the single domain antibody.

Embodiment 15 comprises a polypeptide or polypeptide complex of any one of embodiments 1-14, wherein the single domain antibody comprises a single chain variable fragment (scFv), a heavy chain variable domain (VH domain), a light chain variable domain (VL domain), or a variable domain (VHH) of a camelid derived single domain antibody.

Embodiment 16 comprises a polypeptide or polypeptide complex of any one of embodiments 1-15, wherein A₁ comprises an anti-CD3e single chain variable fragment.

Embodiment 17 comprises a polypeptide or polypeptide complex of any one of embodiments 1-16, wherein A₁ comprises an anti-CD3e single chain variable fragment that has a K_{D} binding of 1 µM or less to CD3 on CD3 expressing cells.

Embodiment 18 comprises a polypeptide or polypeptide complex of any one of embodiments 1-17, wherein A₁ comprises a variable light chain and variable heavy chain each of which is capable of specifically binding to human CD3.

Embodiment 19 comprises a polypeptide or polypeptide complex of any one of embodiments 1-18, wherein A₁ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, UCHT1, and 15865v19.

Embodiment 20 comprises a polypeptide or polypeptide complex of any one of embodiments 1-19, wherein the polypeptide or polypeptide complex of formula I binds to an effector cell when L₁ is cleaved by the tumor specific protease.

Embodiment 21 comprises a polypeptide or polypeptide complex of any one of embodiments 1-20, wherein the polypeptide or polypeptide complex of formula I binds to an effector cell when L₁ is cleaved by the tumor specific protease and A₁binds to the effector cell.

Embodiment 22 comprises a polypeptide or polypeptide complex of any one of embodiments 1-21, wherein the effector cell is a T cell.

Embodiment 23 comprises a polypeptide or polypeptide complex of any one of embodiments 1-22, wherein A₁ binds to a polypeptide that is part of a TCR-CD3 complex on the effector cell.

Embodiment 24 comprises a polypeptide or polypeptide complex of any one of embodiments 1-23, wherein the polypeptide that is part of the TCR-CD3 complex is human CD3ε.

Embodiment 25 comprises a polypeptide or polypeptide complex of any one of embodiments 1-24, wherein the effector cell antigen comprises CD3, and the scFv comprises an amino acid sequence according to SEQ ID NO: 86 or 8.

Embodiment 26 comprises a polypeptide or polypeptide complex of any one of embodiments 1-25, wherein A₂ is a soluble T cell receptor (TCR).

Embodiment 27 comprises a polypeptide or polypeptide complex of any one of embodiments 1-26, wherein the soluble TCR is a single chain TCR comprising a variable region of a TCR alpha extracellular domain, or fragment thereof, and a variable region of a TCR beta extracellular domain, or fragment thereof.

Embodiment 28 comprises a polypeptide or polypeptide complex of any one of embodiments 1-27, wherein the soluble TCR comprises an alpha TCR polypeptide comprising a TCR alpha extracellular domain and a beta TCR polypeptide comprising a TCR beta extracellular domain.

Embodiment 29 comprises a polypeptide or polypeptide complex of any one of embodiments 1-28, wherein A₁ is bound to C-terminus of the alpha TCR polypeptide.

Embodiment 30 comprises a polypeptide or polypeptide complex of any one of embodiments 1-29, wherein A₁ is bound to C-terminus of the beta TCR polypeptide.

Embodiment 31 comprises a polypeptide or polypeptide complex of any one of embodiments 1-30, wherein A₁ is bound to N-terminus of the beta TCR polypeptide.

Embodiment 32 comprises a polypeptide or polypeptide complex of any one of embodiments 1-31, wherein the TCR alpha extracellular domain comprises three hypervariable complementarity determining regions (CDRs).

Embodiment 33 comprises a polypeptide or polypeptide complex of any one of embodiments 1-32, wherein at least one CDR comprises a mutation to increase binding affinity or binding specificity to the tumor cell antigen.

Embodiment 34 comprises a polypeptide or polypeptide complex of any one of embodiments 1-33, wherein the TCR beta extracellular domain comprises three hypervariable complementarity determining regions (CDRs).

Embodiment 35 comprises a polypeptide or polypeptide complex of any one of embodiments 1-34, wherein at least one CDR comprises a mutation to increase binding affinity or binding specificity to the tumor cell antigen.

Embodiment 36 comprises a polypeptide or polypeptide complex of any one of embodiments 1-35, wherein the TCR alpha extracellular domain, or fragment thereof, and the TCR beta extracellular domain, or fragment thereof, are connected by a disulfide bond.

Embodiment 37 comprises a polypeptide or polypeptide complex of any one of embodiments 1-36, wherein A₂ comprises a MAGEA3 binding TCR alpha domain.

Embodiment 38 comprises a polypeptide or polypeptide complex of any one of embodiments 1-37, wherein A₂ comprises a MAGEA3 binding TCR beta domain.

Embodiment 39 comprises a polypeptide or polypeptide complex of any one of embodiments 1-38, wherein A₂ comprises a MART1 binding TCR alpha domain.

Embodiment 40 comprises a polypeptide or polypeptide complex of any one of embodiments 1-39, wherein A₂ comprises a MART1 binding TCR beta domain.

Embodiment 41 comprises a polypeptide or polypeptide complex of any one of embodiments 1-40, wherein the tumor cell antigen comprises MAGEA3 or MART1.

Embodiment 42 comprises a polypeptide or polypeptide complex of any one of embodiments 1-41, wherein the tumor cell antigen comprises MAGEA3, and the alpha TCR polypeptide comprises an amino acid sequence according to SEQ ID NOs: 1, 5, 73, 75, 76, 79, 80, 85, 91, 92, 95, 96, 97, or 98.

Embodiment 43 comprises a polypeptide or polypeptide complex of any one of embodiments 1-42, wherein the tumor cell antigen comprises MAGEA3, and the beta TCR polypeptide an amino acid sequence according to SEQ ID NOs: 7, 9, 74, 77, 78, 81, 82, 83, 84, 87, 88, 89, 90, 93, or 94.

Embodiment 44 comprises a polypeptide or polypeptide complex of any one of embodiments 1-43, wherein the alpha TCR polypeptide of A₂ is bound to a C-terminus of the single chain variable fragment (scFv) of A₁.

Embodiment 45 comprises a polypeptide or polypeptide complex of any one of embodiments 1-44, wherein the beta TCR polypeptide of A₂ is bound to a C-terminus of the single chain variable fragment (scFv) A₁.

Embodiment 46 comprises a polypeptide or polypeptide complex of any one of embodiments 1-45, wherein the alpha TCR polypeptide of A₂ is bound to a N-terminus of the single chain variable fragment (scFv) of A₁.

Embodiment 47 comprises a polypeptide or polypeptide complex of any one of embodiments 1-46, wherein the beta TCR polypeptide of A₂ is bound to a N-terminus of the single chain variable fragment (scFv) A₁.

Embodiment 48 comprises a polypeptide or polypeptide complex of any one of embodiments 1-47, wherein the beta TCR polypeptide of A₂ is bound to the scFv heavy chain polypeptide of A₁.

Embodiment 49 comprises a polypeptide or polypeptide complex of any one of embodiments 1-48, wherein the beta TCR polypeptide of A₂ is bound to the scFv heavy chain polypeptide of A₁ at the C-terminus of the scFv.

Embodiment 50 comprises a polypeptide or polypeptide complex of any one of embodiments 1-49, wherein the alpha TCR polypeptide of A₂ is bound to the scFv heavy chain polypeptide of A₁.

Embodiment 51 comprises a polypeptide or polypeptide complex of any one of embodiments 1-50, wherein the alpha TCR polypeptide of A₂ is bound to the scFv heavy chain polypeptide of A₁ at the C-terminus of the scFv.

Embodiment 52 comprises a polypeptide or polypeptide complex of any one of embodiments 1-51, wherein the beta TCR polypeptide of A₂ is bound to the scFv light chain polypeptide of A₁.

Embodiment 53 comprises a polypeptide or polypeptide complex of any one of embodiments 1-52, wherein the alpha TCR polypeptide of A₂ is bound to the scFv light chain polypeptide of A₁ at the C-terminus of the scFv.

Embodiment 54 comprises a polypeptide or polypeptide complex of any one of embodiments 1-53, wherein A₂ further comprises P₂ and L₂, wherein P₂ comprises a peptide that binds to A₂; and L₂ comprises a linking moiety that connects A₂ to P₂ and is a substrate for a tumor specific protease.

Embodiment 55 comprises the polypeptide or polypeptide complex according to any one of embodiments 1-54 wherein the polypeptide or polypeptide complex is according to Formula Ia:

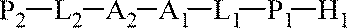

Embodiment 56 comprises a polypeptide or polypeptide complex of any one of embodiments 1-55, wherein the beta TCR polypeptide of A₂ is bound to the scFv heavy chain polypeptide of A₁ and L₂ is bound to the alpha TCR polypeptide of A₂.

Embodiment 57 comprises a polypeptide or polypeptide complex of any one of embodiments 1-56, wherein the beta TCR polypeptide of A₂ is bound to the scFv heavy chain polypeptide of A₁ at the C-terminus of the scFv and L₂ is bound to the alpha TCR polypeptide of A₂ at the N-terminus of the alpha TCR polypeptide.

Embodiment 58 comprises a polypeptide or polypeptide complex of any one of embodiments 1-57, wherein the alpha TCR polypeptide of A₂ is bound to the scFv heavy chain polypeptide of A₁ and L₂ is bound to the beta TCR polypeptide of A₂.

Embodiment 59 comprises a polypeptide or polypeptide complex of any one of embodiments 1-58, wherein the alpha TCR polypeptide of A₂ is bound to the scFv heavy chain polypeptide of A₁ at the C-terminus of the scFv and L₂ is bound to the beta TCR polypeptide of A₂ at the N-terminus of the beta TCR polypeptide.

Embodiment 60 comprises a polypeptide or polypeptide complex of any one of embodiments 1-59, wherein the beta TCR polypeptide of A₂ is bound to the scFv light chain polypeptide of A₁ and L₂ is bound to the alpha TCR polypeptide of A₂.

Embodiment 61 comprises a polypeptide or polypeptide complex of any one of embodiments 1-60, wherein the beta TCR polypeptide of A₂ is bound to the scFv light chain polypeptide of A₁ at the C-terminus of the scFv and L₂ is bound to the alpha TCR polypeptide of A₂ at the N-terminus of the alpha TCR polypeptide.

Embodiment 62 comprises a polypeptide or polypeptide complex of any one of embodiments 1-61, wherein the alpha TCR polypeptide of A₂ is bound to the scFv light chain polypeptide of A₁ and L₂ is bound to the beta TCR polypeptide of A₂.

Embodiment 63 comprises a polypeptide or polypeptide complex of any one of embodiments 1-62, wherein the alpha TCR polypeptide of A₂ is bound to the scFv light chain polypeptide of A₁ at the C-terminus of the scFv and L₂ is bound to the beta TCR polypeptide of A₂ at the N-terminus of the beta TCR polypeptide.

Embodiment 64 comprises a polypeptide or polypeptide complex of any one of embodiments 1-63, wherein the first target antigen comprises a tumor cell antigen and the second target antigen comprises an effector cell antigen.

Embodiment 65 comprises a polypeptide or polypeptide complex of any one of embodiments 1-64, wherein the tumor cell antigen comprises MAGEA3 or MART1.

Embodiment 66 comprises a polypeptide or polypeptide complex of any one of embodiments 1-65, wherein the effector cell antigen comprises CD3.

Embodiment 67 comprises a polypeptide or polypeptide complex of any one of embodiments 1-66, wherein A₁ is a soluble T cell receptor (TCR).

Embodiment 68 comprises a polypeptide or polypeptide complex of any one of embodiments 1-67, wherein the soluble TCR is a single chain TCR comprising a variable region of a TCR alpha extracellular domain, or fragment thereof, and a variable region of a TCR beta extracellular domain, or fragment thereof.

Embodiment 69 comprises a polypeptide or polypeptide complex of any one of embodiments 1-68, wherein the soluble TCR comprises an alpha TCR polypeptide comprising a TCR alpha extracellular domain and a beta TCR polypeptide comprising a TCR beta extracellular domain.

Embodiment 70 comprises a polypeptide or polypeptide complex of any one of embodiments 1-69, wherein the tumor cell antigen comprises MAGEA3, and the alpha TCR polypeptide comprises an amino acid sequence according to SEQ ID NOs: 1, 5, 73, 75, 76, 79, 80, 85, 91, 92, 95, 96, 97, or 98.

Embodiment 71 comprises a polypeptide or polypeptide complex of any one of embodiments 1-70, wherein the tumor cell antigen comprises MAGEA3, and the beta TCR polypeptide comprises an amino acid sequence according to SEQ ID NOs: 7, 9, 74, 77, 78, 81, 82, 83, 84, 87, 88, 89, 90, 93, or 94.

Embodiment 72 comprises a polypeptide or polypeptide complex of any one of embodiments 1-71, wherein A₂ comprises an antibody or antibody fragment.

Embodiment 73 comprises a polypeptide or polypeptide complex of any one of embodiments 1-72, wherein A₂ comprises an antibody or antibody fragment that is human or humanized.

Embodiment 74 comprises a polypeptide or polypeptide complex of any one of embodiments 1-73, wherein the antibody or antibody fragment comprises a single chain variable fragment, a single domain antibody, or a Fab fragment.

Embodiment 75 comprises a polypeptide or polypeptide complex of any one of embodiments 1-74, wherein A₂ is the single chain variable fragment (scFv).

Embodiment 76 comprises a polypeptide or polypeptide complex of any one of embodiments 1-75, wherein the scFv comprises a scFv heavy chain polypeptide and a scFv light chain polypeptide.

Embodiment 77 comprises a polypeptide or polypeptide complex of any one of embodiments 1-76, wherein A₂ is the single domain antibody.

Embodiment 78 comprises a polypeptide or polypeptide complex of any one of embodiments 1-77, wherein the single domain antibody comprises a single chain variable fragment (scFv), a heavy chain variable domain (VH domain), a light chain variable domain (VL domain), or a variable domain (VHH) of a camelid derived single domain antibody.

Embodiment 79 comprises a polypeptide or polypeptide complex of any one of embodiments 1-78, wherein A₂ comprises an anti-CD3e single chain variable fragment.

Embodiment 80 comprises a polypeptide or polypeptide complex of any one of embodiments 1-79, wherein A₂ comprises an anti-CD3e single chain variable fragment that has a K_{D} binding of 1 µM or less to CD3 on CD3 expressing cells.

Embodiment 81 comprises a polypeptide or polypeptide complex of any one of embodiments 1-80, wherein A₂ comprises a variable light chain and variable heavy chain each of which is capable of specifically binding to human CD3.

Embodiment 82 comprises a polypeptide or polypeptide complex of any one of embodiments 1-81,wherein A₂ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19.

Embodiment 83 comprises a polypeptide or polypeptide complex of any one of embodiments 1-82, wherein the polypeptide or polypeptide complex of formula I binds to an effector cell.

Embodiment 84 comprises a polypeptide or polypeptide complex of any one of embodiments 1-83, wherein the effector cell is a T cell.

Embodiment 85 comprises a polypeptide or polypeptide complex of any one of embodiments 1-84,wherein A₂ binds to a polypeptide that is part of a TCR-CD3 complex on the effector cell.

Embodiment 86 comprises a polypeptide or polypeptide complex of any one of embodiments 1-85, wherein the polypeptide that is part of the TCR-CD3 complex is human CD3ε.

Embodiment 87 comprises a polypeptide or polypeptide complex of any one of embodiments 1-86, wherein the effector cell antigen comprises CD3, and the scFv comprises an amino acid sequence according to SEQ ID NO: 86 or 8.

Embodiment 88 comprises a polypeptide or polypeptide complex of any one of embodiments 1-87, wherein L₁ is bound to N-terminus of the alpha TCR polypeptide.

Embodiment 89 comprises a polypeptide or polypeptide complex of any one of embodiments 1-88, wherein L₁ is bound to N-terminus of the beta TCR polypeptide.

Embodiment 90 comprises a polypeptide or polypeptide complex of any one of embodiments 1-89, wherein A₂ is bound to C-terminus of the alpha TCR polypeptide.

Embodiment 91 comprises a polypeptide or polypeptide complex of any one of embodiments 1-90, wherein A₂ is bound to N-terminus of the alpha TCR polypeptide.

Embodiment 92 comprises a polypeptide or polypeptide complex of any one of embodiments 1-91, wherein A₂ is bound to C-terminus of the beta TCR polypeptide.

Embodiment 93 comprises a polypeptide or polypeptide complex of any one of embodiments 1-92, wherein A₂ is bound to N-terminus of the beta TCR polypeptide.

Embodiment 94 comprises a polypeptide or polypeptide complex of any one of embodiments 1-93, wherein L₁ is bound to N-terminus of the alpha TCR polypeptide and A₂ is bound to N-terminus of the beta TCR polypeptide.

Embodiment 95 comprises a polypeptide or polypeptide complex of any one of embodiments 1-94, wherein L₁ is bound to N-terminus of the alpha TCR polypeptide and A₂ is bound to C-terminus of the beta TCR polypeptide.

Embodiment 96 comprises a polypeptide or polypeptide complex of any one of embodiments 1-95, wherein L₁ is bound to N-terminus of the alpha TCR polypeptide and A₂ is bound to C-terminus of the alpha TCR polypeptide.

Embodiment 97 comprises a polypeptide or polypeptide complex of any one of embodiments 1-96, wherein L₁ is bound to N-terminus of the beta TCR polypeptide and A₂ is bound to N-terminus of the alpha TCR polypeptide.

Embodiment 98 comprises a polypeptide or polypeptide complex of any one of embodiments 1-97, wherein L₁ is bound to N-terminus of the beta TCR polypeptide and A₂ is bound to C-terminus of the beta TCR polypeptide.

Embodiment 99 comprises a polypeptide or polypeptide complex of any one of embodiments 1-98, wherein L₁ is bound to N-terminus of the beta TCR polypeptide and A₂ is bound to C-terminus of the alpha TCR polypeptide.

Embodiment 100 comprises a polypeptide or polypeptide complex of any one of embodiments 1-99, wherein the alpha TCR polypeptide of A₁ is bound to a C-terminus of the single chain variable fragment (scFv) of A₂.

Embodiment 101 comprises a polypeptide or polypeptide complex of any one of embodiments 1-100, wherein the beta TCR polypeptide of A₁ is bound to a C-terminus of the single chain variable fragment (scFv) A₂.

Embodiment 102 comprises a polypeptide or polypeptide complex of any one of embodiments 1-101, wherein the alpha TCR polypeptide of A₁ is bound to a N-terminus of the single chain variable fragment (scFv) of A₂.

Embodiment 103 comprises a polypeptide or polypeptide complex of any one of embodiments 1-102, wherein the beta TCR polypeptide of A₁ is bound to a N-terminus of the single chain variable fragment (scFv) A₂.

Embodiment 104 comprises a polypeptide or polypeptide complex of any one of embodiments 1-103, wherein the beta TCR polypeptide of A₁ is bound to the scFv heavy chain polypeptide of A₂ and L₁ is bound to the alpha TCR polypeptide of A₁.

Embodiment 105 comprises a polypeptide or polypeptide complex of any one of embodiments 1-104, wherein the beta TCR polypeptide of A₁ is bound to the scFv heavy chain polypeptide of A₂ at the C-terminus of the scFv and L₁ is bound to the alpha TCR polypeptide of A₁ at the N-terminus of the alpha TCR polypeptide.

Embodiment 106 comprises a polypeptide or polypeptide complex of any one of embodiments 1-105, wherein the beta TCR polypeptide of A₁ is bound to the scFv heavy chain polypeptide of A₂ at the C-terminus of the scFv and L₁ is bound to the alpha TCR polypeptide of A₁ at the N-terminus of the alpha TCR polypeptide and the polypeptide complex comprises amino acid sequences of TCR-20alpha and TCR-20-beta.

Embodiment 107 comprises a polypeptide or polypeptide complex of any one of embodiments 1-106, wherein the beta TCR polypeptide of A₁ is bound to the scFv heavy chain polypeptide of A₂ at the N-terminus of the scFv and L₁ is bound to the alpha TCR polypeptide of A₁ at the N-terminus of the alpha TCR polypeptide.

Embodiment 108 comprises a polypeptide or polypeptide complex of any one of embodiments 1-107, wherein the alpha TCR polypeptide of A₁ is bound to the scFv heavy chain polypeptide of A₂ and L₁ is bound to the beta TCR polypeptide of A₁.

Embodiment 109 comprises a polypeptide or polypeptide complex of any one of embodiments 1-108, wherein the alpha TCR polypeptide of A₁ is bound to the scFv heavy chain polypeptide of A₂ at the C-terminus of the scFv and L₁ is bound to the beta TCR polypeptide of A₁ at the N-terminus of the beta TCR polypeptide.

Embodiment 110 comprises a polypeptide or polypeptide complex of any one of embodiments 1-109, wherein the alpha TCR polypeptide of A₁ is bound to the scFv heavy chain polypeptide of A₂ at the N-terminus of the scFv and L₁ is bound to the beta TCR polypeptide of A₁ at the N-terminus of the beta TCR polypeptide.

Embodiment 111 comprises a polypeptide or polypeptide complex of any one of embodiments 1-110, wherein the beta TCR polypeptide of A₁ is bound to the scFv light chain polypeptide of A₂ and L₁ is bound to the alpha TCR polypeptide of A₁.

Embodiment 112 comprises a polypeptide or polypeptide complex of any one of embodiments 1-111, wherein the beta TCR polypeptide of A₁ is bound to the scFv light chain polypeptide of A₂ at the C-terminus of the scFv and L₁ is bound to the alpha TCR polypeptide of A₁ at the N-terminus of the alpha TCR polypeptide.

Embodiment 113 comprises a polypeptide or polypeptide complex of any one of embodiments 1-112, wherein the beta TCR polypeptide of A₁ is bound to the scFv light chain polypeptide of A₂ at the N-terminus of the scFv and L₁ is bound to the alpha TCR polypeptide of A₁ at the N-terminus of the alpha TCR polypeptide.

Embodiment 114 comprises a polypeptide or polypeptide complex of any one of embodiments 1-113, wherein the alpha TCR polypeptide of A₁ is bound to the scFv light chain polypeptide of A₂ and L₁ is bound to the beta TCR polypeptide of A₁.

Embodiment 115 comprises a polypeptide or polypeptide complex of any one of embodiments 1-114, wherein the alpha TCR polypeptide of A₁ is bound to the scFv light chain polypeptide of A₂ at the C-terminus of the scFv and L₁ is bound to the beta TCR polypeptide of A₁ at the N-terminus of the beta TCR polypeptide.

Embodiment 116 comprises a polypeptide or polypeptide complex of any one of embodiments 1-115, wherein the alpha TCR polypeptide of A₁ is bound to the scFv light chain polypeptide of A₂ at the N-terminus of the scFv and L₁ is bound to the beta TCR polypeptide of A₁ at the N-terminus of the beta TCR polypeptide.

Embodiment 117 comprises a polypeptide or polypeptide complex of any one of embodiments 1-116, wherein the alpha TCR polypeptide of A₁ is bound to the scFv heavy chain polypeptide of A₂ at the N-terminus of the scFv and L₁ is bound to the alpha TCR polypeptide of A₁ at the N-terminus of the alpha TCR polypeptide.

Embodiment 118 comprises a polypeptide or polypeptide complex of any one of embodiments 1-117, wherein the beta TCR polypeptide of A₁ is bound to the scFv heavy chain polypeptide of A₂ at the N-terminus of the scFv and L₁ is bound to the beta TCR polypeptide of A₁ at the N-terminus of the beta TCR polypeptide.

Embodiment 119 comprises a polypeptide or polypeptide complex of any one of embodiments 1-118, wherein the alpha TCR polypeptide of A₁ is bound to the scFv light chain polypeptide of A₂ at the N-terminus of the scFv and L₁ is bound to the alpha TCR polypeptide of A₁ at the N-terminus of the alpha TCR polypeptide.

Embodiment 120 comprises a polypeptide or polypeptide complex of any one of embodiments 1-119, wherein the beta TCR polypeptide of A₁ is bound to the scFv light chain polypeptide of A₂ at the N-terminus of the scFv and L₁ is bound to the beta TCR polypeptide of A₁ at the N-terminus of the beta TCR polypeptide.

Embodiment 121 comprises a polypeptide or polypeptide complex of any one of embodiments 1-120, wherein A₂ further comprises P₂ and L₂, wherein P₂ comprises a peptide that binds to A₂; and L₂ comprises a linking moiety that connects A₂ to P₂ and is a substrate for a tumor specific protease.

Embodiment 122 comprises the polypeptide or polypeptide complex according to any one of embodiments 1-121 wherein the polypeptide or polypeptide complex is according to Formula Ia

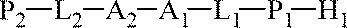

Embodiment 123 comprises a polypeptide or polypeptide complex of any one of embodiments 1-122, wherein the beta TCR polypeptide of A₁ is bound to the scFv heavy chain polypeptide of A₂ and L₁ is bound to the alpha TCR polypeptide of A₁ and L₂ is bound to the scFv light chain polypeptide of A₂.

Embodiment 124 comprises a polypeptide or polypeptide complex of any one of embodiments 1-123, wherein the beta TCR polypeptide of A₁ is bound to the scFv heavy chain polypeptide of A₂ at the C-terminus of the scFv and L₁ is bound to the alpha TCR polypeptide of A₁ at the N-terminus of the alpha TCR polypeptide and L₂ is bound to the scFv light chain polypeptide of A₂ at the N-terminus of the scFv.

Embodiment 125 comprises a polypeptide or polypeptide complex of any one of embodiments 1-124, wherein the alpha TCR polypeptide of A₁ is bound to the scFv heavy chain polypeptide of A₂ and L₁ is bound to the beta TCR polypeptide of A₁ and L₂ is bound to the scFv light chain polypeptide of A₂.

Embodiment 126 comprises a polypeptide or polypeptide complex of any one of embodiments 1-125, wherein the alpha TCR polypeptide of A₁ is bound to the scFv heavy chain polypeptide of A₂ at the C-terminus of the scFv and L₁ is bound to the beta TCR polypeptide of A₁ at the N-terminus of the beta TCR polypeptide and L₂ is bound to the scFv light chain polypeptide of A₂ at the N-terminus of the scFv.

Embodiment 127 comprises a polypeptide or polypeptide complex of any one of embodiments 1-126, wherein the beta TCR polypeptide of A₁ is bound to the scFv light chain polypeptide of A₂ and L₁ is bound to the alpha TCR polypeptide of A₁ and L₂ is bound to the scFv heavy chain polypeptide of A₂.

Embodiment 128 comprises a polypeptide or polypeptide complex of any one of embodiments 1-127, wherein the beta TCR polypeptide of A₁ is bound to the scFv light chain polypeptide of A₂ at the C-terminus of the scFv and L₁ is bound to the alpha TCR polypeptide of A₁ at the N-terminus of the alpha TCR polypeptide and L₂ is bound to the scFv heavy chain polypeptide of A₂ at the N-terminus of the scFv.

Embodiment 129 comprises a polypeptide or polypeptide complex of any one of embodiments 1-128,wherein the alpha TCR polypeptide of A₁ is bound to the scFv light chain polypeptide of A₂ and L₁ is bound to the beta TCR polypeptide of A₁ and L₂ is bound to the scFv heavy chain polypeptide of A₂.

Embodiment 130 comprises a polypeptide or polypeptide complex of any one of embodiments 1-129, wherein the alpha TCR polypeptide of A₁ is bound to the scFv light chain polypeptide of A₂ at the C-terminus of the scFv and L₁ is bound to the beta TCR polypeptide of A₁ at the N-terminus of the beta TCR polypeptide and L₂ is bound to the scFv heavy chain polypeptide of A₂ at the N-terminus of the scFv.

Embodiment 131 comprises a polypeptide or polypeptide complex of any one of embodiments 1-130,wherein P₁ impairs binding of A₁ to the first target antigen.

Embodiment 132 comprises a polypeptide or polypeptide complex of any one of embodiments 1-131, wherein P₁ is bound to A₁ through ionic interactions, electrostatic interactions, hydrophobic interactions, Pi-stacking interactions, and H-bonding interactions, or a combination thereof.

Embodiment 133 comprises a polypeptide or polypeptide complex of any one of embodiments 1-132, wherein P₁ has less than 70% sequence homology to the first target antigen.

Embodiment 134 comprises a polypeptide or polypeptide complex of any one of embodiments 1-133, wherein P₂ impairs binding of A₂ to the second target antigen.

Embodiment 135 comprises a polypeptide or polypeptide complex of any one of embodiments 1-134, wherein P₂ is bound to A₂ through ionic interactions, electrostatic interactions, hydrophobic interactions, Pi-stacking interactions, and H-bonding interactions, or a combination thereof.

Embodiment 136 comprises a polypeptide or polypeptide complex of any one of embodiments 1-135, wherein P₂ is bound to A₂ at or near an antigen binding site.

Embodiment 137 comprises a polypeptide or polypeptide complex of any one of embodiments 1-136, wherein P₂ has less than 70% sequence homology to the second target antigen.

Embodiment 138 comprises a polypeptide or polypeptide complex of any one of embodiments 1-137, wherein P₁ or P₂ comprises a peptide sequence of at least 10 amino acids in length.

Embodiment 139 comprises a polypeptide or polypeptide complex of any one of embodiments 1-138, wherein P₁ or P₂ comprises a peptide sequence of at least 10 amino acids in length and no more than 20 amino acids in length.

Embodiment 140 comprises a polypeptide or polypeptide complex of any one of embodiments 1-139, wherein P₁ or P₂ comprises a peptide sequence of at least 16 amino acids in length.

Embodiment 141 comprises a polypeptide or polypeptide complex of any one of embodiments 1-140, wherein P₁ or P₂ comprises a peptide sequence of no more than 40 amino acids in length.

Embodiment 142 comprises a polypeptide or polypeptide complex of any one of embodiments 1-141, wherein P₁ or P₂ comprises at least two cysteine amino acid residues.

Embodiment 143 comprises a polypeptide or polypeptide complex of any one of embodiments 1-142, wherein P₁ or P₂ comprises a cyclic peptide or a linear peptide.

Embodiment 144 comprises a polypeptide or polypeptide complex of any one of embodiments 1-143, wherein P₁ or P₂ comprises a cyclic peptide.

Embodiment 145 comprises a polypeptide or polypeptide complex of any one of embodiments 1-144, wherein P₁ or P₂ comprises a linear peptide.

Embodiment 146 comprises a polypeptide or polypeptide complex of any one of embodiments 1-145, wherein the tumor cell antigen comprises MAGEA3, and the and the P₁ or P₂ comprises an amino acid sequence selected from the group consisting of GGESCQSVYDSSFCYD (SEQ ID NO: 13), GGNACEMTYDHTFCDP (SEQ ID NO: 14), GGRICEEVYDWIFCES (SEQ ID NO: 15), GGRRCVDVYDNAFCLI (SEQ ID NO: 16), GGVSCKDVYDEAFCWT (SEQ ID NO: 12), GGTSCAQIYDFEFCYS (SEQ ID NO: 17), GGSLCSLVYDQDFCES (SEQ ID NO: 18), GGNSCSLVYDKAFCLF (SEQ ID NO: 19), GGNQCWEVYDQEFCSL (SEQ ID NO: 20), GGSACSRIYDFAFCHT (SEQ ID NO: 21), GGTFCYFDHGLVNCQW (SEQ ID NO: 22), GGHCFVSPASGEWWCV (SEQ ID NO: 23), GGCSWIFDGLRYFSKC (SEQ ID NO: 24), VRTWFEKFPELV (SEQ ID NO: 25), LVWGCIWDDMCS (SEQ ID NO: 26), WHWEPSMVWGML (SEQ ID NO: 27), GGGCFVSPATGFTWCV (SEQ ID NO: 28), GGDCQPDSVWSYWYCR (SEQ ID NO: 29), GGCTFVDWWVLGSPYC (SEQ ID NO: 30), GGCLMNDYYYLWGGHC (SEQ ID NO: 31), GGASCKDVYDEAFCWT (SEQ ID NO: 32), GGVACKDVYDEAFCWT (SEQ ID NO: 33), GGVSAKDVYDEAFCWT (SEQ ID NO: 34), GGVSCADVYDEAFCWT (SEQ ID NO: 35), GGVSCKAVYDEAFCWT (SEQ ID NO: 36), GGVSCKDAYDEAFCWT (SEQ ID NO: 37), GGVSCKDVADEAFCWT (SEQ ID NO: 38), GGVSCKDVYAEAFCWT (SEQ ID NO: 39), GGVSCKDVYDAAFCWT (SEQ ID NO: 40), GGVSCKDVYDEAACWT (SEQ ID NO: 41), GGVSCKDVYDEAFAWT (SEQ ID NO: 42), GGVSCKDVYDEAFCAT (SEQ ID NO: 43), GGVSCKDVYDEAFCWA (SEQ ID NO: 44), EVDPIGHLY (SEQ ID NO: 45), ESDPIVAQY (SEQ ID NO: 46), and GGASCAASASAAACAS (SEQ ID NO: 47).

Embodiment 147 comprises a polypeptide or polypeptide complex of any one of embodiments 1-146, wherein L₁ is bound to N-terminus of A₁.

Embodiment 148 comprises a polypeptide or polypeptide complex of any one of embodiments 1-147, wherein L₁ is bound to C-terminus of A₁.

Embodiment 149 comprises a polypeptide or polypeptide complex of any one of embodiments 1-148, wherein L₂ is bound to N-terminus of A₂.

Embodiment 150 comprises a polypeptide or polypeptide complex of any one of embodiments 1-149, wherein L₂ is bound to C-terminus of A₂.

Embodiment 151 comprises a polypeptide or polypeptide complex of any one of embodiments 1-150, wherein L₁ or L₂ is a peptide sequence having at least 5 to no more than 50 amino acids.

Embodiment 152 comprises a polypeptide or polypeptide complex of any one of embodiments 1-151, wherein L₁ or L₂ is a peptide sequence having at least 10 to no more than 30 amino acids.

Embodiment 153 comprises a polypeptide or polypeptide complex of any one of embodiments 1-152, wherein L₁ or L₂ is a peptide sequence having at least 10 amino acids.

Embodiment 154 comprises a polypeptide or polypeptide complex of any one of embodiments 1-153, wherein L₁ or L₂ is a peptide sequence having at least 18 amino acids.

Embodiment 155 comprises a polypeptide or polypeptide complex of any one of embodiments 1-154, wherein L₁ or L₂ is a peptide sequence having at least 26 amino acids.

Embodiment 156 comprises a polypeptide or polypeptide complex of any one of embodiments 1-155, wherein L₁ or L₂ has a formula comprising (G₂S)ₙ, wherein n is an integer from 1 to 3 (SEQ ID NO: 48).

Embodiment 157 comprises a polypeptide or polypeptide complex of any one of embodiments 1-156, wherein L₁ has a formula selected from the group consisting of (G₂S)ₙ, (GS)ₙ, (GSGGS)ₙ (SEQ ID NO: 49), (GGGS)ₙ (SEQ ID NO: 50), (GGGGS)ₙ (SEQ ID NO: 51), and (GSSGGS)ₙ (SEQ ID NO: 52), wherein n is an integer of at least 1.

Embodiment 158 comprises a polypeptide or polypeptide complex of any one of embodiments 1-157, wherein P₁ becomes unbound from A₁ when L₁ is cleaved by the tumor specific protease thereby exposing A₁ to the first target antigen.

Embodiment 159 comprises a polypeptide or polypeptide complex of any one of embodiments 1-158, wherein P₂ becomes unbound from A₂ when L₂ is cleaved by the tumor specific protease thereby exposing A₂ to the second target antigen.

Embodiment 160 comprises a polypeptide or polypeptide complex of any one of embodiments 1-159, wherein the tumor specific protease is selected from the group consisting of metalloprotease, serine protease, cysteine protease, threonine protease, and aspartic protease.

Embodiment 161 comprises a polypeptide or polypeptide complex of any one of embodiments 1-160, wherein L₁ or L₂ comprises a urokinase cleavable amino acid sequence, a matriptase cleavable amino acid sequence, matrix metalloprotease cleavable amino acid sequence, or a legumain cleavable amino acid sequence.

Embodiment 162 comprises a polypeptide or polypeptide complex of any one of embodiments 1-161, wherein L₁ or L₂ comprises an amino acid sequence selected from the group consisting of GGGGSLSGRSDNHGSSGT (SEQ ID NO: 53), GGGGSSGGSGGSGLSGRSDNHGSSGT (SEQ ID NO: 54), ASGRSDNH (SEQ ID NO: 55), LAGRSDNH (SEQ ID NO: 56), ISSGLASGRSDNH (SEQ ID NO: 57), ISSGLLAGRSDNH (SEQ ID NO: 58), LSGRSDNH (SEQ ID NO: 4), ISSGLLSGRSDNP (SEQ ID NO: 59), ISSGLLSGRSDNH (SEQ ID NO: 60), LSGRSDNHSPLGLAGS (SEQ ID NO: 61), SPLGLAGSLSGRSDNH (SEQ ID NO: 62), SPLGLSGRSDNH (SEQ ID NO: 63), LAGRSDNHSPLGLAGS (SEQ ID NO: 64), LSGRSDNHVPLSLKMG (SEQ ID NO: 65), LSGRSDNHVPLSLSMG (SEQ ID NO: 66), GGGGSLSGRSDNHGSSGT (SEQ ID NO: 53), GGGGSSGGSGGSGLSGRSDNHGSSGT (SEQ ID NO: 54), ASGRSDNH (SEQ ID NO: 55), LAGRSDNH (SEQ ID NO: 56), ISSGLASGRSDNH (SEQ ID NO: 57), ISSGLLAGRSDNH (SEQ ID NO: 58), LSGRSDNH (SEQ ID NO: 4), ISSGLLSGRSDNP (SEQ ID NO: 59), ISSGLLSGRSDNH (SEQ ID NO: 60), LSGRSDNHSPLGLAGS (SEQ ID NO: 61), SPLGLAGSLSGRSDNH (SEQ ID NO: 62), SPLGLSGRSDNH (SEQ ID NO: 63), LAGRSDNHSPLGLAGS (SEQ ID NO: 64), LSGRSDNHVPLSLKMG (SEQ ID NO: 65), LSGRSDNHVPLSLSMG (SEQ ID NO: 66), GSSGGSGGSGGSGLSGRSDNHGSSGT (SEQ ID NO: 68), GSSGGSGGSGGSGGGSGGGSGGSSGT (SEQ ID NO: 69), GGGGSGGSGGGSGGSSGT (SEQ ID NO: 70), and GGGGSGGGS (SEQ ID NO: 71).

Embodiment 163 comprises a polypeptide or polypeptide complex of any one of embodiments 1-162, wherein L₁ or L₂ comprises an amino acid sequence ASGRSDNH (SEQ ID NO: 55), LAGRSDNH (SEQ ID NO: 56), ISSGLASGRSDNH (SEQ ID NO: 57), and ISSGLLAGRSDNH (SEQ ID NO: 58).

Embodiment 164 comprises a polypeptide or polypeptide complex of any one of embodiments 1-163, wherein H₁ comprises a polymer.

Embodiment 165 comprises a polypeptide or polypeptide complex of any one of embodiments 1-164, wherein the polymer is polyethylene glycol (PEG).

Embodiment 166 comprises a polypeptide or polypeptide complex of any one of embodiments 1-165, wherein H₁ comprises albumin.

Embodiment 167 comprises a polypeptide or polypeptide complex of any one of embodiments 1-166, wherein H₁ comprises an Fc domain.

Embodiment 168 comprises a polypeptide or polypeptide complex of any one of embodiments 1-167, wherein the albumin is serum albumin.

Embodiment 169 comprises a polypeptide or polypeptide complex of any one of embodiments 1-168,wherein the albumin is human serum albumin.

Embodiment 170 comprises a polypeptide or polypeptide complex of any one of embodiments 1-169, wherein H₁ comprises a polypeptide, a ligand, or a small molecule.

Embodiment 171 comprises a polypeptide or polypeptide complex of any one of embodiments 1-170, wherein the polypeptide, the ligand or the small molecule binds serum protein or a fragment thereof, a circulating immunoglobulin or a fragment thereof, or CD35/CR1.

Embodiment 172 comprises a polypeptide or polypeptide complex of any one of embodiments 1-171, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 173 comprises a polypeptide or polypeptide complex of any one of embodiments 1-172, wherein the circulating immunoglobulin molecule comprises IgG1, IgG2, IgG3, IgG4, slgA, IgM or IgD.

Embodiment 174 comprises a polypeptide or polypeptide complex of any one of embodiments 1-173, wherein the serum protein is albumin.

Embodiment 175 comprises a polypeptide or polypeptide complex of any one of embodiments 1-174, wherein the polypeptide is an antibody.

Embodiment 176 comprises a polypeptide or polypeptide complex of any one of embodiments 1-175, wherein the single domain antibody comprises a single domain antibody, a single chain variable fragment or a Fab.

Embodiment 177 comprises a polypeptide or polypeptide complex of any one of embodiments 1-176, wherein the single domain antibody comprises a single domain antibody that binds to albumin.

Embodiment 178 comprises a polypeptide or polypeptide complex of any one of embodiments 1-177, wherein the single domain antibody is a human or humanized antibody.

Embodiment 179 comprises a polypeptide or polypeptide complex of any one of embodiments 1-178, wherein the single domain antibody is 645gH1gL1.

Embodiment 180 comprises a polypeptide or polypeptide complex of any one of embodiments 1-179, wherein the single domain antibody is 645dsgH5gL4.

Embodiment 181 comprises a polypeptide or polypeptide complex of any one of embodiments 1-180, wherein the single domain antibody is 23-13-A01 -sc02.

Embodiment 182 comprises a polypeptide or polypeptide complex of any one of embodiments 1-181, wherein the single domain antibody is A10m3 or a fragment thereof.

Embodiment 183 comprises a polypeptide or polypeptide complex of any one of embodiments 1-182, wherein the single domain antibody is DOM7r-31.

Embodiment 184 comprises a polypeptide or polypeptide complex of any one of embodiments 1-183, wherein the single domain antibody is DOM7h-11-15.

Embodiment 185 comprises a polypeptide or polypeptide complex of any one of embodiments 1-184, wherein the single domain antibody is Alb-1, Alb-8, or Alb-23.

Embodiment 186 comprises a polypeptide or polypeptide complex of any one of embodiments 1-185, wherein the single domain antibody is 10G or 10GE.

Embodiment 187 comprises a polypeptide or polypeptide complex of any one of embodiments 1-186, wherein the single domain antibody is 10G, and the single domain antibody comprises an amino acid sequence

MEVQLVESGGGLVQPGNSLRLSCAASGFTFSKFGMSWVRQAPGKGLEWVS
SISGSGRDTLYADSVKGRFTISRDNAKTTLYLQMNSLRPEDTAVYYCTIG
GSLSVSSQGTLVTVSS (SEQ ID NO: 2)

or

EVQLVESGGGLVQPGNSLRLSCAASGFTFSKFGMSWVRQAPGKGLEWVSS
ISGSGRDTLYADSVKGRFTISRDNAKTTLYLQMNSLRPEDTAVYYCTIGG
SLSVSSQGTLVTVSS (SEQ ID NO: 72).

Embodiment 188 comprises a polypeptide or polypeptide complex of any one of embodiments 1-187, wherein the single domain antibody is SA21.

Embodiment 189 comprises a polypeptide or polypeptide complex of any one of embodiments 1-188, wherein the polypeptide or polypeptide complex comprises a modified amino acid, a non-natural amino acid, a modified non-natural amino acid, or a combination thereof.

Embodiment 190 comprises a polypeptide or polypeptide complex of any one of embodiments 1-189, wherein the modified amino acid or modified non-natural amino acid comprises a post-translational modification.

Embodiment 191 comprises a polypeptide or polypeptide complex of any one of embodiments 1-190, wherein H₁ comprises a linking moiety (L₃) that connects H₁ to P₁.

Embodiment 192 comprises a polypeptide or polypeptide complex of any one of embodiments 1-191, wherein L₃ is a peptide sequence having at least 5 to no more than 50 amino acids.

Embodiment 193 comprises a polypeptide or polypeptide complex of any one of embodiments 1-192, wherein L₃ is a peptide sequence having at least 10 to no more than 30 amino acids.

Embodiment 194 comprises a polypeptide or polypeptide complex of any one of embodiments 1-193, wherein L₃ is a peptide sequence having at least 10 amino acids.

Embodiment 195 comprises a polypeptide or polypeptide complex of any one of embodiments 1-194, wherein L₃ is a peptide sequence having at least 18 amino acids.

Embodiment 196 comprises a polypeptide or polypeptide complex of any one of embodiments 1-195, wherein L₃ is a peptide sequence having at least 26 amino acids.

Embodiment 197 comprises a polypeptide or polypeptide complex of any one of embodiments 1-196, wherein L₃ has a formula selected from the group consisting of (G₂S)ₙ, (GS)ₙ, (GSGGS)ₙ (SEQ ID NO: 49), (GGGS)ₙ (SEQ ID NO: 50), (GGGGS)ₙ (SEQ ID NO: 51), and (GSSGGS)ₙ (SEQ ID NO: 52), wherein n is an integer of at least 1.

Embodiment 198 comprises a polypeptide or polypeptide complex of any one of embodiments 1-197, wherein L₃ comprises an amino acid sequence of SSGGGGSGGGS (SEQ ID NO: 67).

Embodiment 199 comprises a polypeptide or polypeptide complex of any one of embodiments 1-198, wherein the polypeptide or polypeptide complex has weaker binding affinity for its pMHC as compared to the binding affinity for the pMHC of a polypeptide or polypeptide complex that does not have P₁ or L₁.

Embodiment 200 comprises a polypeptide or polypeptide complex of any one of embodiments 1-199, wherein the polypeptide or polypeptide complex has weaker binding affinity for its pMHC that is at least 10X higher than the binding affinity for the pMHC of a form of the polypeptide or polypeptide complex that does not have P₁ or L₁.

Embodiment 201 comprises a polypeptide or polypeptide complex of any one of embodiments 1-200, wherein the polypeptide or polypeptide complex has weaker binding affinity for its pMHC that is at least 100X higher than the binding affinity for the pMHC of a form of the polypeptide or polypeptide complex that does not have P₁ or L₁.

Embodiment 202 comprises a polypeptide or polypeptide complex of any one of embodiments 1-201, wherein the polypeptide or polypeptide complex has weaker binding affinity for its pMHC as compared to the binding affinity for the pMHC of the polypeptide or polypeptide complex in which L₁ has been cleaved by the tumor specific protease.

Embodiment 203 comprises a polypeptide or polypeptide complex of any one of embodiments 1-202, wherein the polypeptide or polypeptide complex has weaker binding affinity for its pMHC that is at least 10X higher than the binding affinity for the pMHC of the polypeptide or polypeptide complex in which L₁ has been cleaved by the tumor specific protease.

Embodiment 204 comprises a polypeptide or polypeptide complex of any one of embodiments 1-203, wherein the polypeptide or polypeptide complex has weaker binding affinity for its pMHC that is at least 100X higher than the binding affinity for the pMHC of the polypeptide or polypeptide complex in which L₁ has been cleaved by the tumor specific protease.

Embodiment 205 comprises a polypeptide or polypeptide complex of any one of embodiments 1-204, wherein the polypeptide or polypeptide complex has an increased EC₅₀ in a T-cell cytolysis assay as compared to the EC₅₀ in a T-cell cytolysis assay of a polypeptide or polypeptide complex that does not have P₁ or L₁.

Embodiment 206 comprises a polypeptide or polypeptide complex of any one of embodiments 1-205, wherein the polypeptide or polypeptide complex has an increased EC₅₀ in a T-cell cytolysis assay that is at least 10X higher than the EC₅₀ in a T-cell cytolysis assay of a form of the polypeptide or polypeptide complex that does not have P₁ or L₁.

Embodiment 207 comprises a polypeptide or polypeptide complex of any one of embodiments 1-206, wherein the polypeptide or polypeptide complex has an increased EC₅₀ in a T-cell cytolysis assay that is at least 100X higher than the EC₅₀ in a T-cell cytolysis assay of a polypeptide or polypeptide complex that does not have P₁ or L₁.

Embodiment 208 comprises a polypeptide or polypeptide complex of any one of embodiments 1-207, wherein the polypeptide or polypeptide complex has an increased EC₅₀ in a T-cell cytolysis assay as compared to the EC₅₀ in a T-cell cytolysis assay of the polypeptide or polypeptide complex in which L₁ has been cleaved by the tumor specific protease.

Embodiment 209 comprises a polypeptide or polypeptide complex of any one of embodiments 1-208, wherein the polypeptide or polypeptide complex has an increased EC₅₀ in a T-cell cytolysis assay that is at least 10X higher than the EC₅₀ in a T-cell cytolysis assay of the polypeptide or polypeptide complex in which L₁ has been cleaved by the tumor specific protease.

Embodiment 210 comprises a polypeptide or polypeptide complex of any one of embodiments 1-209, wherein the polypeptide or polypeptide complex has an increased EC₅₀ in a T-cell cytolysis assay that is at least 100X higher than the EC₅₀ in a T-cell cytolysis assay of the polypeptide or polypeptide complex in which L₁ has been cleaved by the tumor specific protease.

Embodiment 211 comprises a polypeptide or polypeptide complex of any one of embodiments 1-210, wherein the polypeptide or polypeptide complex has an increased EC₅₀ in an IFNγ release T-cell activation assay as compared to the EC₅₀ in an IFNγ release T-cell activation assay of a polypeptide or polypeptide complex that does not have P₁ or L₁.

Embodiment 212 comprises a polypeptide or polypeptide complex of any one of embodiments 1-211, wherein the polypeptide or polypeptide complex has an increased EC₅₀ in an IFNγ release T-cell activation assay that is at least 10X higher than the EC₅₀ in an IFNγ release T-cell activation assay of a form of the polypeptide or polypeptide complex that does not have P₁ or L₁.

Embodiment 213 comprises a polypeptide or polypeptide complex of any one of embodiments 1-212, wherein the polypeptide or polypeptide complex has an increased EC₅₀ in an IFNγ release T-cell activation assay that is at least 100X higher than the EC₅₀ in an IFNγ release T-cell activation assay of a form of the polypeptide or polypeptide complex that does not have P₁ or L₁.

Embodiment 214 comprises a polypeptide or polypeptide complex of any one of embodiments 1-213, wherein the polypeptide or polypeptide complex has an increased EC₅₀ in an IFNγ release T-cell activation assay as compared to the EC₅₀ in an IFNγ release T-cell activation assay of the polypeptide or polypeptide complex in which L₁ has been cleaved by the tumor specific protease.

Embodiment 215 comprises a polypeptide or polypeptide complex of any one of embodiments 1-214, wherein the polypeptide or polypeptide complex has an increased EC₅₀ in an IFNγ release T-cell activation assay that is at least 10X higher than the EC₅₀ in an IFNγ release T-cell activation assay of the polypeptide or polypeptide complex in which L₁ has been cleaved by the tumor specific protease.

Embodiment 216 comprises a polypeptide or polypeptide complex of any one of embodiments 1-215, wherein the polypeptide or polypeptide complex has an increased EC₅₀ in an IFNγ release T-cell activation assay that is at least 100X higher than the EC₅₀ in an IFNγ release T-cell activation assay of the polypeptide or polypeptide complex in which L₁ has been cleaved by the tumor specific protease.

Embodiment 217 comprises a pharmaceutical composition comprising: the polypeptide or polypeptide complex of any one of embodiments 1-216; and a pharmaceutically acceptable excipient.

Embodiment 218 comprises an isolated recombinant nucleic acid molecule encoding the polypeptide or polypeptide complex of any one of embodiments 1-217.

Embodiment 219 comprises a polypeptide or polypeptide complex according to Formula II:

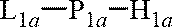

wherein: L₁ₐ comprises a tumor specific protease-cleaved linking moiety that when uncleaved connects P₁ₐ to an antigen recognizing molecule that binds to a target antigen and; P₁ₐ comprises a peptide that binds to the antigen recognizing molecule when L₁ₐ is uncleaved; and H₁ₐ comprises a half-life extending molecule.

Embodiment 220 comprises a polypeptide or polypeptide complex of any one of embodiments 1-219, wherein P₁ₐ when L₁ is uncleaved impairs binding of the antigen recognizing molecule to the target antigen.

Embodiment 221 comprises a polypeptide or polypeptide complex of any one of embodiments 1-220, wherein the antigen recognizing molecule comprises an antibody or antibody fragment.

Embodiment 222 comprises a polypeptide or polypeptide complex of any one of embodiments 1-221, wherein the target antigen is an anti-CD3 effector cell antigen.

Embodiment 223 comprises a polypeptide or polypeptide complex of any one of embodiments 1-222, wherein the target antigen is a tumor cell antigen.

Embodiment 224 comprises a polypeptide or polypeptide complex of any one of embodiments 1-223, wherein the tumor cell antigen MAGEA3 or MART1.

Embodiment 225 comprises a polypeptide or polypeptide complex of any one of embodiments 1-224, wherein P₁ₐ has less than 70% sequence homology to the target antigen.

Embodiment 226 comprises a polypeptide or polypeptide complex of any one of embodiments 1-225, wherein P₁ₐ comprises a peptide sequence of at least 10 amino acids in length.

Embodiment 227 comprises a polypeptide or polypeptide complex of any one of embodiments 1-226, wherein P₁ₐ comprises a peptide sequence of at least 10 amino acids in length and no more than 20 amino acids in length.

Embodiment 228 comprises a polypeptide or polypeptide complex of any one of embodiments 1-227, wherein P₁ₐ comprises a peptide sequence of at least 16 amino acids in length.

Embodiment 229 comprises a polypeptide or polypeptide complex of any one of embodiments 1-228, wherein P₁ₐ comprises a peptide sequence of no more than 40 amino acids in length.

Embodiment 230 comprises a polypeptide or polypeptide complex of any one of embodiments 1-229, wherein P₁ₐ comprises at least two cysteine amino acid residues.

Embodiment 231 comprises a polypeptide or polypeptide complex of any one of embodiments 1-230, wherein P₁ₐ comprises a cyclic peptide or a linear peptide.

Embodiment 232 comprises a polypeptide or polypeptide complex of any one of embodiments 1-231, wherein P₁ₐ comprises a cyclic peptide.

Embodiment 233 comprises a polypeptide or polypeptide complex of any one of embodiments 1-232, wherein P₁ₐ comprises a linear peptide.

Embodiment 234 comprises a polypeptide or polypeptide complex of any one of embodiments 1-233, wherein the wherein the target antigen comprises MAGEA3, and the and the P₁ or P₂ comprises an amino acid sequence selected from the group consisting of GGESCQSVYDSSFCYD (SEQ ID NO: 13), GGNACEMTYDHTFCDP (SEQ ID NO: 14), GGRICEEVYDWIFCES (SEQ ID NO: 15), GGRRCVDVYDNAFCLI (SEQ ID NO: 16), GGVSCKDVYDEAFCWT (SEQ ID NO: 12), GGTSCAQIYDFEFCYS (SEQ ID NO: 17), GGSLCSLVYDQDFCES (SEQ ID NO: 18), GGNSCSLVYDKAFCLF (SEQ ID NO: 19), GGNQCWEVYDQEFCSL (SEQ ID NO: 20), GGSACSRIYDFAFCHT (SEQ ID NO: 21), GGTFCYFDHGLVNCQW (SEQ ID NO: 22), GGHCFVSPASGEWWCV (SEQ ID NO: 23), GGCSWIFDGLRYFSKC (SEQ ID NO: 24), VRTWFEKFPELV (SEQ ID NO: 25), LVWGCIWDDMCS (SEQ ID NO: 26), WHWEPSMVWGML (SEQ ID NO: 27), GGGCFVSPATGFTWCV (SEQ ID NO: 28), GGDCQPDSVWSYWYCR (SEQ ID NO: 29), GGCTFVDWWVLGSPYC (SEQ ID NO: 30), GGCLMNDYYYLWGGHC (SEQ ID NO: 31), GGASCKDVYDEAFCWT (SEQ ID NO: 32), GGVACKDVYDEAFCWT (SEQ ID NO: 33), GGVSAKDVYDEAFCWT (SEQ ID NO: 34), GGVSCADVYDEAFCWT (SEQ ID NO: 35), GGVSCKAVYDEAFCWT (SEQ ID NO: 36), GGVSCKDAYDEAFCWT (SEQ ID NO: 37), GGVSCKDVADEAFCWT (SEQ ID NO: 38), GGVSCKDVYAEAFCWT (SEQ ID NO: 39), GGVSCKDVYDAAFCWT (SEQ ID NO: 40), GGVSCKDVYDEAACWT (SEQ ID NO: 41), GGVSCKDVYDEAFAWT (SEQ ID NO: 42), GGVSCKDVYDEAFCAT (SEQ ID NO: 43), GGVSCKDVYDEAFCWA (SEQ ID NO: 44), EVDPIGHLY (SEQ ID NO: 45), ESDPIVAQY (SEQ ID NO: 46), and GGASCAASASAAACAS (SEQ ID NO: 47).

Embodiment 235 comprises a polypeptide or polypeptide complex of any one of embodiments 1-234, wherein H₁ₐ comprises a polymer.

Embodiment 236 comprises a polypeptide or polypeptide complex of any one of embodiments 1-235, wherein the polymer is polyethylene glycol (PEG).

Embodiment 237 comprises a polypeptide or polypeptide complex of any one of embodiments 1-236, wherein H₁ₐ comprises albumin.

Embodiment 238 comprises a polypeptide or polypeptide complex of any one of embodiments 1-237, wherein H₁ₐ comprises an Fc domain.

Embodiment 239 comprises a polypeptide or polypeptide complex of any one of embodiments 1-238, wherein the albumin is serum albumin.

Embodiment 240 comprises a polypeptide or polypeptide complex of any one of embodiments 1-239, wherein the albumin is human serum albumin.

Embodiment 241 comprises a polypeptide or polypeptide complex of any one of embodiments 1-240, wherein H₁ₐ comprises a polypeptide, a ligand, or a small molecule.

Embodiment 242 comprises a polypeptide or polypeptide complex of any one of embodiments 1-241, wherein the polypeptide, the ligand or the small molecule binds a serum protein or a fragment thereof, a circulating immunoglobulin or a fragment thereof, or CD35/CR1.

Embodiment 243 comprises a polypeptide or polypeptide complex of any one of embodiments 1-242, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 244 comprises a polypeptide or polypeptide complex of any one of embodiments 1-243, wherein the circulating immunoglobulin molecule comprises IgG1, IgG2, IgG3, IgG4, slgA, IgM or IgD.

Embodiment 245 comprises a polypeptide or polypeptide complex of any one of embodiments 1-244, wherein the serum protein is albumin.

Embodiment 246 comprises a polypeptide or polypeptide complex of any one of embodiments 1-245, wherein the polypeptide is an antibody.

Embodiment 247 comprises a polypeptide or polypeptide complex of any one of embodiments 1-246, wherein the antibody comprises a single domain antibody, a single chain variable fragment or a Fab.

Embodiment 248 comprises a polypeptide or polypeptide complex of any one of embodiments 1-247, wherein the antibody comprises a single domain antibody that binds to albumin.

Embodiment 249 comprises a polypeptide or polypeptide complex of any one of embodiments 1-248, wherein the antibody is a human or humanized antibody.

Embodiment 250 comprises a polypeptide or polypeptide complex of any one of embodiments 1-249, wherein the single domain antibody is 645gH1gL1.

Embodiment 251 comprises a polypeptide or polypeptide complex of any one of embodiments 1-250, wherein the single domain antibody is 645dsgH5gL4.

Embodiment 252 comprises a polypeptide or polypeptide complex of any one of embodiments 1-251, wherein the single domain antibody is 23-13-A01 -sc02.

Embodiment 253 comprises a polypeptide or polypeptide complex of any one of embodiments 1-252, wherein the single domain antibody is A10m3 or a fragment thereof.

Embodiment 254 comprises a polypeptide or polypeptide complex of any one of embodiments 1-253, wherein the single domain antibody is DOM7r-31.

Embodiment 255 comprises a polypeptide or polypeptide complex of any one of embodiments 1-254, wherein the single domain antibody is DOM7h-11-15.

Embodiment 256 comprises a polypeptide or polypeptide complex of any one of embodiments 1-255, wherein the single domain antibody is Alb-1, Alb-8, or Alb-23.

Embodiment 257 comprises a polypeptide or polypeptide complex of any one of embodiments 1-256, wherein the single domain antibody is 10G or 10GE.

Embodiment 258 comprises a polypeptide or polypeptide complex of any one of embodiments 1-257, wherein the single domain antibody is 10G, and the single domain antibody comprises an amino acid sequence

MEVQLVESGGGLVQPGNSLRLSCAASGFTFSKFGMSWVRQAPGKGLEWVS
SISGSGRDTLYADSVKGRFTISRDNAKTTLYLQMNSLRPEDTAVYYCTIG
GSLSVSSQGTLVTVSS (SEQ ID NO: 2)

or

EVQLVESGGGLVQPGNSLRLSCAASGFTFSKFGMSWVRQAPGKGLEWVSS
ISGSGRDTLYADSVKGRFTISRDNAKTTLYLQMNSLRPEDTAVYYCTIGG
SLSVSSQGTLVTVSS (SEQ ID NO: 72).

Embodiment 259 comprises a polypeptide or polypeptide complex of any one of embodiments 1-258, wherein the single domain antibody is SA21.

Embodiment 260 comprises a polypeptide or polypeptide complex of any one of embodiments 1-259, wherein H₁ₐ comprises a linking moiety (L₃ₐ) that connects H₁ₐ to P₁ₐ.

Embodiment 261 comprises a polypeptide or polypeptide complex of any one of embodiments 1-260, wherein L₁ₐ is a peptide sequence having at least 5 to no more than 50 amino acids.

Embodiment 262 comprises a polypeptide or polypeptide complex of any one of embodiments 1-261, wherein L₁ₐ is a peptide sequence having at least 10 to no more than 30 amino acids.

Embodiment 263 comprises a polypeptide or polypeptide complex of any one of embodiments 1-262, wherein L₁ₐ is a peptide sequence having at least 10 amino acids.

Embodiment 264 comprises a polypeptide or polypeptide complex of any one of embodiments 1-263, wherein L₁ₐ is a peptide sequence having at least 18 amino acids.

Embodiment 265 comprises a polypeptide or polypeptide complex of any one of embodiments 1-264, wherein L₁ₐ is a peptide sequence having at least 26 amino acids.

Embodiment 266 comprises a polypeptide or polypeptide complex of any one of embodiments 1-265, wherein L₁ₐ has a formula selected from the group consisting of (G₂S)ₙ, (GS)ₙ, (GSGGS)ₙ (SEQ ID NO: 49), (GGGS)ₙ (SEQ ID NO: 50), (GGGGS)ₙ (SEQ ID NO: 51), and (GSSGGS)ₙ (SEQ ID NO: 52), wherein n is an integer of at least 1.

Embodiment 267 comprises a polypeptide or polypeptide complex of any one of embodiments 1-266,wherein L₃ₐ comprises an amino acid sequence of SSGGGGSGGGS (SEQ ID NO: 67).

Embodiment 268 comprises a polypeptide or polypeptide complex comprising a structural arrangement according to the configuration shown in FIG. 70A, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the beta TCR polypeptide is linked to a C terminus of the heavy chain variable domain of the scFv, and wherein the soluble TCR is linked to P₂ and L₂, wherein P₂ comprises a peptide that impairs binding of the soluble TCR to the tumor cell antigen; and L₂ comprises a linking moiety that connects the alpha TCR polypeptide to P₂ and is a substrate for a tumor specific protease.

Embodiment 269 comprises a polypeptide or polypeptide complex comprising a structural arrangement according to the configuration shown in FIG. 70B, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the beta TCR polypeptide is linked to a C terminus of the heavy chain variable domain of the scFv.

Embodiment 270 comprises a polypeptide or polypeptide complex comprising a structural arrangement according to the configuration shown in FIG. 70C, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the alpha TCR polypeptide is linked to a C terminus of the heavy chain variable domain of the scFv, and wherein the soluble TCR is linked to P₂ and L₂, wherein P₂ comprises a peptide that impairs binding of the soluble TCR to the tumor cell antigen; and L₂ comprises a linking moiety that connects the beta TCR polypeptide to P₂ and is a substrate for a tumor specific protease.

Embodiment 271 comprises a polypeptide or polypeptide complex comprising a structural arrangement according to the configuration shown in FIG. 70D, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the alpha TCR polypeptide is linked to a C terminus of the heavy chain variable domain of the scFv.

Embodiment 272 comprises a polypeptide or polypeptide complex comprising a structural arrangement according to the configuration shown in FIG. 70E, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the beta TCR polypeptide is linked to a C terminus of the light chain variable domain of the scFv, and wherein the soluble TCR is linked to P₂ and L₂, wherein P₂ comprises a peptide that impairs binding of the soluble TCR to the tumor cell antigen; and L₂ comprises a linking moiety that connects the alpha TCR polypeptide to P₂ and is a substrate for a tumor specific protease.

Embodiment 273 comprises a polypeptide or polypeptide complex comprising a structural arrangement according to the configuration shown in FIG. 70F, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the beta TCR polypeptide is linked to a C terminus of the light chain variable domain of the scFv.

Embodiment 274 comprises a polypeptide or polypeptide complex comprising a structural arrangement according to the configuration shown in FIG. 70G, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the alpha TCR polypeptide is linked to a C terminus of the light chain variable domain of the scFv, and wherein the soluble TCR is linked to P₂ and L₂, wherein P₂ comprises a peptide that impairs binding of the soluble TCR to the tumor cell antigen; and L₂ comprises a linking moiety that connects the beta TCR polypeptide to P₂ and is a substrate for a tumor specific protease.

Embodiment 275 comprises a polypeptide or polypeptide complex comprising a structural arrangement according to the configuration shown in FIG. 70H, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a soluble T cell receptor (TCR) that binds to a tumor cell antigen, wherein the soluble TCR comprises an alpha TCR polypeptide and a beta TCR polypeptide, wherein the alpha TCR polypeptide is linked to a C terminus of the light chain variable domain of the scFv.

Embodiment 276 comprises a polypeptide or polypeptide complex comprising a structural arrangement according to the configuration shown in FIG. 70I, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to an N-terminus of the beta TCR polypeptide, and wherein the scFv is linked to P₂ and L₂, wherein P₂ comprises a peptide that impairs binding of the scFv to an effector cell antigen; and L₂ comprises a linking moiety that connects the light chain variable domain of the scFv to P₂ and is a substrate for a tumor specific protease.

Embodiment 277 comprises a polypeptide or polypeptide complex comprising a structural arrangement according to the configuration shown in FIG. 70J, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to an N-terminus of the beta TCR polypeptide and the scFv binds to an effector cell antigen.

Embodiment 278 comprises a polypeptide or polypeptide complex comprising a structural arrangement according to the configuration shown in FIG. 70K, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to an N-terminus of the alpha TCR polypeptide, and wherein the scFv is linked to P₂ and L₂, wherein P₂ comprises a peptide that impairs binding of the scFv to an effector cell antigen; and L₂ comprises a linking moiety that connects the light chain variable domain of the scFv to P₂ and is a substrate for a tumor specific protease.

Embodiment 279 comprises a polypeptide or polypeptide complex comprising a structural arrangement according to the configuration shown in FIG. 70L, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to an N-terminus of the alpha TCR polypeptide and the scFv binds to an effector cell antigen.

Embodiment 280 comprises a polypeptide or polypeptide complex comprising a structural arrangement according to the configuration shown in FIG. 70M, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to an N-terminus of the beta TCR polypeptide, and wherein the scFv is linked to P₂ and L₂, wherein P₂ comprises a peptide that impairs binding of the scFv to an effector cell antigen; and L₂ comprises a linking moiety that connects the heavy chain variable domain of the scFv to P₂ and is a substrate for a tumor specific protease.

Embodiment 281 comprises a polypeptide or polypeptide complex comprising a structural arrangement according to the configuration shown in FIG. 70N, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to an N-terminus of the beta TCR polypeptide and the scFv binds to an effector cell antigen.

Embodiment 282 comprises a polypeptide or polypeptide complex comprising a structural arrangement according to the configuration shown in FIG. 70O, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to an N-terminus of the alpha TCR polypeptide, and wherein the scFv is linked to P₂ and L₂, wherein P₂ comprises a peptide that impairs binding of the scFv to an effector cell antigen; and L₂ comprises a linking moiety that connects the heavy chain variable domain of the scFv to P₂ and is a substrate for a tumor specific protease.

Embodiment 283 comprises a polypeptide or polypeptide complex comprising a structural arrangement according to the configuration shown in FIG. 70P, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to an N-terminus of the alpha TCR polypeptide and the scFv binds to an effector cell antigen.

Embodiment 284 comprises a polypeptide or polypeptide complex comprising a structural arrangement according to the configuration shown in FIG. 70Q, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to a C-terminus of the beta TCR polypeptide and the scFv binds to an effector cell antigen.

Embodiment 285 comprises a polypeptide or polypeptide complex comprising a structural arrangement according to the configuration shown in FIG. 70R, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to a C-terminus of the alpha TCR polypeptide and the scFv binds to an effector cell antigen.

Embodiment 286 comprises a polypeptide or polypeptide complex comprising a structural arrangement according to the configuration shown in FIG. 70S, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to a C-terminus of the alpha TCR polypeptide and the scFv binds to an effector cell antigen.

Embodiment 287 comprises a polypeptide or polypeptide complex comprising a structural arrangement according to the configuration shown in FIG. 70T, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the heavy chain variable domain is linked to a C-terminus of the beta TCR polypeptide and the scFv binds to an effector cell antigen.

Embodiment 288 comprises a polypeptide or polypeptide complex comprising a structural arrangement according to the configuration shown in FIG. 70U, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to a C-terminus of the beta TCR polypeptide and the scFv binds to an effector cell antigen.

Embodiment 289 comprises a polypeptide or polypeptide complex comprising a structural arrangement according to the configuration shown in FIG. 70V, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the alpha TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to a C-terminus of the alpha TCR polypeptide and the scFv binds to an effector cell antigen.

Embodiment 290 comprises a polypeptide or polypeptide complex comprising a structural arrangement according to the configuration shown in FIG. 70W, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to a C-terminus of the alpha TCR polypeptide and the scFv binds to an effector cell antigen.

Embodiment 291 comprises a polypeptide or polypeptide complex comprising a structural arrangement according to the configuration shown in FIG. 70X, wherein the polypeptide or polypeptide complex comprises a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen and the peptide is linked to a N-terminus of the beta TCR polypeptide with a linking moiety that is a substrate for a tumor specific protease, and the peptide is further linked to a half-life extending molecule; and a single chain variable fragment (scFv) comprising a light chain variable domain and a heavy chain variable domain, wherein the light chain variable domain is linked to a C-terminus of the beta TCR polypeptide and the scFv binds to an effector cell antigen.

### EXAMPLES

### Example 1. Preparation of Soluble TCRs

Expression plasmids encoding the TCR alpha and beta chains are produced using standard molecular biology techniques. Plasmids are transformed into chemically-competent cells and grown overnight at 37° C. Protein expression is induced by the addition of Isopropyl β-D-1 -thiogalactopyranoside (IPTG) to 1 mM and bacteria are grown for a further 3 hours at 37° C. Bacteria are harvested by centrifugation at 4000 × g for 15 minutes and lysed in a protein extraction reagent containing DNAse. Lysis proceeds for 1 hour at room temperature with agitation before inclusion bodies are harvested by centrifugation at 10000 × g for 5 minutes. Pellets are washed twice with a detergent buffer containing 1% Triton X100 and resuspended in a buffered saline solution.

Soluble TCRs are prepared by dissolving alpha and beta inclusion bodies in 6 M guanidine-HCI containing 10 mM dithiothreitol and incubating at 37°Cfor 30 minutes. Samples are diluted into 50 ml urea folding buffer (5 M urea; 0.4 M L-arginine; 0.1 M Tris-CI, pH 8.1; 2 mM EDTA; 6.5 mM β-mercapthoethylamine; 1.9 mM cystamine) and dialyzed against eight volumes of water overnight at 4° C., followed by dialysis for a further 24 hours in eight volumes of 10 mM Tris (8.1), with one buffer change. The resultant TCR complexes will be concentrated and purified using Ni-NTA, and size-exclusion chromatography. Isolated proteins were characterized using standard size exclusion chromatography, SDS PAGE, and LC-MS procedures. TCR fusion constructs can also be produced in mammalian cells, insect cells, or yeast cells according to known methods.

### Example 2. In Vitro Screening of a Modified TCR Produced in Example 1 for Antigen Recognition

A modified TCR is tested for its ability to recognize antigens when separately expressed in CD8⁺ T cells and CD4⁺ T cells. PBMC from a subject is transfected as described in Zhao et al. (2006), et al., Mol. Ther. 13: 151-159 (2006) with (i) RNA encoding the WT alpha chain of the TCR and (ii) RNA encoding the WT beta chain of the TCR, or DNA encoding Green Fluorescence Protein (GFP).

Transfected cells are washed and stimulated with or without (T alone) one of the following cells: T2+ pulsed with antigen. Responder cells (1 × 10⁵ electroporated PBLs) and 1 × 10⁵ stimulator cells are incubated in a 0.2-ml culture volume in individual wells of 96-well plates. Stimulator cells and responder cells are co-cultured for 16 to 24 h. Cytokine secretion of culture supernatants diluted to the linear range of the assay is measured using commercially available ELISA kits (IFN-γ Endogen, Cambridge, Mass.).The amount of IFN-γ (pg/ml) produced by transfected CD8⁺ T cells is determined, while the amount of IFN-γ (pg/ml) produced by transfected CD4⁺ T cells is determined.

### Example 3. Bacterial Expression of Reformatted Bispecific T Cell Receptors

This example outlines an exemplary way to reformat peptides and scFv into bispecific recombinant TCR fusions. T cell receptors are comprised of an alpha chain complexed with a beta chain. Each alpha and beta chains include the entire extracellular domain and lack the membrane spanning and intracellular domains. The individual T cell receptor chains were overexpressed in E. coli and recovered from inclusion bodies. Specifically, genes encoding the alpha or beta subunits with or without additional peptide or protein fusions added to either the amino or carboxy-termini were synthesized using E. coli codon optimization. Additionally, the C-terminus of the alpha subunit has appended a poly histidine epitope for protein purification purposes and to the C-terminus of the beta subunit a BirA biotinylation substrate (“Avitag”) has been appended for enzymatic site specific biotin conjugation. Following protein expression inclusion bodies were isolated and then dissolved in solubilization buffer (8 M urea, 25 mM MES pH 6.0, 10 mM EDTA, 0.1 mM DTT), while TCR was dissolved in the solubilization buffer containing 6 M guanidine hydrochloride (GnHCl). Ninety milligrams total of TCR alpha and TCR beta were diluted into 500 mL refolding buffer [3 M urea, 0.2 M Arg-HCl, 150 mM Tris-HCl pH 8.0, 1.5 mM reduced glutathione, 0.15 mM oxidized glutathione and stirred at 4° C. for 72 h. The subunits with CD3 scFv fusions were added in a two-fold excess by weight compared to chains lacking scFv fusions. Specifically, sixty milligrams of each of the CD3 scFv containing TCR chains were combined with thirty milligrams of the complementary TCR chain to complete heterodimeric TCR. Refolded TCR was dialyzed at 4° C. for 24 h in 4 L dialysis buffer (10 mM Tris pH 8.5, 50 mM NaCl) and then for an additional 24 h in fresh 4 L dialysis buffer. The resultant TCR complexes are concentrated and purified using Ni-NTA, and size-exclusion chromatography. Isolated proteins were characterized using standard size exclusion chromatography, SDS PAGE, and LC-MS procedures.

### Example 4. Screening of Peptides/masks

Biopanning with m13 phagemid p8 or p3 displayed peptide libraries was performed with biotin-conjugated target immobilized on streptavidin coated paramagnetic beads. The targets were either recombinant proteins enzymatically biotinylated by a birA directed process onto an engineered substrate (Avitag) or through chemical biotin conjugation, typically through random labeling of primary amines. Following binding to bead bound target and washing steps, specifically bound phage were recovered by elution at pH 2.2. Enrichment of specific binding clones was generally accomplished by 2-4 rounds of successive biopanning and amplification. After 2 rounds of biopanning the resulting phage pools were infected into TG1 cells and plated out on LB-ampicillin/agar plates for clonal isolation and subsequent characterization.

For hit identification, individual colonies were grown in 96-deep well plates for 2-4 hours and infected with helper phage and induced to produce peptide displayed phagemid following an overnight growth. The next day the deep well plates were centrifuged to separate the soluble phagemid from the E. coli cells. The phagemid containing supernatants were then combined with PBS-Tween 20 (0.05%) + BSA (1%) blocking buffer and incubated in ELISA wells containing Neutravidin captured biotin-conjugated target or Neutravidin alone. After binding at 4 degrees the plates were washed, and specifically bound phage were detected by anti-m13 HRP conjugated antibodies using standard TMB-based chromogenic ELISA procedures. Daughter plates or individual wells were subjected to standard DNA sequencing for clonal peptide sequence identification.

### Example 5. Generating Peptide-Half-Life Extending Molecule Conjugate

### Llama Immunization and Panning Recovery

Outbred llamas were immunized with purified human serum albumin first in Freund’s Complete Adjuvant (FCA) and subsequently boosted with Incomplete Freund’s Adjuvant. Upon completion of the immunization regimen immune Vhh p3 phagemid display libraries were generated from cDNA prepared using total RNA isolated from llama PBMCs. The phage display libraries were biopanned against human serum albumin immobilized on paramagnetic beads. Random clones isolated following round 2 and subsequent rounds were grown in 96-deep well culture were tested by either phagemid or soluble single domain ELISA for specific reactivity to human serum albumin, cynomolgus serum albumin, and mouse serum albumin.

Single domain antibodies derived from clones with desired specificity profiles were purified from either scaled up production in E. coli and purification from periplasmic fractions and Ni-NTA chromatography or mammalian transient expression in HEK293 cells. Resulting protein was analyzed by SDS-PAGE for relative purity and protein concentrations quantitated by A280 analysis. The resulting proteins were available for subsequent quantitative binding assessments by either ELISA-based methods or kinetic-based methods.

### Example 6. Generating Peptide-Anti-Albumin Conjugate

Llama single domain antibodies (SDAs) that bind serum albumin or their humanized variants were cloned as recombinant constructs with carboxy terminal fusions comprising peptides of specific binding profiles from the example above. The fusions can either be recombinantly fused to the SDA terminus or with an intervening peptide linker. The resulting recombinant constructs were then produced from periplasmic production in E.coli as follows. A single colony or small portion of frozen starter was inoculated into 10 ml LB/Ampicillin (100 mcg/mL) + 2% glucose and grown overnight at 37 degrees. Next day 500 mL of LB/Ampicillin (100 mcg/mL) + 0.1% glucose was inoculated with 5 mL of the preculture and grown at 37 degrees until reaching OD 600 nm ~0.7. Next protein expression was induced by the addition of IPTG (final concentration 1 mM) and shifted to growth overnight at 28 degrees, or alternatively induction was grown at for 4 hours at 37 degrees. Bacteria were harvested by centrifugation. The cell pellet was next resuspended in 7.5 mL TES buffer (TES buffer 24.22 g Tris, 0.19 g EDTA and 171.15 g sucrose in 1 liter ddH2O. Adjust to pH 8.0 with HCl.) and incubate for at least 1 hr on ice on an orbital shaking platform. Add 30 ml of TES/4 to the resuspended pellet and shake for 45 min on ice on an orbital shaking platform. Centrifuge 30 min at 10,000 g at 4° C. and recover the supernatant as the periplasmic extract. Purify the protein through a nickel chelate support (Ni/NTA), followed by size exclusion chromatography on Superdex200 columns. Resulting protein was analyzed by SDS-PAGE for relative purity and protein concentrations quantitated by A280 analysis.

### Example 7. Generating Modified TCRs With Mask on TCR

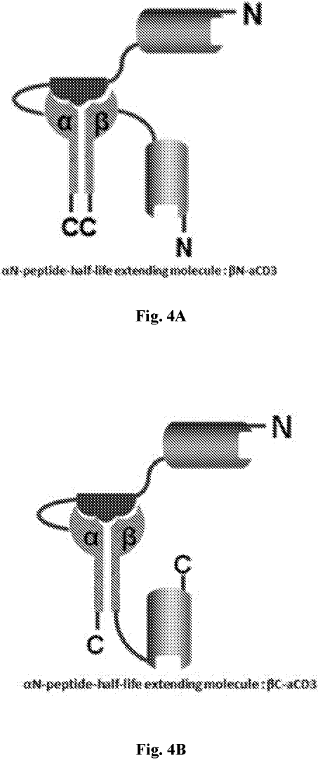

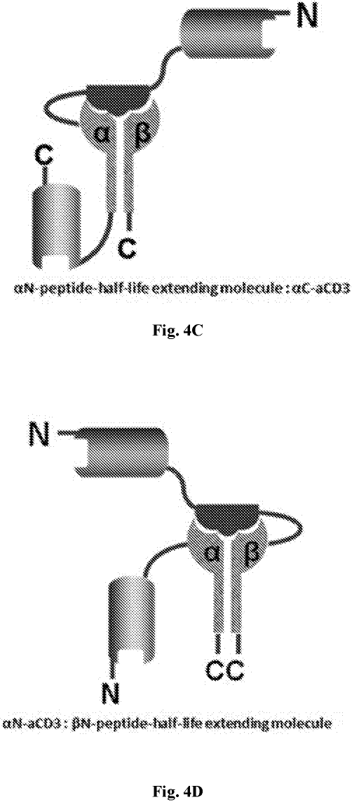

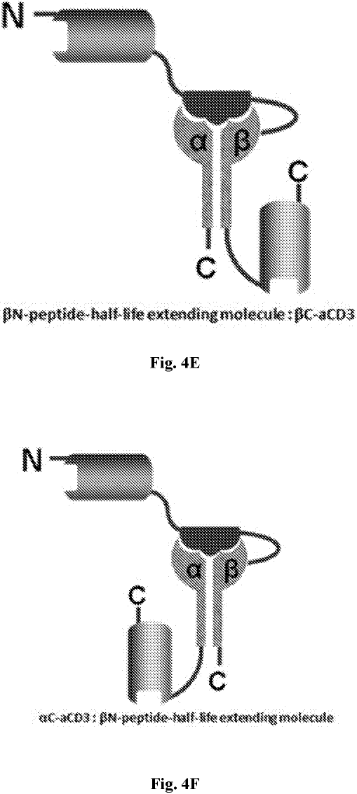

The following procedure was used to reformat peptides found above into recombinant TCR fusions represented in FIG. 4A- FIG. 4F. Notably, T cell receptors are comprised of an alpha chain complexed with a beta chain. Each alpha and beta chains include the entire extracellular domain and lack the membrane spanning and intracellular domains. Bispecific soluble T cell receptors (TCRs) were generated with fusions of anti-CD3 binding modules to engender simultaneous binding of cells presenting corresponding pMHC and CD3 positive cells with the goal of forming a cytolytic response against the pMHC targeted cell. To generate these desired bispecific proteins, either anti-CD3 antibody fragments or anti-CD3 single domain antibodies were fused to the TCR termini. To provide further functionality, TCR peptide masks identified above were recombinantly fused to either the alpha or beta N-termini. To assess the effects of masking, peptide masked TCRs including anti-CD3 antibody fusions were integrated into single heterodimeric protein constructs. In each of the masked descriptions, protein linkers of defined proteolytic lability were incorporated as intervening sequence between the mask and the TCR subunit terminus to provide the TCR targeted binding only when preferentially cleaved and subsequently activated within an opportunistic tumor environment. In additional constructs each of these masks were extended at their respective N-termini to contain an albumin binding single domain antibody to extend the systemic half-life of the resulting proteins.

Each of the individual T cell receptor chains necessary for the above constructs were overexpressed in E. coli and recovered from inclusion bodies. As described in FIG. 4A, a genetic construct was made containing the modified bispecific TCR that was composed of an anti-MAGE-A3 alpha subunit with recombinant extension at the N-terminus of a competitive peptide mask fused via proteolytic linker and to the N-terminus of the peptide mask a further extension is made with a single domain antibody that binds to serum albumin (SEQ ID NO: 1). To complement the alpha TCR subunit, a second genetic construct was made composed of an anti-MAGE-A3 beta subunit with a recombinant extension at the N-terminus to include an anti-CD3 scFv. Each of the genes encoding each of the described alpha or beta subunits were synthesized using E. coli codon optimization. Additionally, the C-terminus of each alpha subunit has appended a poly histidine epitope for protein purification purposes and to the C-terminus of each of the beta subunits, a BirA biotinylation substrate (“Avitag”) was appended for enzymatic site specific biotin conjugation. Following protein expression, inclusion bodies were isolated and then dissolved in solubilization buffer (8 M urea, 25 mM MES pH 6.0, 10 mM EDTA, 0.1 mM DTT), while TCRs were dissolved in the solubilization buffer containing 6 M guanidine hydrochloride (GnHCl). Thirty milligrams each of TCR alpha and TCR beta were diluted into 500 mL refolding buffer [3 M urea, 0.2 M Arg-HCl, 150 mM Tris-HCl pH 8.0, 1.5 mM reduced glutathione, 0.15 mM oxidized glutathione and stirred at 4° C. for 72 h. Refolded TCR was dialyzed at 4° C. for 24 h in 4 L dialysis buffer (10 mM Tris pH 8.5, 50 mM NaCl) and then for an additional 24 h in fresh 4 L dialysis buffer. The resultant TCR complexes are concentrated and purified using Ni-NTA, and size-exclusion chromatography. The resulting proteins were analyzed by SDS-PAGE under reducing and non-reducing conditions. Final protein concentrations quantitated by A280 analysis. Purified proteins were stored in aliquots at -80 degrees until used. A similar construct described in FIG. 4B used the same alpha subunit, but required a new beta subunit with the anti-CD3 antibody recombinantly fused to the C-terminus of the anti-MAGE-A3 beta subunit. The variant described in FIG. 4C placed the anti-CD3 antibody fusion to the C-terminus of the anti-MAGE-A3 alpha subunit that also contained a proteolytically labile competitive mask with a further N-terminal anti-serum albumin extension. In the variant shown in FIG. 4D the albumin binding domain is recombinantly fused to the N-terminus of the alpha subunit of anti-MAGE-A3 TCR, while the corresponding beta subunit C-terminus bears an anti-CD3 scFv. The variant depicted in FIG. 4F contains a recombinant extension to the N-terminus of the anti-MAGE-A3 beta subunit composed of competitive peptide mask fused via a proteolytically labile linker with a further N-terminal extension of an anti-serum albumin binding domain. It is complexed with an anti-MAGE-A3 beta subunit containing a C-terminally fused anti-CD3 antibody.

### Example 8. Generating Modified Bispecific TCRs With Mask on Anti-CD3

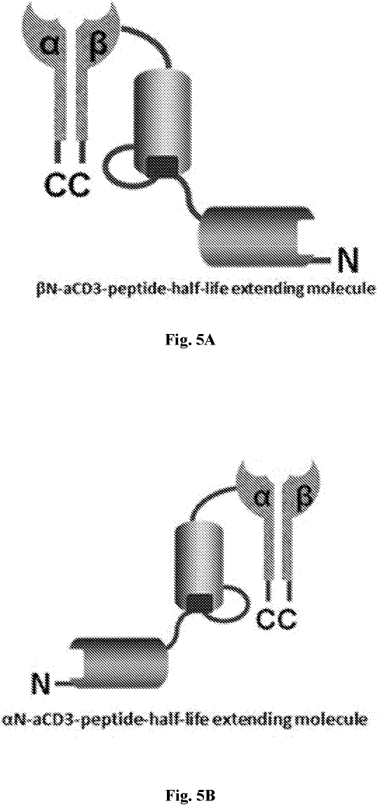

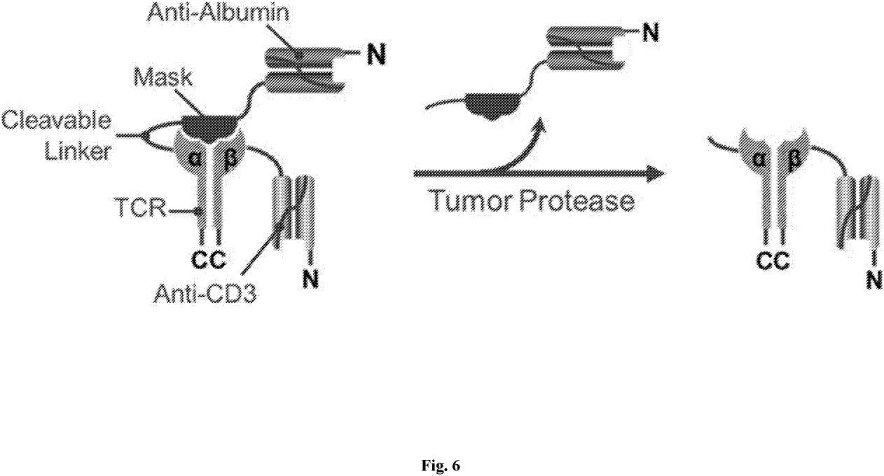

The following procedure was used to reformat peptides found above into recombinant TCR fusions represented in FIG. 5A- FIG. 5B. Notably, T cell receptors are comprised of an alpha chain complexed with a beta chain. Each alpha and beta chains include the entire extracellular domain and lack the membrane spanning and intracellular domains. Bispecific soluble T cell receptors (TCRs) were generated with fusions of anti-CD3 binding modules to engender simultaneous binding of cells presenting corresponding pMHC and CD3 positive cells with the goal of forming a cytolytic response against the pMHC targeted cell. To generate these desired bispecific proteins, either anti-CD3 antibody fragments or anti-CD3 single domain antibodies were recombinantly fused to TCR subunit N-termini. To provide further functionality, anti-CD3 peptide masks identified above were recombinantly fused to anti-CD3. Finally, to assess the effects of masking, peptide masked anti-CD3 antibody fusions were integrated into single heterodimeric protein constructs. In each of the masked descriptions, protein linkers of defined proteolytic lability were incorporated as intervening sequence between the mask and the anti-CD3 N-terminus to provide the anti-CD3 targeted binding only when preferentially cleaved and subsequently activated within an opportunistic tumor environment. In additional constructs these masks were extended at their respective N-termini to contain an albumin binding single domain antibody to extend the systemic half-life of the resulting proteins.

Each of the individual T cell receptor chains necessary for the above constructs were overexpressed in E. coli and recovered from inclusion bodies. As described in FIG. 5A a genetic construct was made containing the modified bispecific TCR that was composed of an anti-MAGE-A3 beta subunit with recombinant extension at the N-terminus to include an anti-CD3 antibody that is further extended at its N-terminus with a competitive peptide mask fused via proteolytic linker and still further extended from the N-terminus of the peptide mask is a single domain antibody to bind to serum albumin. To complement the beta TCR, a second genetic construct was made composed of an anti-MAGE-A3 alpha. In contrast, for the construct depicted in FIG. 5B a genetic construct was made containing the modified bispecific TCR that was composed of an anti-MAGE-A3 alpha subunit with recombinant extension at the N-terminus to include an anti-CD3 antibody that is further extended with a competitive peptide mask fused via proteolytic linker and still further extended from the N-terminus of the peptide mask a single domain antibody to bind to serum albumin. To complement the beta TCR a second genetic construct composed of an anti-MAGE-A3 alpha was made. The necessary genes encoding the described subunits above were synthesized using E. coli codon optimization. Additionally, the C-terminus of each alpha subunit has appended a poly histidine epitope for protein purification purposes and to the C-terminus of each of the beta subunit a BirA biotinylation substrate (“Avitag”) was appended for enzymatic site specific biotin conjugation. Following protein expression, inclusion bodies were isolated and then dissolved in solubilization buffer (8 M urea, 25 mM MES pH 6.0, 10 mM EDTA, 0.1 mM DTT), while TCRs were dissolved in the solubilization buffer containing 6 M guanidine hydrochloride (GnHCl). Thirty milligrams each of TCR alpha and TCR beta were diluted into 500 mL refolding buffer [3 M urea, 0.2 M Arg-HCl, 150 mM Tris-HCl pH 8.0, 1.5 mM reduced glutathione, 0.15 mM oxidized glutathione and stirred at 4° C. for 72 h. Refolded TCR was dialyzed at 4° C. for 24 h in 4 L dialysis buffer (10 mM Tris pH 8.5, 50 mM NaCl) and then for an additional 24 h in fresh 4 L dialysis buffer. The resultant TCR complexes are concentrated and purified using Ni-NTA, and size-exclusion chromatography. The resulting proteins were analyzed by SDS-PAGE under reducing and non-reducing conditions. Final protein concentrations quantitated by A280 analysis. Purified proteins were stored in aliquots at -80 degrees until used. For the construct depicted in FIG. 5B a genetic construct was made containing the modified bispecific TCR that was composed of an anti-MAGE-A3 alpha subunit with recombinant extension at the N-terminus of an anti-CD3 antibody that is further extended with a competitive peptide mask fused via proteolytic linker and further to the N-terminus of the peptide mask a further extension is made with a single domain antibody that binds to serum albumin. To complement the alpha TCR beta a second genetic construct composed of an anti-MAGE-A3 alpha was made.

### Example 9. Generating Modified TCRs With “Dual Mask”

The following procedure was used to reformat peptides found above into recombinant TCR fusions. Notably, T cell receptors are comprised of an alpha chain complexed with a beta chain. Each alpha and beta chains include the entire extracellular domain and lack the membrane spanning and intracellular domains. Bispecific soluble T cell receptors (TCRs) were generated with fusions of anti-CD3 binding modules to engender simultaneous binding of cells presenting corresponding pMHC and CD3 positive cells with the goal of forming a cytolytic response against the pMHC targeted cell. To generate these desired bispecific proteins either anti-CD3 antibody fragments or anti-CD3 single domain antibodies were fused to either of the heteromeric TCR amino-termini. Dual control was provided by masking both TCR and the anti-CD3 antibody. TCR peptide masks identified above are appended as recombinant fusions to either the alpha or beta N-termini, and secondly recombinantly fused with anti-CD3 peptide masks to the free amino termini of the anti-CD3 antibody. Finally to assess the combined effects of dual masking in single constructs the peptide masked TCRs and peptide masked anti-CD3 antibodies were integrated into single heterodimeric protein constructs. In each of the masked descriptions protein linkers of defined proteolytic lability were incorporated as intervening sequence between the mask and either the TCR or anti-CD3 N-termini to provide the TCR and anti-CD3 targeted binding only when preferentially cleaved and subsequently activated within an opportunistic tumor environment. In additional constructs either of these masks were extended at their respective N-termini to contain an albumin binding single domain antibody to extend the systemic half-life of the resulting proteins.

Each of the individual T cell receptor chains necessary for the above constructs were overexpressed in E. coli and recovered from inclusion bodies. Specifically, genes encoding the alpha or beta subunits with or without additional peptide or protein fusions were synthesized using E. coli codon optimization. Additionally, the C-terminus of the alpha subunit has appended a poly histidine epitope for protein purification purposes and to the C-terminus of the beta subunit a BirA biotinylation substrate (“Avitag”) was appended for enzymatic site specific biotin conjugation. Following protein expression, inclusion bodies were isolated and then dissolved in solubilization buffer (8 M urea, 25 mM MES pH 6.0, 10 mM EDTA, 0.1 mM DTT), while TCRs were dissolved in the solubilization buffer containing 6 M guanidine hydrochloride (GnHCl). Thirty milligrams each of TCR alpha and TCR beta were diluted into 500 mL refolding buffer [3 M urea, 0.2 M Arg-HCl, 150 mM Tris-HCl pH 8.0, 1.5 mM reduced glutathione, 0.15 mM oxidized glutathione and stirred at 4° C. for 72 h. Refolded TCR was dialyzed at 4° C. for 24 h in 4 L dialysis buffer (10 mM Tris pH 8.5, 50 mM NaCl) and then for an additional 24 h in fresh 4 L dialysis buffer. The resultant TCR complexes are concentrated and purified using Ni-NTA, and size-exclusion chromatography. The resulting proteins were analyzed by SDS-PAGE under reducing and non-reducing conditions. Final protein concentrations quantitated by A280 analysis. Purified proteins were stored in aliquots at -80 degrees until used.

### Example 10. Production and Characterization of Bispecific T Cell Receptor TCR-19

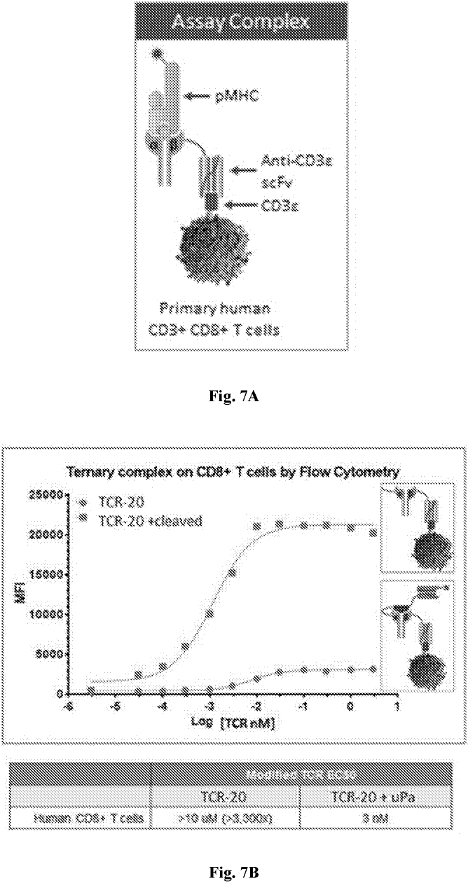

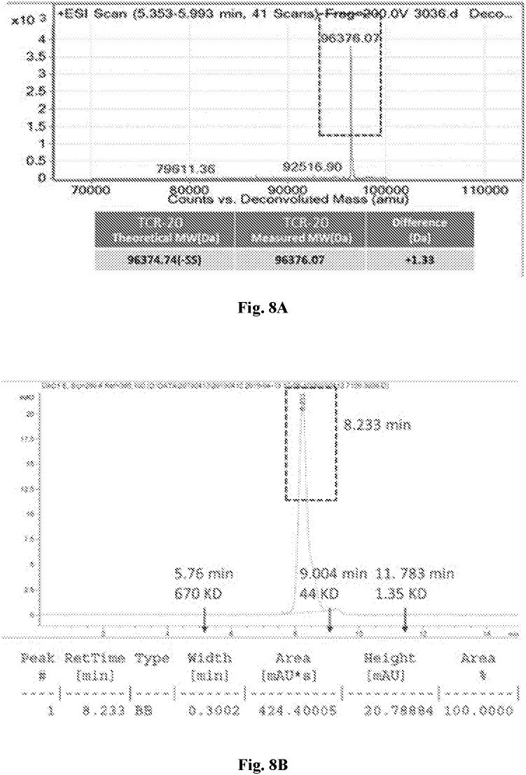

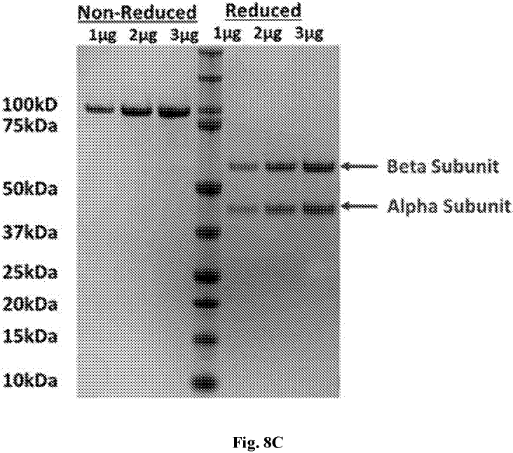

After expression, purification, and reconstitution of the heterodimeric masked, half-life extended, bispecific T-cell recruiting T cell receptor, the resulting protein was analyzed to determine the quality and integrity of the preparation. To do so TCR-19 was examined by determining intact mass through mass spectroscopy, aggregate content by HPLC size exclusion chromatography (SEC), and finally a qualitative analysis of dimeric integrity and purity by SDS-PAGE analysis. Specifically, TCR-19 is composed of the extracellular domains of the alpha and beta subunits of the MAGE-A3 affinity optimized T cell receptor, IC-3. To the amino terminus of the IC-3 extracellular alpha subunit (SEQ ID NO: 5) a pMHC competitive mask corresponding to a Peptide-5 was recombinantly fused via a split flexible linker containing an intervening protease cleavage site (SEQ ID NO: 4). To the amino terminus of the Peptide-5 mask (SEQ ID NO: 3) an anti-human serum albumin single domain antibody was additionally fused to enable half-life extension properties (SEQ ID NO: 2). For purification purposes a poly histidine extension was appended to the C-terminus. For the complementary IC-3 beta subunit an anti-human CD3 scFv (SEQ ID NO: 8) was recombinantly appended via a short flexible linker to the amino terminus. Finally, a biotin accepting substrate (Avitag) for birA was recombinantly appended to the C-terminus (SEQ ID NO: 10). Again, following the reconstitution as described above, the assembled resulting protein complex accurate correct mass and complete disulfide bond formation was examined via intact mass spectroscopy (FIG. 8A). Results from MS analysis confirmed correct sizes of both subunits and full formation of cysteine-cysteine disulfide bonds. To determine if the resulting heterodimer was present predominantly as a single heterodimeric unit or rather in some higher order aggregates, the resulting protein was examined by HPLC size exclusion chromatography (HPLC-SEC) (FIG. 8B). The protein was found to elute as a single peak corresponding to the expected heterodimeric mass, indicating virtually no presence of higher order aggregates. Finally, to qualitatively examine purity and stability the protein was analyzed by SDS-PAGE under non-reducing and reducing conditions (FIG. 8C). Under non-reducing conditions the protein was present as a single band corresponding to the intact heterodimer, while under reducing conditions the two protein subunits migrated at their respective expected masses. Together the composite of analysis supports desired physical properties and biophysical disposition of the bispecific T cell receptor.

### TCR-19 Subunit Sequences

TCR-19 alpha subunit with N-terminal HSA and Peptide-5 cleavable mask, and C-terminal His-tag (SEQ ID NO: 1)

MEVQLVESGGGLVQPGNSLRLSCAASGFTFSKFGMSWVRQAPGKGLEWVS
SISGSGRDTLYADSVKGRFTISRDNAKTTLYLQMNSLRPEDTAVYYCTIG
GSLSVSSQGTLVTVSSGGGGSGGGSGGVSCKDVYDEAFCWTGGGGSLSGR
SDNHGSSGTKQEVTQIPAALSVPEGENLVLNCSFTDSAIYNLQWFRQDPG
KGLTSLLYVRPYQREQTSGRLNASLDKSSGRSTLYIAASQPGDSATYLCA
VRPGGAGPFFVVFGKGTKLSVIPNIQNPDPAVYQLRDSKSSDKSVCLFTD
FDSQTNVSQSKDSDVYITDKCVLDMRSMDFKSNSAVAWSNKSDFACANAF
NNSIIPEDTFFPSPESSGGHHHHHHHH

Anti-albumin SDA (SEQ ID NO: 2)

MEVQLVESGGGLVQPGNSLRLSCAASGFTFSKFGMSWVRQAPGKGLEWVS
SISGSGRDTLYADSVKGRFTISRDNAKTTLYLQMNSLRPEDTAVYYCTIG
GSLSVSSQGTLVTVSS

P MAGE-A3 peptide mask (SEQ ID NO: 3)

VSCKDVYDEAFCWT

Protease substrate (SEQ ID NO: 4)

LSGRSDNH

IC-3 alpha subunit (SEQ ID NO: 5)

KQEVTQIPAALSVPEGENLVLNCSFTDSAIYNLQWFRQDPGKGLTSLLYV
RPYQREQTSGRLNASLDKSSGRSTLYIAASQPGDSATYLCAVRPGGAGPF
FVVFGKGTKLSVIPNIQNPDPAVYQLRDSKSSDKSVCLFTDFDSQTNVSQ
SKDSDVYITDKCVLDMRSMDFKSNSAVAWSNKSDFACANAFNNSIIPEDT
FFPSPESS

Polyhistidine (SEQ ID NO: 6)

HHHHHHHH

TCR-19 beta subunit with N-terminal anti-CD3 scFv and C-terminal AviTag (SEQ ID NO: 7)

MAIQMTQSPSSLSASVGDRVTITCRASQDIRNYLNWYQQKPGKAPKLLIY
YTSRLESGVPSRFSGSGSGTDYTLTISSLQPEDFATYYCQQGNTLPWTFG
QGTKVEIKGGGGSGGGGSGGGGSGGGGSGGGSEVQLVESGGGLVQPGGSL
RLSCAASGYSFTGYTMNWVRQAPGKGLEWVALINPYKGVSTYNQKFKDRF
TISVDKSKNTAYLQMNSLRAEDTAVYYCARSGYYGDSDWYFDVWGQGTLV
TVSSGGGGSKAGVTQTPRYLIKTRGQQVTLSCSPISGHRSVSWYQQTPGQ
GLQFLFEYFSETQRNKGNFPGRFSGRQFSNSRSEMNVSTLELGDSALYLC
ASSFNMATGQYFGPGTRLTVTEDLKNVFPPEVAVFEPSEAEISHTQKATL
VCLATGFYPDHVELSWWVNGKEVHSGVCTDPQPLKEQPALNDSRYALSSR
LRVSATFWQNPRNHFRCQVQFYGLSENDEWTQDRAKPVTQIVSAEAWGRA
DggGLNDIFEAQKIEWHE

Anti-CD3 scFv (SEQ ID NO: 8)

MAIQMTQSPSSLSASVGDRVTITCRASQDIRNYLNWYQQKPGKAPKLLIY
YTSRLESGVPSRFSGSGSGTDYTLTISSLQPEDFATYYCQQGNTLPWTFG
QGTKVEIKGGGGSGGGGSGGGGSGGGGSGGGSEVQLVESGGGLVQPGGSL
RLSCAASGYSFTGYTMNWVRQAPGKGLEWVALINPYKGVSTYNQKFKDRF
TISVDKSKNTAYLQMNSLRAEDTAVYYCARSGYYGDSDWYFDVWGQGTLV
TVSS

IC-3 beta subunit (SEQ ID NO: 9)

KAGVTQTPRYLIKTRGQQVTLSCSPISGHRSVSWYQQTPGQGLQFLFEYF
SETQRNKGNFPGRFSGRQFSNSRSEMNVSTLELGDSALYLCASSFNMATG
QYFGPGTRLTVTEDLKNVFPPEVAVFEPSEAEISHTQKATLVCLATGFYP
DHVELSWWVNGKEVHSGVCTDPQPLKEQPALNDSRYALSSRLRVSATFWQ
NPRNHFRCQVQFYGLSENDEWTQDRAKPVTQIVSAEAWGRAD

Avitag (SEQ ID NO: 10)

GLNDIFEAQKIEWHE

### Example 11. Kinetic Binding of Masked Bispecific TCRs to Cognate pMHC Tumor Antigens

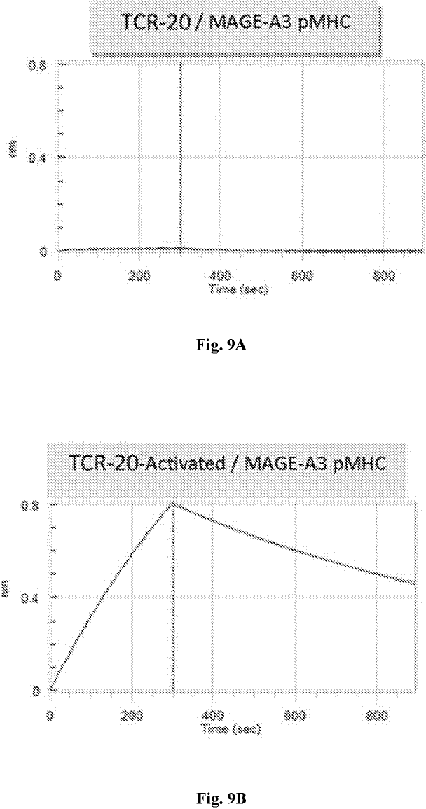

BLI based kinetic binding of masked bispecific TCR constructs to relevant tumor antigen pMHC was measured using a ForteBio Octet RED96 instrument. Biotinylated pMHC was first captured on streptavidin biosensors. Sensors were quenched using excess biocytin and then baselined in buffer. Masked bispecific TCRs were unmasked with protease where indicated. TCRs were associated onto the pMHC loaded biosensor. Association signal was monitored in real-time. Biosensors were then transferred to buffer and the dissociation of TCR was measured in real-time. Kinetic rates of association and dissociation were calculated using a one to one binding model within the instrument software (FIG. 9A and FIG. 9B).

### Example 12. Binding of Masked Bispecific TCRs to Cognate pMHC Tumor Antigens by ELISA

Masked bispecific TCRs were also evaluated in an ELISA format. Briefly, biotinylated pMHC was captured on neutravidin coated plates. Masked bispecific TCRs were diluted in bovine serum albumin buffer or human serum albumin buffer, titrated onto the pMHC captured plates, washed, and incubated with secondary antibody. The His-tag present at the C-terminus of the TCRs allowed for anti-His-tag horse radish peroxidase conjugated secondary antibody recognition. When indicated masked bispecific TCRs were unmasked with protease before binding to pMHC coated plates. Plates were then developed using tetramethylbenzidine (TMB) and stopped using acid. Absorbance at 450 nm was measured and plotted versus log-scale TCR concentration. The concentration of TCR required for half maximal saturation signal was calculated in Graphpad Prism software and reported as EC50 (FIG. 10). Binding was unaffected by the presence of human serum albumin or bovine serum albumin indicating that human serum albumin binding did not modulate binding affinity to pMHC.

### Example 13. Binding of Masked Bispecific TCRs to Albumin by ELISA

Masked bispecific TCR binding to albumin was evaluated in an ELISA format. Briefly, high binding plates were coated with albumin overnight. Masked bispecific TCRs were titrated onto the plates, washed, and incubated with secondary antibody. The His-tag present at the C-terminus of the TCRs allowed for anti-His-tag horse radish peroxidase conjugated secondary antibody recognition. Plates were then developed using tetramethylbenzidine (TMB) and stopped using acid. Absorbance at 450 nm was measured and plotted versus log-scale TCR concentration. The concentration of TCR required for half maximal saturation signal was calculated in Graphpad Prism software and reported as EC50 (FIG. 11). Constructs readily bind to human, cyno, and mouse serum albumin but do not bind bovine serum albumin.

### Example 14. Binding of Masked Bispecific TCRs to CD3 on the Surface of Human T Cells by Flow Cytometry

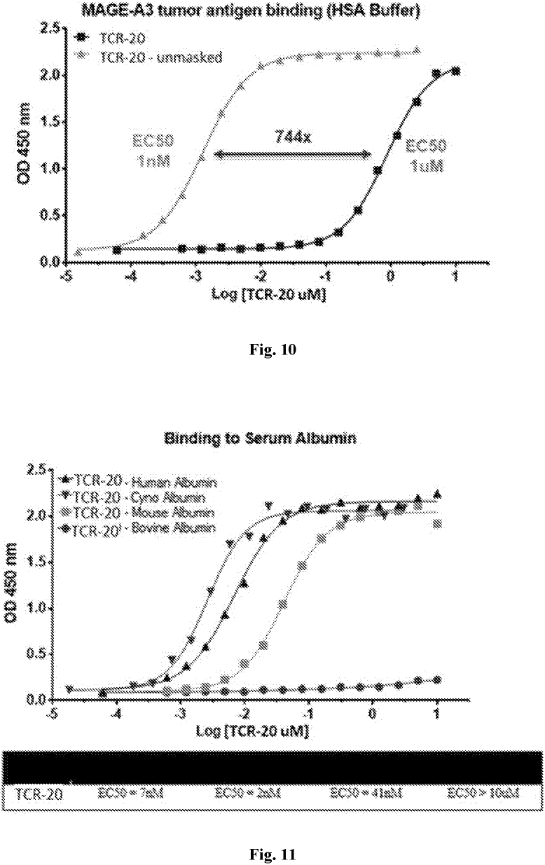

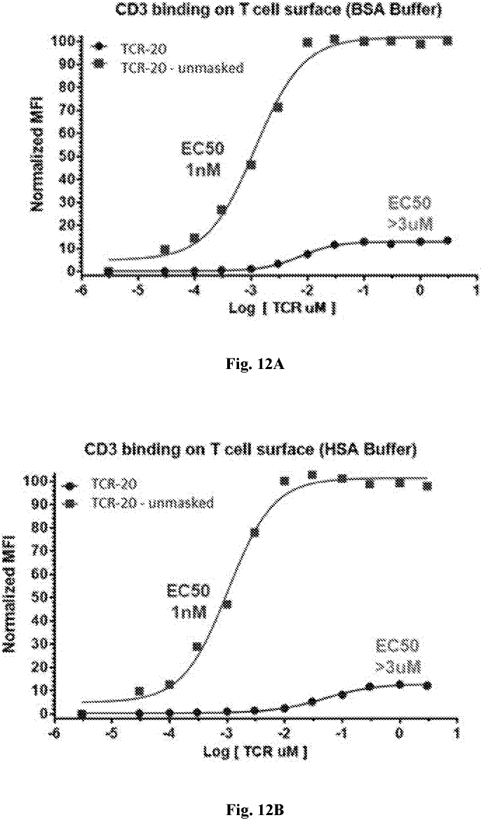

Masked bispecific TCRs ability to bind CD3 was evaluated on the surface of human T cells. T cells were thawed from frozen stock and diluted in buffer. 200,000 cells per well were loaded onto a 96 deep well round bottom polypropylene plate. Masked bispecific TCRs were unmasked with protease where indicated and serially diluted into buffer. Bispecific TCRs were diluted in bovine serum albumin buffer or human serum albumin buffer and incubated with CD8+ T cells for one hour on ice in a total volume of 100 uL. Cells were then pelleted, supernatant removed, and washed with 2 mL of buffer. Cells were then pelleted again, supernatant removed, and resuspended in 100 uL of cold diluted MAGE-A3 pMHC PE labeled tetramer. Cells were incubated on ice for 30 min with the fluorescent MAGE-A3 pMHC tetramer dilution before washing. Cells were pelleted, supernatant removed, and washed with 2 mL buffer. Cells were pelleted again, resuspended in buffer, and immediately run on a NovoCyte flow cytometer. The mean fluorescence intensity (MFI) was measured for each cell staining distribution, normalized to the maximum signal (100%), and plotted against the log concentration of bispecific TCR. The concentration of bispecific TCR that resulted in half maximal signal was calculated in GraphPad Prism and reported as EC50 (FIG. 12A-FIG. 12B). Binding was unaffected by the presence of human serum albumin or bovine serum albumin indicating that human serum albumin binding did not modulate binding affinity to CD3.

### Example 15. Masked Bispecific TCR Mediated T Cell Activation

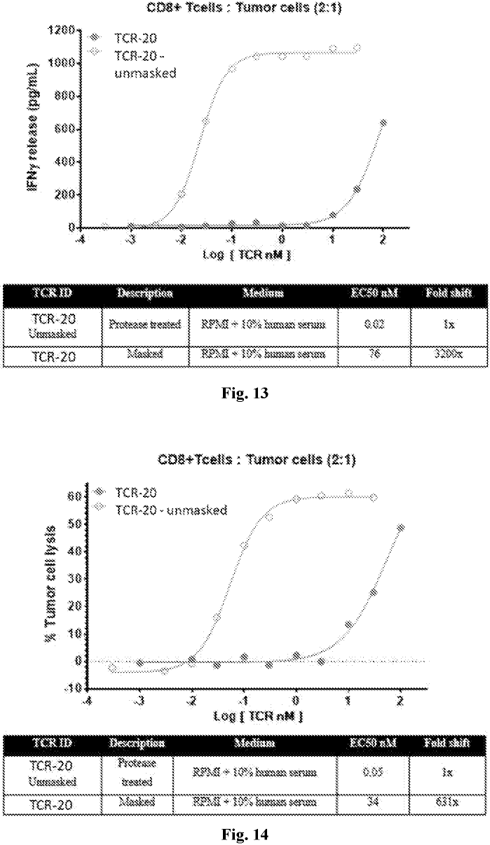

Tumor cells were seeded onto 96 well tissue culture treated flat bottom plates and allowed to adhere overnight. The following day, culture medium was removed from the cells, and replaced with medium containing serially diluted masked bispecific TCRs and CD8+ T cells. Masked bispecific TCRs were unmasked with protease when indicated. CD8+ T cells were added in at an effector cell: target cell ratio of 2:1 using the number of target cells seeded the day prior. CD8+ T cells and bispecific TCRs were co-cultured with target cells for 48 hours. Plates were gently spun down to collect cells at the bottom of the plate and the clarified supernatants collected. Amount of interferon gamma (IFNγ) was quantified using an ELISA kit and a human IFNγ protein standard following manufacturer’s instructions. Briefly, plates were coated with an anti IFNγ capture antibody, washed, and protein standard or diluted test supernatants were added to the plate and incubated overnight at 4° C. Plates were washed, and a secondary biotinylated detection antibody was added to the plate for one hour at room temperature. Plates were washed, streptavidin HRP loaded, washed again, and developed using TMB for 10 min. Plates were stopped in acid and absorbance was measured at 450 nm. The amount of IFNγ in test samples was quantified using a calibration curve generated using known amounts of IFNγ protein standard. The concentration of TCR bispecific required to generate half maximal IFNγ production was calculated using Graphpad Prism and reported as EC50 (FIG. 13).

### Example 16. Masked Bispecific TCR Mediated Tumor Cytotoxicity

Tumor cells were seeded onto 96 well tissue culture treated flat bottom plates and allowed to adhere overnight. The following day, culture medium was removed from the cells, and replaced with medium containing serially diluted bispecific TCRs, and CD8+ T cells. Masked bispecific TCRs were treated with protease when indicated. CD8+ T cells were added in an effector cell: Target cell ratio of 2:1 using the number of target cells seeded the day prior. CD8+ T cells and TCR bispecifics were co-cultured with target cells for 48 hours. Plates were gently spun down to collect cells at the bottom of the plate and the clarified supernatants collected. The relative amount of lactate dehydrogenase (LDH) present in the supernatants was quantified using the Promega LDH-Glo assay kit following the manufacturer’s instructions. Briefly, supernatants were diluted 500× in LDH storage buffer (200 mM Tris, 10% glycerol, 1% BSA, pH 7.3) of which 50 uL was added to white opaque 96 well assay plates. LDH detection enzyme mix was prepared with reductase substrate and added in equal volume of 50 uL per well. Luminescence was then measured on a luminometer. Signals were corrected for spontaneous LDH release from tumor cells alone and T cells alone. Maximum tumor cell lysis was measured in cells treated with lysis buffer for 45 min prior to supernatant harvest. Percent tumor cell lysis was calculated as corrected signal divided by corrected target maximum signal. The concentration of TCR bispecifics required to generate half maximal percent tumor cell lysis was calculated in Graphpad Prism and reported as EC50 (FIG. 14).

### Example 17. Masked Bispecific TCR Mediated Anti-Tumor in Vivo Efficacy

Masked bispecific TCRs in vivo efficacy is evaluated in human tumor bearing immunodeficient mice. Female NOD/SCID (NSG) mice (N=10 per group were) are subcutaneously implanted with a mixture of human donor PBMCs and target tumor cells. HCT116 (5 × 10⁶ viable cells per inoculum) are injected s.c. together with PBMCs from healthy human donors at an E:T cell ratio of 1:2 in the right dorsal flank of female NSG mice. As indicated, different conditions are tested for their influence on tumor outgrowth: vehicle alone, non-masked bispecific TCR, and various masked bispecific TCRs. TCRs are injected every day for 10 days starting the day of implantation. External calipers are used to measure tumor volumes twice weekly for five weeks.

### Example 18. Validation of Increased Half-Life; and Decreased Toxicity

### Mouse PK

BALB/c mice weighing 25-30 g are dosed intravenously with a single dose at 3 mg/kg bodyweight. Serial blood samples (35 uL) are collected via lateral tail sampling at 0.5, 4, 7, 24, 48, 72 and 96 h (anti-albumin binding fusion containing constructs) and at 0.033, 0.25, 0.75 and 1.66 h (non anti-albumin binding fusion constructs). To obtain sera, blood samples are centrifuged for 5 min at 10,000 rpm at room temperature. The presence of bispecific TCR in mouse serum samples are analyzed in a quantitative ELISA format. Bispecific TCR in serum is captured on CD3 coated plates, followed by detection using an anti-TCR variable beta antibody that recognizes the beta chain of bispecific TCR of interest. A final horseradish peroxidase conjugated secondary antibody is then added followed by development using TMB. ELISAs are stopped in acid and measured for OD 450 nm. Amount of bispecific TCR in mouse serum is calculated relative to a standard curve using relevant bispecific TCR spiked into mouse serum at known concentrations. Standard pharmacokinetic parameters are calculated based upon these quantitative measurements.

### Cyno PK

Cynomolgus monkeys are dosed intravenously with a single dose at 0.3 mg/kg bodyweight. Serial blood samples are collected at 15 minutes, 30 minutes, 1 hour, 2 hours, 4 hours and 8 hours post dose, then daily for three additional days, followed by weekly until study termination. The presence of bispecific TCR in serum samples is analyzed in a quantitative ELISA format. Bispecific TCR in cyno serum is captured on CD3 coated plates, followed by detection using an anti-TCR variable beta antibody that recognizes the beta chain of bispecific TCR of interest. A final horseradish peroxidase conjugated secondary antibody is then added followed by development using TMB. ELISAs are stopped in acid and measured for OD 450 nm. Amount of bispecific TCR in cyno serum is calculated relative to a standard curve using relevant bispecific TCR spiked into cyno serum at known concentrations. Standard pharmacokinetic parameters are calculated based upon these quantitative measurements.

### Example 19. Preparation of Soluble TCRs

Expression plasmids encoding the TCR alpha and beta chains or the TCR gamma and delta chains were produced using standard molecular biology techniques. Plasmids were transformed into chemically-competent cells and grown overnight at 37° C. Protein expression was induced by the addition of Isopropyl -D-1 -thiogalactopyranoside (IPTG) to 1 mM and bacteria were grown for a further 3 hours at 37° C. Bacteria were harvested by centrifugation at 4000 × g for 15 minutes and lysed in a protein extraction reagent containing DNAse. Lysis proceeded for 1 hour at room temperature with agitation before inclusion bodies were harvested by centrifugation at 10,000 × g for 5 minutes. Pellets were washed twice with a detergent buffer containing 1% Triton X100 and resuspended in a buffered saline solution.

Soluble TCRs were prepared by dissolving alpha and beta inclusion bodies in 6 M guanidine- HCI containing 10 mM dithiothreitol and incubating at 37° C. for 30 minutes. Samples were diluted into 1ml urea folding buffer (5 M urea; 0.4 ML-arginine; 0.1 M Tris-CI, pH 8.1; 2 mM EDTA; 6.5 mM -mercapthoethylamine; 1.9 mM cystamine) and dialysed against eight volumes of water overnight at 4° C., followed by dialysis for a further 24 hours in eight volumes of 10 mM Tris (8.1), with one buffer change. Dialysate (30 ml) was concentrated to 1 ml. Concentrated protein was diluted to 5 ml in phosphate-buffered saline and concentrated to 0.5 ml.

The resulting soluble TCRs were tested for their biochemical integrity by three methods. First, portions of the resulting TCRs were tested by heating in loading buffer in the presence or absence of reducing agent. Several concentrations of total protein were then examined by SDS- PAGE analysis to insure consistent results (FIG. 15A). Second, a portion of the resulting TCR was tested by size exclusion chromatography to determine whether there were smaller or larger than expected molecular weight components, indicating undimerized monomer or aggregating protein, respectively. Finally, the molecular mass by LC-MS methods was measured to further prove correct disulfide pairing was present in the reconstituted heterodimeric TCR (FIG. 15B, FIG. 15C).

TCR fusion constructs were either produced in E. coli cells similar to methods described above or transiently produced in suspension mammalian HEK293 cells according to known methods.

A representative example of the preparation of a MAGE-A3 TCR (TCR-1) is shown in Fig. TBD.

### Example 20. Binding Verification of Soluble TCR to Its Cognate pMHC

Kinetic binding of soluble TCR to pMHC was measured using a ForteBio Octet RED96 instrument. Biotinylated pMHC was first captured on streptavidin biosensors. Sensors were quenched using excess biocytin and then baselined in buffer. TCR was titrated in a 2-fold dilution series starting from 50 nM and was associated onto the pMHC loaded biosensor. Association signal was monitored in real-time. Biosensors were then transferred to buffer and the dissociation of TCR was measured in real-time. Data was background corrected, fit to a classic 1:1 binding model, and used to calculate kinetic rate constants.

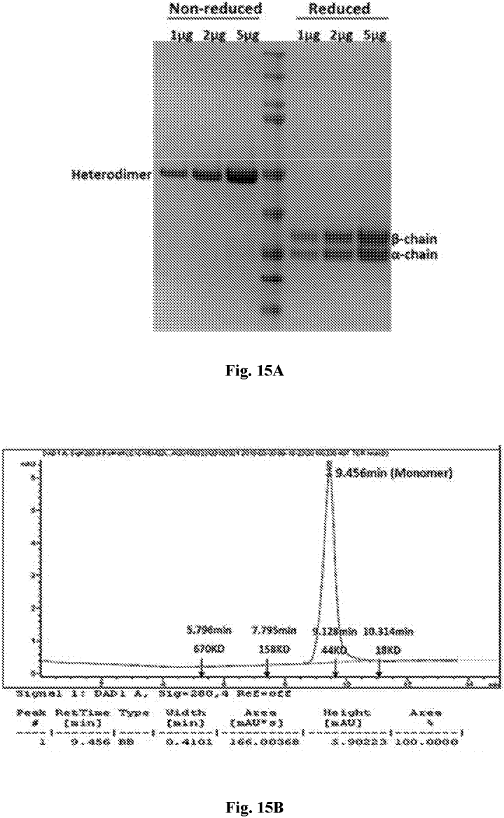

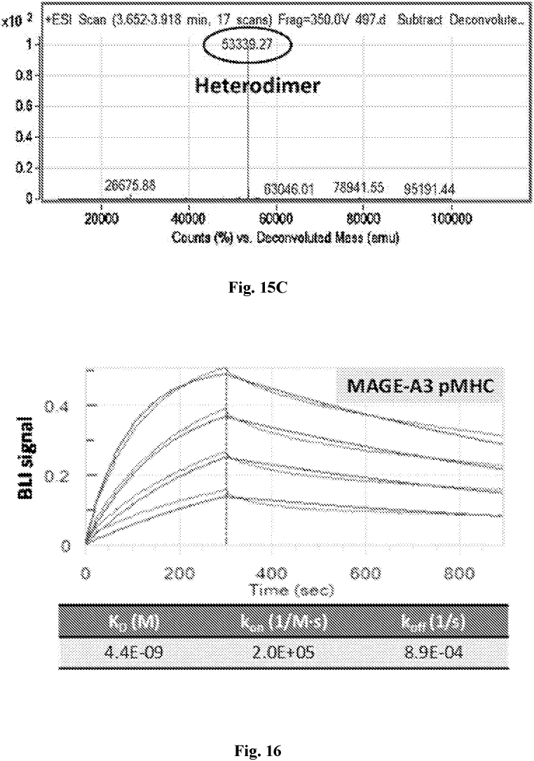

A representative example of the binding verification of a prepared MAGE-A3 TCR (TCR-1) is shown in FIG. 16.

### Example 21. Binding Verification of Soluble TCR to Its Cognate pMHC

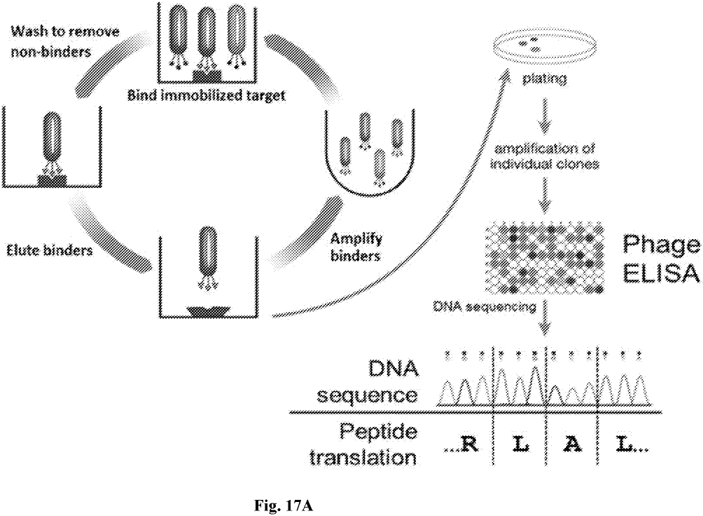

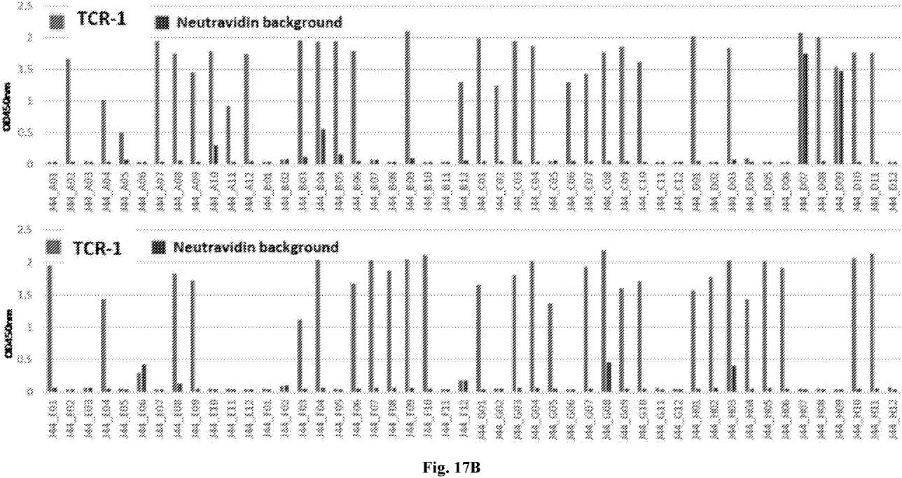

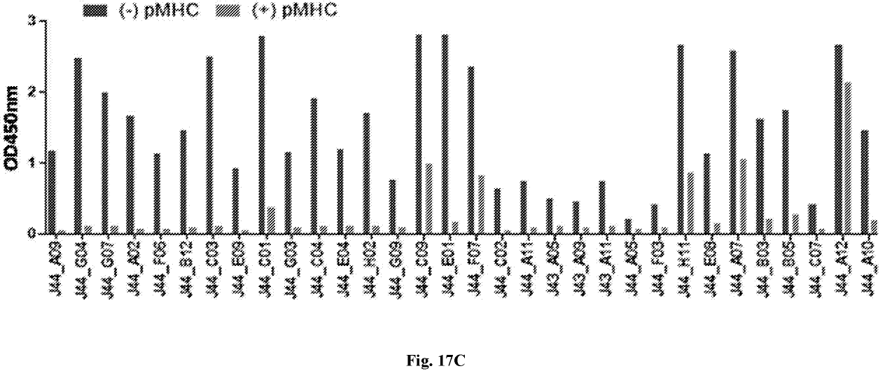

Peptides with the ability to bind to a T cell receptor (TCR) of interest are identified by biopanning a phagemid-display libraries of candidate peptides (FIG. 17A). Libraries are created via the introduction of recombinant expression of peptides fused to the m13 bacteriophage coat protein III (p3), resulting in display of the candidate peptides on the surface of the secreted bacteriophage. The candidate peptide libraries have variable amino acid sequences and collectively variable amino acid lengths.

Biopanning of m13 phagemid p3 displayed peptide libraries is performed with biotin conjugated TCR immobilized on streptavidin coated paramagnetic beads. Following binding to the target at pH 7.4 and subsequent washing steps, specifically bound phage are recovered by elution at pH 2.2, or at pH 11.0. Though individual clones can be sequenced or tested after a single round, enrichment of specific binding clones is typically accomplished by 2-4 rounds of successive biopanning and amplification. Following the enrichment of pools, phage biopanning phage pools are infected into TG1 cells and plated out on LB-ampicillin/agar plates for subsequent clonal isolation, DNA sequencing, and characterization (FIG. 17A).

### Phagemid Hit Identification ELISA

For hit identification, individual colonies were grown in 96-deep well plates for 2-4 hours and infected with helper phage to produce peptide displayed phagemid following an overnight growth. The next day the deep well plates were centrifuged to separate the soluble phagemid from the E. coli cells. The phagemid containing supernatants were then combined with PBS-Tween 20 (0.05%) + BSA (1%) pH neutral blocking buffer and incubated in previously TCR coated and blocked wells. After binding at 4° C. the plates were washed and specifically bound phage were detected by anti-m13 HRP conjugated antibodies using standard TMB-based chromogenic ELISA procedures (FIG. 17B). Daughter plates or individual wells were subjected to standard DNA sequencing for peptide identification.

### Phagemid Competition ELISA Assay

Phagemid peptide clones were next tested to determine whether they bound within the cognate pMHC binding space of the TCR, by target-based competition assay. We prepared biotin conjugated TCR immobilized and blocked 96-well ELISA plates similar to above. Next we added cognate pMHC to the well to block the antigen binding site. After a brief incubation period, phagemid supernatants were next added to the wells. Following an incubation at 4° C. the plates were washed and specifically bound phage were detected by anti-ml3 HRP conjugated antibodies using standard TMB-based chromogenic ELISA procedures. Phagemid clones binding within the pMHC binding pocket of the TCR would be blocked and be identified by a decreased ELISA signal, compared to a well lacking previous antigen blockade. A representative example of the phagemid competition ELISA is seen in FIG. 17Cfrom a collection of enriched clones isolated after three rounds of biopanning against MAGE-A3 TCR (TCR-1).

### Example 22. Synthetic Peptide Evaluation via TCR Binding, Inhibition, and Specificity

Peptides expressed on clonal phage that exhibit TCR specific binding and inhibition were chosen for further characterization. Exemplary phagemid peptides that bind to MAGE-A3 TCR (TCR-1) are listed in Table 2A and selected for peptide synthesis. Peptides selected for additional evaluation were first chemically synthesized and then evaluated for TCR binding, pMHC competition, and TCR selectivity.

TABLE 2A

Clonal phages that exhibited strong binding to MAGE-A3. Table discloses SEQ ID NOS 13-16, 12, and 17-31, respectively, in order of appearance

| Phage Clone | Peptid e ID | Phage ELISA | | | Peptide amino acid sequence from clonal phage | | | | | | | | | | | | | | | |
|---|---|---|---|---|---|---|---|---|---|---|---|---|---|---|---|---|---|---|---|---|
| | | NAv bkg d | MAG E-A3 TCR | pMHC comp. %bou nd | N-term Glyci ne | | 1 | 2 | 3 | 4 | 5 | 6 | 7 | 8 | 9 | 1 0 | 1 1 | 1 2 | 1 3 | 1 4 |
| J44A0 9 | Peptide -1 | 0.04 2 | 1.452 | 4.8% | G | G | E | S | C | Q | S | V | Y | D | S | S | F | C | Y | D |
| J44A0 2 | Peptide -2 | 0.04 6 | 1.661 | 4.4% | G | G | N | A | C | E | M | T | Y | D | H | T | F | C | D | P |
| J44F0 6 | Peptide -3 | 0.05 8 | 1.672 | 5.8% | G | G | R | I | C | E | E | V | Y | D | W | I | F | C | E | S |
| J44C0 3 | Peptide -4 | 0.05 6 | 1.951 | 4.5% | G | G | R | R | C | V | D | V | Y | D | N | A | F | C | L | I |
| J44C0 1 | Peptide -5 | 0.05 2 | 1.989 | 13.6% | G | G | V | S | C | K | D | V | Y | D | E | A | F | C | W | T |
| J44C0 9 | Peptide -6 | 0.05 4 | 1.857 | 35.4% | G | G | T | S | C | A | Q | I | Y | D | F | E | F | C | Y | S |
| J44E0 1 | Peptide -7 | 0.06 7 | 1.956 | 6.1% | G | G | S | L | C | S | L | V | Y | D | Q | D | F | C | E | S |
| J44C0 2 | Peptide -8 | 0.05 3 | 1.247 | 9.0% | G | G | N | S | C | S | L | V | Y | D | K | A | F | C | L | F |
| J44G0 3 | Peptide -9 | 0.05 9 | 1.806 | 7.1% | G | G | N | Q | C | w | E | V | Y | D | Q | E | F | C | S | L |
| J44H0 2 | Peptide -10 | 0.06 5 | 1.771 | 6.4% | G | G | S | A | C | S | R | I | Y | D | F | A | F | C | H | T |
| J44F0 3 | Peptide -11 | 0.05 4 | 1.118 | 20.0% | G | G | T | F | C | Y | F | D | H | G | L | V | N | C | Q | W |
| J44H1 1 | Peptide -12 | 0.05 1 | 2.137 | 32.6% | G | G | H | C | F | V | S | P | A | S | G | E | W | W | C | V |
| J44A1 0 | Peptide -13 | 0.29 7 | 1.781 | 13.3% | G | G | C | S | W | I | F | D | G | L | R | Y | F | S | K | C |
| J43A1 1 | Peptide -14 | 0.11 6 | 1.314 | 14.3% | | | V | R | T | W | F | E | K | F | P | E | L | V | | |
| J43A0 5 | Peptide -15 | 0.05 9 | 1.339 | 21.6% | | | L | V | W | G | C | I | W | D | D | M | C | S | | |
| J43A0 9 | Peptide -16 | 0.06 9 | 1.307 | 21.3% | | | W | H | W | E | P | S | M | V | W | G | M | L | | |
| J44E0 8 | Peptide -17 | 0.13 | 1.826 | 13.5% | G | G | G | C | F | V | S | P | A | T | G | F | T | W | C | V |
| J44B0 3 | Peptide -18 | 0.11 2 | 1.955 | 13.8% | G | G | D | C | Q | P | D | S | V | W | S | Y | W | Y | C | R |
| J44B0 5 | Peptide -19 | 0.15 8 | 1.953 | 15.9% | G | G | C | T | F | V | D | W | W | V | L | G | S | P | Y | C |
| J44C0 7 | Peptide -20 | 0.05 5 | 1.430 | 19.2% | G | G | C | L | M | N | D | Y | Y | Y | L | W | G | G | H | C |

Peptides were synthesized via standard peptide chemistry. Peptides were synthesized as linear or cyclic as appropriate. A C-terminal linker consisting of Gly4Ser (SEQ ID NO: 139), PEG4, and Lys(Biotin) was added to the phagemid peptide sequence identified from panning and DNA sequencing. The C-terminal acids were also capped via amidation. Peptides were purified by HPLC to ≥95% purity and verified by liquid chromatography assisted mass spectrometry (LC-MS). Peptides were lyophilized prior to dissolution in DMSO.

Synthetic peptides were initially screened for binding to their panning target. As an example, peptides listed bind to MAGE-A3 TCR, TCR-1. Peptide binding was evaluated using both kinetic measurements via Bio-layer Interferometry (BLI) or equilibrium measurements using enzyme linked immunosorbent assays (ELISAs).

### Kinetic Binding of TCR to Peptides

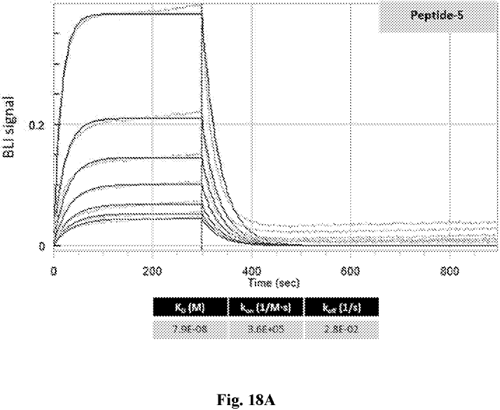

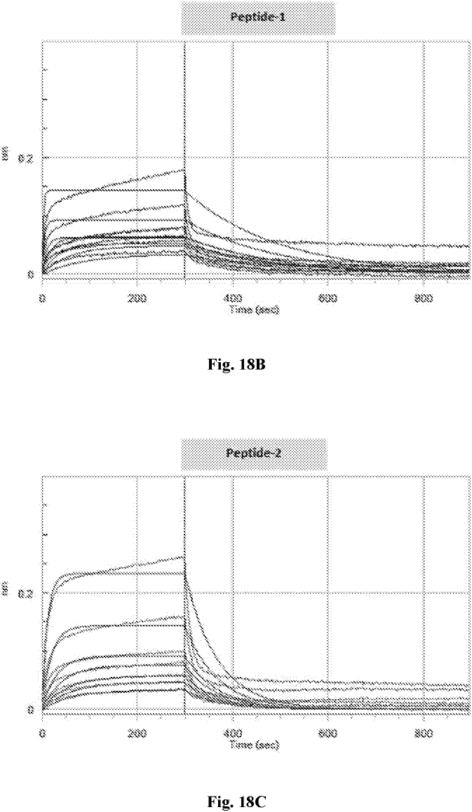

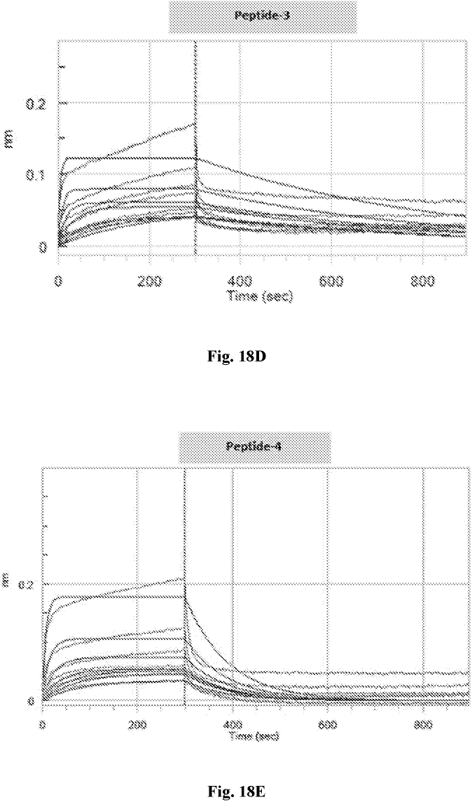

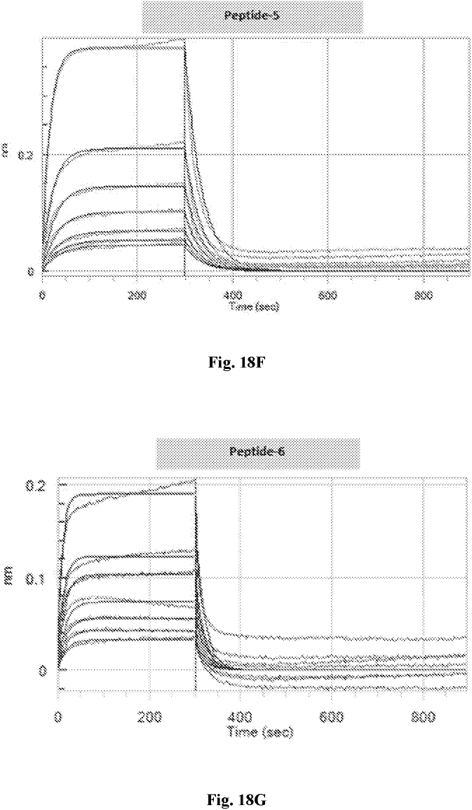

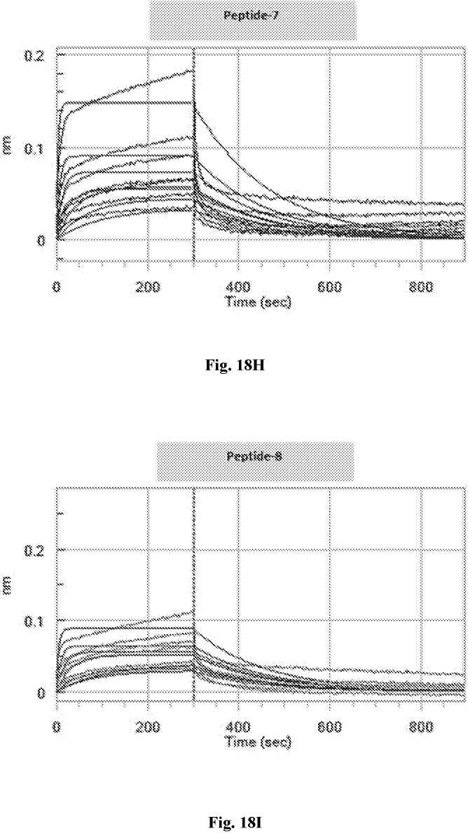

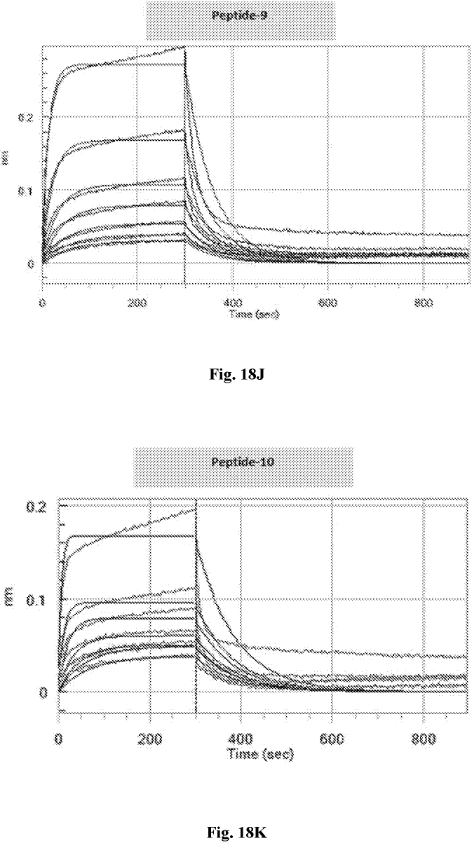

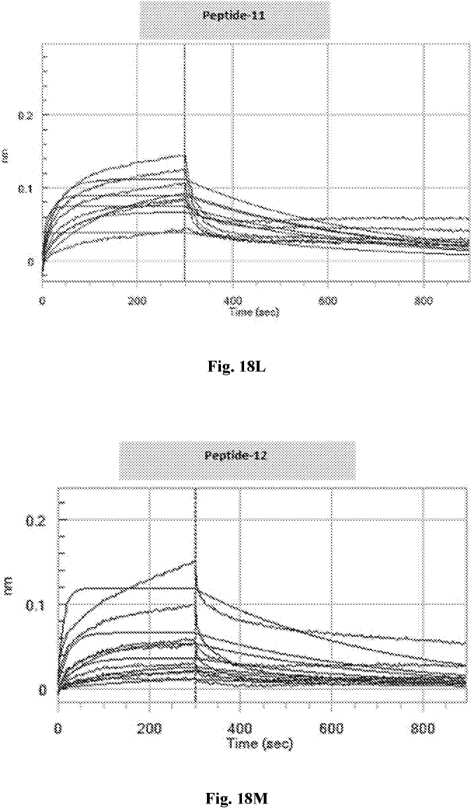

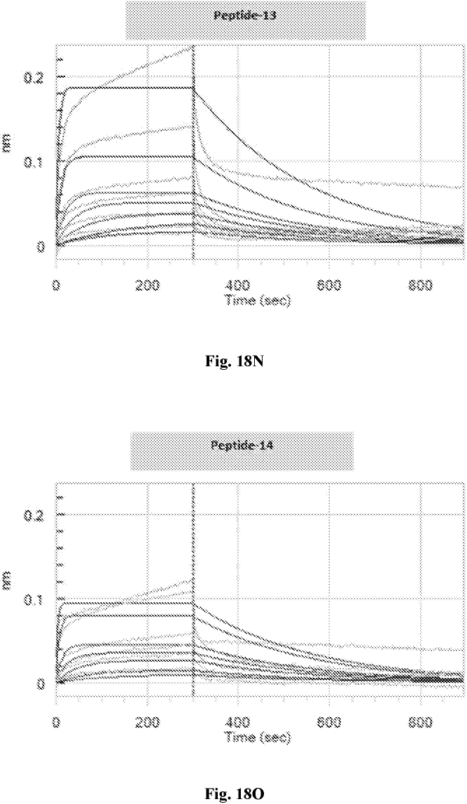

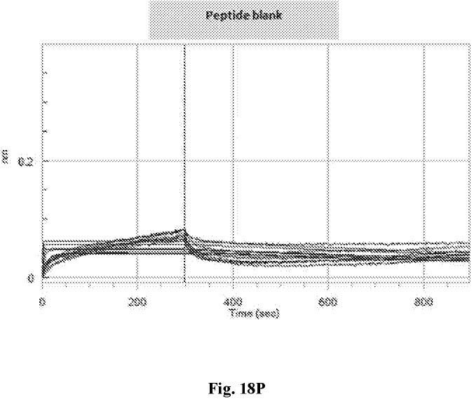

BLI based kinetic binding of TCR to peptides was measured using a ForteBio Octet RED96 instrument. Biotinylated peptides were first directly captured on streptavidin biosensors. Sensors were quenched using excess biocytin and then baselined in buffer. A dilution series of TCR was made and associated onto the peptide loaded biosensor. Association and dissociation signals were monitored in real-time. Signals were fit to a 1:1 binding model in order to derive binding constants, kon and koff, as well as KD. Exemplary kinetic binding sensorgrams are shown in FIG. 18Ausing the peptide Peptide-5 and MAGE-A3 TCR (TCR-1).

### Equilibrium Binding of TCR to Peptides

Peptide binding was also examined in an ELISA format. Biotinylated peptides were captured on neutravidin coated plates. TCR was then prepared in a half-log dilution series starting from 10 uM and titrated onto the peptide captured plates. A secondary horse radish peroxidase (HRP) antibody conjugate that recognizes 6x histidine tag (SEQ ID NO: 99) was used to detect bound TCR-1. The concentration of TCR required to observe half maximal binding signal (EC50) was then calculated using Graphpad Prism. Example binding of MAGE-A3 TCR (TCR-1) to captured peptide or MAGE-A3 pMHC is shown in FIGS. 18B-18P. A wide range of peptide EC50s was observed from 10 nM to >10 uM whereas the cognate MAGE-A3 pMHC EC50 was 0.8 nM (FIG. 19A). EC50 binding data for peptides is summarized in Table 2. Promising peptides that exhibited reasonable binding by both BLI and ELISA were progressed into competitive binding experiments.

TABLE 2B

Binding characteristics of example peptides

| Peptide ID | Peptide Amino Acid Sequence | Kinetic binding-Octet BLI | | | ELISA | |
|---|---|---|---|---|---|---|
| | | Bind | Competes | | EC50 | IC50 |
| | | (Y/N) | 100 um (Y/N) | IC50 uM | uM | uM |
| Peptide-1 | GGESCQSVYDSSFCYDGGGGS [PEG4] Lys (biotin) -NH2 (SEQ ID NO: 100) | Y | Y | 11.2 | 0.10 | 7.79 |
| Peptide- | GGNACEMTYDHTFCDPGGGGS [PEG4] Lys (biotin) - | Y | Y | 5.1 | 0.17 | 3.13 |
| 2 | NH2 (SEQ ID NO: 101) | | | | | |
| Peptide-3 | GGRICEEVYDWIFCESGGGGS [PEG4] Lys (biotin) -NH2 (SEQ ID NO: 102) | Y | Y | 35.7 | 0.31 | |
| Peptide-4 | GGRRCVDVYDNAFCLIGGGGS [PEG4] Lys (biotin) -NH2 (SEQ ID NO: 103) | Y | Y | 13.9 | 0.25 | |
| Peptide-5 | GGVSCKDVYDEAFCWTGGGGS [PEG4] Lys (biotin) -NH2 (SEQ ID NO: 104) | Y | Y | 8.3 | 0.01 | 0.60 |
| Peptide-6 | GGTSCAQIYDFEFCYSGGGGS [PEG4] Lys (biotin) -NH2 (SEQ ID NO: 105) | Y | Y | 5.5 | 0.17 | 2.25 |
| Peptide-7 | GGSLCSLVYDQDFCESGGGGS [PEG4] Lys (biotin) -NH2 (SEQ ID NO: 106) | Y | Y | 6.8 | 0.16 | 4.55 |
| Peptide-8 | GGNSCSLVYDKAFCLFGGGGS [PEG4] Lys (biotin) -NH2 (SEQ ID NO: 107) | N | | | 0.43 | |
| Peptide-9 | GGNQCWEVYDQEFCSLGGGGS [PEG4] Lys (biotin) -NH2 (SEQ ID NO: 108) | Y | Y | 5.5 | 0.05 | 3.10 |
| Peptide-10 | GGSACSRIYDFAFCHTGGGGS [PEG4] Lys (biotin) -NH2 (SEQ ID NO: 109) | Y | N | | 0.33 | |
| Peptide-11 | GGTFCYFDHGLVNCQWGGGGS [PEG4] Lys (biotin) -NH2 (SEQ ID NO: 110) | Y | N | | 0.50 | |
| Peptide-12 | GGHCFVSPASGEWWCVGGGGS [PEG4] Lys (biotin) -NH2 (SEQ ID NO: 111) | Y | N | | 0.08 | >100 |
| Peptide-13 | GGCSWIFDGLRYFSKCGGGGS [PEG4] Lys (biotin) -NH2 (SEQ ID NO: 112) | Y | N | | 0.15 | 90.89 |
| Peptide-14 | VRTWFEKFPELVGGGGS [PEG4] Lys (biotin) -NH2 (SEQ ID NO: 113) | N | | | 0.67 | |
| Peptide-15 | LVWGCIWDDMCSGGGGS [PEG4] Lys (biotin) -NH2 (SEQ ID NO: 114) | Y | N | | 0.10 | >100 |
| Peptide-16 | WHWEPSMVWGMLGGGGS [PEG4] Lys (biotin) -NH2 (SEQ ID NO: 115) | Y | N | | 7.85 | |
| Peptide-17 | GGGCFVSPATGFTWCVGGGGS [PEG4] Lys (biotin) -NH2 (SEQ ID NO: 116) | N | | | 2.34 | |
| Peptide-18 | GGDCQPDSVWSYWYCRGGGGS [PEG4] Lys (biotin) -NH2 (SEQ ID NO: 117) | Y | N | | 0.27 | |
| Peptide- | GGCTFVDWWVLGSPYCGGGGS [PEG4] Lys (biotin) -NH2 (SEQ ID NO: 118) | Y | N | | 1.12 | |
| 19 | | | | | | |
| Peptide-20 | GGCLMNDYYYLWGGHCGGGGS [PEG4] Lys (biotin) -NH2 (SEQ ID NO: 119) | Y | N | | 3.90 | |

### Inhibition of Kinetic Binding for TCR to Its Cognate pMHC Using Inhibitory Peptides

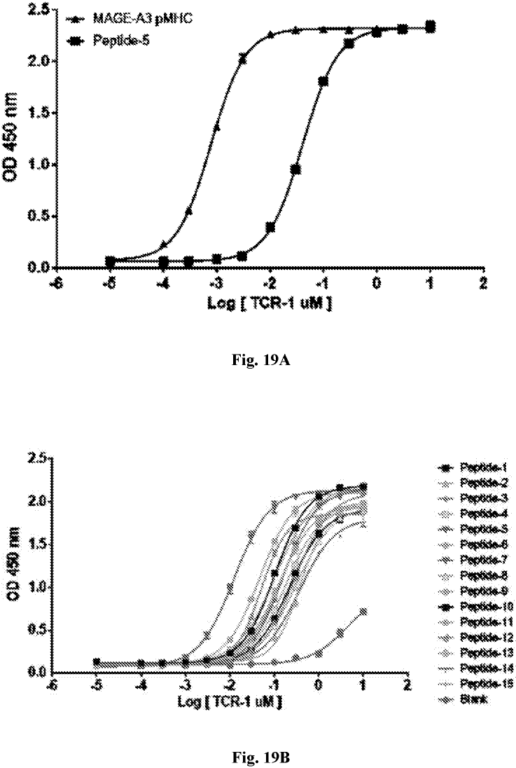

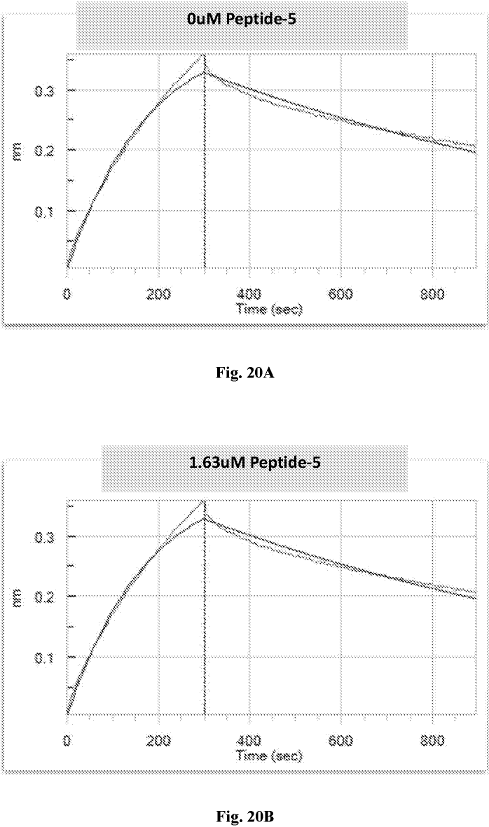

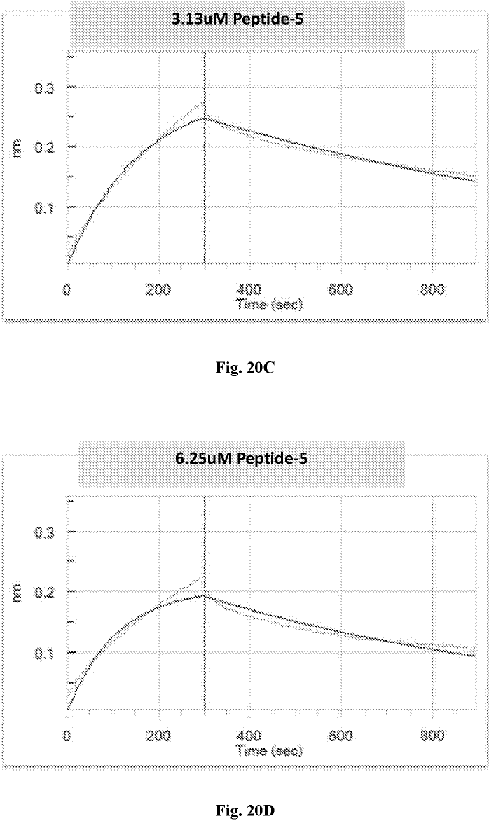

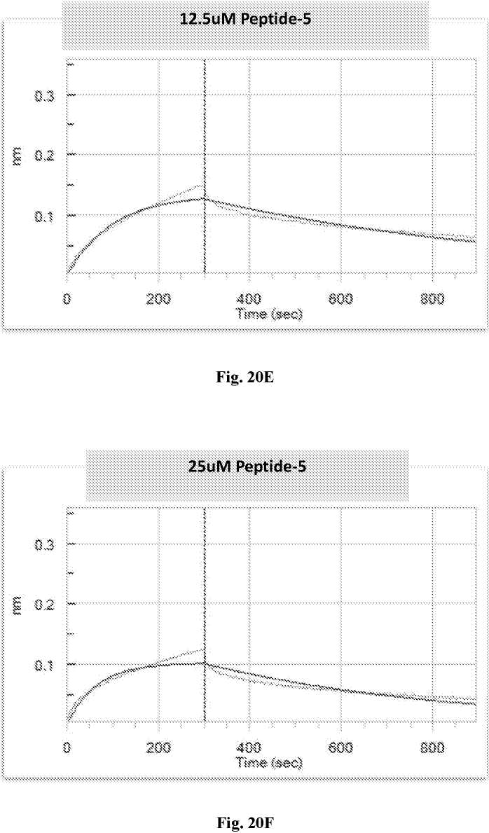

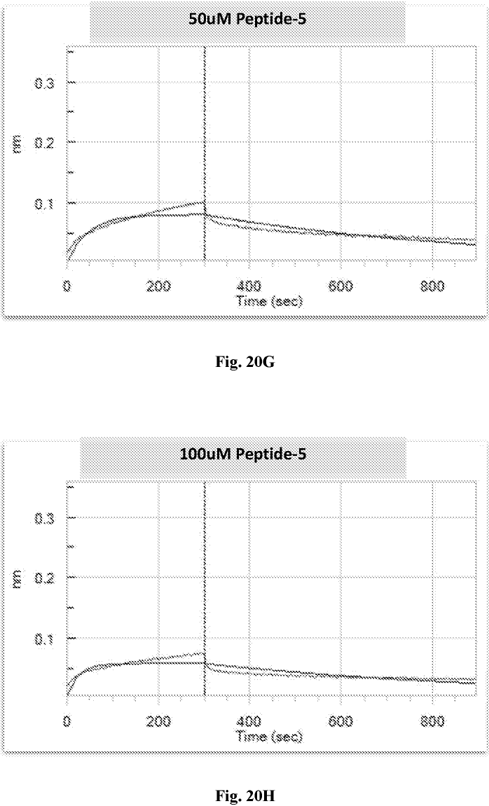

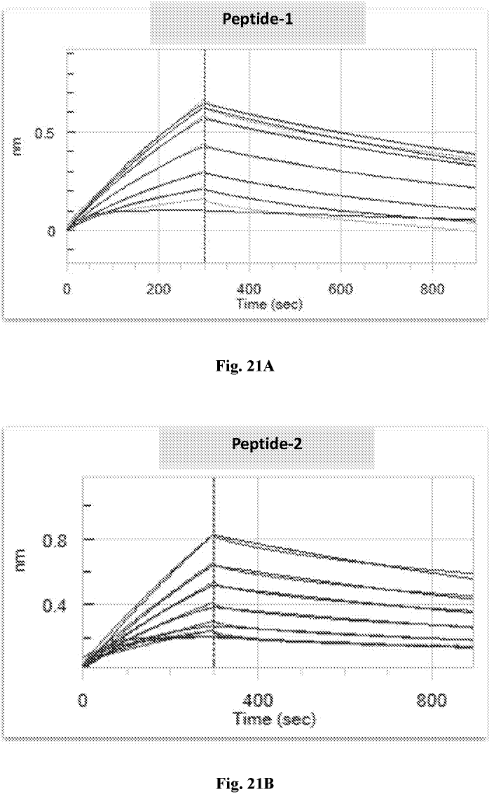

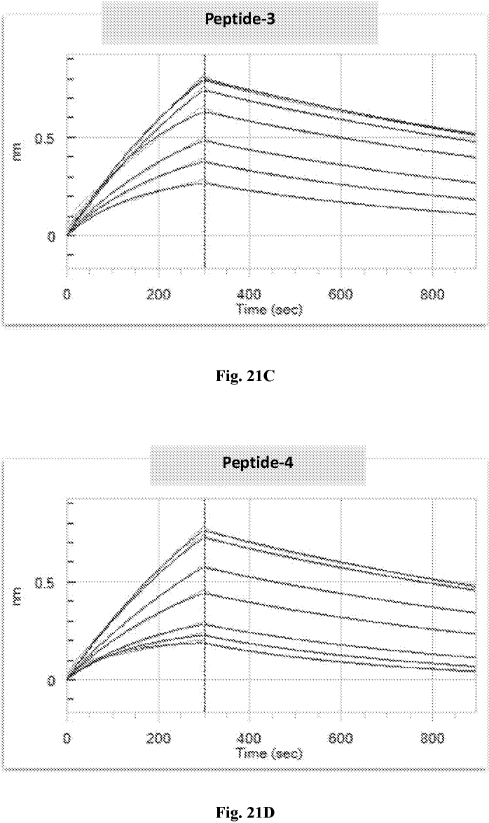

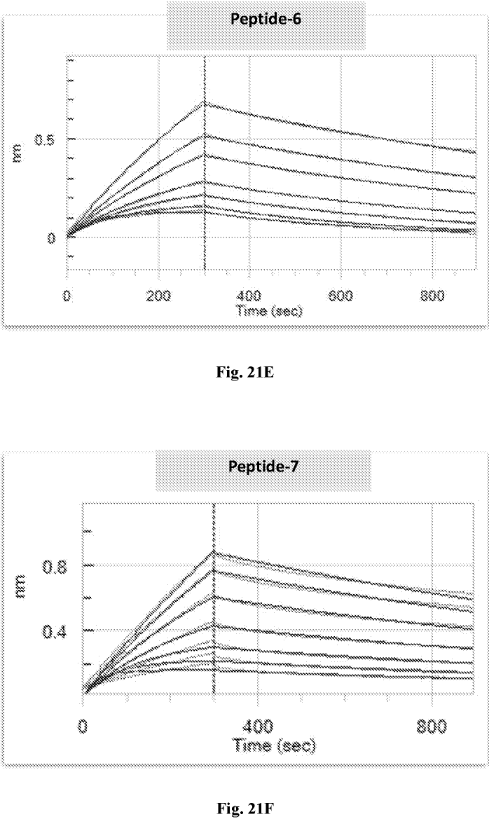

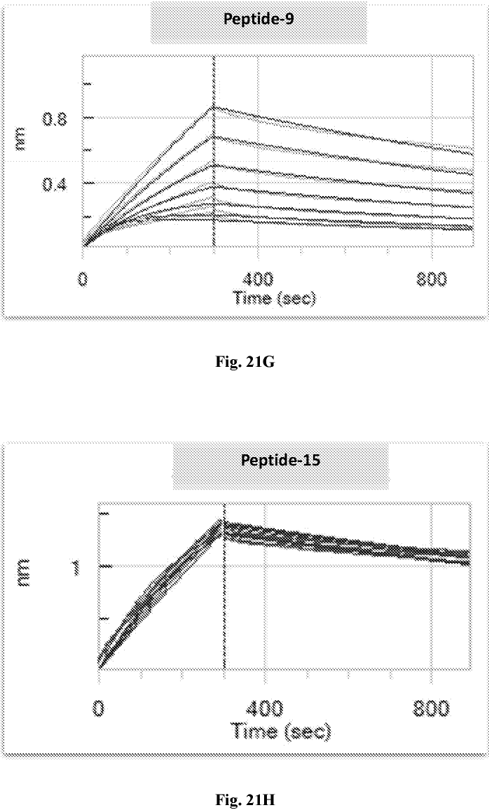

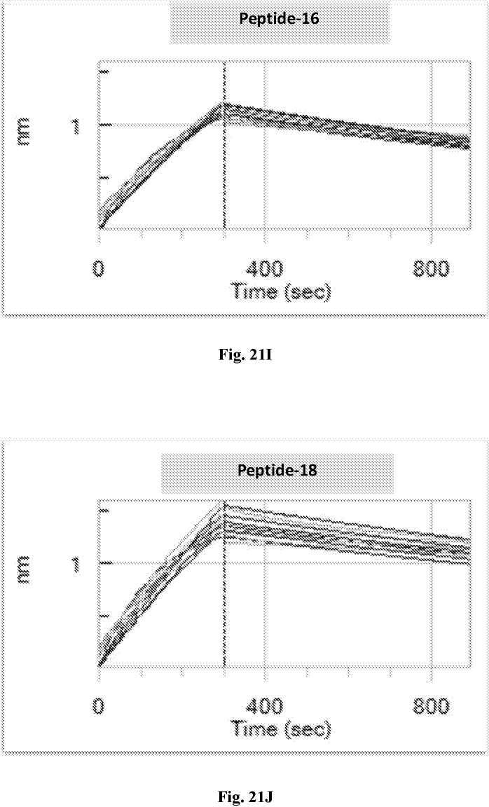

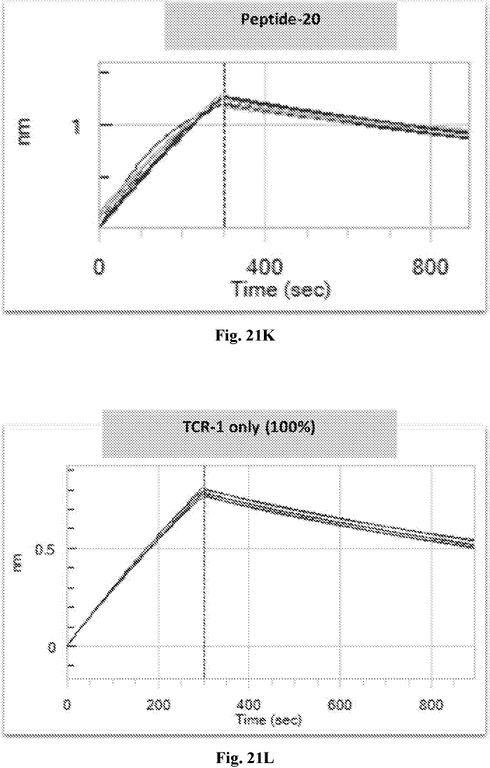

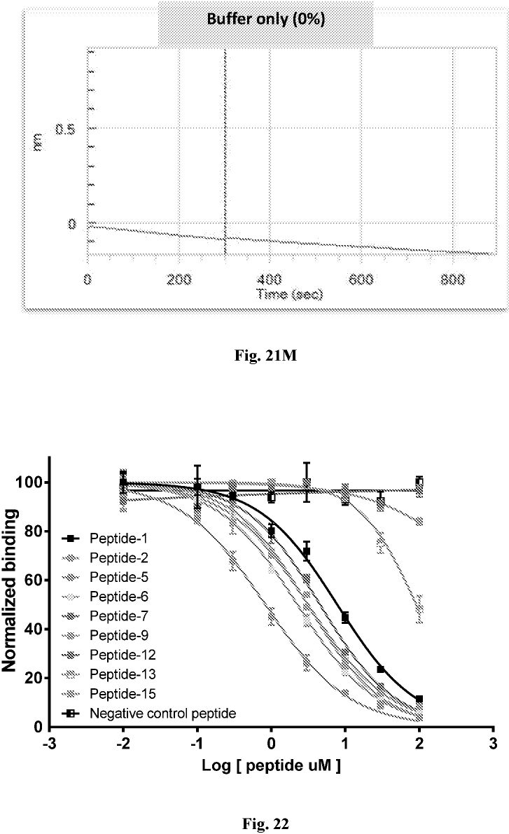

Peptides that bind do not necessarily exhibit desired function. For a peptide to function as a mask it must by definition inhibit the TCR of interest from binding its cognate pMHC. Therefore, peptides that bind the example MAGE-A3 TCR, TCR-1, were progressed into competitive inhibition studies designed to test the inhibitory function of each peptide. Multiple peptides were evaluated for masking function via BLI and ELISA. FIGS. 20A-20H and FIGS. 21A-21J provide example peptide inhibition of TCR-1 binding to MAGE-A3 pMHC in a dose dependent manner. IC50 data for all peptides is summarized in Table 2.

Dose dependent kinetic inhibition of TCR-1 binding to MAGE-A3 pMHC using the identified peptide binders was measured via BLI using a ForteBio Octet RED96 instrument. First, biotinylated pMHC was captured on streptavidin biosensors. Sensors were quenched using excess biocytin and then baselined in buffer. Inhibitory peptide was titrated in a two fold dilution series starting from 100 uM and pre-incubated with a constant concentration of 50 nM TCR. Peptide and TCR mixtures were then associated onto the pMHC loaded biosensor. Zero concentration of inhibitory peptide or zero concentration of TCR were used as controls. Association and dissociation signals were monitored in real-time. The maximal association signal was normalized from 100% (0 uM inhibitory peptide control) to 0% (0 nM TCR control) and plotted versus log-scale inhibitory peptide concentration. Graphpad Prism was used to calculate the inhibitory concentration of peptide required to achieve 50% maximal signal (IC50) (Table 2).

### Inhibition of Equilibrium Binding for TCR to Its Cognate pMHC Using Inhibitory Peptides

Inhibition of TCR binding to its cognate pMHC was also measured in an ELISA format. Biotinylated pMHC was captured on neutravidin coated plates, quenched using excess biocytin, and washed. Inhibitory peptide was titrated in a half-log dilution series starting from 100 uM and pre-incubated with a constant concentration of 1 nM TCR. Inhibitory peptide and TCR mixtures were then incubated on the pMHC captured plates. A secondary HRP antibody conjugate that recognized the histag of the TCR was then used to detect the plate bound TCR. The ELISA signal was normalized from 100% (0 nM inhibitory peptide control) to 0% (0 nM TCR control) and plotted versus log-scale inhibitory peptide concentration (FIG. 22). Dose dependent decrease of signal was indicative of peptides that compete for TCR binding to its cognate pMHC. Graphpad Prism was used to calculate the inhibitory concentration of peptide required to achieve 50% maximal signal (IC50) summarized in Table 2.

### Inhibitory Peptide Specificity Evaluations

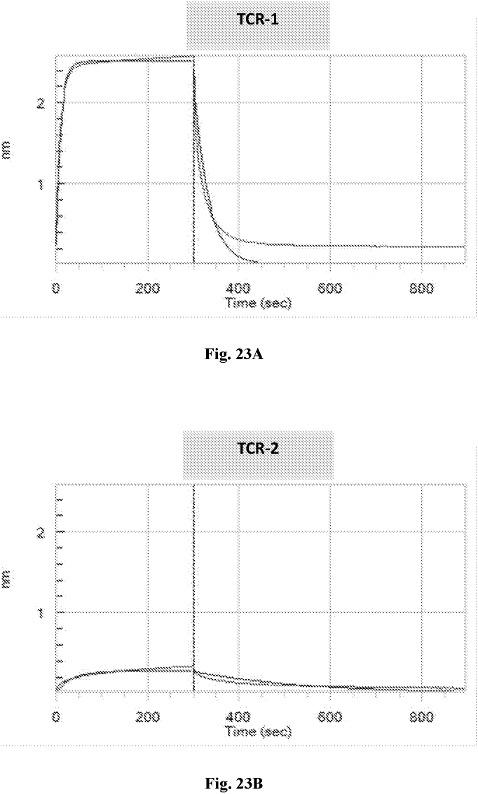

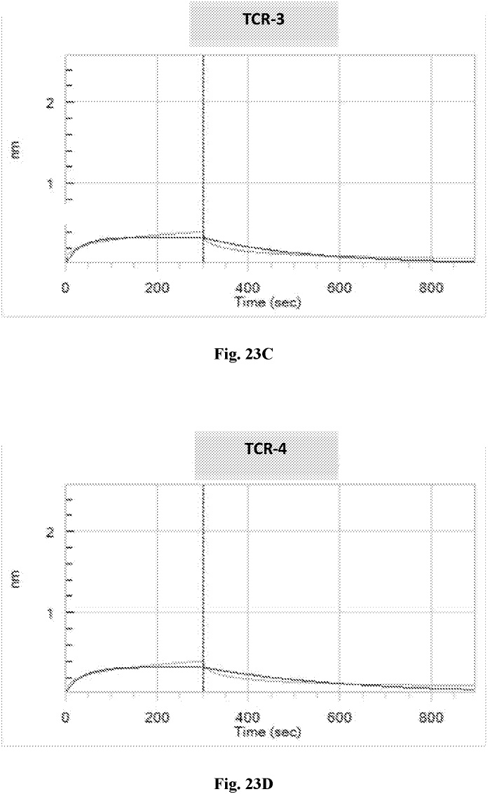

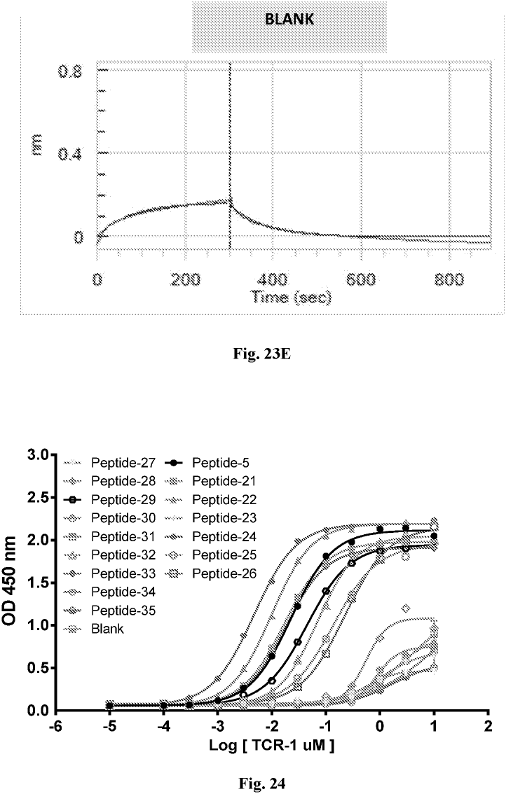

Potent inhibitory peptides were tested for TCR specific binding. For example, the TCR specificity of Peptide-5 was tested using closely related TCRs, TCR-2, TCR-3, and TCR-4, of the same family that also recognize the cognate MAGE-A3 pMHC. This family of TCRs differ in sequence by point mutations in the CDR domains know to contribute to binding affinity and specificity for cognate MAGE-A3 pMHC. Similar to previous kinetic binding experiments, binding of TCRs to Peptide-5was evaluated by BLI at a loading concentration of Peptide-5 that saturated the streptavidin biosensor. TCRs were associated at the extreme concentration of 100 uM onto the inhibitory peptide loaded biosensor. In parallel, 100 uM TCRs were associated on a blank control sensor to establish background signal related to non-specific binding of the TCRs. Association and dissociation signals were monitored in real-time. While TCR-1 readily bound Peptide-5 with a high signal well above background, closely related A3a TCRs do not as their signal was identical to that of background TCR binding to a blank sensor (FIGS. 23A-23E). This suggested that the example peptide Peptide-5 binds TCR-1 with high specificity.

### Example 23. Sequence Activity Relationships for Inhibitory Peptide Peptide-5 Against Target MAGE-A3 TCR

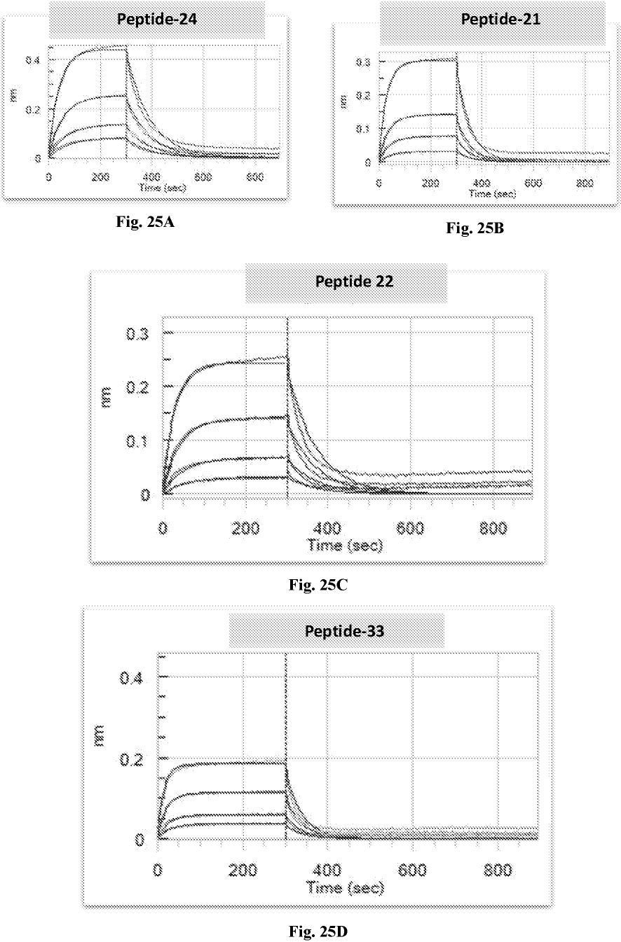

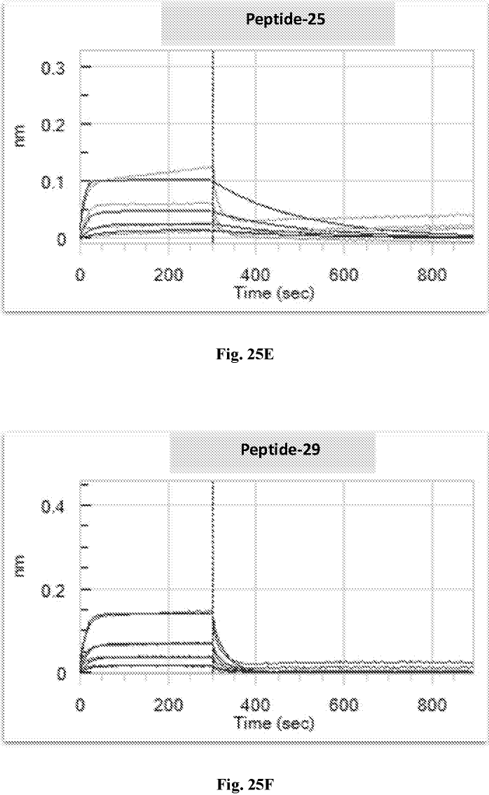

Given the selective binding of Peptide-5and TCR-1 relative to TCR-2, TCR-3, and TCR-4 TCRs, it is likely that Peptide-5interacts with residues present in the CDR2α, CDR3α, or CDR3β domains of TCR-1. Since TCR CDR domains form key binding interactions with cognate pMHC, binding of Peptide-5 at or near the CDR2 and CDR3 domains could explain why Peptide-5 blocks TCR-1 recognition of MAGE-A3 pMHC. In order to understand the key residues within Peptide-5 that drive binding interaction with TCR-1, all residues of Peptide-5 were mutated one by one to alanine. Not surprisingly, Ala scan peptides revealed key residues corresponding to the consensus sequence identified from phage panning, “CXXXYDXXFC” (SEQ ID NO: 120), required for TCR-1 binding (FIG. 24 and FIGS. 25A-25F). Additional control peptides, linear MAGE-A3 and Titin absent MHC presentation, were used as controls and exhibited a lack of binding to TCR-1. Interestingly, one peptide, Peptide-24, exhibited improved binding and inhibition of TCR-1 (FIG. 24 and FIGS. 25A-25F), suggesting further optimization post inhibitory peptides discovery could be fruitful. Binding and inhibition data of the Ala mutated peptides of FIG. 24 and FIGS. 25A-25F against TCR target TCR-1 are shown in Table 3.

Ala mutated versions of Peptide-5 were evaluated in kinetic and equilibrium binding and inhibition studies as previously described in Example 22.

TABLE 3

Binding and inhibition data

| Peptide ID | Sequence | Octet BLI | | ELISA | |
|---|---|---|---|---|---|
| | | Binds TCR | Competes at 100 uM | EC50 uM | IC50 uM |
| Peptide-5 | GGVSCKDVYDEAFCWTGGGGS [PEG4] Lys (biotin) -NH2 (SEQ ID NO: 104) | Yes | Yes | 0.01 | 0.60 |
| Peptide-21 | GGASCKDVYDEAFCWTGGGGS [PEG4] Lys (biotin) -NH2 (SEQ ID NO: 121) | Yes | Yes | 0.03 | 0.55 |
| Peptide-22 | GGVACKDVYDEAFCWTGGGGS [PEG4] Lys (biotin) -NH2 (SEQ ID NO: 122) | No | | 0.01 | 0.52 |
| Peptide-23 | GGVSAKDVYDEAFCWTGGGGS [PEG4] Lys (biotin) -NH2 (SEQ ID NO: 123) | Yes | Yes | >10 | |
| Peptide-24 | GGVSCADVYDEAFCWTGGGGS [PEG4] Lys (biotin) -NH2 (SEQ ID NO: 124) | Yes | Yes | 0.005 | 0.32 |
| Peptide-25 | GGVSCKAVYDEAFCWTGGGGS [PEG4] Lys (biotin) -NH2 (SEQ ID NO: 125) | Weak | | 0.21 | |
| Peptide-26 | GGVSCKDAYDEAFCWTGGGGS [PEG4] Lys (biotin) -NH2 (SEQ ID NO: 126) | No | | 0.24 | |
| Peptide-27 | GGVSCKDVADEAFCWTGGGGS [PEG4] Lys (biotin) -NH2 (SEQ ID NO: 127) | No | | >10 | |
| Peptide-28 | GGVSCKDVYAEAFCWTGGGGS [PEG4] Lys (biotin) -NH2 (SEQ ID NO: 128) | Yes | Yes | 6.70 | |
| Peptide-29 | GGVSCKDVYDAAFCWTGGGGS [PEG4] Lys (biotin) -NH2 (SEQ ID NO: 129) | No | | 0.07 | 1.46 |
| Peptide-30 | GGVSCKDVYDEAACWTGGGGS [PEG4] Lys (biotin) -NH2 (SEQ ID NO: 130) | No | | >10 | |
| Peptide-31 | GGVSCKDVYDEAFAWTGGGGS [PEG4] Lys (biotin) -NH2 (SEQ ID NO: 131) | Weak | | >10 | |
| Peptide-32 | GGVSCKDVYDEAFCATGGGGS [PEG4] Lys (biotin) -NH2 (SEQ ID NO: 132) | Yes | Yes | 0.07 | 1.33 |
| Peptide-33 | GGVSCKDVYDEAFCWAGGGGS [PEG4] Lys (biotin) -NH2 (SEQ ID NO: 133) | Yes | Yes | 0.02 | 0.66 |
| Peptide-34 | EVDPIGHLYGGGGS [PEG4] Lys (biotin) -NH2(MAGE-A3) (SEQ ID NO: 134) | No | | >10 | >100 |
| Peptide-35 | ESDPIVAQYGGGGS [PEG4] Lys (biotin) -NH2(Titin) (SEQ ID NO: 135) | No | | >10 | |

### Example 24. Design, Synthesis, and Evaluation of Exemplary Masked TCRs

Peptide-5 was fused to the N-terminus of the alpha or beta chain of TCR-1 via a flexible linker to assess functional masking. In some instances, the flexible linker was of different lengths and incorporated of a tumor protease specific substrate between the inhibitory peptide mask (Peptide-5) and the TCR alpha or beta chain. The proteolytic substrate within the linker enables tumor actuated binding that requires tumor restricted protease activity for therapeutic activation. Masked TCRs were produced and qualified as described in Example 19.

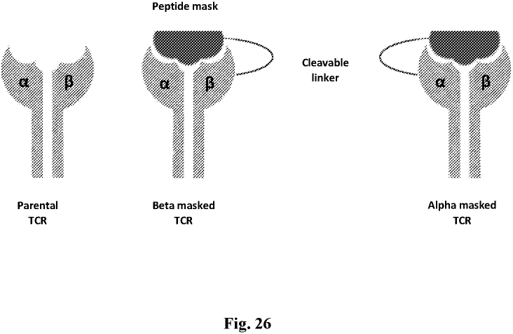

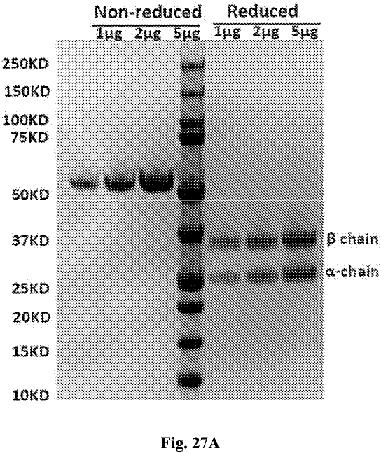

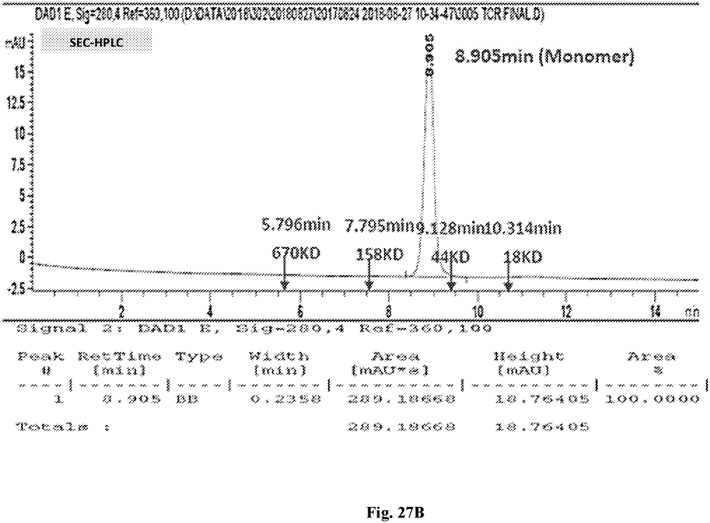

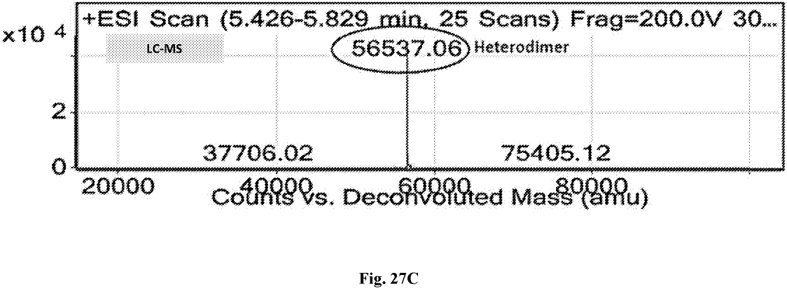

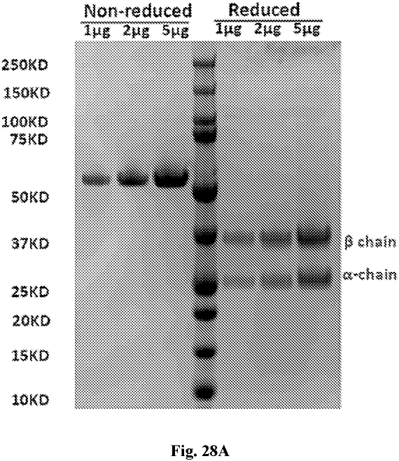

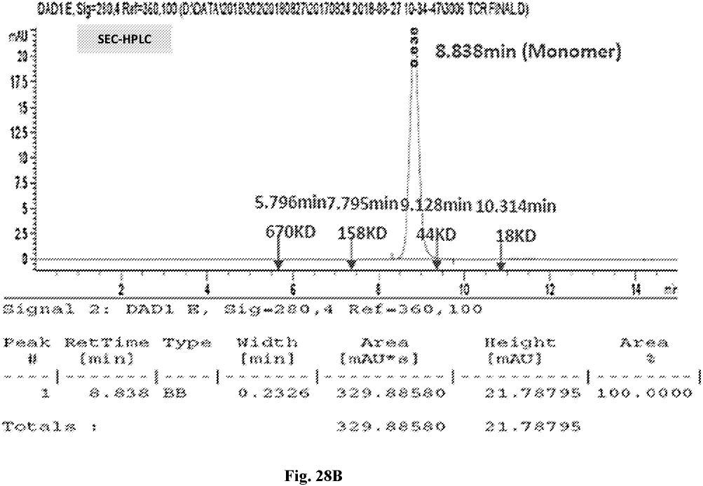

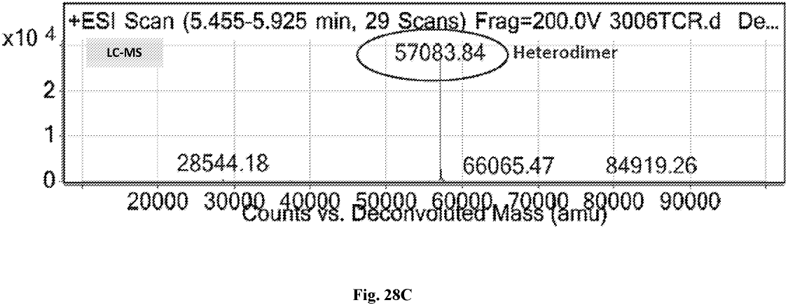

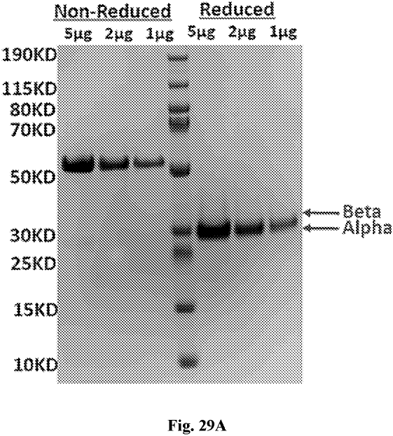

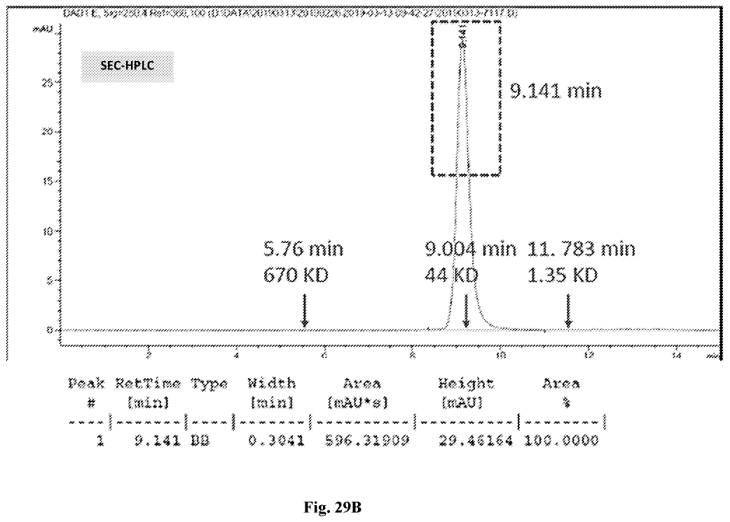

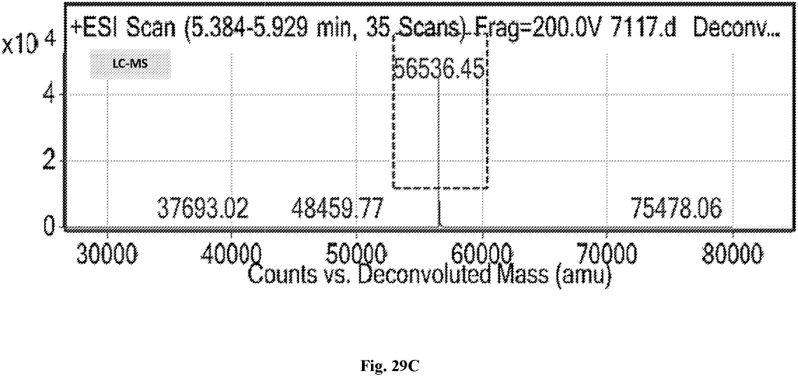

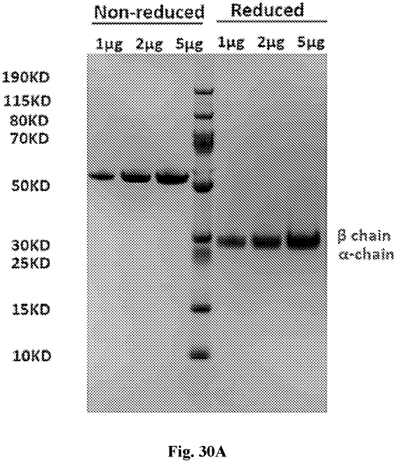

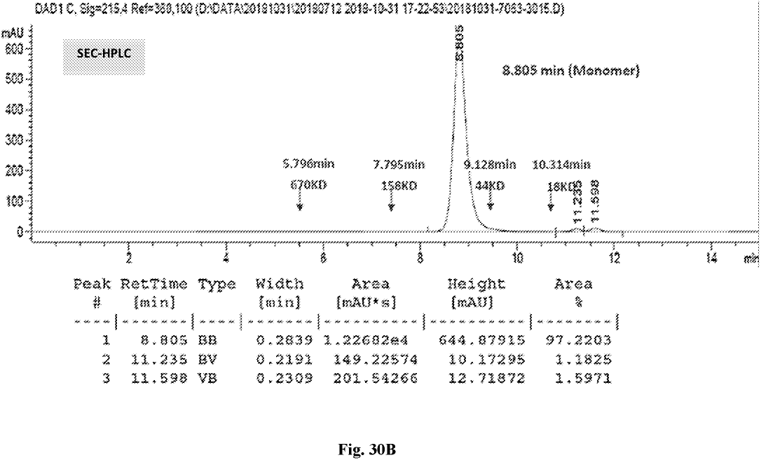

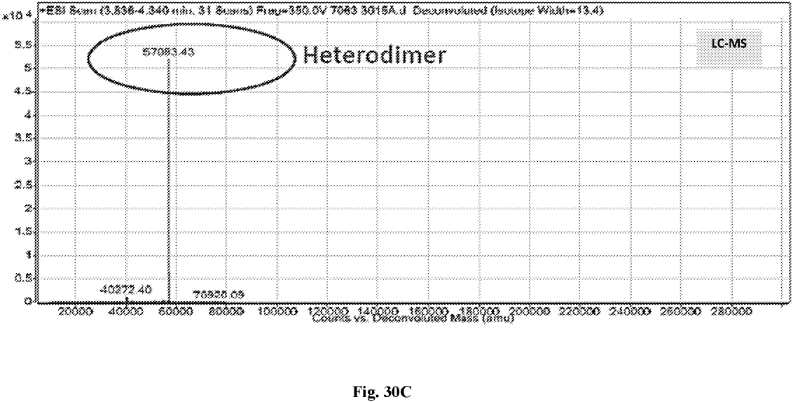

Masked TCR constructs were designed using inhibitory peptide Peptide-5 fused to the N-terminal alpha or beta chain of the parental TCR, TCR-1, using a protease cleavable linker (FIG. 26) containing the published substrate sequence LSGRSDNH (SEQ ID NO: 4). The LSGRSDNH (SEQ ID NO: 4) substrate is recognized and cleaved by several proteases, including urokinase and matriptase, that are upregulated in the tumor microenvironment across multiple tumor types. FIGS. 27A-27C, 28A-28C, 29A-29C, and 30A-30C highlight the production quality of the example masked TCRs.

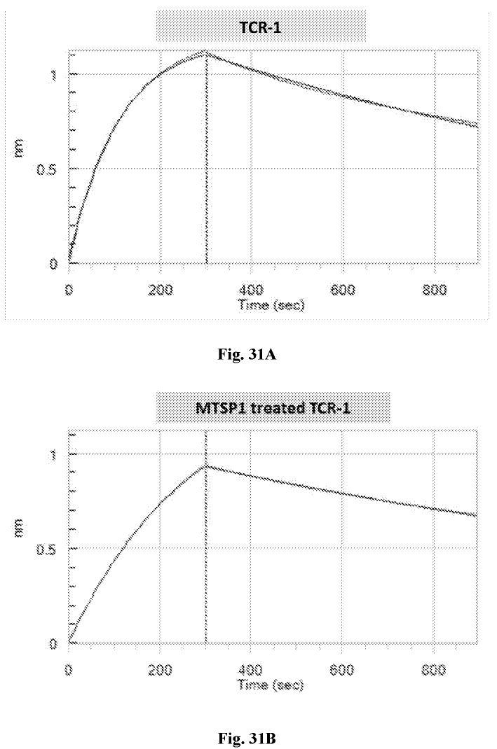

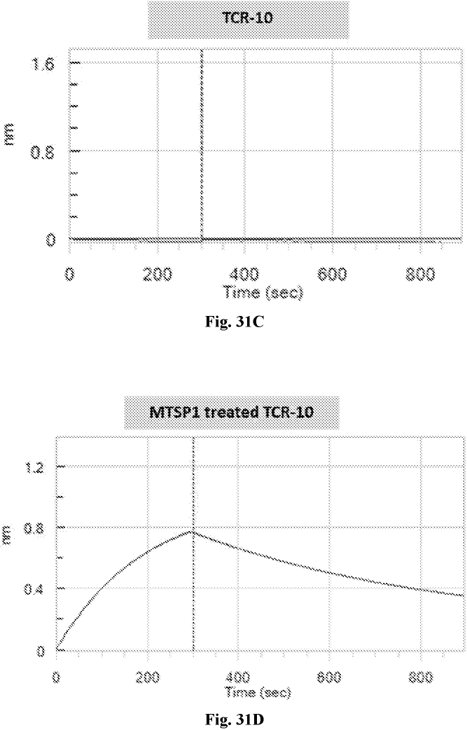

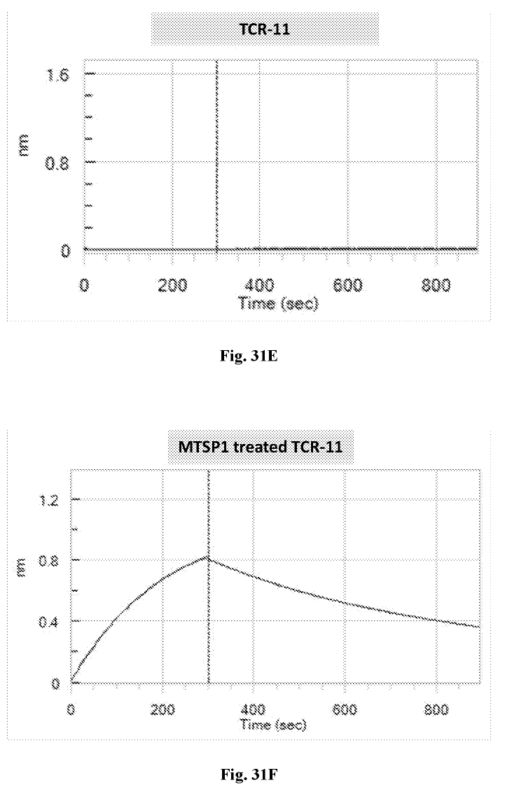

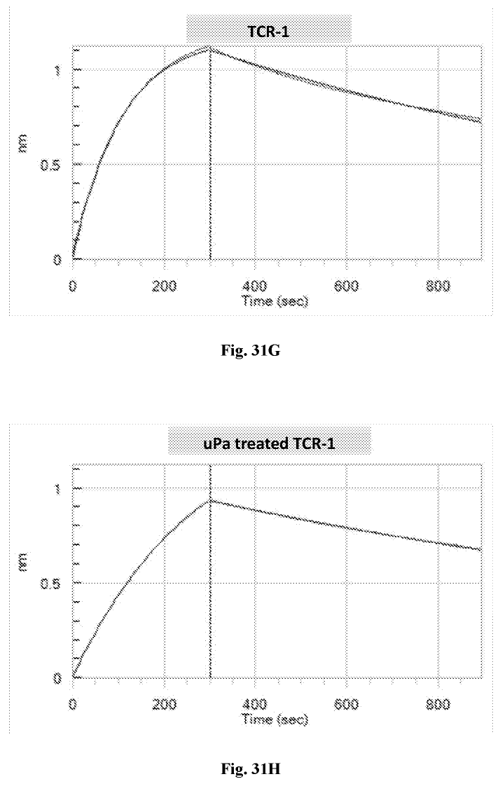

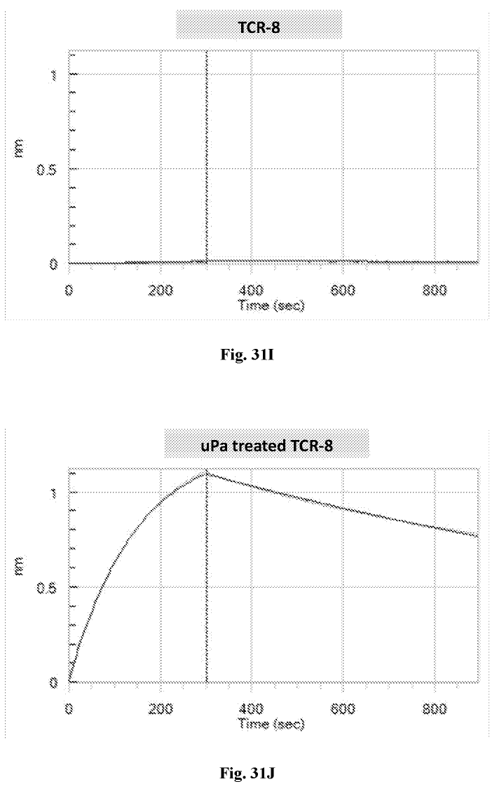

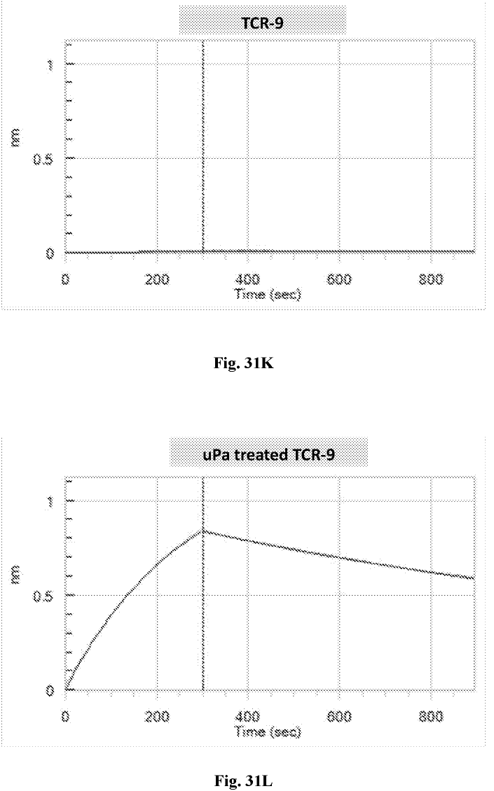

Recognition of Peptide-5 TCR fusions for cognate pMHC were evaluated in kinetics based BLI binding experiments similar to those described in Example 20. In some instances, TCRs were pre-treated with urokinase (uPa) or matriptase (MTSP1) where indicated. Briefly, biotinylated MAGE-A3 pMHC was loaded onto BLI streptavidin biosensors, quenched with excess biocytin and baselined in buffer. TCRs were then associated onto the pMHC loaded sensors at a single concentration of 50 nM. Sensors were transferred back to buffer to measure dissociation. Kinetic binding signals suggest that while the parental non-masked TCR-1 control exhibits full binding to its cognate pMHC, fusion of Peptide-5 to the N-terminal beta chain, TCR-8 and TCR-9, or the N-terminal alpha chain, TCR-10 and TCR-11, of the TCR completely blocked the TCR recognition of MAGE -A3 pMHC (FIGS. 31A-31L). Treatment of masked TCRs with protease fully restored pMHC binding signal equivalent to that of the parental non-masked TCR, TCR-1 (FIGS. 31A-31L).

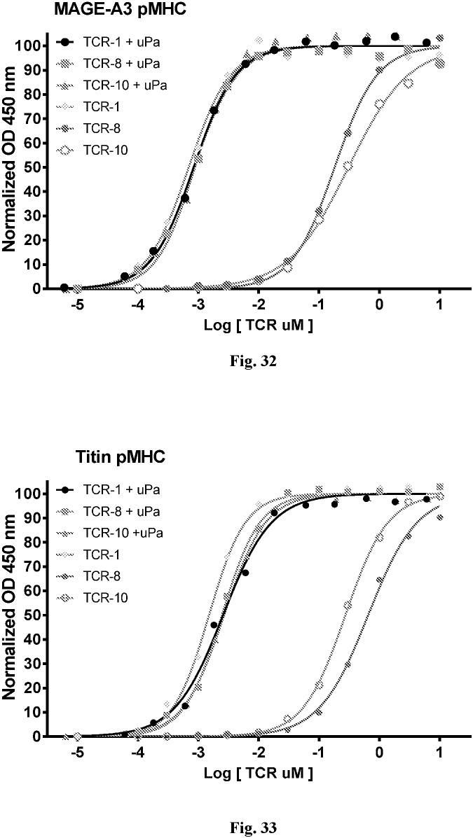

Recognition of Peptide-5 TCR fusions for cognate pMHC were also evaluated in equilibrium based ELISA binding experiments similar to those described in Example 20. In some instances the TCRs were treated with protease where indicated. Briefly, biotinylated pMHCs were captured on neutravidin coated plates followed by the addition of titrated TCRs. Plates were then incubated for a short time followed by a wash. A secondary anti-histag HRP conjugate antibody was used to detect bound TCR to the plate. The ELISA data in FIGS. 32-33 demonstrate hindered ability of masked TCRs to bind MAGE-A3 and Titin pMHCs respectively, that is otherwise restored to non-masked parental control levels by treatment with protease.

The BLI and ELISA data for masked TCRs suggests that the masking ability of Peptide-5 was maintained regardless of fusion to the alpha or beta chain of the TCR using different length linkers. Likewise, the Peptide-5 peptide fusion was able to hinder TCR binding to cognate MAGE-A3 pMHC as well as the off target Titin pMHC known to contribute to toxicity in the clinic.

Importantly however, fusion of MAGE-A3 antigen peptide (EVDPIGHLY (SEQ ID NO: 45); Peptide 34) to TCR-1 TCR in the identical formats with and without the protease cleavage site, TCR-6 and TCR-7 respectively, did not inhibit the TCR kinetic binding of MAGE-A3 pMHC (FIGS. 34A-34C). This highlights that a small cyclic peptide fusion must form specific interactions with the TCR in order to function as a mask. While the Peptide-5peptide alone or as a fusion was able to specifically bind and inhibit TCR-1 presumably due to specific contacts in the TCR CDR domains, the MAGE-A3 antigen peptide was not able under any circumstances tested. Therefore, non-specific steric occlusion of the TCR pMHC binding interface is an unlikely explanation of functional peptide masking.

### Example 25. High Resolution Crystal Structure of Masked MAGE-A3 TCR Determined via X-Ray Diffraction

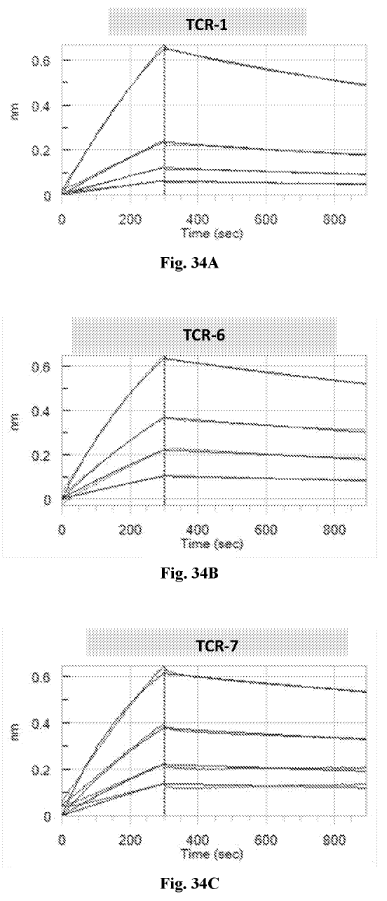

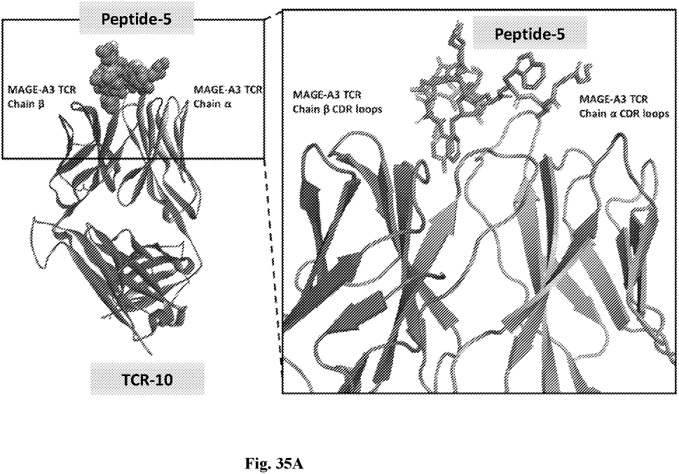

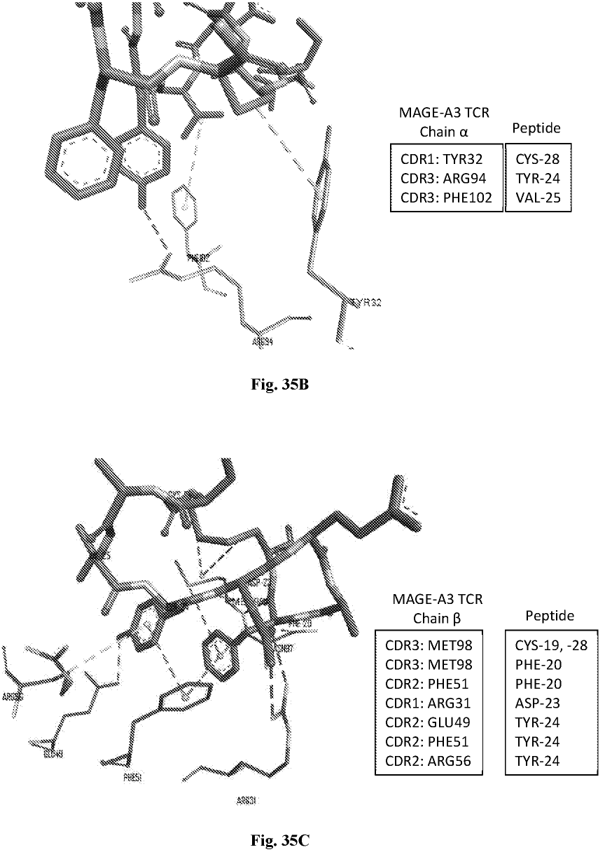

In order to further elucidate the specific interactions of Peptide-5 and MAGE-A3 TCR, the high resolution crystal structure of TCR-10 with Peptide-5 fused at the N-terminal alpha chain of MAGE-A3 TCR was determined via x-ray diffraction (FIG. 35A). Review of the resolved structure revealed specific interactions within the TCR CDR1, CDR2, and CDR3 domains and the conserved residues within the peptide sequence, Cys-19, Cys-28, Tyr-24, Asp-23, Phe-20, and Val-25 of Peptide-5. The peptide clearly forms specific interaction within the TCR CDR domains whose residues are responsible for the recognition of cognate MAGE-A3 pMHC antigen (FIGS. 35B-35C).

### Crystallization Followed by Structure Determination of Masked TCR, TCR-10

The PACT Premier crystallization screen was set at 22C with protein TCR-10 at 9 mg/ml. Several conditions yielded crystal hits. The PEG + NaNO3 condition was selected for optimization (15.5%peg3350, 0.2 M NaNO3). Several crystals were selected and frozen in different cryoprotectant for data collection. Cryoprotection with higher PEG concentration (25%) and the addition of 20% glycerol provided the best data set. A complete dataset was collected at the Advanced Light Source in Berkeley, CA USA on BCSB beamline 5.0.2 from a single crystal. Data was processed using XDS software and scaled with the CCP4 suite to a resolution of 2.3 Å resolution. The space group is P21 with cell dimension: 64.45 114.53 80.70 90 113 90 and 2 molecules in the asymmetric unit. Structure was solved by molecular replacement (MR) with Phaser (CCP4) using chains D and E of the 5BRZ structural model (100% sequence homology with the target sequence). The MR search provided a unique solution with an initial Rfactor of 48.6%. Automatic fitting followed by manual rebuilding of the model and refinement in Refmac5 decreased the Rfactor/Rfree. Clear density was visible for the cyclic peptide between the α and β TCR subunits (FIG. 5). No clear density was visible for the flexible linker between the peptide and TCR alpha chain. The peptide sequence was built into the observed density and the model further refined to a final Rfactor/Rfree of 19.7% / 25.7%. The expected intrapeptide disulfide bond was clearly visible between the two cysteines of Peptide-5.

### Example 26. Design, Synthesis, and Evaluation of Masked MAGE-A3 TCR Anti-CD3 Bispecific T Cell Engagers

Successfully masked, Peptide-5 fused TCRs were progressed into anti-CD3 bispecific T cell engager construction. Various constructs were made by fusing an anti-CD3 single-chain variable fragment (scFv) to the TCRs (FIG. 36). Functional binding, tumor cell killing, and T cell activation of the masked TCR anti-CD3 bispecifics were then evaluated. Masked MAGE-A3 TCR anti-CD3 bispecifics were tested against MAGE-A3 positive tumor cells lines A375 and HCT16 as well as the Titin positive human skeletal muscle myoblasts (HSMM).

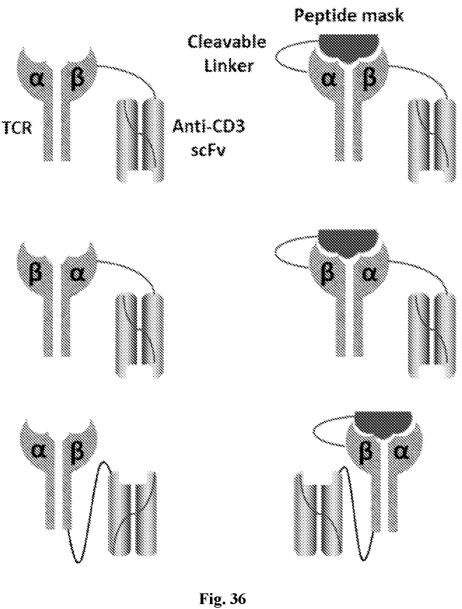

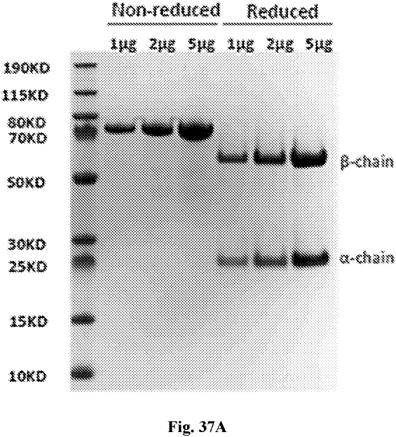

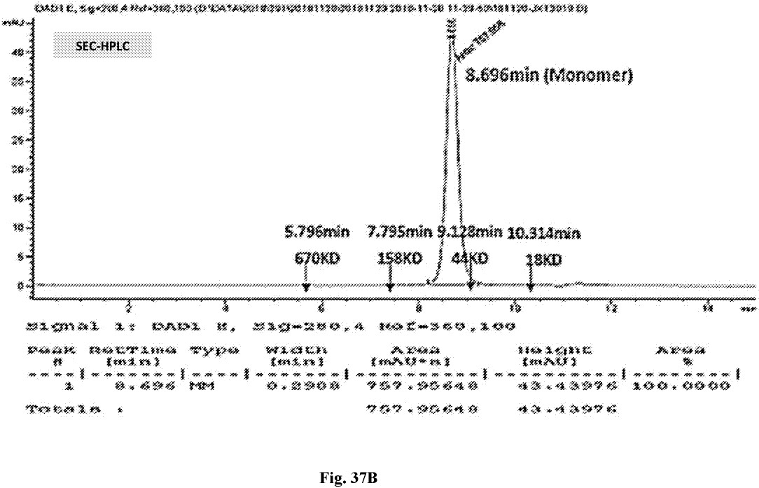

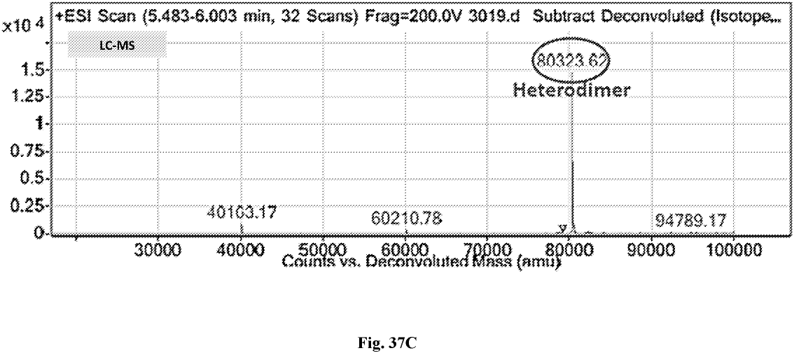

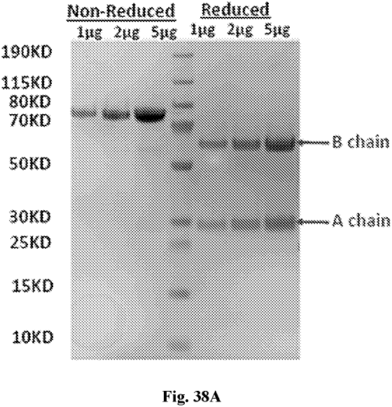

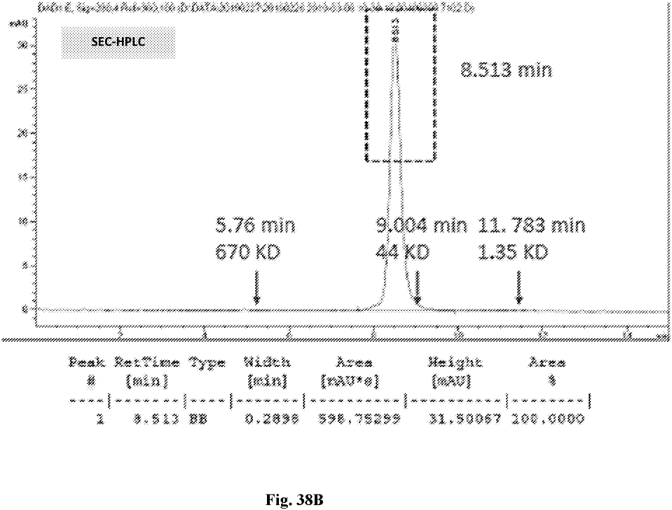

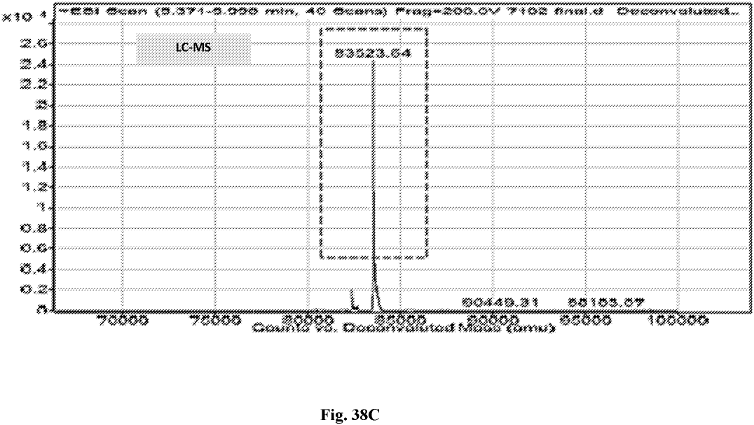

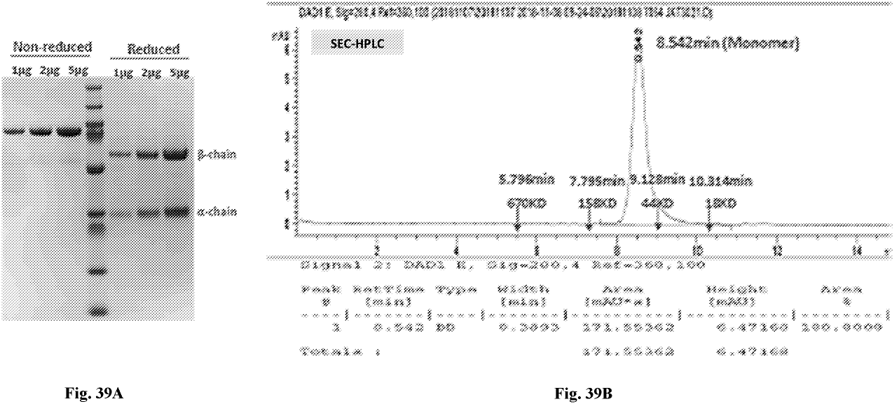

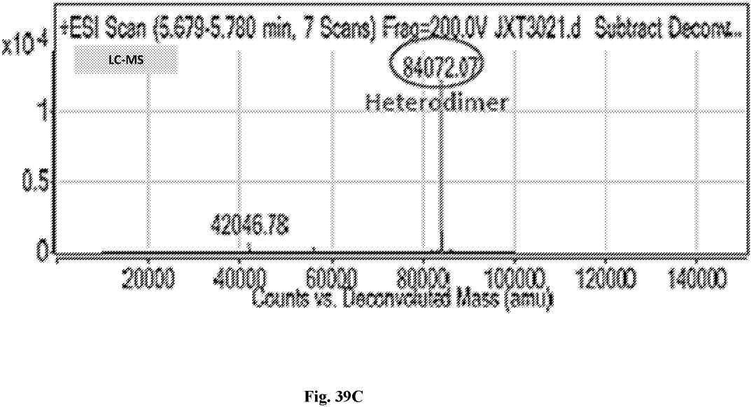

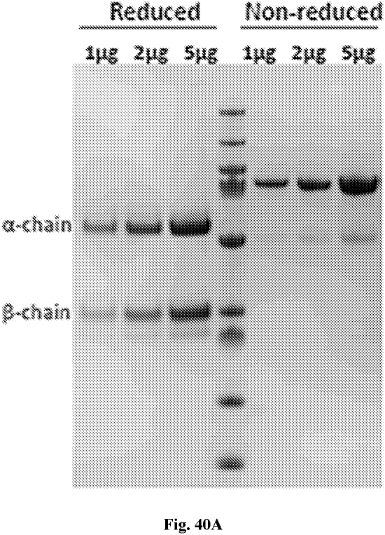

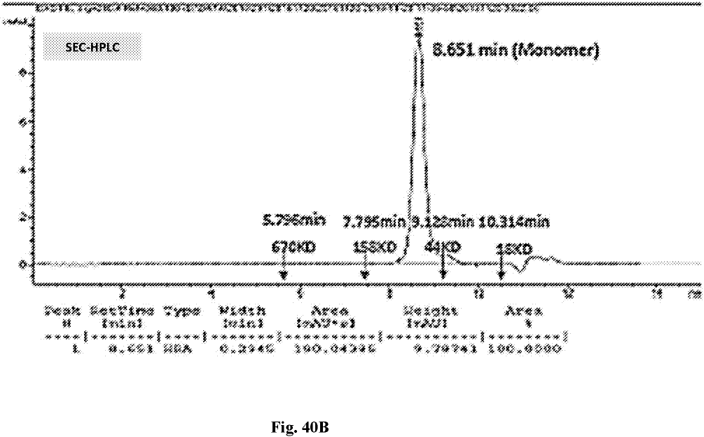

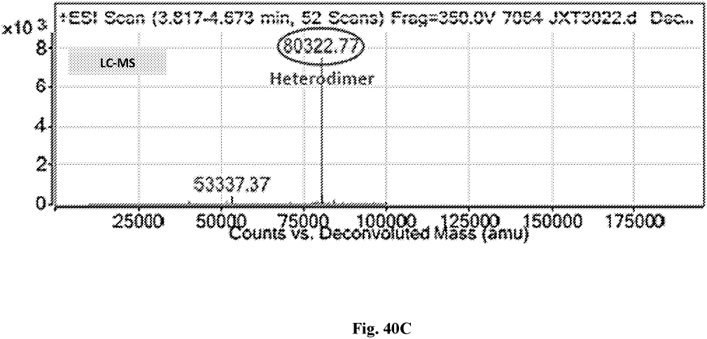

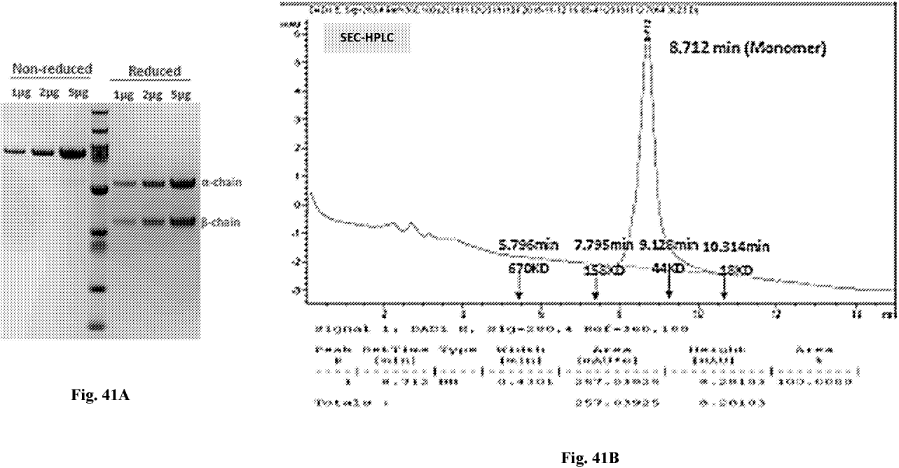

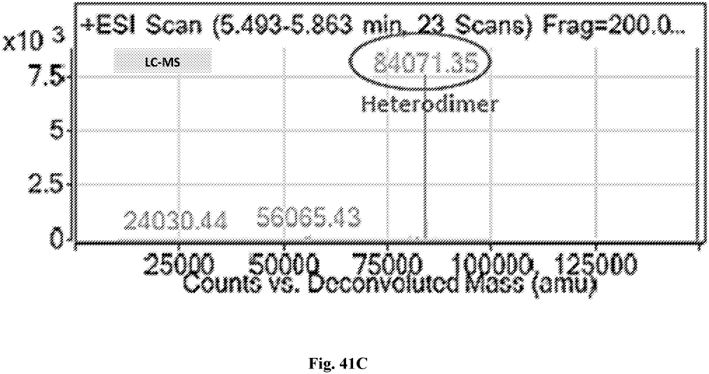

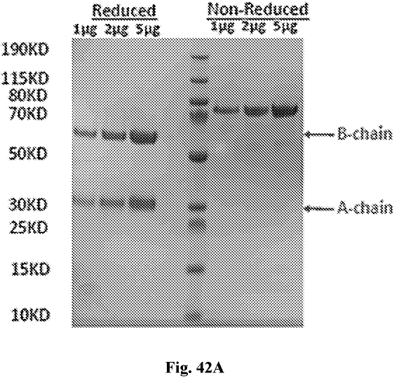

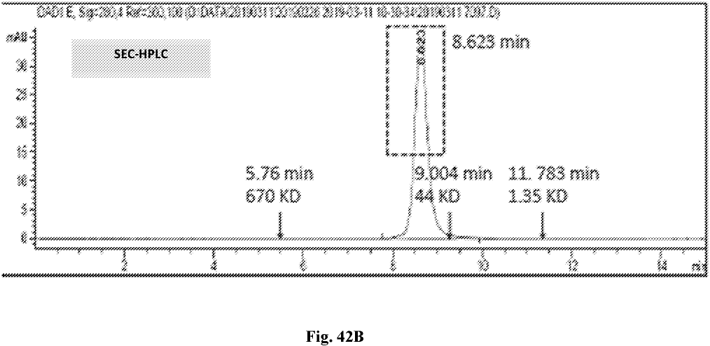

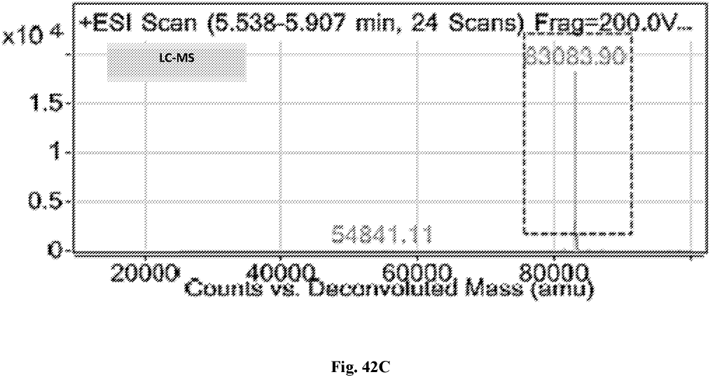

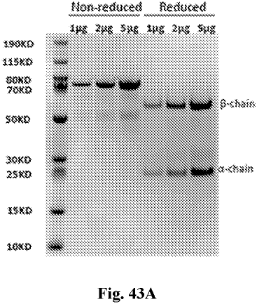

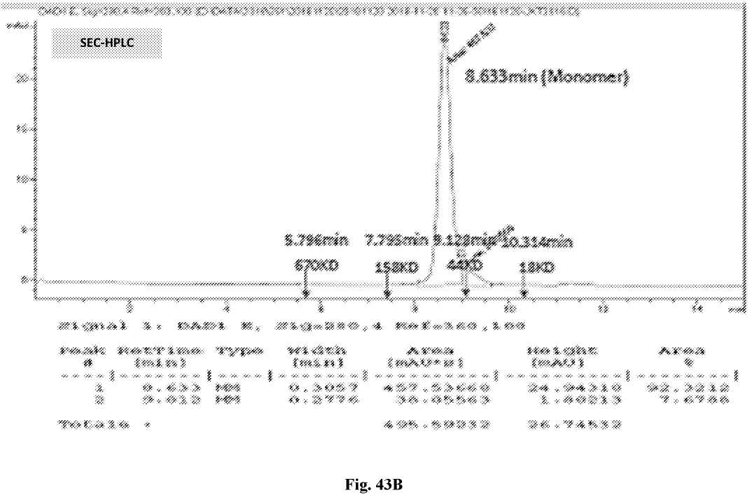

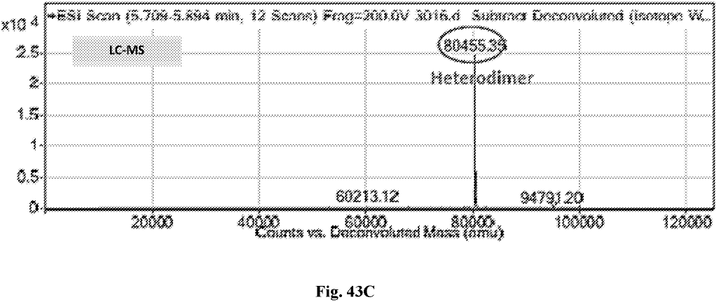

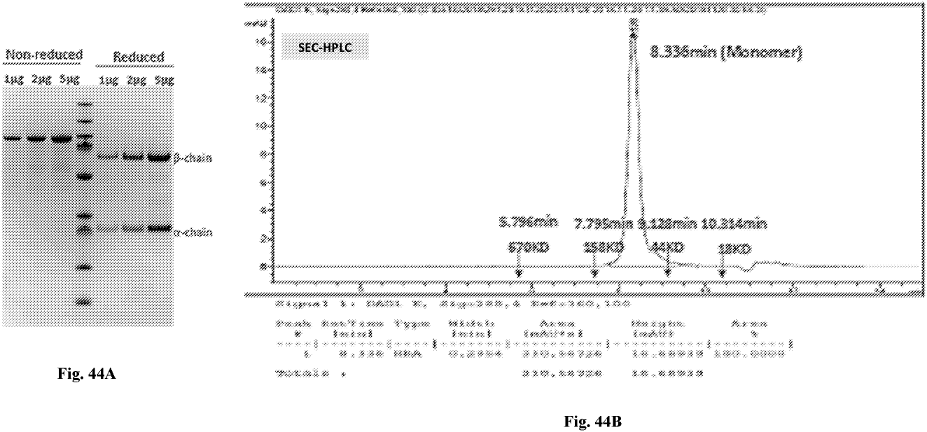

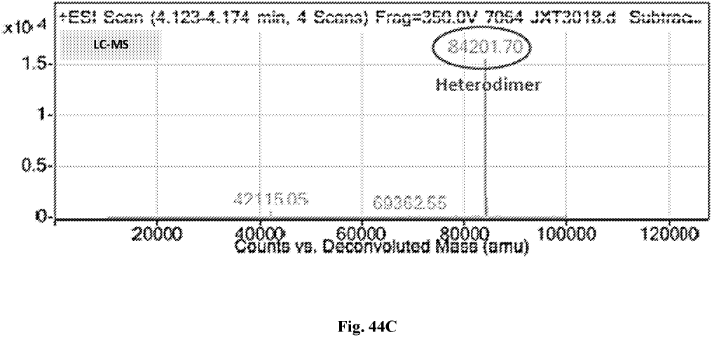

MAGE-A3 TCR anti-CD3 bispecifics were constructed by fusion of the anti-CD3 scFv at the N-terminus of the TCR alpha or beta chain with or without Peptide-5 fused to the N-terminus of the alternative TCR chain. Additional TCR bispecifics were explored with the anti-CD3 scFv fused to the C-terminal Anti-CD3 scFv was derived from published antibody UCHT1 and separated from the TCR chain by a short Gly4Ser linker (SEQ ID NO: 139). Peptide-5 on the other hand was fused to the alternative TCR chain at the N-terminus using the previously described linker (Example 24) containing the protease recognition sequence, LSGRSDNH (SEQ ID NO: 4), or a non-cleavable linker composed of GlySer repeats. Masked MAGE-A3 TCR anti-CD3 bispecific molecule designs are illustrated in FIG. 36. Proteins were produced recombinantly, purified, and refolded as previously described (Example 1). FIGS. 37A-37C through FIGS. 43A-43C highlight the production quality of the example masked TCR bispecifics.

### Kinetic Binding of Masked TCR Bispecifics to Cognate pMHC via BLI

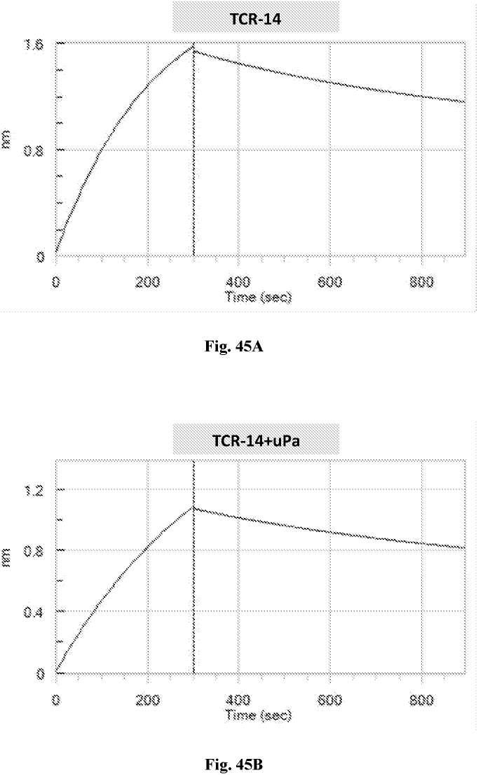

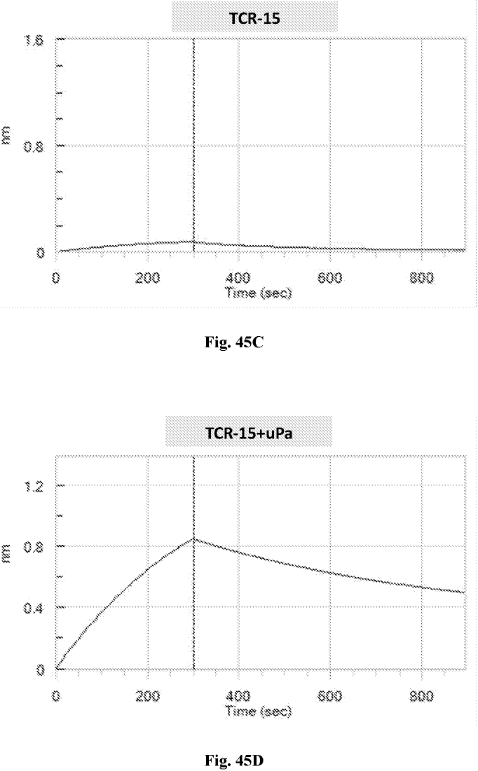

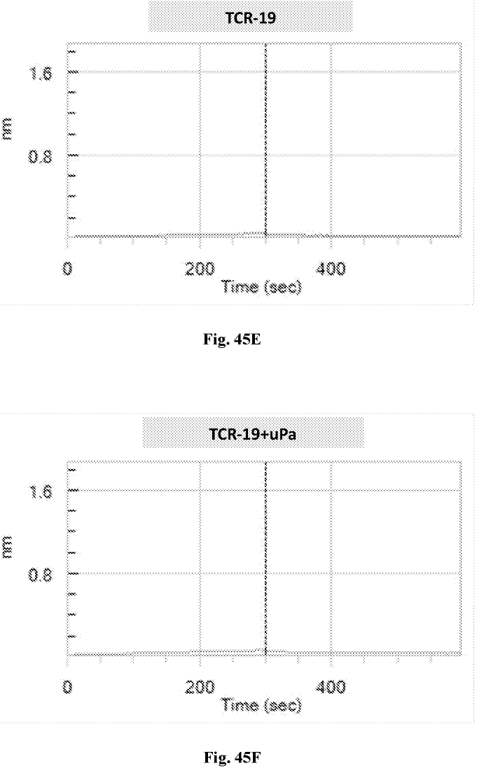

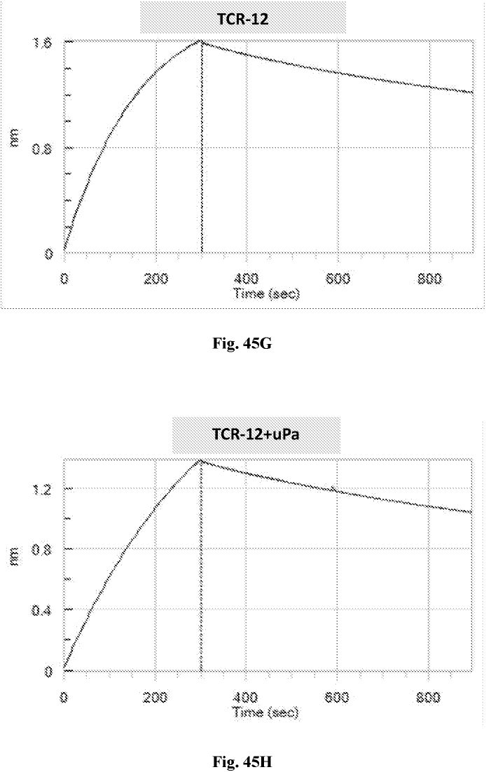

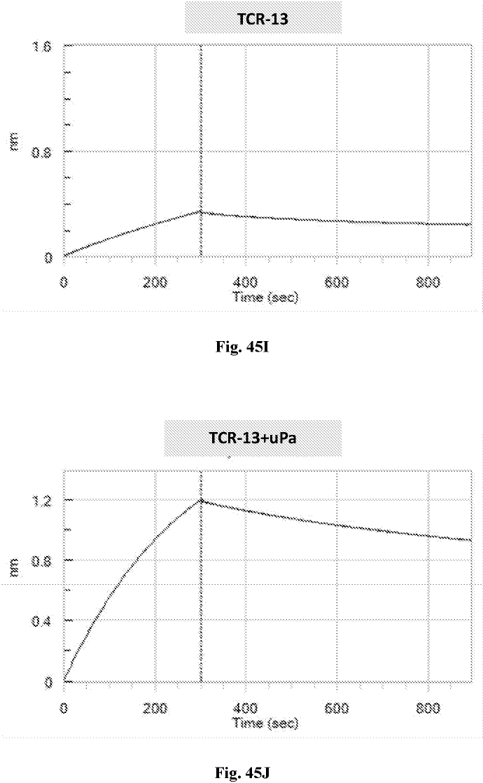

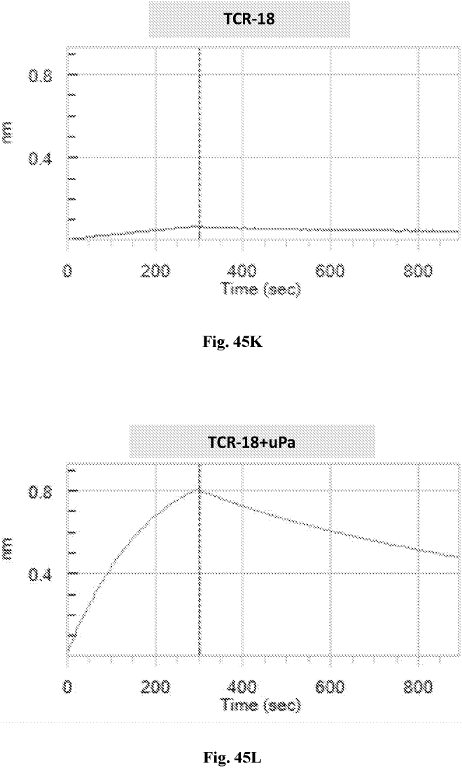

TCR bispecifics were first characterized for their ability to recognize cognate pMHC in BLI based binding experiments (FIGS. 45A-45L). Briefly, biotinylated MAGE-A3 pMHC was loaded onto BLI streptavidin biosensors, quenched with excess biocytin and baselined in buffer. TCRs bispecifics were then associated onto the pMHC loaded sensors at a single concentration of 50 nM. Sensors were transferred back to buffer to measure dissociation. Kinetic binding signals suggest Peptide-5 mask hindered the TCR bispecific constructs recognition of pMHC when fused to the TCR via the described protease cleavable linker. Likewise, treatment of TCR bispecifics with urokinase (uPa) recovered binding signals to equivalent levels measured for their non-masked parental controls (TCR-14 and TCR-17). All masked TCR bispecific constructs utilizing Peptide-5 N-terminal fusions required protease treatment and a cleavable linker in order to observe potent cognate pMHC recognition. By comparison, replacement of LSGRSDNH (SEQ ID NO: 4) protease substrate sequence with non-cleavable GlySer repeats (TCR-19) eliminated the observed protease dependent binding of TCR bispecifics (FIGS. 45A-45L).

### Equilibrium Binding of Masked TCR Bispecifics to Cognate pMHC via ELISA

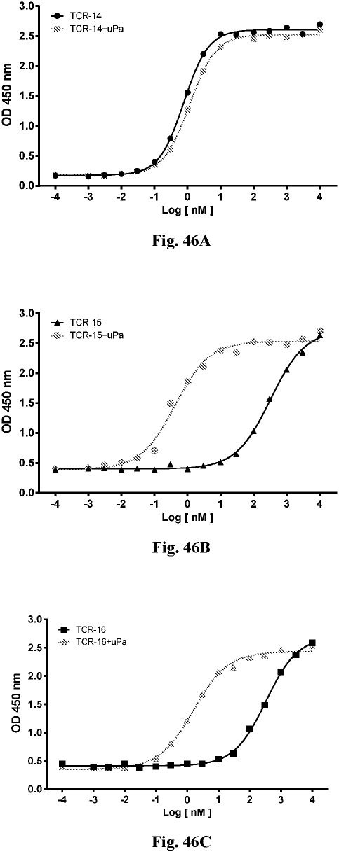

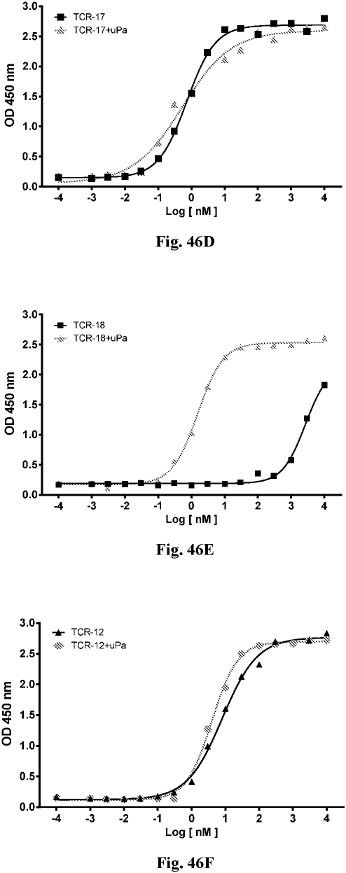

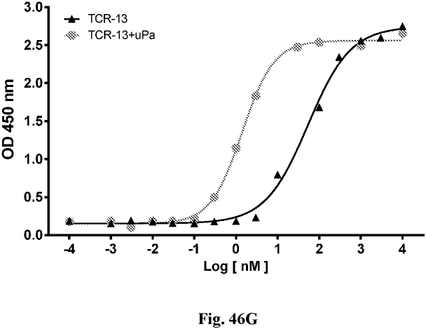

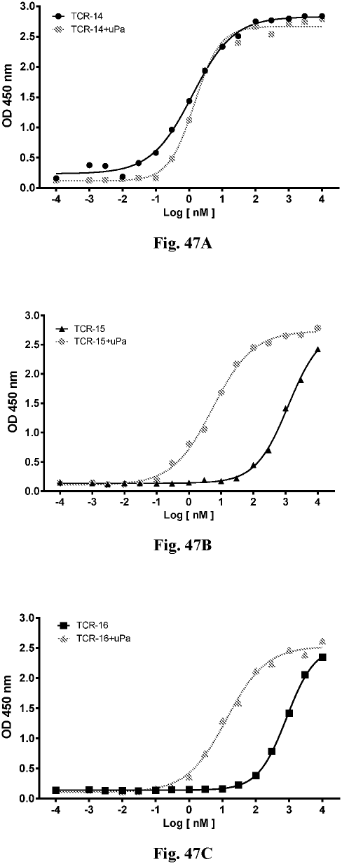

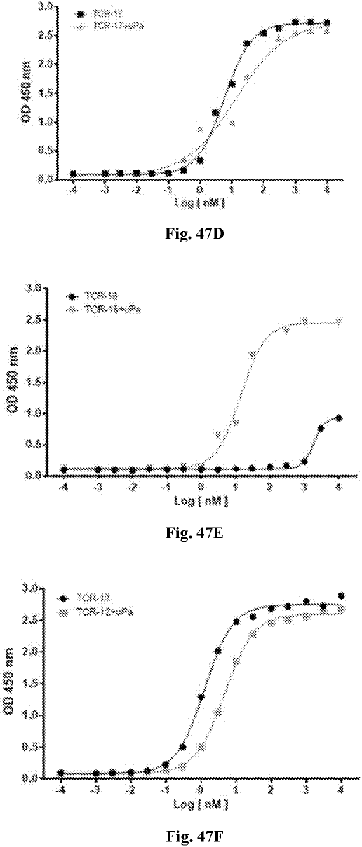

TCR bispecifics were also characterized for their ability to recognize cognate pMHC in ELISA based binding experiments (FIGS. 46A-46G and 47A-47G). In some instances the TCR bispecifics were treated with protease where indicated. Briefly, biotinylated pMHCs were captured on neutravidin coated plates followed by the addition of titrated TCR bispecifics. Plates were then incubated for a short time followed by a wash. A secondary anti-histag HRP conjugate antibody was used to detect bound TCR bispecifics to the plate. Concentrations of TCR bispecifics required to achieve half maximal ELISA signal (EC50) were calculated in Graphpad Prism 6.0. The ELISA data in FIGS. 46A-46G and 47A -47Gdemonstrate hindered ability of masked TCR bispecifics to bind MAGE-A3 (FIGS. 46A-46G) and Titin pMHCs (FIGS. 47A-47G) respectively, that is otherwise restored to non-masked parental control levels by treatment with protease.

All masked TCR bispecific constructs utilizing Peptide-5 N-terminal fusions required protease treatment and a cleavable linker in order to observe potent cognate pMHC recognition similar to the non-masked TCR bispecific controls. By comparison, replacement of LSGRSDNH (SEQ ID NO: 4) protease substrate sequence with non-cleavable GlySer repeats (TCR-19) eliminated the observed protease dependent binding of TCR bispecifics.

### Ternary Complex Formation of Masked TCR Bispecifics on the Surface of Human T Cells via Flow Cytometry

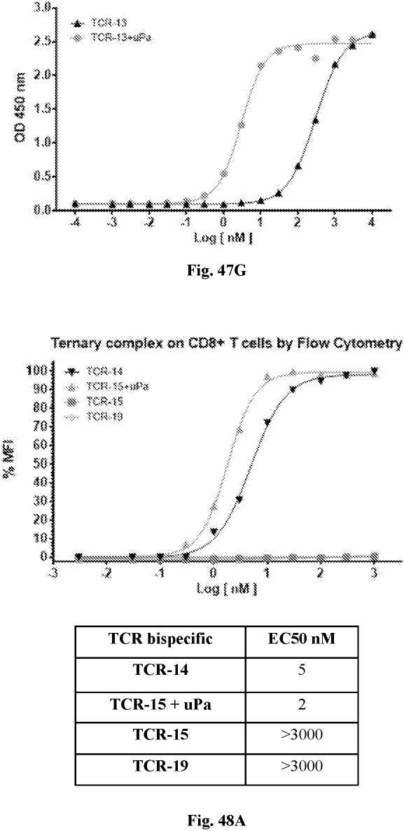

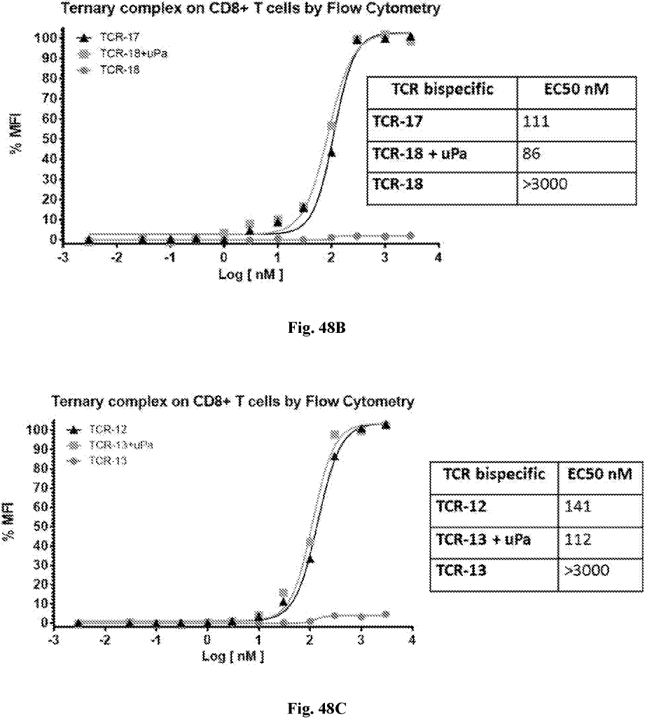

TCR bispecifics were further characterized for their ability to form a ternary complex on the surface of human CD8+ T cells via binding of cellular CD3 and subsequently stained using fluorescently labeled MAGE-A3 pMHC tetramer (FIGS. 48A-48B). Cellular fluorescence measured by flow cytometry was indicative of complex formation between T cell and MAGE-A3 pMHC tetramer where the TCR bispecific acts as the bridging molecule.

Briefly, 100,000 T cells per well were distributed in a 96 well plate, washed cold, followed by incubation with the indicated concentration of non-masked, masked, or protease treated TCR bispecifics. Cells were incubated cold for a few hours, then washed with cold buffer, followed by a short incubation with cold MAGE-A3 pMHC tetramer formed using fluorescently labeled streptavidin. Cells were washed cold, resuspended in cold buffer, and run on a Novocyte flow cytometer. Scattering signals were gated in the typical fashion to exclude debris of incorrect cellular shape and size. Mean fluorescent intensity was normalized, plotted against TCR concentration, and Graphpad Prism 6.0 was used to calculate the concentration of TCR bispecific required to achieve 50% maximal signal (EC50).

In general, all non-masked or protease treated TCR bispecifics were able to form a ternary complex in a dose dependent fashion on the surface of human CD8+ T cells. Closer examination suggests that N-terminal fusion of the anti-CD3 scFv on the TCR beta chain was most efficient and had the lowest EC50 for ternary complex formation. The masked TCR bispecifics had little to no observable binding at the highest concentrations tested. For example, the non-masked control, TCR-14, as well as urokinase treated TCR-15 bound to human CD8+ T cells with low nanomolar EC50s, whereas the fully masked TCR-15 and TCR-19 signals remained at background levels despite testing up to 3 uM. The flow cytometry data demonstrated a >300× shift in the ability of TCR-15 to bridge soluble MAGE-A3 pMHC tetramer and CD3 on the surface of human CD8+ T cells (FIGS. 48A-48B) when treated with tumor specific protease or compared to non-masked control, TCR-14. The EC50s of non-masked or protease treated molecules where the anti-CD3 scFv was located on the N-terminal alpha chain (TCR-17, TCR-18) or the C-terminal beta chain (TCR-12, TCR-13) were less efficient at forming the ternary complex relative to placement of the anti-CD3 scFv on the N-terminal beta chain (TCR-14, TCR-15, TCR-19).

### TCR Bispecific Mediated Tumor Cytotoxicity and T Cell Activation

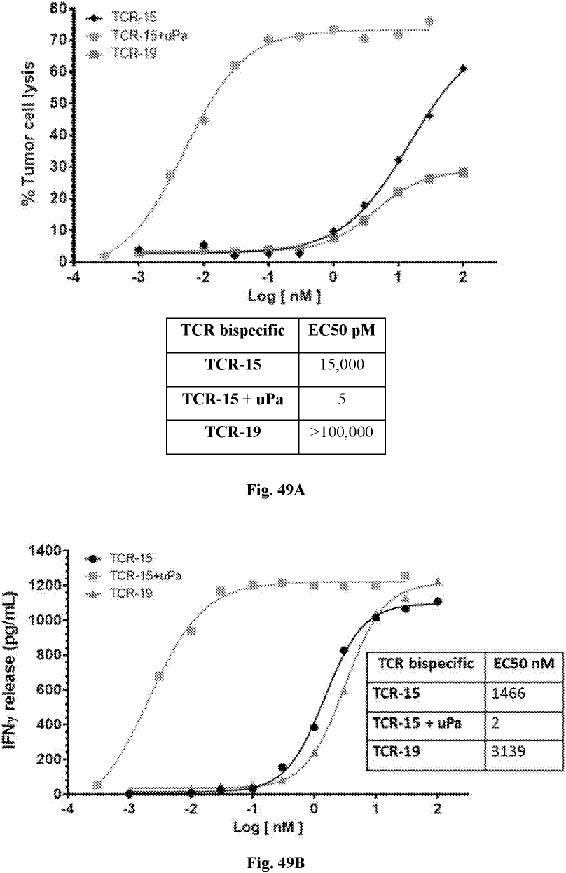

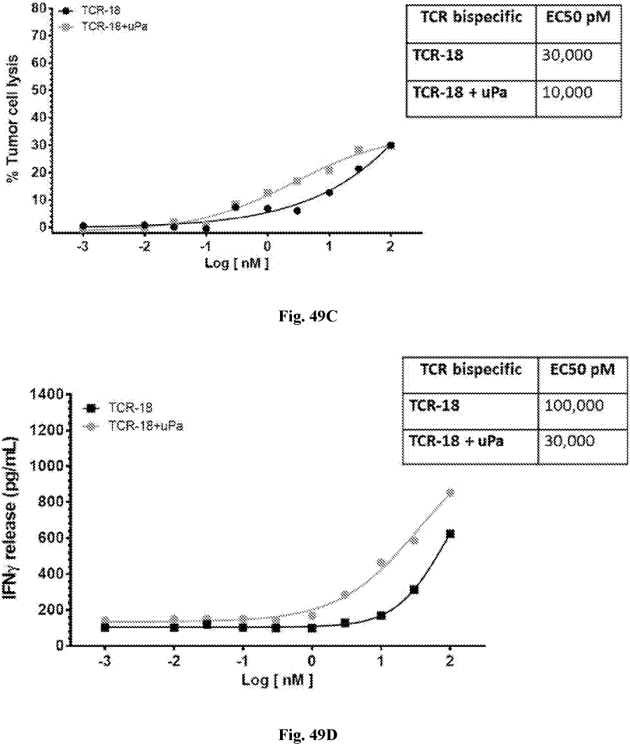

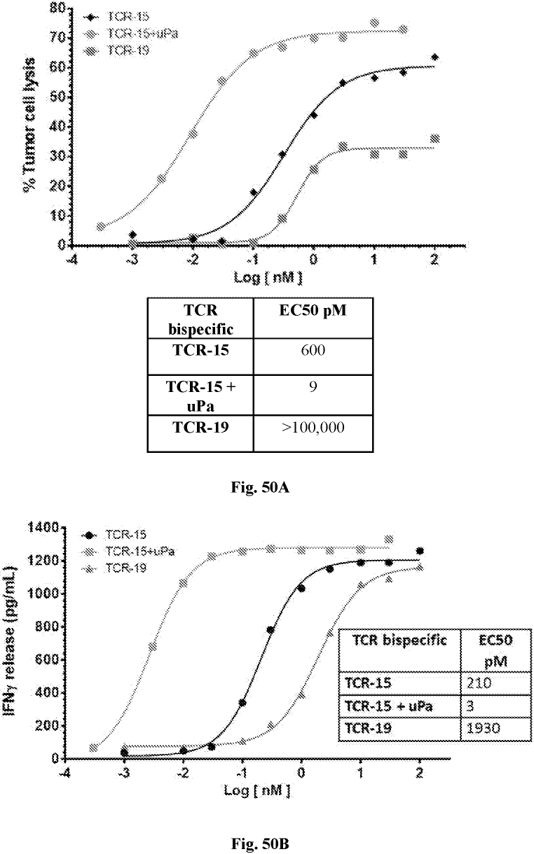

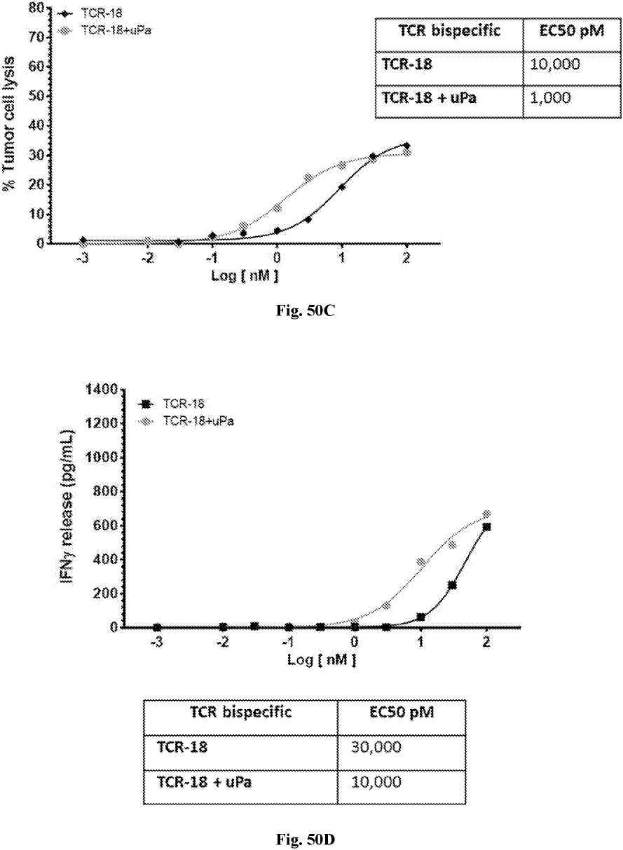

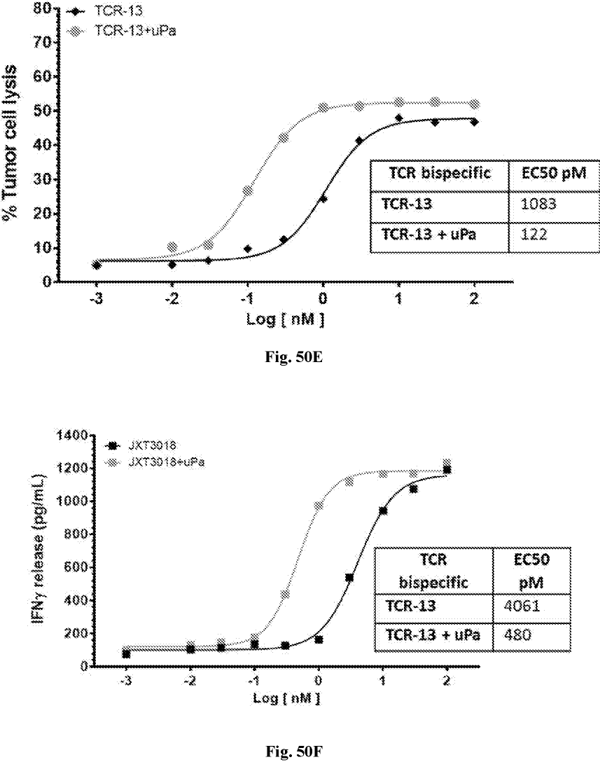

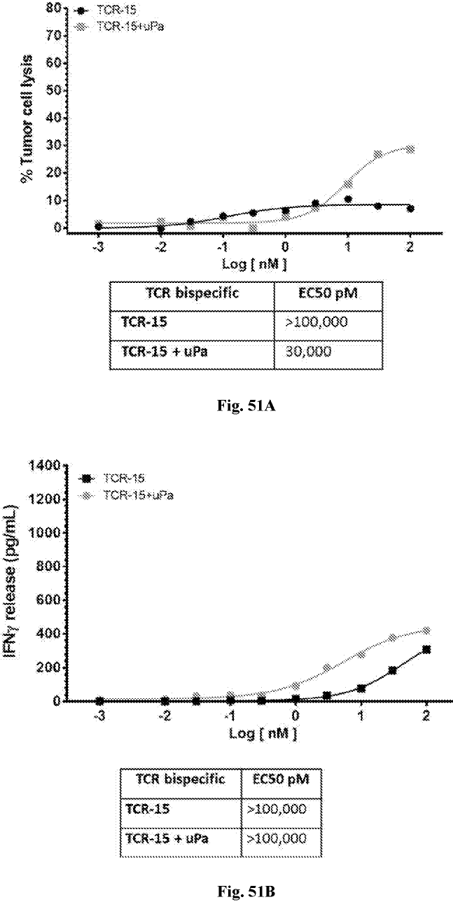

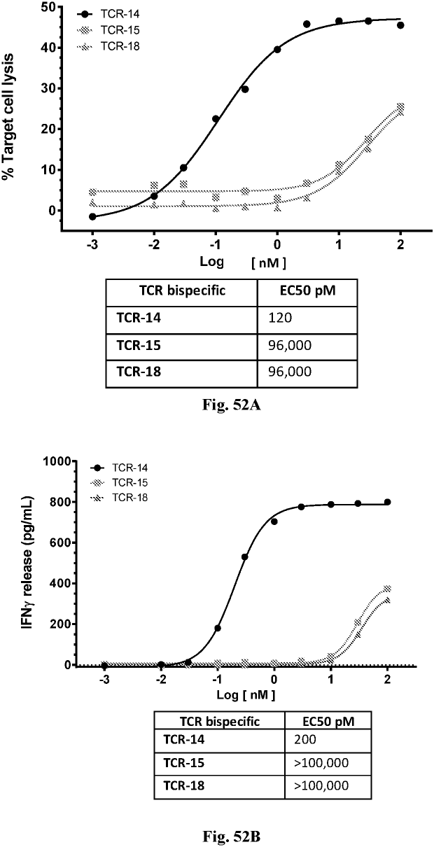

TCR bispecifics were next evaluated in functional in vitro tumor cell killing and related T cell activation studies (FIGS. 49A-49D through FIGS. 51A-51B). In addition, dose dependent killing and related T cell activation using TCR bispecifics were evaluated against the Titin pMHC positive healthy tissue target cells, human skeletal muscle myoblasts (HSMM) (FIGS. 52A-52B). Briefly, MAGE-A3 positive tumor cell lines, A375 (FIGS. 49A-49D), HCT116 (FIGS. 50A-50F), or HT29 (FIGS. 51A-51B), or Titin positive HSMM (FIGS. 52A-52B) were seeded onto 96 well tissue culture treated flat bottom plates and allowed to adhere overnight. The following day, culture medium and nonadherent cells were removed and replaced with fresh medium containing titrated TCR bispecifics at concentrations indicated. In some instances, TCR bispecifics were treated with protease prior to addition to target cells. CD8+ T cells were then added in an effector cell: target cell ratio of 2:1 for tumor cell and 5:1 for HSMM relative to seeding density. Adherent tumor cells or HSMM, CD8+ T cells, and TCR bispecifics were co-cultured for 48 hours. Plates were gently spun down to collect cells at the bottom of the plate and the clarified supernatants collected. Lactate dehydrogenase (LDH) dependent cytotoxicity was measured using the Promega LDH-Glo assay kit. Interferon-gamma (IFNγ) released by activated T cells was measured in the supernatants using an Invitrogen ELISA kit. LDH or IFNy signals were plotted against concentration of TCR bispecifics in order to calculate the concentration of TCR bispecifics required to achieve 50% maximal signal (EC50) calculated using Graphpad Prism 6.0.

In general, TCR bispecifics required >500× higher concentrations in order to register a 50% maximal cytotox or IFNy signal relative to those activated by protease. For example, parental non-masked TCR bispecific TCR-14, performed similarly to protease treated TCR bispecific with a cleavable linker substrate, TCR-15, indicating full activation post proteolysis. The differences in functional potency between cleavable masked (TCR-15) and non-cleavable masked (TCR-19) TCR bispecifics in the A375 and HCT116 cytotoxicity assays likely indicated differential proteolytic activity from the target cells (FIGS. 49A-49D and FIGS. 50A-50F). Similar to that observed in the ternary complex flow cytometry experiments (Example 25), TCR bispecific constructs with N-terminal alpha chain (TCR-17, TCR-18) or C-terminal beta chain fusions (TCR-13) of the anti-CD3 scFv were less active than the N-terminal beta anti-CD3 scFv fusion (TCR-14, TCR-15). Nonetheless, TCR bispecific constructs masked via Peptide-5 N-terminal fusions required tumor specific protease activation and a cleavable linker in order to observe potent and functional cognate pMHC recognition, target cell killing, and effector cell activation. Furthermore, TCR masking using an inhibitory peptide fusion was a reliable way to limit off tumor activity of a TCR anti-CD3 bispecific (FIGS. 52A-52B). While non-masked MAGE-A3 Titin cross reactive TCR bispecifics killed the healthy HSMM, the analogous masked TCR bispecifics required a much higher concentration to achieve similar levels of in vitro healthy tissue killing (>500x shift in EC50s).

### Example 27. Design, Synthesis, and Evaluation of Half Life Extended Masked TCR Bispecific T Cell Engagers

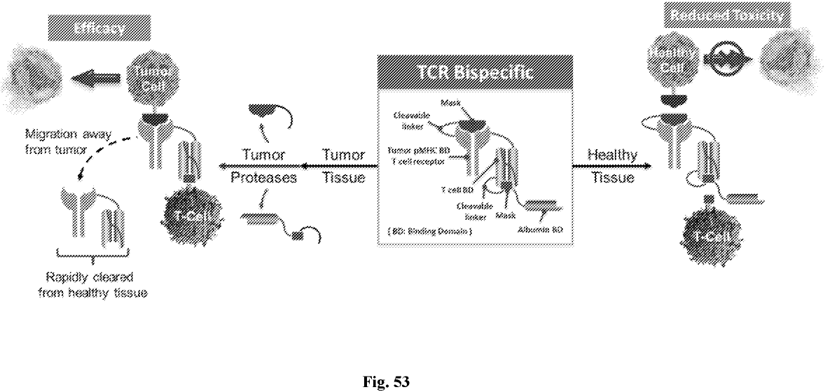

S Bispecific T cell engagers typically have poor pharmacokinetics (PK) properties. We hypothesized that adding a half-life extension molecule in tandem with the proteolytically cleavable mask would exhibit crossover PK defined by a long half-life in systemic circulation but fast clearance after mask and PK extender cleavage at the tumor site due to specific proteolytic activity. Thus these cross over PK molecules would have an additional safety switch preventing accumulation in healthy tissue once activated at the tumor site. FIG. 53 illustrates the tumor specific activity and cross over PK concepts.

The best configurations of masked TCR bispecifics were progressed into analogous crossover PK construction. Various constructs were made by fusing an anti-albumin single domain antibody (SDA) in tandem to the Peptide-5mask separated by a short GlySer linker. The SDA and Peptide-5 tandem mask was fused to the TCR bispecifics using the same cleavable or non-cleavable linkers previously described (Example 26). Functional binding, tumor cell killing, and T cell activation of the masked TCR anti-CD3 bispecifics were then evaluated. Masked MAGE-A3 TCR anti-CD3 bispecifics were tested against MAGE-A3 positive tumor cells lines A375 and HCT16 as well as the Titin positive human skeletal muscle myoblasts (HSMM). In addition, mouse and cynomologus monkey PK was evaluated for the constructs.

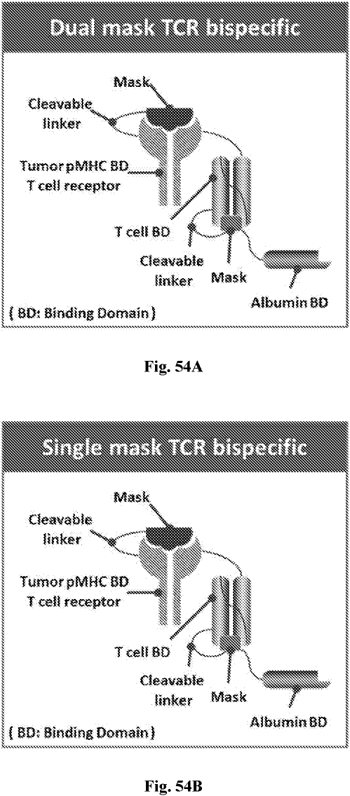

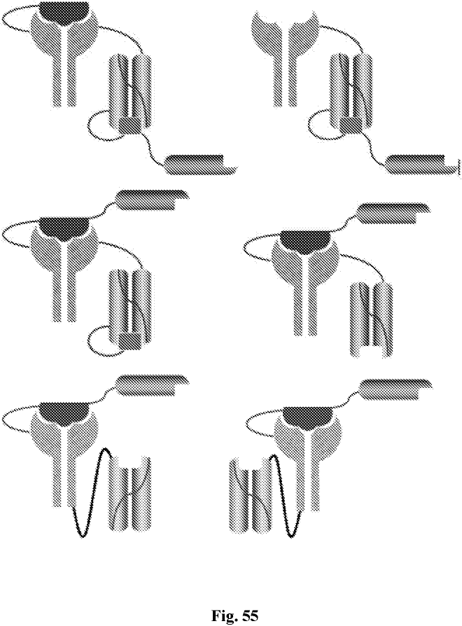

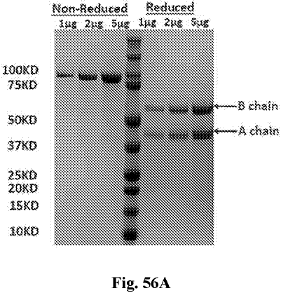

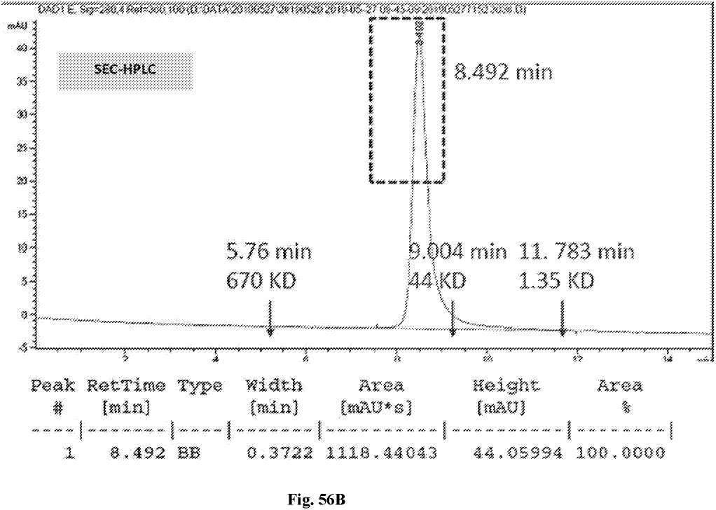

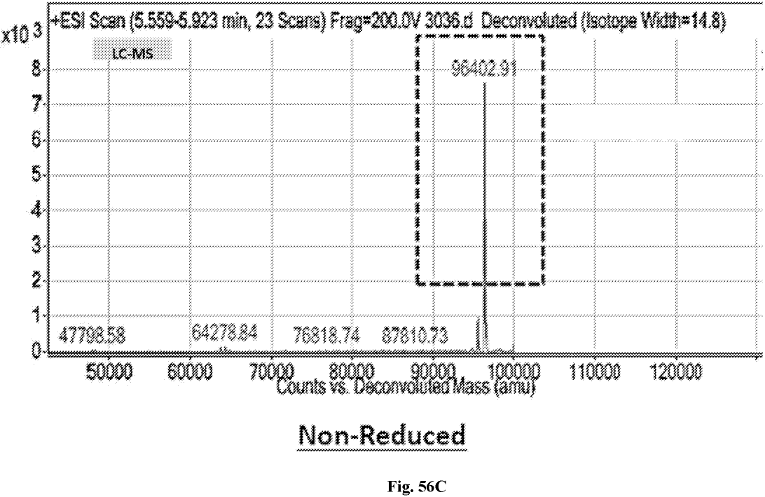

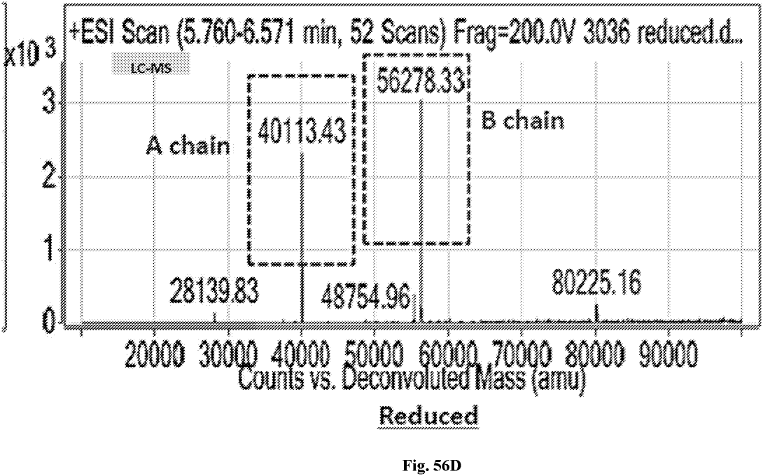

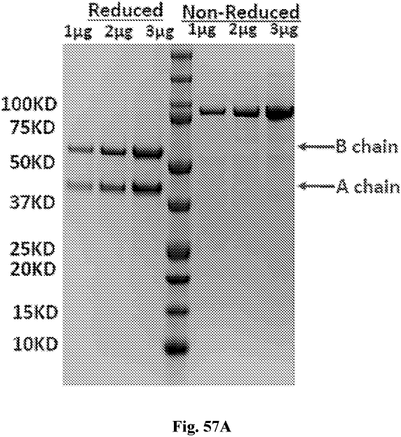

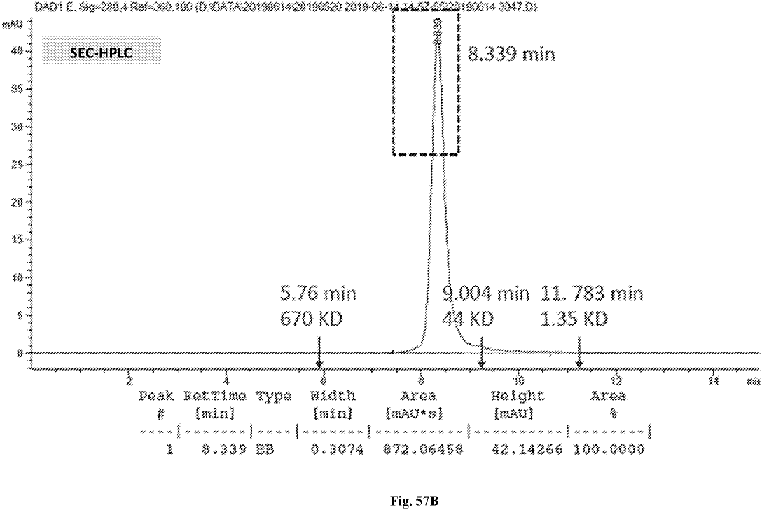

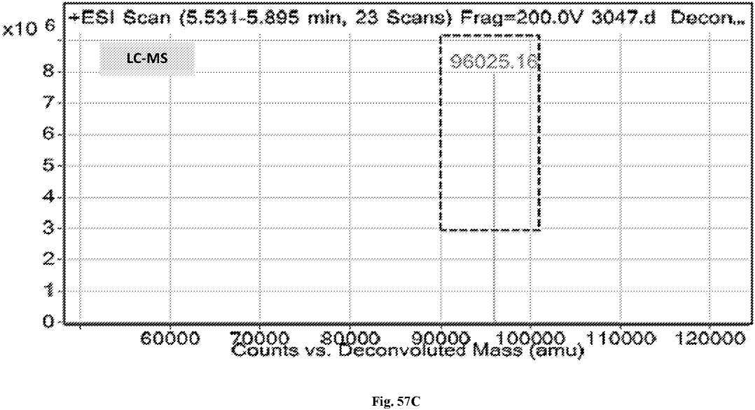

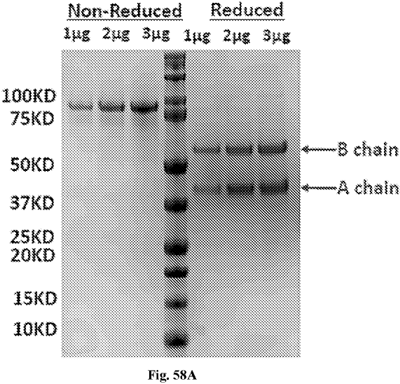

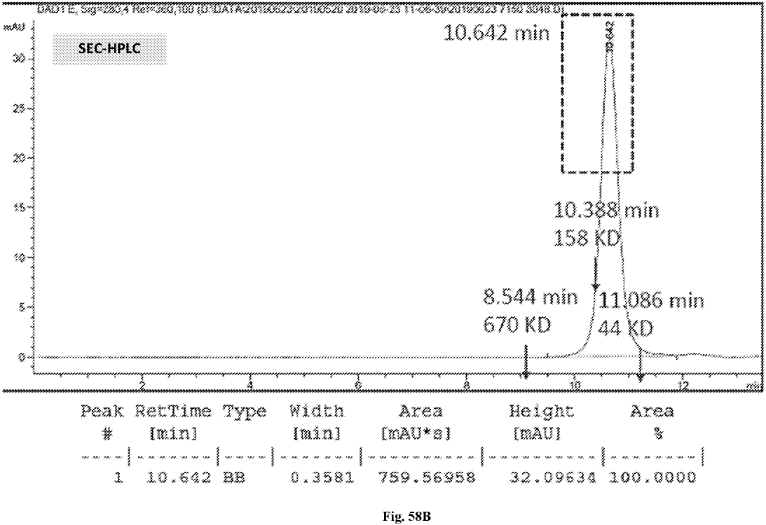

Generalized dual mask and single mask TCR bispecific molecule designs are shown in FIGS. 54A-54B. Additional TCR bispecific molecule designs are illustrated in FIG. 55 Proteins were produced recombinantly, purified, and refolded as previously described (Example 1). FIGS. 56A-56D through FIGS. 58A-58C highlight the production quality of TCR bispecific constructs.

### TCR Bispecific Equilibrium Binding of Albumin via ELISA

Anti-albumin single domain antibody (SDA) was tethered in tandem to the TCR mask attached to the core TCR bispecific structure to form a complete TCR bispecific molecule of various formats (FIG. 55). In order to test the functional binding of the anti-albumin domain while tethered to the TCR bispecific molecule, TCR bispecific molecules were first tested for their ability to bind albumin of several species. FIG. 59 illustrates TCR-20 binding to bovine, mouse, cyno, and human albumin. Briefly, serum albumin from different species were coated directly on high binding ELISA plates, washed, blocked in non-fat dry milk, and washed again. TCR bispecific molecules were diluted in non-fat dry milk to the desired concentrations, added to the albumin coated plates, and washed. A secondary anti-histag HRP conjugate was used to detect bound TCR bispecific. After washing, plates were developed, stopped, and measured using standard ELISA techniques. OD450 nm signals were plotted against logarithmic TCR bispecific concentration. The concentration of TCR bispecific to achieve half maximal signal (EC50) was calculated in Graphpad Prism 6.0. TCR-20 bound mouse, cyno, and human albumin with negligible signal detected against bovine albumin (FIG. 59). EC50s are summarized in Table 4. Despite tethering the anti-albumin SDA to the TCR mask within the TCR bispecific molecule, potent albumin recognition was maintained.

TABLE 4

Summary of TCR-20 invitro binding and functional activity data

| | | |
|---|---|---|
| Binding or Activity | TCR-20 | TCR-20+MTSP1 |
| Bovine albumin, EC50 | >10 uM | -- |
| Mouse albumin, EC50 | 41 nM | -- |
| Cyno albumin, EC50 | 2 nM | -- |
| Human albumin, EC50 | 7 nM | -- |
| MAGE-A3 pMHC, EC50 (BSA / HSA) | 2 uM / 1 uM | 3 nM / 1 nM |
| Cellular CD3, EC50 (BSA / HSA) | >3 uM / >3 uM | 1 nM / 1 nM |
| HCT116 Cytotox EC50 (FBS / HS) | 1.7 nM / 4.6 nM | 0.02 nM / 0.04 nM |
| HCT116 IFNg EC50 (FBS / HS) | 1.4 nM / 8.9 nM | 0.02 nM / 0.05 nM |
| A375 Cytotox EC50 (FBS / HS) | 11 nM / 34 nM | 0.02 nM / 0.05 nM |
| A375 IFNg EC50 (FBS / HS) | 13 nM / 76 nM | 0.01 nM / 0.02 nM |

### Kinetic Binding of TCR Bispecifics to Cognate pMHC via BLI

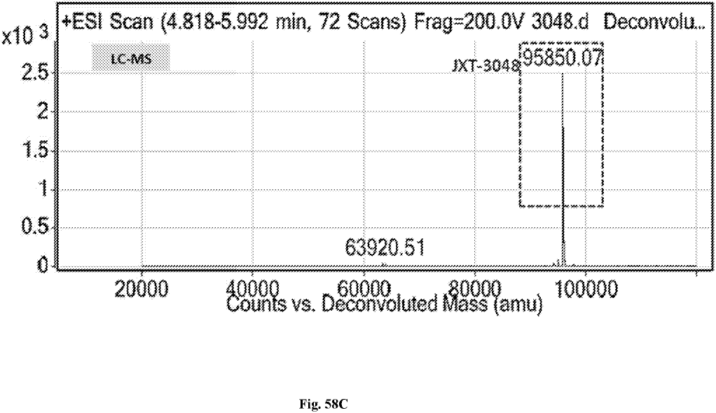

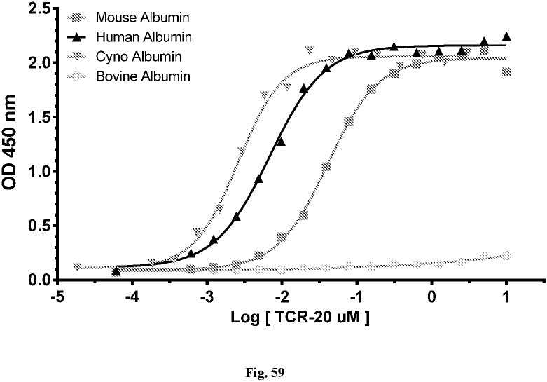

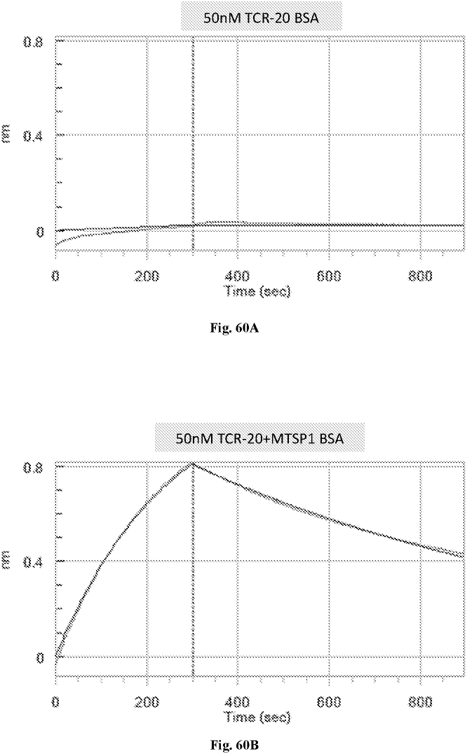

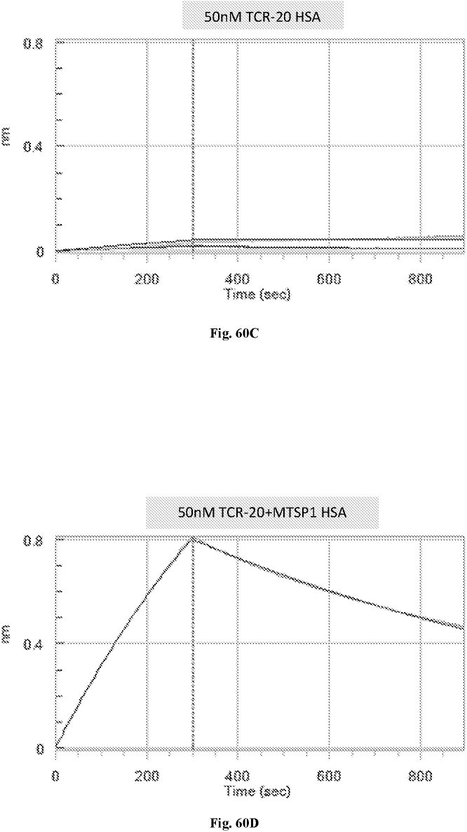

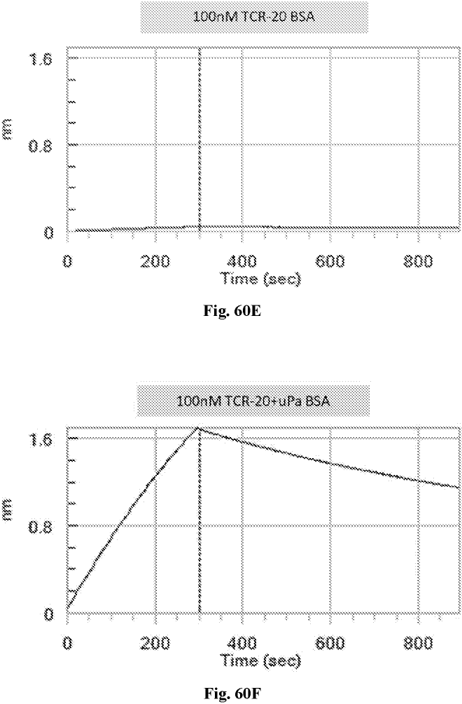

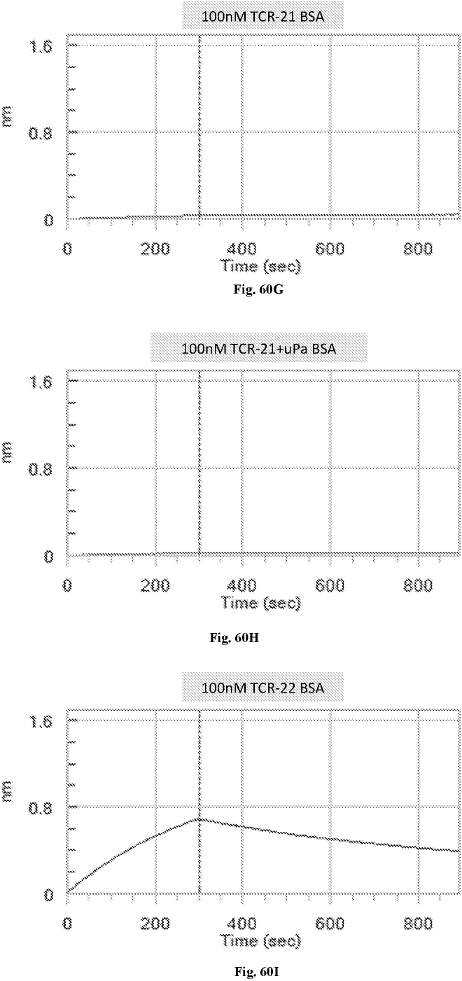

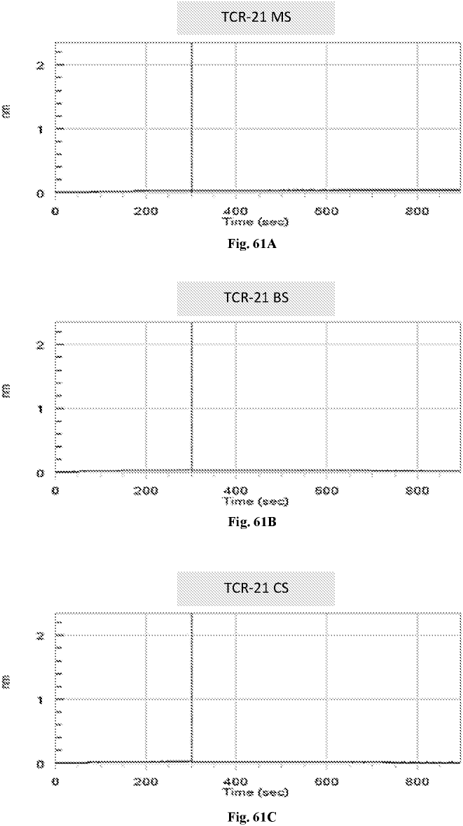

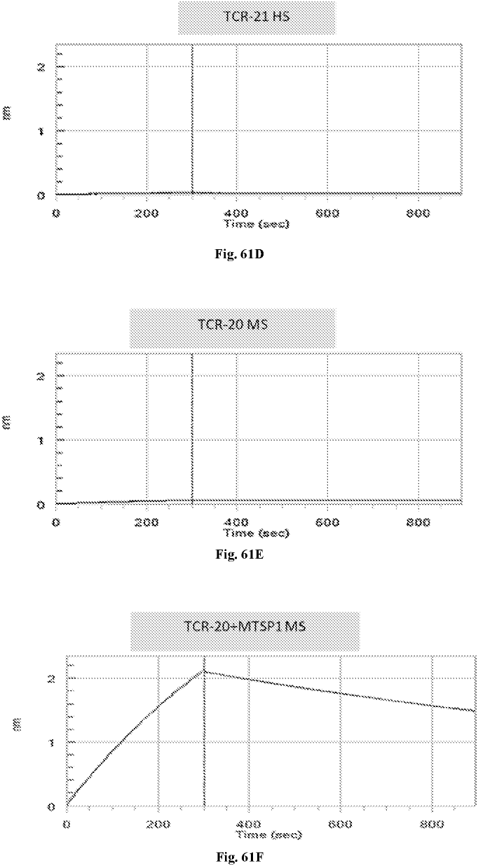

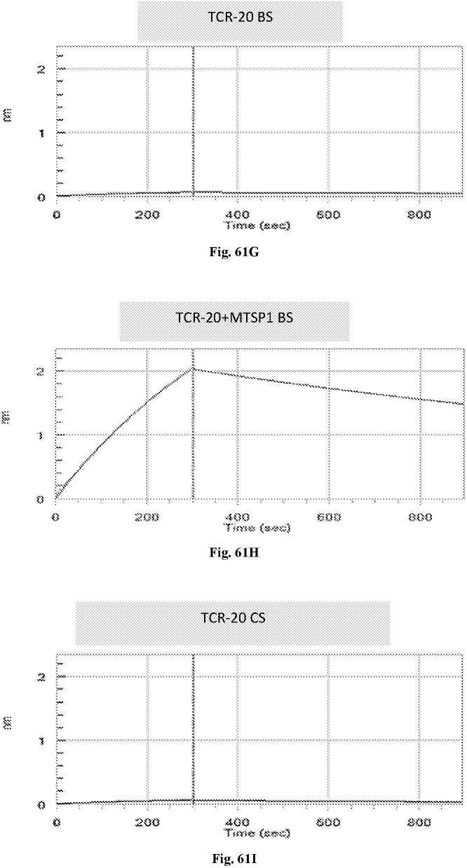

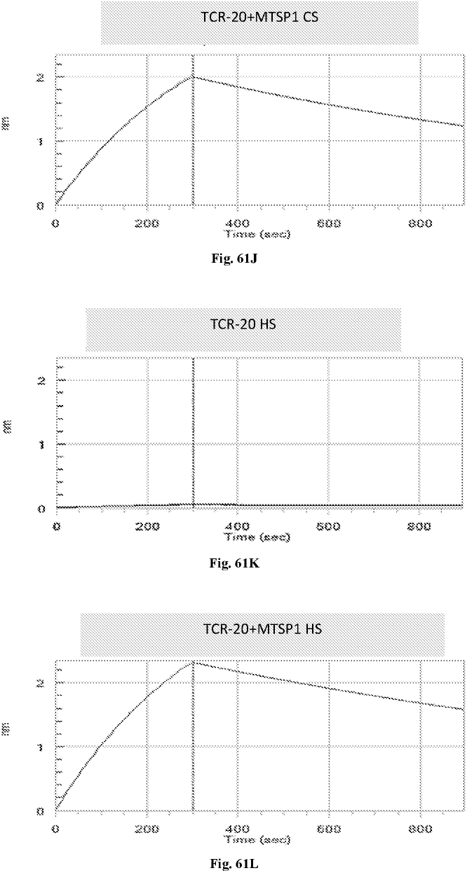

TCR bispecific molecules were evaluated for their ability to bind the cognate MAGE-A3 pMHC via BLI. TCR bispecific binding kinetics of MAGE-A3 pMHC was measured before and after protease treatment in the presence of bovine albumin (BSA), human albumin (HSA), mouse serum (MS), bovine serum (BS), cynomolgus monkey serum (CS), or human serum (HS). Briefly, biotinylated MAGE-A3 pMHC was loaded onto streptavidin coated biosensors, quenched in biocytin, and baselined in buffer containing appropriate albumin or serum. The concentration of albumin or serum used was at a level expected to saturate the TCR bispecific albumin binding site. TCR BISPECIFIC molecules were treated with active matriptase (MTSP1) or urokinase (uPa) where indicated. TCR bispecific molecules diluted in albumin or serum supplemented buffer to 50 nM or 100 nM were then associated onto the MAGE-A3 pMHC loaded biosensors. Sensors were then transferred to the appropriate albumin or serum supplemented buffer where TCR bispecific molecules then dissociate from the sensors. Association and dissociation rates were measured in real time using an OCTET RED96 instrument. Example sensorgrams are shown in FIGS. 60A-60I and FIGS. 61A-61L. TCR bispecific TCR-20 contains the cleavable substrate, LSGRSDNH (SEQ ID NO: 4), between the anti-albumin SDA tethered peptide mask and the TCR alpha chain. Kinetic binding data suggests that TCR-20 requires treatment with protease in order to bind MAGE-A3 pMHC in turn suggesting that tethering the anti-albumin SDA to the peptide mask does not hinder the TCR inhibition properties of peptide mask, Peptide-5. TCR bispecific TCR-21 is the analogous non-cleavable version of TCR-20, where the LSGRSDNH sequence (SEQ ID NO: 4) has been replaced with GlySer repeats. Kinetic binding data suggests that TCR-21 maintains its inability to bind MAGE-A3 pMHC despite treatment with protease. TCR-21 related data further supports the ability to use different linkers sequences between the SDA tethered mask and the TCR without giving up masking efficiency. TCR bispecific TCR-22 is the analogous mock-mask version of TCR-20 where a portion of the cyclic peptide mask Peptide-5 (VSCKDVYDEAFCWT (SEQ ID NO: 3)) has been replaced with a cyclic AlaSer repeat peptide (ASCAASASAAACAS (SEQ ID NO: 136)). TCR-22 kinetic binding data of MAGE-A3 pMHC shows a significant loss in masking efficiency suggesting that specific interaction between the inhibitory mask and the TCR are required to block the interactions with the TCRs cognate pMHC. Non-specific steric occlusion using a tethered SDA is unable to adequately block TCR recognition of its cognate pMHC.

### TCR Bispecific Equilibrium Binding to Cognate pMHC by ELISA

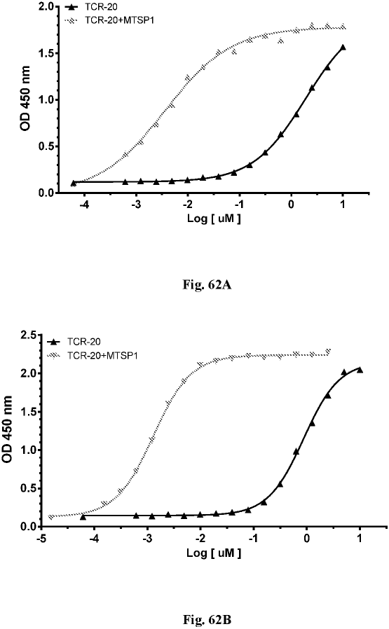

TCR bispecifics were also characterized for their ability to recognize cognate pMHC in ELISA based binding experiments (FIGS. 62A-62B). In some instances the TCR bispecifics were treated with protease where indicated. Briefly, biotinylated pMHCs were captured on neutravidin coated plates followed by the addition of titrated TCR bispecific in bovine or human albumin buffer. Plates were then incubated for a short time followed by a wash. A secondary anti-histag HRP conjugate antibody was used to detect bound TCR bispecific to the plate. Concentrations of TCR bispecifics required to achieve half maximal ELISA signal (EC50) were calculated in Graphpad Prism 6.0. The ELISA data in FIGS. 62A-62B demonstrate hindered ability of masked TCR bispecific to bind MAGE-A3 pMHCs independent of albumin occupying the SDA binding site. TCR bispecific masking efficiency was not significantly influenced by use of bovine versus human albumin buffer, indicated by the similar EC50s with either of the two buffers. Human albumin buffer is expected to saturate the TCR bispecific albumin binding site. In contrast, TCR bispecifics do not bind bovine albumin and are therefore expected to have unoccupied SDA sites during experiments using bovine albumin or bovine serum. In addition, TCR bispecific binding signals are restored to low nanomolar levels after treatment with protease regardless of bovine or human albumin buffer.

### Ternary Complex Formation of TCR Bispecifics on the Surface of Human T Cells via Flow Cytometry

TCR bispecifics were further characterized for their ability to form a ternary complex on the surface of human CD8+ T cells via binding of cellular CD3 and subsequently stained using fluorescently labeled MAGE-A3 pMHC tetramer (FIG. 63). Cellular fluorescence measured by flow cytometry was indicative of complex formation between T cell and MAGE-A3 pMHC tetramer where the TCR bispecific acts as the bridging molecule.

Briefly, 100,000 T cells per well were distributed in a 96 well plate, washed cold, followed by incubation with the indicated concentration of non-masked TCR bispecific (TCR-14), TCR bispecifics, or protease treated TCR bispecifics in human albumin buffer. Cells were incubated cold for a few hours, then washed with cold buffer, followed by a short incubation with cold MAGE-A3 pMHC tetramer formed using fluorescently labeled streptavidin. Cells were washed cold, resuspended in cold buffer, and run on a Novocyte flow cytometer. Scattering signals were gated in the typical fashion to exclude debris of incorrect cellular shape and size. Mean fluorescent intensity was normalized, plotted against TCR bispecific concentration, and the concentration of TCR bispecific required to achieve 50% maximal signal (EC50) was calculated in GraphPad Prism 6.0. In general, treatment of TCR bispecific molecules with protease enabled potent ternary complex formation equivalent to the non-masked TCR bispecific controls with low nanomolar EC50s. In contrast, minimal ternary complex formation could be detected using TCR bispecific molecules at the highest concentrations tested. Data suggests functional TCR bispecific ternary complex formation requires specific protease activity.

### TCR Bispecific Mediated Tumor Cytotoxicity and T Cell Activation

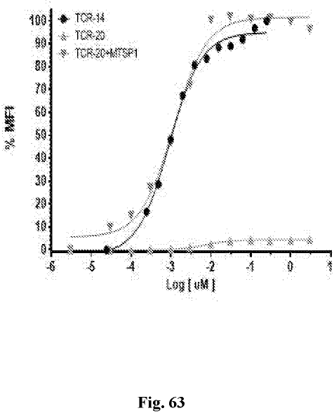

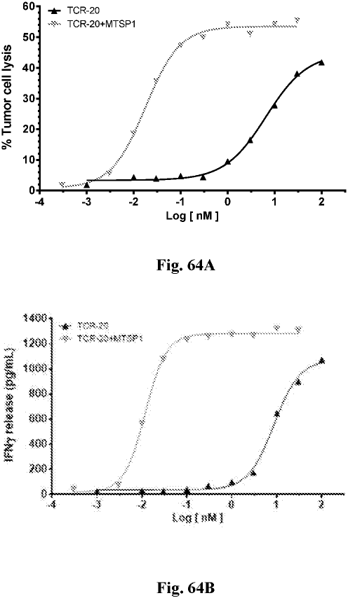

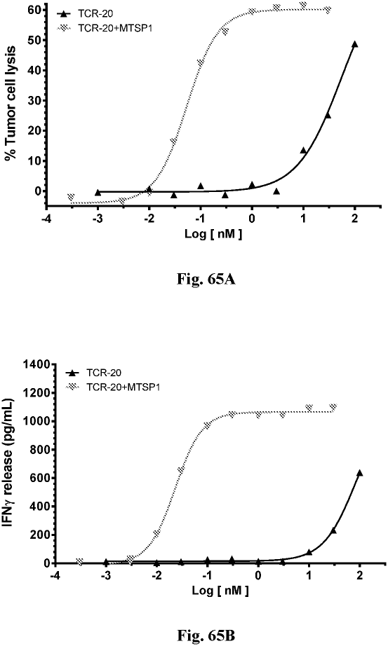

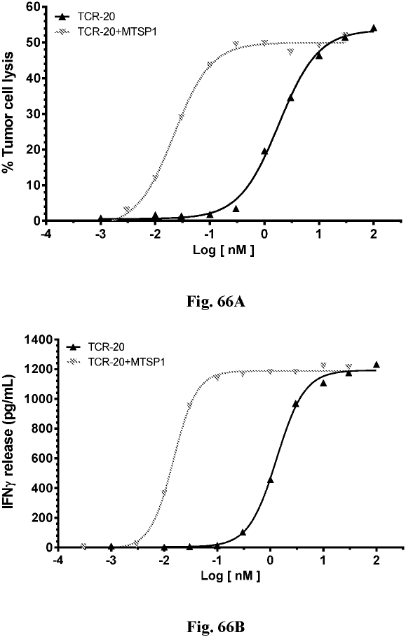

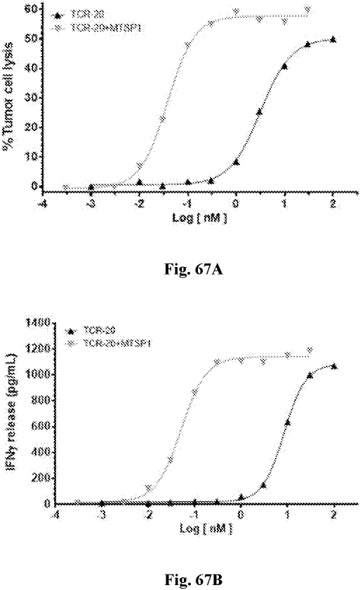

TCR bispecifics were evaluated in functional in vitro tumor cell killing and related T cell activation studies using the MAGE-A3 positive A375 (FIGS. 64A-64B and FIGS. 65A-65B) and HCT116 (FIGS. 66A-66B and FIGS. 67A-67B) tumor cell lines using bovine or human serum supplemented medium. Briefly, MAGE-A3 positive tumor cell lines were seeded onto 96 well tissue culture treated flat bottom plates and allowed to adhere overnight. The following day, culture medium and nonadherent cells were removed and replaced with fresh medium containing titrated TCR bispecific at concentrations indicated. In some instances, TCR bispecifics were treated with protease prior to addition to target cells. CD8+ T cells were then added in an effector cell : target cell ratio of 2:1 relative to tumor cell seeding density. Adherent tumor cells, CD8+ T cells, and TCR bispecifics were co-cultured for 48 hours. Plates were gently spun down to collect cells at the bottom of the plate and the clarified supernatants collected. Lactate dehydrogenase (LDH) dependent cytotoxicity was measured using the Promega LDH-Glo assay kit. Interferon-gamma (IFNγ) released by activated T cells was measured in the supernatants using an Invitrogen ELISA kit. LDH or IFNy signals were plotted against concentration of TCR bispecifics in order to calculate the concentration of TCR bispecific required to achieve 50% maximal signal (EC50). EC50s were calculated using Graphpad Prism 6.0. EC50s are summarized in Table 4. In general, TCR bispecifics functional shifts were similar to their analogous TCR bispecifics (Example 26) where TCR bispecifics treated with protease had EC50s in the low picomolar range. This suggests that the anti-albumin SDA in tandem with the peptide mask had little influence on the functional activity of TCR bispecific relative to the analogous TCR bispecifics that lack the SDA. The differences in masking efficiency of TCR-20 against A375 compared to HCT116 tumor cells was likely due to different tumor cell proteolytic activities. HCT116 cells likely had higher proteolytic activity against the LSGRSDNH (SEQ ID NO: 4) linker substrate than the A375 cells as indicated by the compression of the functional cytotoxicity shifts of TCR-20 relative to TCR-20 fully cleaved by pretreatment with MTSP1.

### TCR Bispecific Pharmacokinetics in Balb/c Mice

TCR bispecific pharmacokinetics were determined in male 6-8 week old Balb/c mice. Briefly, animals were assessed for their general health by a member of veterinary staff or other designated personnel upon arrival and allowed to acclimate for at least 3 days before study commencement. Animals were group housed during acclimation and throughout the study. The animal room environment was controlled according to facility operation with temperature between 20 to 26° C. and relative humidity between 30 and 70%. Lighting was controlled on a 12 hour light dark cycle. Animals were fed certified pellet diet (Certified Rodent Diet #5002, LabDiet). Purified water (reverse osmosis) was provided to the animals ad libitum. Periodic analyses of water quality was performed.

Concentrated test articles were diluted to appropriate dosing volume in sterile phosphate buffered saline and administered intravenously via tail vein at 10 mL/kg. Dose volume was determined individually by body weight obtained immediately prior to dosing for each animal.

Blood samples were collected before and after dosing at the indicated time points. For each timepoint a subset of 3 mice were euthanized by carbon dioxide inhalation. Following confirmation of death, blood samples were collected through the inferior vena cava using a syringe. The blood samples were placed in pre-labeled serum separation tube and incubated for 30 min before being processed to serum. After 30 min, the blood samples were centrifuged cold at 3000×g for 10 min to separate clots from serum. The serum supernatant was harvested and stored frozen prior to analysis.

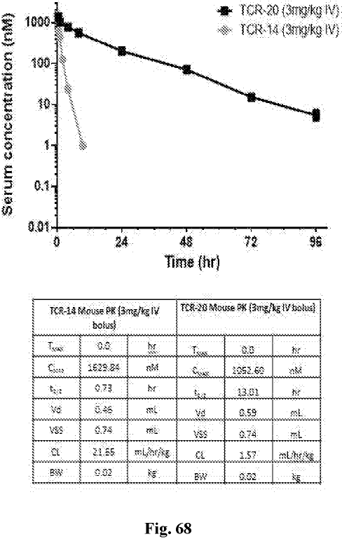

The concentration of TCR bispecifics in serum samples was determined by ELISA. Briefly, CD3 was captured on neutravidin coated plates, washed, blocked, quenched with biocytin, and washed again. Standard dilutions of TCR bispecifics in mouse serum were used to generate a calibration curve to which animal PK test samples could be compared. Standards and test samples were added to the plate and incubated cold overnight. Several different dilutions of test samples were used to make sure signals landed within appropriate dynamic range of the standard curve. Plates were washed and incubated with a FITC labeled anti-TCR secondary antibody for a brief time followed by another wash. A tertiary anti-FITC HRP conjugate antibody was used to detect bound TCR bispecifics. Plates were washed, developed, and stopped using standard ELISA techniques. Standard curves plotting absorbance at 450 nm versus known TCR bispecifics concentration were used to calculate the concentration of unknown test articles in each mouse PK serum sample. Concentration of TCR bispecifics were plotted versus time and fit to a standard two stage distribution and elimination pharmacokinetic model. The calculated pharmacokinetic parameters for TCR bispecific TCR-14 and TCR bispecific TCR-20, from Balb/c mice are shown in FIG. 68.

### TCR Bispecific Pharmacokinetics in Cynomolgus Monkey

TCR bispecific pharmacokinetics were determined in naive male cynomolgus monkeys weighing 2-3 kg. Briefly, two group housed monkeys were used per dosing group and allowed to acclimate to their surroundings prior to dosing. Animals were sedated with Ketamine HCL 10-20 mg/kg IM prior to dosing and bleeding. Concentrated test articles were diluted in sterile phosphate buffered saline and administered to animals at a quantity relative to the animals’ mass in kg. The dose for each test article was 0.2 mg/kg administered intravenously at 1 mL/kg dosing volume. For dosing, the left and right limbs were clipped and prepped with alcohol. The saphenous vein was identified and a standard catheter was placed for IV bolus infusion (in either the left or right limb). The test article dosing solution was attached to the catheter via syringe and the bolus infusion occurred via manual compression of the syringe.

For blood collections, animals were sedated using ketamine, the femoral triangle was prepared, and blood was collected from the femoral vein using a 22G 1.5 inch needle, vacutainer sheath, and collection tube. Following venipuncture, manual compression of the vein was maintained until hemostasis was achieved. Blood collections were based on weight of the animals and did not exceed AGI maximum bleeds as set forth by IACUC. Blood was collected in SST tubes and processed to serum. Serum samples were frozen prior to analysis.

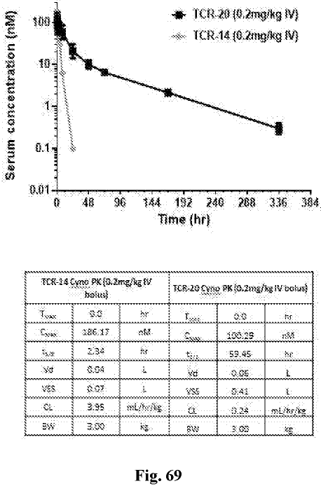

The concentration of TCR bispecifics in serum samples was determined by ELISA. Briefly, CD3 was captured on neutravidin coated plates, washed, blocked, quenched with biocytin, and washed again. Standard dilutions of TCR bispecifics in cyno serum were used to generate a calibration curve to which animal PK test samples could be compared. Standards and test samples were added to the plate and incubated cold overnight. Several different dilutions of test samples were used to make sure signals landed within appropriate dynamic range of the standard curve. Plates were washed and incubated with a FITC labeled anti-TCR secondary antibody for a brief time followed by another wash. A third anti-FITC HRP conjugate antibody was used to detect bound TCR bispecifics. Plates were washed, developed, and stopped using standard ELISA techniques. Standard curves plotting absorbance at 450 nm versus known TCR bispecifics concentration were used to calculate the concentration of test articles in each PK serum sample. Concentration of TCR bispecifics were plotted versus time and fit to a standard two stage distribution and elimination pharmacokinetic model. The calculated pharmacokinetic parameters for TCR bispecific TCR-14 and TCR bispecific TCR-20, from cynomolgus monkey are shown in FIG. 69.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A polypeptide complex comprising [image] a soluble T cell receptor (TCR) that binds to a tumor cell antigen, wherein the soluble TCR comprises an alpha TCR polypeptide and a beta TCR polypeptide wherein the soluble TCR is linked to a peptide that impairs binding of the soluble TCR to the tumor cell antigen at an N-terminus of the soluble TCR with a linking moiety that is a substrate for a tumor specific protease, and the peptide is further linked to a half-life extending molecule; and a single chain variable fragment (scFv) that is linked to the soluble TCR wherein the scFv comprises a light chain variable domain and heavy chain variable domain and the scFv binds to an effector cell antigen.

2. The polypeptide complex of claim 1, wherein the peptide is linked to an N-terminus of the alpha TCR polypeptide.

3. The polypeptide of complex of claim 2, wherein the peptide is linked to an N-terminus of the alpha TCR polypeptide and the beta TCR polypeptide is linked to a C-terminus of the heavy chain variable domain.

4. The polypeptide of complex of claim 2, wherein the peptide is linked to an N-terminus of the alpha TCR polypeptide and the beta TCR polypeptide is linked to a C-terminus of the light chain variable domain.

5. The polypeptide complex of claim 1, wherein the peptide is linked to an N-terminus of the beta TCR polypeptide.

6. The polypeptide complex of claim 5, wherein the peptide is linked to an N-terminus of the beta TCR polypeptide and the alpha TCR polypeptide is linked to a C-terminus of the heavy chain variable domain.

7. The polypeptide complex of claim 5, wherein the peptide is linked to an N-terminus of the beta TCR polypeptide and the alpha TCR polypeptide is linked to a C-terminus of the light chain variable domain.

8. The polypeptide complex of claim 1, wherein the tumor cell antigen comprises MAGEA3.

9. The polypeptide complex of claim 2, wherein the alpha TCR polypeptide comprises a TCR alpha extracellular domain and the beta TCR polypeptide comprises a TCR beta extracellular domain.

10. The polypeptide complex of claim 2, wherein the alpha TCR polypeptide comprises an amino acid sequence that has at least 85% sequence identity to SEQ ID NOs: 5, 73, 75, 76, 79, 80, 85, or 91.

11. The polypeptide complex of claim 4, wherein the beta TCR polypeptide comprises an amino acid sequence that has at least 85% sequence identity to SEQ ID NOs: 9, 74, 77, 78, 81, 82, 83, or 84.

12. The polypeptide complex of claim 1, wherein the peptide has less than 70% sequence identity to an amino acid sequence of the tumor cell antigen.

13. The polypeptide complex of claim 8, wherein the peptide has less than 70% sequence identity to an amino acid sequence of the MAGEA3.

14. The polypeptide complex of claim 1, wherein the peptide is bound to the soluble TCR through ionic interactions, electrostatic interactions, hydrophobic interactions, Pi-stacking interactions, and H-bonding interactions.

15. The polypeptide complex of claim 1, wherein the peptide is a cyclic peptide.

16. The polypeptide complex of claim 1, wherein the peptide is at least 10 amino acids in length.

17. The polypeptide complex of claim 16, wherein the peptide is no more than 40 amino acids in length.

18. The polypeptide complex of claim 17, wherein the peptide comprises an amino acid sequence of at least 10 amino acids in length and no more than 20 amino acids in length.

19. The polypeptide complex of claim 1, wherein the peptide comprises an amino acid sequence according to SEQ ID NOs: 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44.

20. The polypeptide complex of claim 1, wherein the linking moiety comprises a urokinase cleavable amino acid sequence, a matriptase cleavable amino acid sequence, matrix metalloprotease cleavable amino acid sequence, or a legumain cleavable amino acid sequence.

21. The polypeptide complex of claim 1, wherein the linking moiety has a formula comprising (G₂S)ₙ, (GS)ₙ, (GSGGS)ₙ (SEQ ID NO: 49), (GGGS)ₙ (SEQ ID NO: 50), (GGGGS)ₙ (SEQ ID NO: 51), or (GSSGGS)ₙ (SEQ ID NO: 52), wherein n is an integer of at least 1.

22. The polypeptide complex of claim 1, wherein the linking moiety comprises an amino acid sequence according to SEQ ID NOs: 4, 53, 54, 55, 56, 57, 58, 59, 60, 61, 62, 63, 64, 65, 66, 68, 69, or 70.

23. The polypeptide complex of claim 1, wherein the half-life extending molecule comprises a linking moiety (L₃) that connects the half-life extending molecule to the peptide.

24. The polypeptide complex of claim 23, wherein L₃ has a formula selected from the group consisting of (G₂S)ₙ, (GS)ₙ, (GSGGS)ₙ (SEQ ID NO: 49), (GGGS)ₙ (SEQ ID NO: 50), (GGGGS)ₙ (SEQ ID NO: 51), and (GSSGGS)ₙ (SEQ ID NO: 52), wherein n is an integer of at least 1.

25. The polypeptide complex of claim 23, wherein L₃ comprises an amino acid sequence according to SEQ ID NO: 71.

26. The polypeptide complex of claim 23, wherein the half-life extending molecule comprises an antibody.

27. The polypeptide complex of claim 26, wherein the antibody comprises a single domain antibody, a single chain variable fragment, or a Fab.

28. The polypeptide complex of claim 27, wherein the single domain antibody binds to albumin.

29. The polypeptide complex of claim 27, wherein the single domain antibody comprises 10G or 10GE.

30. The polypeptide complex of claim 29, wherein the single domain antibody comprises 10G, and the single domain antibody comprises an amino acid sequence according SEQ ID NOs: 2 or 72.

31. The polypeptide complex of claim 1, wherein the effector cell antigen comprises cluster of differentiation 3 (CD3).

32. The polypeptide complex of claim 1, wherein the scFv comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19.

33. The polypeptide complex of claim 32, wherein the scFv comprises CDRs of UCHT1.

34. The polypeptide complex of claim 1, wherein the scFv comprises an amino acid sequence that has at least 85% sequence identity to SEQ ID NO: 86 or SEQ ID NO: 8.

35. A pharmaceutical composition comprising:
(i) the polypeptide complex of claim 1; and
(ii) a pharmaceutically acceptable excipient.

36. An isolated recombinant nucleic acid molecule encoding the polypeptide complex of claim 1.
